(19) United States
(12) Patent Application Publication
(10) Pub. No.: US 2024/0252655 A1
(43) Pub. Date: Aug. 1, 2024

(54) DEUBIQUITINASE-TARGETING CHIMERAS AND RELATED METHODS

(71) Applicants: Novartis AG, Basel (CH); THE REGENTS OF THE UNIVERSITY OF CALIFORNIA, Oakland, CA (US)

(72) Inventors: Lydia Boike, Oakland, CA (US); Dustin Leard Dovala, San Leandro, CA (US); Nathaniel James Henning, Berkley, CA (US); Matthew James Hesse, Oakland, CA (US); Gang Liu, Cambridge, MA (US); Jeffrey M. McKenna, Carlisle, MA (US); Daniel K. Nomura, Walnut Creek, CA (US); Markus Eberhard Schirle, Somerville, MA (US); Jessica Nichole Spradlin, Berkley, CA (US); John A. Tallarico, Cambridge, MA (US); Carl C. Ward, Berkeley, CA (US); Melissa Pighetti, Berkeley, CA (US)

(21) Appl. No.: 18/288,550

(22) PCT Filed: Apr. 29, 2022
(86) PCT No.: PCT/US2022/027120

§ 371 Date: Oct. 26, 2023

Related U.S. Application Data
(60) Provisional application No. 63/311,781, filed on Feb. 18, 2022.
(60) Provisional application No. 63/273,118, filed on Oct. 28, 2021.
(60) Provisional application No. 63/186,739, filed on May 10, 2021.
(60) Provisional application No. 63/181,796, filed on Apr. 29, 2021.

(51) Int. Cl. A61K 47/55; C07D 241/08; C07D 405/04; C07D 405/12; C07D 405/14; C07D 487/04

(52) U.S. Cl. CPC A61K 47/55; C07D 241/08; C07D 405/04; C07D 405/12; C07D 405/14; C07D 487/04

(57) ABSTRACT

Described herein are bifunctional compounds, as well as pharmaceutically acceptable salts, hydrates, solvates, prodrugs, stereoisomers, or tautomers thereof, that function to recruit certain deubiquitinases to a target protein for modulation (e.g., stabilization) of the target protein, as well as methods of use thereof.

### RELATED APPLICATIONS

This application claims the benefit of and priority to U.S. Provisional Application No. 63/311,781, filed Feb. 18, 2022; U.S. Provisional Application No. 63/273,118, filed Oct. 28, 2021; U.S. Provisional Application No. 63/186,739, filed May 10, 2021; and U.S. Provisional Application No. 63/181,796, filed on Apr. 29, 2021. The entire contents of each of the foregoing applications is incorporated herein by reference in its entirety.

### FIELD OF THE DISCLOSURE

Described herein are bifunctional compounds that bind to both a target protein and a deubiquitinase, as well as related compositions and methods of use, e.g., for stabilization of the target protein and/or the treatment of a disease, disorder, or condition.

### BACKGROUND

The Ubiquitin-Proteasome Pathway (UPP) is a critical process that plays a role in a variety of cellular functions, including protein degradation, quality control, trafficking, and signaling. Ubiquitin and other ubiquitin-like proteins (collectively, “Ubls”) are covalently attached to specific protein substrates, which depending on the specific modification, either ultimately targets these proteins for degradation by the proteasome or affects protein function in other ways. These Ubls, however, may be removed through the action of deubiquitinases (DUBs), which hydrolyze the Ubl from a target protein. Removal of a Ubl from a ubiquitinated target protein can modulate the function of the target protein in a number of ways, including improving stability and preventing proteasomal degradation. As degradation of certain cellular proteins has been linked to disease progression, there is a need for new tools to stabilize certain proteins and slow or inhibit their degradation.

### BRIEF DESCRIPTION OF DRAWINGS

FIG. 1 is a schematic illustrating the general architecture of exemplary bifunctional compounds described herein, as well as their use in recruiting a deubiquitinase (DUB) to a target protein (e.g., a ubiquitinated target protein) to deubiquitinate and stabilize the levels of the target protein.

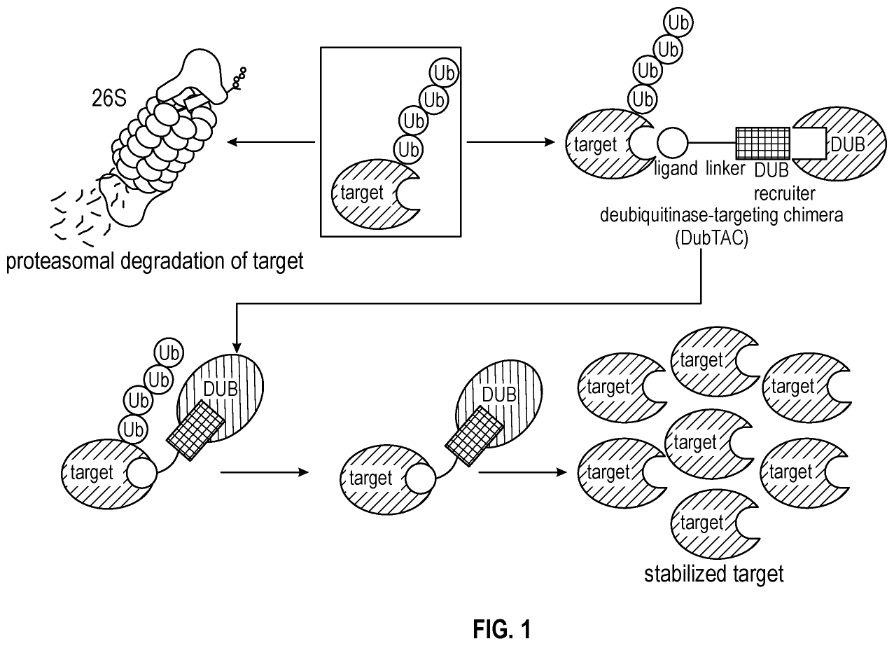

FIGS. 2A-2B are circle graphs illustrating results of activity-based protein profiling (ABPP) screens described herein to identify candidate deubiquitinases. FIG. 2A shows that 65 out of 65 deubiquitinases tested contained a probe-modified cysteine. FIG. 2B shows that 39 of the 65 deubiquitinases tested showed greater than 10 aggregate spectral counts across the ABPP datasets, and 24 out of these 39 deubiquitinases (62%) showed labeling of catalytic or active site cysteines.

FIG. 3A is a graph showing that 10 of the identified deubiquitinases in the ABPP screen contained one probe-modified cysteine that represented greater than 50% of the total aggregate spectral count for probe-modified cysteine peptides for the particular deubiquitinase.

FIG. 3B is a graph that depicts analysis of the chemoproteomic data for the deubiquitinase OTUB1, in which the cysteine 23 (C23) is identified as the dominant site labeled by the probe screen, compared to the catalytic cysteine 91 (C91).

FIG. 4 is a graph depicting the results of a covalent ligand screen of cysteine-reactive libraries competed against IA-rhodamine labeling of a recombinant deubiquitinase (OTUB1) to identify binders to OTUB1 by ABPP. Vehicle DMSO or cysteine-reactive covalent ligands (50 uM) were pre-incubated with OTUB1 for 30 min at room temperature prior to IA-rhodamine labeling (500 nM, 30 min room temperature). OTUB1 was then separated by SDS/PAGE and in-gel fluorescence was assessed and as described.

FIG. 5 is an image of gel-based ABPP confirmation showing dose-responsive inhibition of IA-rhodamine binding of OTUB1. Vehicle (DMSO) or an exemplary DUB Recruiter (Compound 100) were pre-incubated with OTUB1 for 30 min at 37° C. prior to IA-rhodamine labeling (500 nM, 30 min room temperature). OTUB1 was then separated by SDS/PAGE and in-gel fluorescence was assessed. Protein loading is illustrated by silver staining. The gel shown is representative gel of n=3 biologically independent samples/group.

FIG. 6 is a liquid chromatography-tandem mass spectrometry analysis (LC-MS/MS) of tryptic peptides from OTUB1 covalently bound to an exemplary DUB Recruiter (Compound 100) and showed that Compound 100 selectively targets C23, with no detectable modification of the catalytic C91.

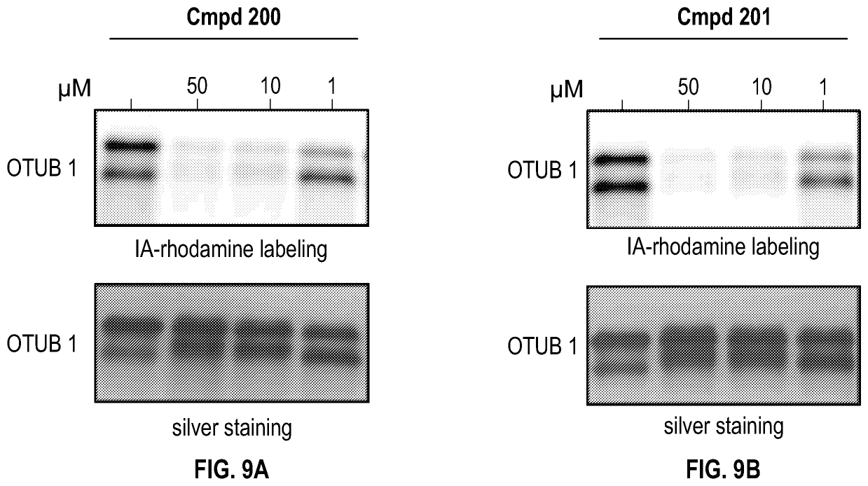

FIG. 7 is gel-based analysis of an in vitro reconstituted OTUB1 deubiquitination activity assay monitoring monoubiquitin release from di-ubiquitin and demonstrated that the exemplary DUB Recruiter (Compound 100) does not inhibit OTUB1 deubiquitination activity. These studies were performed in the presence of OTUB1-stimulating Ubiquitin-conjugating enzyme E2 D1 (UBE2D1), an E2 ubiquitin ligase that engages in a complex with OTUB1 to stimulate OTUB1 activity.

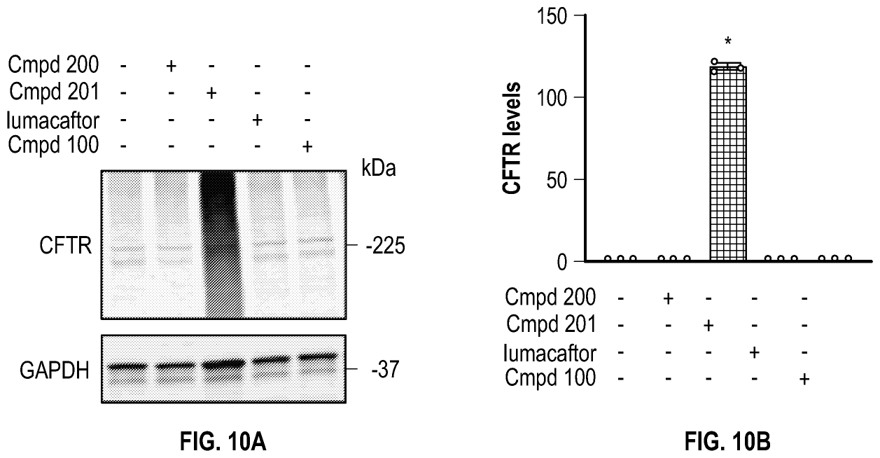

FIG. 8 provides images of gel-based analyses of OTUB1 binding to additional exemplary DUB Recruiters to explore structure-activity relationships (SAR).

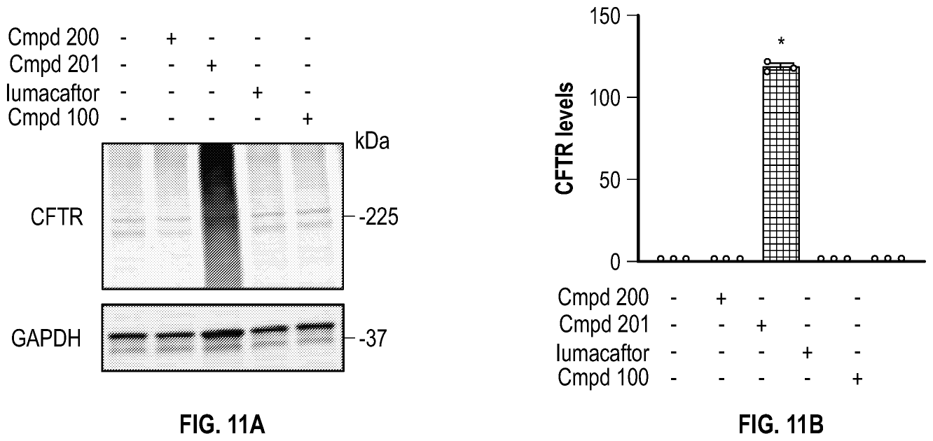

FIGS. 9A-9B show images of the gel-based ABPP analysis of the exemplary bifunctional compounds Compound 200 and Compound 201 against OTUB1. In each experiment, vehicle (DMSO) or the bifunctional compounds were preincubated with recombinant OTUB1 for 30 min at 37° ° C. prior to addition of IA-rhodamine (100 nM) for 30 min at room temperature. OTUB1 was run on SDS/PAGE and in-gel fluorescence was assessed. Protein loading was assessed by silver staining.

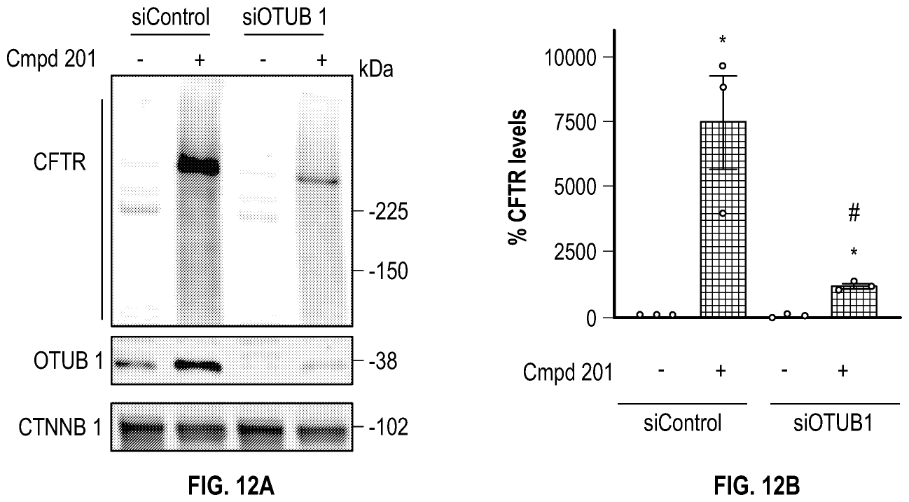

FIGS. 10A-10B are images depicting the effect of exemplary bifunctional compounds on mutant CFTR levels. CFBE410-4.7 cells expressing ΔF508-CFTR were treated with vehicle DMSO, Compound 200 (10 uM), Compound 201 (10 uM), lumacaftor (10 uM), or Compound 100 (10 uM) for 24 h, and mutant CFTR and loading control GAPDH levels were assessed by Western blotting as shown in FIGS. 9A-9B. FIG. 10B shows the quantification of the data acquired from FIG. 10A.

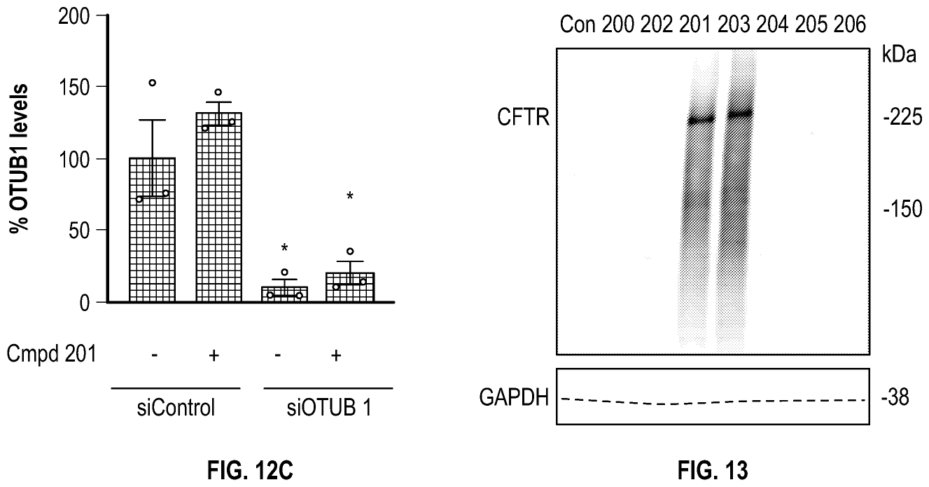

FIGS. 11A-11B are images that show analysis of the mechanism of the exemplary bifunctional compound Compound 201. CFBE410-4.7 cells expressing ΔF508-CFTR were pre-treated with vehicle (DMSO), lumacaftor (100 uM), or Compound 100 (100 uM) for 1 h prior to treatment with Compound 201 (10 uM) for 24 h. Mutant CFTR and loading control GAPDH levels were assessed by Western blotting as shown in FIG. 11A. FIG. 11B shows the quantification of the data acquired from FIG. 11A.

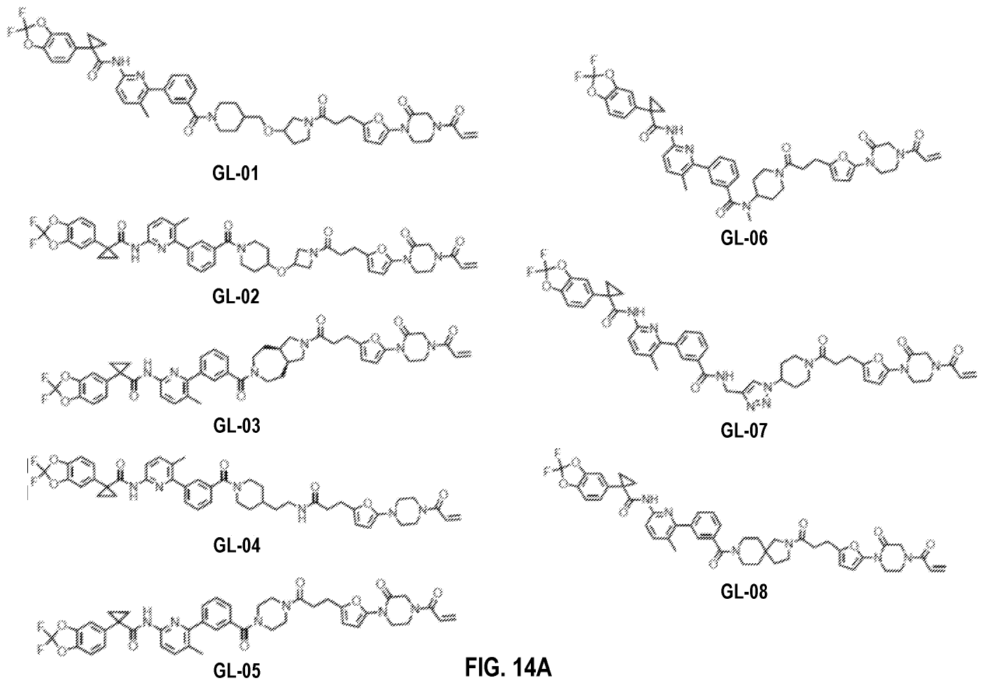

FIGS. 12A-12C are images illustrating the effect of OTUB1 knockdown on bifunctional compound Compound 201-mediated mutant CFTR stabilization. CFBE410-4.7 cells expressing ΔF508-CFTR were transiently transfected with siControl or siOTUB1 oligonucleotides for 48 h prior to treatment of cells with vehicle DMSO or Compound 201 (10 uM) for 16 h. Mutant CFTR, OTUB1, and loading control GAPDH levels were assessed by Western blotting as shown in FIG. 12A. FIG. 12B shows quantification of the data acquired from FIG. 12A for % CFTR levels, while FIG. 12C summarizes the data for % OTUB levels.

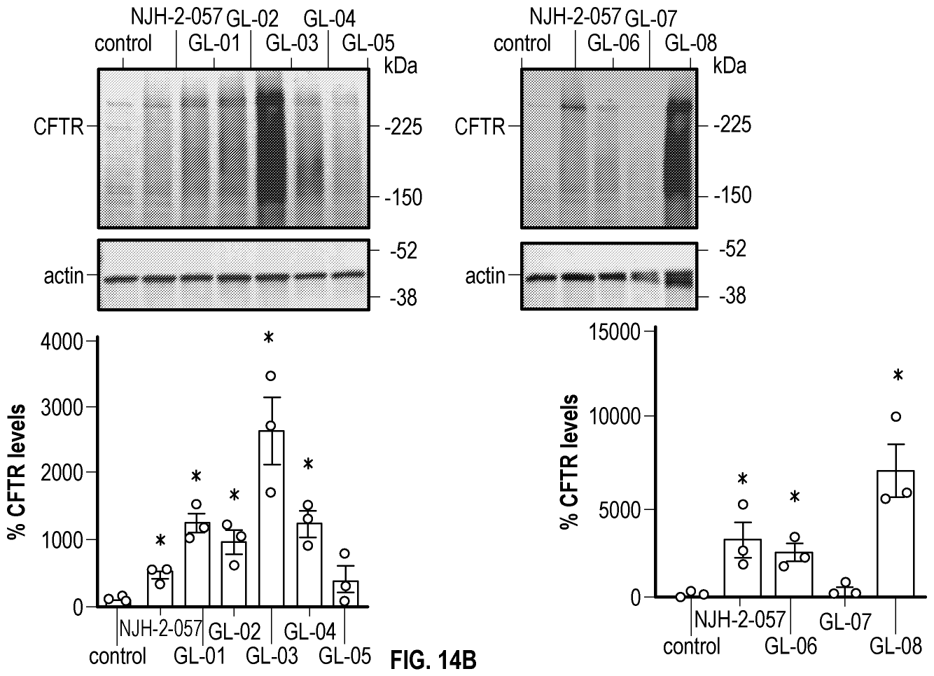

FIG. 13 is an image depicting CFTR pulldown studies with exemplary bifunctional compounds. CFBE410-4.7 cells expressing DF508-CFTR were treated with vehicle DMSO or exemplary bifunctional compounds (10 mM) for 24 h and CFTR and loading control GAPDH levels were assessed by Western blotting. Blot is representative of n=3 biologically independent samples/group.

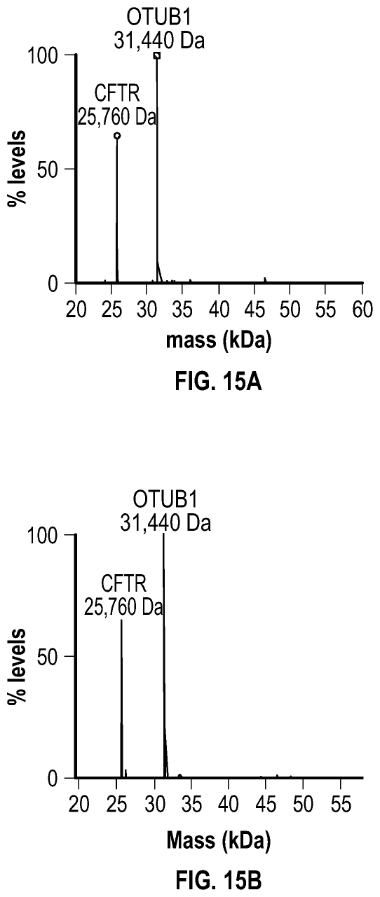

FIG. 14 is an image depicting CFTR pulldown studies with exemplary bifunctional compounds. CFBE410-4.7 cells expressing DF508-CFTR were treated with vehicle DMSO or exemplary bifunctional compounds provided in Table 2 (10 mM) for 24 h and CFTR and loading control GAPDH levels were assessed by Western blotting. In this image, NJH-2-057 refers to Compound 201.

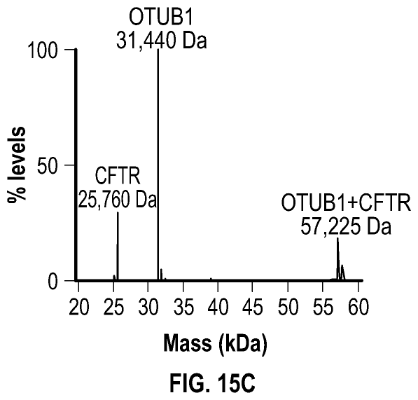

FIGS. 15A-15D are images that confirm formation of a ternary complex between CFTR, an exemplary bifunctional compound (Compound 201), and OTUB1 in vitro using recombinant protein and native mass spectrometry (MS)-based approaches. FIGS. 15A-15C depict native mass spectra of CFTR-OTUB1 complex formation in the presence of DMSO (FIG. 15A), the DUB Recruiter Compound 100 alone (FIG. 15B), or the bifunctional compound Compound 201 (FIG. 15C). While the highest intensity signals corresponded to unmodified OTUB1 and the DF508-harboring CFTR nucleotide-binding domain used in this experiment, potentially indicating low levels of target engagement under these experimental conditions, significant CFTR-OTUB1 complex formation was observed with treatment of Compound 201, but not with DMSO vehicle or Compound 100 treatment.

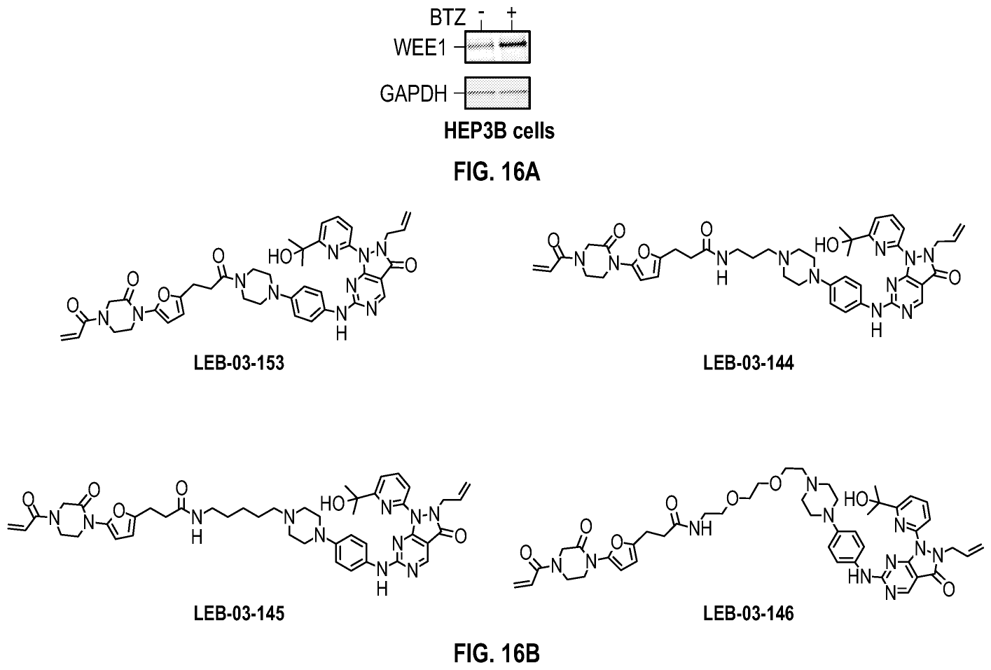

FIGS. 16A-16D are images that illustrate use of exemplary bifunctional compounds described herein to target the tumor suppressor kinase WEE1. HEP3B cells were treated with DMSO vehicle or bortezomib (1 mM) for 24 h. WEE1 and loading control GAPDH levels were assessed by Western blotting. FIG. 16B depicts structures of four exemplary bifunctional compounds designed to target WEE1. FIG. 16C shows the gel-based analysis of an experiment in which HEP3B cells were treated with DMSO vehicle, the four bifunctional compounds, bortezomib, Compound 100, or AZD1775 at 1 mM for 24 h. WEE1 and loading control GAPDH levels were assessed by Western blotting. Blots shown in (a) and (b) are representative blots from n=3 biologically independent samples/group. Data in bar graphs show individual biological replicate values and average±sem from n=3 biologically independent samples/group.

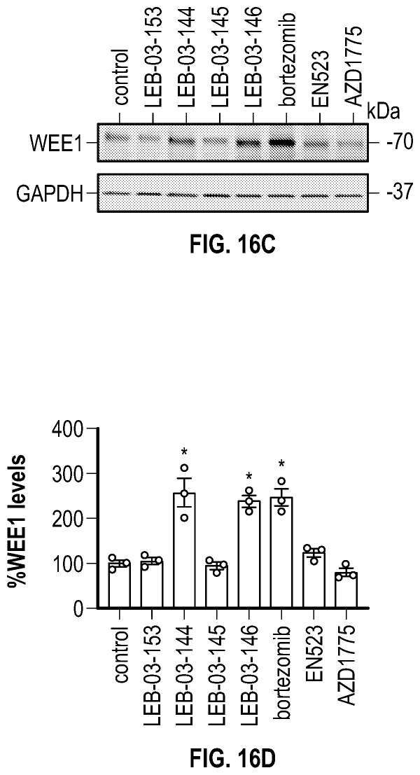

FIG. 17 is an image depicting CFTR pulldown studies with exemplary bifunctional compounds. CFBE410-4.7 cells expressing DF508-CFTR were treated with vehicle DMSO or exemplary bifunctional compounds provided in Table 2 (10 mM) for 24 h and CFTR and loading control GAPDH levels were assessed by Western blotting. In this image, NJH-2-057 refers to Compound 201, LEB-3-162 refers to Compound 230, and NJH-02-153 refers to Compound 231.

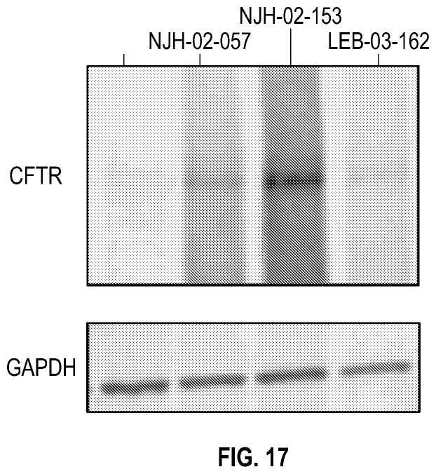

FIGS. 18A-18C are images of gel-based analyses of the deubiquitinase USP15 binding to exemplary DUB Recruiters to explore structure-activity relationships (SAR).

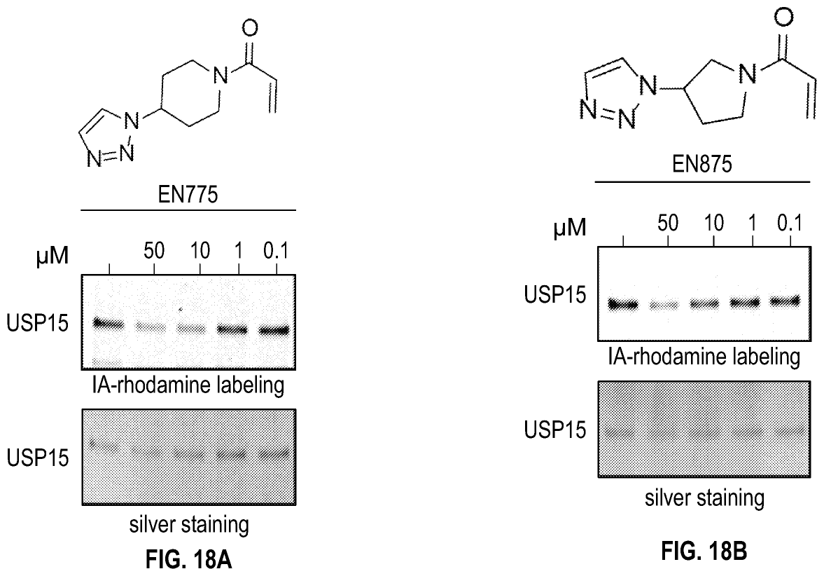

FIG. 18D is a graph that depicts analysis of the chemoproteomic data for the USP15, in which cysteine 264 (C264) and cysteine 381 (C381) are identified as the dominant site labeled by the probe screen, compared to the catalytic cysteine 298 (C298).

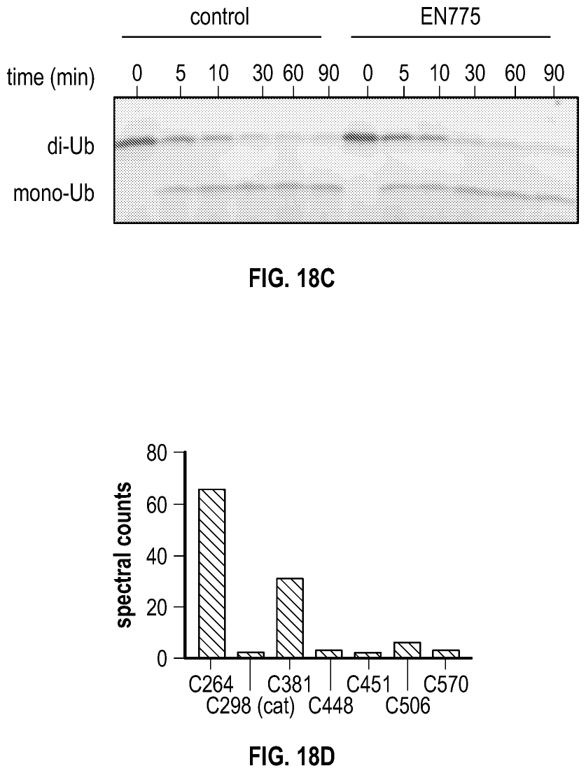

FIGS. 19A-19B are images of gel-based analyses of the deubiquitinase OTUD5 binding to exemplary DUB Recruiters to explore structure-activity relationships (SAR).

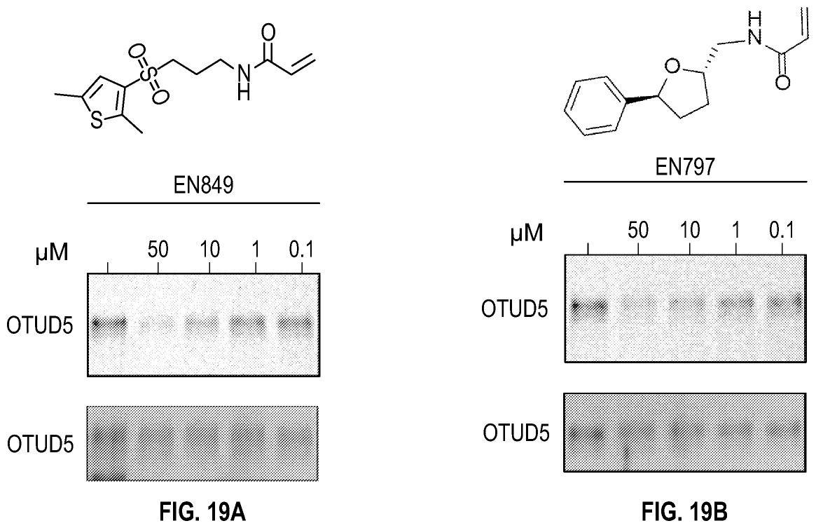

### DETAILED DESCRIPTION

Described herein are bifunctional compounds, as well as pharmaceutically acceptable salts, hydrates, solvates, prodrugs, stereoisomers, or tautomers thereof, that function to recruit certain deubiquitinases to a target protein for modulation (e.g., stabilization) of the target protein, as well as methods of use thereof. The progression of many diseases, such as cancer, respiratory diseases, and neurological diseases, entails the active ubiquitination and degradation of certain key proteins. As such, targeted stabilization of these key proteins through the deliberate deubiquitination may thwart disease progression and impart a therapeutic benefit in a cell or subject. The inventors have used chemoproteomic covalent ligand discovery methods to design a set of bifunctional compounds, which comprise both a Target Ligand, capable of binding to a target protein, and a DUB Recruiter, capable of binding to a deubiquitinase. These bifunctional compounds may, inter alia, bring the deubiquitinase in proximity to a ubiquitinated protein, thus allowing for directed removal of Ubls and potential target protein stabilization.

### Target Proteins

In one aspect, the disclosure provides a bifunctional compound or a pharmaceutically acceptable salt, hydrate, solvate, prodrug, stereoisomer, or tautomer thereof, which is capable of binding to a target protein (e.g., a target protein described herein). The target protein may be any class of protein, for example, any protein found in a cell (e.g., a mammalian cell, a plant cell, a fungal cell, an insect cell, a bacterial cell) or a viral particle. In some embodiments, the protein is a soluble protein or a membrane protein. In some embodiments, the protein is a soluble protein. In some embodiments, the protein is a membrane protein. The target protein may comprise a post-translational modification, e.g., a sugar moiety, acyl moiety, lipid moiety. In some embodiments, the target protein is glycosylated, e.g., at an asparagine, serine, threonine, tyrosine, or tryptophan residue.

Exemplary target proteins include enzymes (e.g., kinases, hydrolases, phosphatases, ligases, isomerases, oxidoreductases), receptors, membrane channels, hormones, transcription factors, tumor suppressors, ion channels, apoptotic factors, oncogenic proteins, epigenetic regulators, or a fragment thereof. In some embodiments, the target protein is an enzyme (e.g., a kinase or phosphatase). In some embodiments, the target protein is a kinase (e.g., PKN1, BCR, MAP4K4, TYK2, MAP4K2, EPHB4, MAP4K5, MAP3K2, DDR1, TGFBR1, RIPK2, TNK1, LYN, STK10, PKMYT1, LYN, EGFR, EPHA1, GAK, SIK2, MAP2K2, SLK, PRKACB, EPHA2, WEE1, or glucokinase). In some embodiments, the target protein is a tumor suppressor kinase (e.g., WEE1). In some embodiments, the target protein is WEE1 or a fragment thereof. In some embodiments, the target protein is a ligase (e.g., an E3 ligase, e.g., MDM2). In some embodiments, the target protein is a receptor. In some embodiments, the target protein is a transcription factor (e.g., MYC). In some embodiments, the target protein is a hormone. In some embodiments, the target protein is a tumor suppressor (e.g., TP53, AXIN1, BAX, CDKN1A, CKDN1C, PTEN, or SMAD4). In some embodiments, the target protein is related to a genetic disorder (e.g., SMN1/2, GLUT1, CFTR, phenylalanine hydroxylase (PAH), fumarylacetoacetate hydrolase (FAH), or acid alpha-glucosidase (GAA)). In some embodiments, the target protein is a membrane channel (e.g., CFTR). In some embodiments, the target protein is CFTR or a fragment thereof. In some embodiments, the CFTR comprises a sequence mutation (e.g., a Class I, Class II, Class III, Class IV, or Class V mutation). In some embodiments, the CFTR, SMN1/2, GLUT1, PAH, FAH, or GAA comprises a sequence mutation, e.g., an addition mutation, deletion mutation, or substitution mutation (e.g., ΔF508-CFTR). In some embodiments, the CFTR comprises a sequence mutation selected from the group consisting of G551D, R177H, and A445E. In some embodiments, the target protein is BAX or a fragment thereof. In some embodiments, the target protein is STING or a fragment thereof.

In some embodiments, the target protein is modified with a ubiquitin or a ubiquitin-like protein (collectively referred to herein as “Ubls”). In some embodiments, the Ubl is ubiquitin. In some embodiments, the Ubl is SUMO, NEDD8, or Agp12. In some embodiments, the target protein is monoubiquitinated or polyubiquitinated. The target protein may contain at least 1, 2, 3, 4, 5, 6, 7, 8, 9, 10 or more Ubl chains, e.g., on a lysine amino acid residue. The target protein may comprise polyubiquitin chains linked in any manner, for example, K48-linked polyubiquitin chains, K63-linked polyubiquitin linked chains, K29-linked polyubiquitin chains, or K33-linked polyubiquitin chains. In some embodiments, the target protein comprises a plurality of polyubiquitin chains. In some embodiments, the target protein comprising a Ubl is capable of binding to a protein comprising a Ubl-binding domain (e.g., a ubiquitin binding domain).

The target protein may comprise a feature that increases its instability or impairs its activity, e.g., relative to the wild-type target protein. For example, the target protein may be mutated or misfolded. In some embodiments, the target protein has a reduced capacity for binding to a binding partner, e.g., by about 1, 2, 3, 4, 5, 10, 15, 20, 25, 30, 35, 40, 45, 50, 55, 60, 65, 70, 75, 80, 85, 90, 95, or 99% relative to the wild type target protein. In some embodiments, the target protein is less active than the wild type target protein, e.g., by about 1, 2, 3, 4, 5, 10, 15, 20, 25, 30, 35, 40, 45, 50, 55, 60, 65, 70, 75, 80, 85, 90, 95, or 99%. In some embodiments, the target protein is more active than the wild type target protein, e.g., by about 1, 2, 3, 4, 5, 10, 15, 20, 25, 30, 35, 40, 45, 50, 55, 60, 65, 70, 75, 80, 85, 90, 95, or 99%.

### Deubiquitinases

Described herein are bifunctional compounds comprising a moiety capable of binding to a deubiquitinase (DUB). Deubiquitinases comprise a large family of proteases responsible for hydrolyzing Ubl-Ubl bonds or Ubl-target protein bonds and play a role in numerous cellular processes. Deubiquitinases serve several functions, including generating free ubiquitin monomers from polyubiquitin chains, modulating the size of polyubiquitin chains, and reversing ubiquitin signaling by removal of a from a ubiquitinated target protein. Misregulation of deubiquitinase function is associated with many diseases, including cancer, metabolic diseases, genetic disorders, haploinsufficiency targets, and neurological diseases. Roughly 80 different functional deubiquitinases have been identified in human cells to date.

The present disclosure features bifunctional compounds comprising a DUB Recruiter capable of binding to a deubiquitinase. The deubiquitinase may be any deubiquitinase, e.g., in a cell, including cysteine protease deubiquitinases and metalloprotease deubiquitinases. In some embodiments, the deubiquitinase is a cysteine protease, e.g., comprising a catalytic site cysteine amino acid residue. The deubiquitinase may be a full-length protein or a fragment thereof. In some embodiments, the deubiquitinase comprises a single active site. In other embodiments, the deubiquitinase is one function of a multifunctional protein. Exemplary deubiquitinases include BAP1, CYLD, OTUB1, OTUB2, OTUD3, OTUD5, OTUD7A, OTUD7B, TNFAIP3, UCHL1, UCHL3, UCHL5, USP10, USP11, USP12, USP13, USP14, USP15, USP16, USP17L1, USP17L2, USP17L24, USP17L3, USP17L5, USP18, USP19, USP2, USP20, USP21, USP22, USP24, USP25, USP26, USP27X, USP28, USP3, USP30, USP31, USP33, USP34, USP35, USP36, USP37, USP38, USP4, USP40, USP41, USP42, USP43, USP44, USP45, USP46, USP47, USP48, USP49, USP5, USP50, USP51, USP54, USP7, USP8, USP9X, VCPIP1, WDR48, YOD1, ZRANB1, and ZUP1, or a fragment or variant thereof. In some embodiments, the deubiquitinase is selected from the group consisting of WDR48, YOD1, OYUD3, OTUB1, USP8, USP5, USP16, UCHL3, UCHL1, and USP14, or a fragment thereof. In some embodiments, the deubiquitinase is selected from the group consisting of WDR48, YOD1, OYUD3, OTUB1, OTUD5, USP8, USP5, USP14, USP15, USP16, UCHL3, and UCHL1, or a fragment thereof. In some embodiments, the deubiquitinase is a deubiquitinase listed in Table 1. In some embodiments, the deubiquitinase comprises OTUB1 or a fragment or variant thereof. In some embodiments, the deubiquitinase comprises OTUD5 or a fragment or variant thereof. In some embodiments, the deubiquitinase comprises USP15 or a fragment or variant thereof.

The bifunctional compounds of the present disclosure may bind to a deubiquitinase in a covalent or non-covalent manner. In some embodiments, the bifunctional compound (e.g., the DUB Recruiter) binds to a site other than a catalytic site within the deubiquitinase. In some embodiments, the bifunctional compound (e.g., the DUB Recruiter) binds to an allosteric site within the deubiquitinase. In some embodiments, binding of the bifunctional compound (e.g., the DUB Recruiter) to the deubiquitinase does not modulate the activity of the deubiquitinase more than 0.1, 0.5, 1, 1.5, 2, 2.5, 3, 4, 5, 6, 7, 8, 9, 10, 15, 20, 25, 30, 35, 40, 45, 50, 55, 60, 65, 70, 75, 80, 85, 90, 95, or 99%, relative to the activity of the deubiquitinase in the absence of the bifunctional compound. In some embodiments, binding of the bifunctional compound (e.g., the DUB Recruiter) to the deubiquitinase does not modulate the activity of the deubiquitinase more than 0.1-50%, 1-50%, 1-25%, 1-10%, 0.1-10%, 1-5%, or 0.1-2%, relative to the activity of the deubiquitinase in the absence of the bifunctional compound. In some embodiments, the binding of the bifunctional compound (e.g., the DUB Recruiter) to the deubiquitinase does not substantially modulate (e.g., inhibit) the activity (e.g., deubiquitinase activity) of the deubiquitinase.

The bifunctional compound (e.g., a bifunctional compound described herein) is capable of binding to a cysteine amino acid residue (e.g., a thiol moiety), e.g., within the deubiquitinase. In some embodiments, the cysteine amino acid residue is an allosteric cysteine amino acid residue. In some embodiments, the cysteine amino acid residue is present on a surface of the deubiquitinase. In some embodiments, the cysteine amino acid residue is present on or in the interior of the deubiquitinase. In some embodiments, the cysteine amino acid residue is not a catalytic cysteine amino acid residue. In some embodiments, the bifunctional compound preferentially binds to an allosteric cysteine amino acid residue over a catalytic cysteine amino acid residue. In some embodiments, the bifunctional compound does not substantially bind to a cysteine amino acid residue in the catalytic site of the deubiquitinase (e.g., a catalytic cysteine).

Exemplary sites of modification within a subset of human deubiquitinases is provided in Table 1 below. In some embodiments, the bifunctional compounds binds to a single site within the deubiquitinase (e.g, one of the cysteine amino acid residues summarized in Table 1). In some embodiments, the bifunctional compounds binds to a plurality of sites within the deubiquitinase (e.g, a plurality of the cysteine amino acid residues summarized in Table 1).

TABLE 1
Exemplary cysteine modifications within deubiquitinases

| Deubiquitinase | Uniprot ID | Site of Modification |
|---|---|---|
| BAP1 | Q92560 | C91, C103, C676, C638, C91, C320 |
| CYLD | Q9NQC7 | C655, C853, C751 |
| OTUB1 | Q96FW1 | C23, C212, C91, C204 |
| OTUB2 | Q96DC9 | C51 |
| OTUD3 | Q5T2D3 | C49, C245, C76 |
| OTUD5 | Q96G74 | C491, C434, C519, C247, C142, C143 |
| OTUD7A | Q8TE49 | C193, C137, C784, C482, C895 |
| OTUD7B | Q6GQQ9 | C720, C133, C708, C819, C821, C345 |
| TNFAIP3 | P21580 | C521, C536, C495, C498, |
| | | C103, C483, C387, C657, |
| | | C158, C590, C627, C612, C404, C407 |
| UCHL1 | P09936 | C152, C47, C90, C220 |
| UCHL3 | P15374 | C95, C50, C209 |
| UCHL5 | Q5LJA9 | C100, C228, C229, C8, C203, |
| | | C203, C112, C21 |
| USP10 | Q14694 | C94, C254, C40, C115, C209, C40, C456 |
| USP11 | P51784 | C694, C951, C468, C894, |
| | | C428, C34, C802, C701 |
| USP12 | O75317 | C83, C226, C14, C186 |
| USP13 | Q92995 | C345, C671, C544, C164, C49, C65 |
| USP14 | P54578 | C257, C203, C415, C359, |
| | | C277, C105, C114 |
| USP15 | Q9Y4E8 | C381, C264, C633, C809, |
| | | C812, C139, C381, C570, |
| | | C306, C298, C462, C506, |
| | | C873, C451, C448, C462, |
| | | C289 |
| USP16 | Q9Y5T5 | C618, C205, C24, C631, |
| | | C191, C191, C471, C104, |
| | | C119 |
| USP17L1 | Q7RTZ2 | C89, C257, C123 |
| USP17L2 | Q6R6M4 | C414, C257, C123 |
| USP17L24 | Q0WX57 | C126, C473, C97 |
| USP17L3 | A6NCW0 | C89, C257, C122, C123, C126 |
| USP17L5 | A8MUK1 | C207, C126, C473, C97 |
| USP18 | Q9UMW8 | C181, C178, C292, C320, C65 |
| USP19 | O94966 | C1042, C492, C219, C506, |
| | | C1138, C102, C466, C1017 |
| USP2 | O75604 | C428, C431, C276, C191 |
| USP20 | Q9Y2K6 | C238, C43, C53, C527, C542, C783 |
| USP21 | Q9UK80 | C564, C229 |
| USP22 | Q9UPT9 | C171, C23, C44, C211, |
| | | C76, C61, C66, C219, C84 |
| USP24 | Q9UPU5 | C1393, C1362, C1556, C116, |
| | | C1080, C1239, C1333, |
| | | C2423, C2425, C456, C1516, |
| | | C1202, C2278, C2281, |
| | | C1580, C153, C873, C1417, |
| | | C1516, C1873, C1433 |
| USP25 | Q9UHP3 | C990, C551, C143, C1037 |
| USP26 | Q9BXU7 | C341, C876, C169, C551, C667 |
| USP27X | A6NNY8 | C113, C180, C38 |
| USP28 | Q96RU2 | C21, C171, C644, C142, |
| | | C308, C329, C203 |
| USP3 | Q9Y614 | C157, C301, C168, C227, |
| | | C301;C302, C168 |
| USP30 | Q70CQ3 | C142, C320, C129, C77 |
| USP31 | Q70CQ4 | C461, C107, C137, C911, |
| | | C862, C1263, C330, C333, |
| | | C292, C307, C93, C507 |
| USP33 | Q8TEY7 | C194, C766, C492, C603, |
| | | C206, C278, C321, C324, |
| | | C216, C79, C613, C338 |
| USP34 | Q70CQ2 | C2567, C741, C2018, C2809, |
| | | C5, C907, C3118, C3193, |
| | | C1674, C3504, C3486, C993, |
| | | C132, C3118, C2762, |
| | | C421, C1530, C228, C1903, |
| | | C1674, C2812, C2941, |
| | | C856, C2416, C654, C3486, |
| | | C85, C1017, C3193 |
| USP35 | Q9P2H5 | C848, C133, C579, C782, |
| | | C785, C177, C865 |
| USP36 | Q9P275 | C159, C938, C1002, C93, |
| | | C224, C977, C994, C777 |
| USP37 | Q86T82 | C594, C436, C625 |
| USP38 | Q8NB14 | C432, C157, C454, C1023, |
| | | C557, C180, C272, C144 |
| USP4 | Q13107 | C143, C154, C799, C958, |
| | | C577, C475, C519, C464, |
| | | C461, C475, C500 |
| USP40 | NVE5 | C277, C220, C50, C223, C50, C871, C810 |
| USP41 | Q3LFD5 | C292, C320, C178 |
| USP42 | Q9H9J4 | C867, C154, C157, C100 |
| USP43 | Q70EL4 | C540, C757, C774, C468, C566 |
| USP44 | Q9HOE7 | C604, C605, C616, C429, C515 |
| USP45 | Q70EL2 | C88, C173, C367, C715, |
| | | C618, C280, C364, C130, |
| | | C138, C719 |
| USP46 | P62068 | C10, C79 |
| USP47 | Q96K76 | C856, C1309, C45, C655, C656, C364 |
| USP48 | Q86UV5 | C986, C39, C850, C1021, |
| | | C80, C658, C451, C214, |
| | | C652, C120, C776, C273, |
| | | C48, C54, C252, C403, C763 |
| USP49 | Q70CQ1 | C25, C405, C29, C38, C584 |
| USP5 | P45974 | C335, C532, C821, C838, |
| | | C219, C46, C195 |
| USP50 | Q70EL3 | C332 |
| USP51 | Q70EK9 | C336, C398, C409 |
| USP54 | Q70EL1 | C532, C1562, C1452, C69, |
| | | C88, C1511, C1004, C1009, |
| | | C1086, C42, C1270, C124, C1096, C225 |
| USP7 | Q93009 | C961, C90, C223, C315, |
| | | C510, C896, C799, C702, |
| | | C917 |
| USP8 | P40818 | C809, C307, C214 |
| USP9X | Q93008 | C2398, C1727, C842, C673, |
| | | C1237, C1566, C2390, |
| | | C2239, C193, C1920, C673, |
| | | C540, C1340, C2293, |
| | | C577, C2107, C692, C739, |
| | | C193, C1344, C182 |
| VCPIP1 | Q96JH7 | C1178, C127, C538, C73, |
| | | C541, C219, C469, C472 |
| YOD1 | Q5VVQ6 | C97, C178, C210 |
| ZRANB1 | Q9UGI0 | C473, C155, C90, C348, |
| | | C270, C653, C443 |
| ZUP1 | Q96AP4 | C83, C159, C230, C440, C507, C515 |

In some embodiments, the deubiquitinase is OTUB1 (Uniprot ID Q96FW1). The bifunctional compound described herein may bind to (e.g., covalently bind to) any cysteine residue within the OTUB1 sequence, e.g., C23, C91, C204, or C212. In some embodiments, the bifunctional compound does not bind to a catalytic cysteine amino acid within the OTUB1 sequence. In some embodiments, the bifunctional compound binds to an allosteric cysteine amino acid residue within the OTUB1 sequence. In some embodiments, the bifunctional compound binds to a cysteine residue on a surface of OTUB1. In some embodiments, the bifunctional compound binds to a cysteine residue on or in the interior of OTUB1.

In some embodiments, the bifunctional compound binds to C23 within the OTUB1 sequence. In some embodiments, the bifunctional compound binds to C91 within the OTUB1 sequence. In some embodiments, the bifunctional compound binds to C204 within the OTUB1 sequence. In some embodiments, the bifunctional compound binds to C212 within the OTUB1 sequence. In some embodiments, the bifunctional compound binds preferentially to C23 over another cysteine amino acid residue within the OTUB1 sequence (e.g., C91, C204, or C212). In some embodiments, the bifunctional compound binds preferentially to C23 over C91 within the OTUB1 sequence. In some embodiments, the bifunctional compound does not substantially bind to C91 within the OTUB1 sequence.

In some embodiments, binding of the bifunctional compound (e.g., the DUB Recruiter) to OTBU1 does not modulate the activity of OTUB1 more than 0.1, 0.5, 1, 1.5, 2, 2.5, 3, 4, 5, 6, 7, 8, 9, 10, 15, 20, 25, 30, 35, 40, 45, 50, 55, 60, 65, 70, 75, 80, 85, 90, 95, or 99%, relative to the activity of OTUB1 in the absence of the bifunctional compound. In some embodiments, binding of the bifunctional compound (e.g., the DUB Recruiter) to C23 within the OTUB1 sequence does not modulate the activity of OTUB1 more than 0.1-50%, 1-50%, 1-25%, 1-10%, 0.1-10%, 1-5%, or 0.1-2%, relative to the activity of the deubiquitinase in the absence of the bifunctional compound. In some embodiments, the binding of the bifunctional compound (e.g., the DUB Recruiter) to OTUB1 does not substantially modulate (e.g., inhibit) the activity (e.g., deubiquitinase activity) of OTUB1. In some embodiments, the binding of the bifunctional compound (e.g., the DUB Recruiter) to C23 within the OTUB1 sequence does not substantially modulate (e.g., inhibit) the activity (e.g., deubiquitinase activity) of OTUB1.

In some embodiments, the deubiquitinase is OTUD5 (Uniprot ID Q96G74). The bifunctional compound described herein may bind to (e.g., covalently bind to) any cysteine residue within the OTUB1 sequence, e.g., C491, C434, C519, C247, C142, or C143. In some embodiments, the bifunctional compound does not bind to a catalytic cysteine amino acid within the OTUD5 sequence. In some embodiments, the bifunctional compound binds to an allosteric cysteine amino acid residue within the OTUD5 sequence. In some embodiments, the bifunctional compound binds to a cysteine residue on a surface of OTUD5. In some embodiments, the bifunctional compound binds to a cysteine residue on or in the interior of OTUD5.

In some embodiments, the bifunctional compound binds to C491 within the OTUD5 sequence. In some embodiments, the bifunctional compound binds to C434 within the OTUD5 sequence. In some embodiments, the bifunctional compound binds to C519 within the OTUD5 sequence. In some embodiments, the bifunctional compound binds to C247 within the OTUD5 sequence. In some embodiments, the bifunctional compound binds to C142 within the OTUD5 sequence. In some embodiments, the bifunctional compound binds to C143 within the OTUD5 sequence. In some embodiments, the bifunctional compound binds preferentially to C434 over another cysteine amino acid residue within the OTUD5 sequence (e.g., C491, C519, C247, C142, or C143). In some embodiments, the bifunctional compound does not substantially bind to C244 within the OTUD5 sequence.

In some embodiments, binding of the bifunctional compound (e.g., the DUB Recruiter) to OTUD5 does not modulate the activity of OTUD5 more than 0.1, 0.5, 1, 1.5, 2, 2.5, 3, 4, 5, 6, 7, 8, 9, 10, 15, 20, 25, 30, 35, 40, 45, 50, 55, 60, 65, 70, 75, 80, 85, 90, 95, or 99%, relative to the activity of OTUD5 in the absence of the bifunctional compound. In some embodiments, binding of the bifunctional compound (e.g., the DUB Recruiter) to C434 within the OTUD5 sequence does not modulate the activity of OTUD5 more than 0.1-50%, 1-50%, 1-25%, 1-10%, 0.1-10%, 1-5%, or 0.1-2%, relative to the activity of the deubiquitinase in the absence of the bifunctional compound. In some embodiments, the binding of the bifunctional compound (e.g., the DUB Recruiter) to OTUD5 does not substantially modulate (e.g., inhibit) the activity (e.g., deubiquitinase activity) of OTUD5. In some embodiments, the binding of the bifunctional compound (e.g., the DUB Recruiter) to C434 within the OTUD5 sequence does not substantially modulate (e.g., inhibit) the activity (e.g., deubiquitinase activity) of OTUD5.

In some embodiments, the deubiquitinase is USP15 (Uniprot ID Q9Y4E8). The bifunctional compound described herein may bind to (e.g., covalently bind to) any cysteine residue within the USP15 sequence, e.g., C139, C264, C289, C298, C306, C381, C448, C451, C462, C506, C570, C633, C809, C812, or C873. In some embodiments, the bifunctional compound does not bind to a catalytic cysteine amino acid within the USP15 sequence. In some embodiments, the bifunctional compound binds to an allosteric cysteine amino acid residue within the USP15 sequence. In some embodiments, the bifunctional compound binds to a cysteine residue on a surface of USP15. In some embodiments, the bifunctional compound binds to a cysteine residue on or in the interior of USP15.

In some embodiments, the bifunctional compound binds to C139 within the USP15 sequence. In some embodiments, the bifunctional compound binds to C264 within the USP15 sequence. In some embodiments, the bifunctional compound binds to C289 within the USP15 sequence. In some embodiments, the bifunctional compound binds to C298 within the USP15 sequence. In some embodiments, the bifunctional compound binds to C306 within the USP15 sequence. In some embodiments, the bifunctional compound binds to C381 within the USP15 sequence. In some embodiments, the bifunctional compound binds to C448 within the USP15 sequence. In some embodiments, the bifunctional compound binds to C451 within the USP15 sequence. In some embodiments, the bifunctional compound binds to C462 within the USP15 sequence. In some embodiments, the bifunctional compound binds to C506 within the USP15 sequence. In some embodiments, the bifunctional compound binds to C570 within the USP15 sequence. In some embodiments, the bifunctional compound binds to C633 within the USP15 sequence. In some embodiments, the bifunctional compound binds to C809 within the USP15 sequence. In some embodiments, the bifunctional compound binds to C812 within the USP15 sequence. In some embodiments, the bifunctional compound binds to C873 within the USP15 sequence. In some embodiments, the bifunctional compound binds preferentially to C264 over another cysteine amino acid residue within the USP15 sequence (e.g., C139, C264, C289, C298, C306, C381, C448, C451, C462, C506, C570, C633, C809, C812, or C873). In some embodiments, the bifunctional compound does not substantially bind to C298 within the USP15 sequence.

In some embodiments, binding of the bifunctional compound (e.g., the DUB Recruiter) to USP15 does not modulate the activity of USP15 more than 0.1, 0.5, 1, 1.5, 2, 2.5, 3, 4, 5, 6, 7, 8, 9, 10, 15, 20, 25, 30, 35, 40, 45, 50, 55, 60, 65, 70, 75, 80, 85, 90, 95, or 99%, relative to the activity of USP15 in the absence of the bifunctional compound. In some embodiments, binding of the bifunctional compound (e.g., the DUB Recruiter) to C264 or C381 within the USP15 sequence does not modulate the activity of USP15 more than 0.1-50%, 1-50%, 1-25%, 1-10%, 0.1-10%, 1-5%, or 0.1-2%, relative to the activity of the deubiquitinase in the absence of the bifunctional compound. In some embodiments, the binding of the bifunctional compound (e.g., the DUB Recruiter) to USP15 does not substantially modulate (e.g., inhibit) the activity (e.g., deubiquitinase activity) of USP15. In some embodiments, the binding of the bifunctional compound (e.g., the DUB Recruiter) to C264 or C381 within the OTUD5 sequence does not substantially modulate (e.g., inhibit) the activity (e.g., deubiquitinase activity) of OTUD5.

### Bifunctional Compounds

The present disclosure describes bifunctional compounds capable of binding to a target protein and a deubiquitinase, e.g., simultaneously binding to a target protein and a deubiquitinase. Without being bound by theory, these bifunctional compounds work to bring a deubiquitinase in proximity with a ubiquitinated target protein, such that the deubiquitinase is capable of removing one or more Ubl proteins from the ubiquitinated target protein to modulate (e.g., stabilize and/or prevent degradation of) the target protein.

In some embodiments, the modulating comprises one or more of: (i) modulating the folding of the target protein; (ii) modulating the half-life of the target protein; (iii) modulating trafficking of the target protein to the proteasome; (iv) modulating the level of ubiquitination of the target protein; (v) modulating degradation (e.g., proteasomal degradation) of the target protein; (vi) modulating target protein signaling; (vii) modulating target protein localization; (viii) modulating trafficking of the target protein to the lysosome; and (ix) modulating target protein interactions with another protein. In an embodiment, the modulating comprises (i). In an embodiment, the modulating comprises (ii). In an embodiment, the modulating comprises (i). In an embodiment, the modulating comprises (iii). In an embodiment, the modulating comprises (iv). In an embodiment, the modulating comprises (v). In an embodiment, the modulating comprises (vi). In an embodiment, the modulating comprises (vii). In an embodiment, the modulating comprises (viii). In an embodiment, the modulating comprises (ix). In an embodiment, the modulating comprises two of (i)-(ix). In an embodiment, the modulating comprises three of (i)-(ix). In an embodiment, the modulating comprises four of (i)-(ix). In an embodiment, the modulating comprises five of (i)-(ix). In an embodiment, the modulating comprises six of (i)-(ix). In an embodiment, the modulating comprises seven of (i)-(ix). In an embodiment, the modulating comprises eight of (i)-(ix). In an embodiment, the modulating comprises each of (i)-(ix).

In some embodiments, the bifunctional compound has the structure of Formula (I):

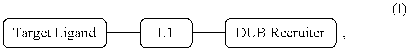

or a pharmaceutically acceptable salt, hydrate, solvate, stereoisomer, or tautomer thereof, wherein (i) the Target Ligand comprises a moiety capable of binding to a target protein; (ii) L1 comprises a linker; and (iii) the DUB Recruiter comprises a moiety capable of binding to a deubiquitinase. Each of the components of the bifunctional compounds of Formula (I) are described herein in turn.

### Target Ligands

The Target Ligand within the bifunctional compound is a small molecule moiety capable of binding to a target protein or other protein of interest. In some embodiment, the Target Ligand binds to a target protein described herein, e.g., an enzyme, receptor, membrane channel, hormone, transcription factor, tumor suppressor, ion channel, apoptotic factor, oncogenic protein, epigenetic regulator, or fragment thereof. In some embodiments, the Target Ligand binds to a kinase (e.g., PKN1, BCR, MAP4K4, TYK2, MAP4K2, EPHB4, MAP4K5, MAP3K2, DDR1, TGFBR1, RIPK2, TNK1, LYN, STK10, PKMYT1, LYN, EGFR, EPHA1, GAK, SIK2, MAP2K2, SLK, PRKACB, EPHA2, WEE1, or glucokinase). In some embodiments, the Target Ligand binds to a tumor suppressor kinase (e.g., WEE1). In some embodiments, the Target Ligand binds to a ligase (e.g., an E3 ligase, e.g., MDM2). In some embodiments, the Target Ligand binds to a transcription factor (e.g., MYC). In some embodiments, the Target Ligand binds to a tumor suppressor (e.g., TP53, AXIN1, BAX, CDKN1A, CKDN1C, PTEN, or SMAD4). In some embodiments, the Target Ligand binds to a haploinsufficiency target (e.g., SMN1/2, GLUT1, CFTR, PAH, FAH, or GAA). In some embodiments, the Target Ligand binds to a membrane channel (e.g., CFTR). In some embodiments, the Target Ligand binds to CFTR or a fragment thereof (e.g., ΔF508-CFTR). In some embodiments, the Target Ligand binds to CFTR comprising a sequence mutation (e.g., a Class I, Class II, Class III, Class IV, or Class V mutation). In some embodiments, the Target Ligand binds to CFTR comprising a sequence mutation selected from the group consisting of G551D, R177H, and A445E.

In some embodiments, the Target Ligand is a CFTR potentiator. In some embodiments, the Target Ligand comprises ivacaftor, lumacaftor, tezacaftor, elexacafor, or icenticaftor, or a derivative thereof. In some embodiments, the Target Ligand is a compound disclosed in one or more of U.S. Pat. Nos. 7,999,113; 8,247,436; 8,410,274; WO 2011/133953; and WO 2018/037350, each of which is incorporated by reference in its entirety.

In some embodiments, the Target Ligand has the structure of Formula (I-a):

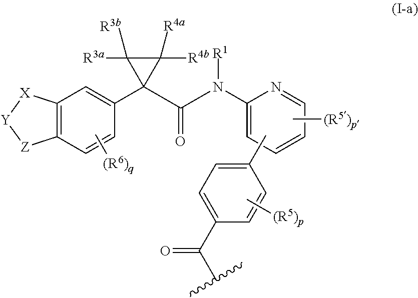

or a pharmaceutically acceptable salt, hydrate, solvate, prodrug, stereoisomer, or tautomer thereof, wherein X and Z are each independently O, S, or C(R^{7a})(R^{7b}); Y is C(R^{7a})(R^{7b}) or NR⁷c; R¹ is H or C₁₋₆ alkyl; R^{3a}, R^{3b}, R^{3c}, R^{3d} are each independently H, C₁₋₆ alkyl, C₁₋₆ haloalkyl, C₁₋₆ heteroalkyl, halo, cyano, or —OR^{A}; each R⁵, R^{5′}, and R⁶ is independently C₁₋₆ alkyl, C₁₋₆ haloalkyl, C₁₋₆ heteroalkyl, halo, cyano, —OR^{A}, —C(O)N(R^{B})(R^{C}), or —N(R^{B})CO(R^{D}); R^{7a} and R^{7b} are each independently H, C₁₋₆ alkyl, C₁₋₆ haloalkyl, C₁₋₆ heteroalkyl, or halo; R^{7C} is H or C₁₋₆ alkyl; RA, R^{B}, R^{C}, and R^{D} are each independently H, C₁₋₆ alkyl, C₂₋₆ alkenyl, C₂₋₆ alkynyl, C₁₋₆ haloalkyl, C₁₋₆ heteroalkyl, cycloalkyl, heterocyclyl, aryl, or heteroaryl; p is 0, 1, 2, 3, or 4; p′ is 0, 1, 2, 3, or 4; q is 0, 1, 2, or 3; and [symbol] denotes the point of attachment to L1 in Formula (I). In some embodiments, X is O. In some embodiments, Z is O. In some embodiments, each of X and Z is independently O. In some embodiments, Y is C(R^{7a})(R^{7b}). In some embodiments, each of R^{7a} and R^{7b} is independently halo (e.g., fluoro). In some embodiments, X is O, Z is O, and Y is C(R^{7a})(R^{7b}). In some embodiments, X is O, Z is O, and Y is CF₂. In some embodiments, R^{3a} and R^{3b} are each independently H. In some embodiments, R^{3c} and R^{3d} are each independently H. In some embodiments, each of R^{3a}, R^{3b}, R^{3c}, R^{3d} is independently H. In some embodiments, R⁵ is C₁-6 alkyl (e.g., methyl). In some embodiments, R¹ is H. In some embodiments, p is 0. In some embodiments, p′ is 1. In some embodiments, q is 0. In some embodiments, each of p and q is independently 0. In some embodiments, p is 0, q is 0, p′ is 1, and R^{5′} is C₁₋₆ alkyl. In some embodiments, p is 0, q is 0, p′ is 1, and R⁵ is methyl.

In some embodiments, the Target Ligand has the structure of Formula (I-b):

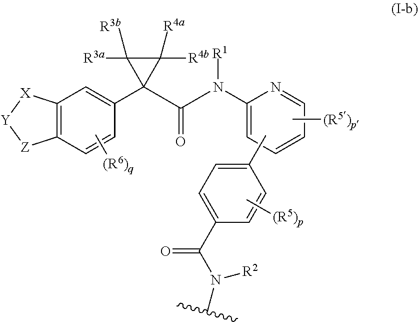

or a pharmaceutically acceptable salt, hydrate, solvate, prodrug, stereoisomer, or tautomer thereof, wherein X and Z are each independently O, S, or C(R^{7a})(R^{7b}); Y is C(R^{7a})(R^{7b}) or NR⁷c; R¹ is H or C₁₋₆ alkyl; R² is H or C₁₋₆ alkyl; R^{3a}, R^{3b}, R+a, R^{3d} are each independently H, C₁₋₆ alkyl, C₁₋₆ haloalkyl, C₁₋₆ heteroalkyl, halo, cyano, or —OR^{A}; each R⁵, R^{5′}, and R⁶ is independently C₁₋₆ alkyl, C₁₋₆ haloalkyl, C₁₋₆ heteroalkyl, halo, cyano, —OR^{A}, —C(O)N(R^{B})(R^{C}), or —N(R^{B})CO(R^{D}); R^{7a} and R^{7b} are each independently H, C₁₋₆ alkyl, C₁₋₆ haloalkyl, C₁₋₆ heteroalkyl, or halo; R^{7c} is H or C₁₋₆ alkyl; R^{A}, R^{B}, R^{C}, and R^{D} are each independently H, C₁₋₆ alkyl, C₂₋₆ alkenyl, C₂₋₆ alkynyl, C₁₋₆ haloalkyl, C₁₋₆ heteroalkyl, cycloalkyl, heterocyclyl, aryl, or heteroaryl; p is 0, 1, 2, 3, or 4; p′ is 0, 1, 2, 3, or 4; q is 0, 1, 2, or 3; and [symbol] denotes the point of attachment to L1 in Formula (I).

In some embodiments, X is O. In some embodiments, Z is O. In some embodiments, each of X and Z is independently O. In some embodiments, Y is C(R^{7a})(R^{7b}). In some embodiments, each of R^{7a} and R^{7b} is independently halo (e.g., fluoro). In some embodiments, X is O, Z is O, and Y is C(R^{7a})(R^{7b}). In some embodiments, X is O, Z is O, and Y is CF₂. In some embodiments, R^{3a} and R^{3b} are each independently H. In some embodiments, R^{3c} and R^{3d} are each independently H. In some embodiments, each of R^{3a}, R^{3b}, R^{4a}, R^{3d} is independently H. In some embodiments, R⁵ is C₁-6 alkyl (e.g., methyl). In some embodiments, R¹ is H. In some embodiments, R² is H. In some embodiments, each of R¹ and R² is independently H. In some embodiments, p is 0. In some embodiments, p′ is 1. In some embodiments, q is 0. In some embodiments, each of p and q is independently 0. In some embodiments, p is 0, q is 0, p′ is 1, and R⁵ is C₁-6 alkyl. In some embodiments, p is 0, q is 0, p′ is 1, and R⁵ is methyl.

In some embodiments, the Target Ligand has the structure of Formula (I-c):

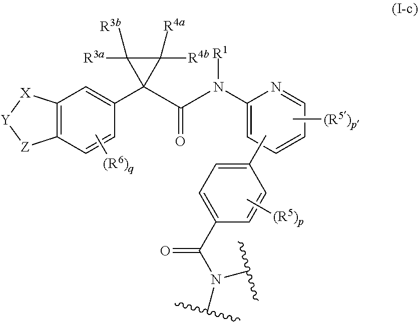

or a pharmaceutically acceptable salt, hydrate, solvate, prodrug, stereoisomer, or tautomer thereof, wherein X and Z are each independently O, S, or C(R⁷ª)(R^{7b}); Y is C(R^{7a})(R^{7b}) or NR⁷c; R¹ is H or C₁₋₆ alkyl; R^{3a}, R^{3b}, R^{4a}, R^{4b} are each independently H, C₁₋₆ alkyl, C₁₋₆ haloalkyl, C₁₋₆ heteroalkyl, halo, cyano, or —OR^{A}; each R⁵, R^{5′}, and R⁶ is independently C₁₋₆ alkyl, C₁₋₆ haloalkyl, C₁₋₆ heteroalkyl, halo, cyano, —OR^{A}, —C(O)N(R^{B})(R^{C}), or —N(R^{B})CO(R^{D}); R^{7a} and R^{7b} are each independently H, C₁₋₆ alkyl, C₁₋₆ haloalkyl, C₁₋₆ heteroalkyl, or halo; R^{7c} is H or C₁₋₆ alkyl; R^{A}, R^{B}, R^{C}, and R^{D} are each independently H, C₁₋₆ alkyl, C₂₋₆ alkenyl, C₂₋₆ alkynyl, C₁₋₆ haloalkyl, C₁₋₆ heteroalkyl, cycloalkyl, heterocyclyl, aryl, or heteroaryl; p is 0, 1, 2, 3, or 4; p′ is 0, 1, 2, 3, or 4; q is 0, 1, 2, or 3; and each [symbol] denotes the point of attachment to L1 in Formula (I).

In some embodiments, the Target Ligand is lumacaftor or a derivative thereof. In some embodiments, the Target Ligand has the structure of Formula (I-d):

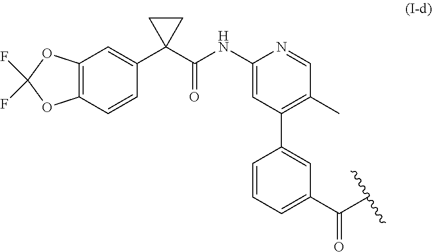 [SMILES: *C(=O)c1cccc(-c2cc(NC(=O)C3(c4ccc5c(c4)OC(F)(F)O5)CC3)ncc2C)c1]

or a pharmaceutically acceptable salt, hydrate, solvate, prodrug, stereoisomer, or tautomer thereof, wherein [symbol] denotes the point of attachment to L1 in Formula (I).

In some embodiments, the Target Ligand is lumacaftor or a derivative thereof. In some embodiments, the Target Ligand has the structure of Formula (I-ei):

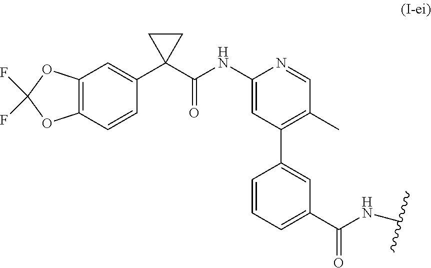 [SMILES: *NC(=O)c1cccc(-c2cc(NC(=O)C3(c4ccc5c(c4)OC(F)(F)O5)CC3)ncc2C)c1]

or a pharmaceutically acceptable salt, hydrate, solvate, prodrug, stereoisomer, or tautomer thereof, wherein [symbol] denotes the point of attachment to L1 in Formula (I).

In some embodiments, the Target Ligand is lumacaftor or a derivative thereof. In some embodiments, the Target Ligand has the structure of Formula (I-e-ii):

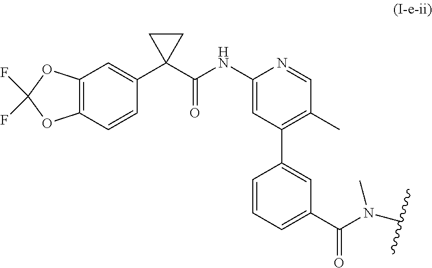 [SMILES: *N(C)C(=O)c1cccc(-c2cc(NC(=O)C3(c4ccc5c(c4)OC(F)(F)O5)CC3)ncc2C)c1]

or a pharmaceutically acceptable salt, hydrate, solvate, prodrug, stereoisomer, or tautomer thereof, wherein [symbol] denotes the point of attachment to L1 in Formula (I).

In some embodiments, the Target Ligand is lumacaftor or a derivative thereof. In some embodiments, the Target Ligand has the structure of Formula (I-e-iii):

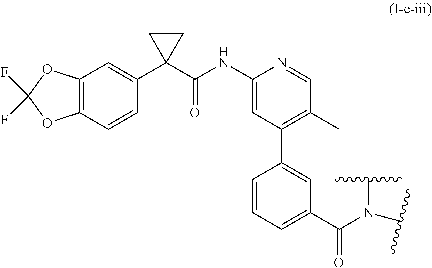 [SMILES: *N(*)C(=O)c1cccc(-c2cc(NC(=O)C3(c4ccc5c(c4)OC(F)(F)O5)CC3)ncc2C)c1]

or a pharmaceutically acceptable salt, hydrate, solvate, prodrug, stereoisomer, or tautomer thereof, wherein each of independently [symbol] denotes a point of attachment to L1 in Formula (I).

In some embodiments, the Target Ligand is lumacaftor or a derivative thereof. In some embodiments, the Target Ligand has the structure of Formula (I-f):

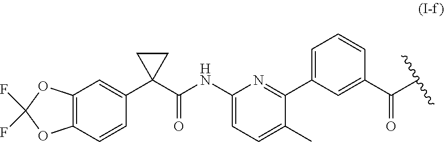 [SMILES: *C(=O)c1cccc(-c2nc(NC(=O)C3(c4ccc5c(c4)OC(F)(F)O5)CC3)ccc2C)c1]

or a pharmaceutically acceptable salt, hydrate, solvate, prodrug, stereoisomer, or tautomer thereof, wherein [symbol] denotes the point of attachment to L1 in Formula (I).

In some embodiments, the Target Ligand is lumacaftor or a derivative thereof. In some embodiments, the Target Ligand has the structure of Formula (I-g-i):

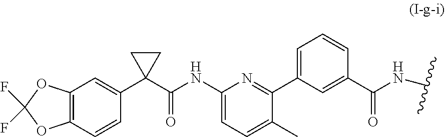 [SMILES: *NC(=O)c1cccc(-c2nc(NC(=O)C3(c4ccc5c(c4)OC(F)(F)O5)CC3)ccc2C)c1]

or a pharmaceutically acceptable salt, hydrate, solvate, prodrug, stereoisomer, or tautomer thereof, wherein [symbol] denotes the point of attachment to L1 in Formula (I).

In some embodiments, the Target Ligand is lumacaftor or a derivative thereof. In some embodiments, the Target Ligand has the structure of Formula (I-g-ii):

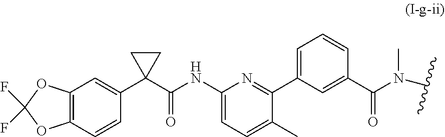 [SMILES: *N(C)C(=O)c1cccc(-c2nc(NC(=O)C3(c4ccc5c(c4)OC(F)(F)O5)CC3)ccc2C)c1]

or a pharmaceutically acceptable salt, hydrate, solvate, prodrug, stereoisomer, or tautomer thereof, wherein [symbol] denotes the point of attachment to L1 in Formula (I).

In some embodiments, the Target Ligand is lumacaftor or a derivative thereof. In some embodiments, the Target Ligand has the structure of Formula (I-g-iii):

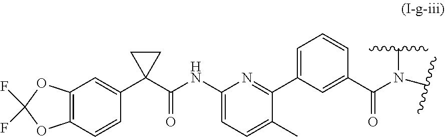 [SMILES: *N(*)C(=O)c1cccc(-c2nc(NC(=O)C3(c4ccc5c(c4)OC(F)(F)O5)CC3)ccc2C)c1]

or a pharmaceutically acceptable salt, hydrate, solvate, prodrug, stereoisomer, or tautomer thereof, wherein [symbol] denotes the point of attachment to L1 in Formula (I).

In some embodiments, the bifunctional compound of Formula (I) has the structure (II-a):

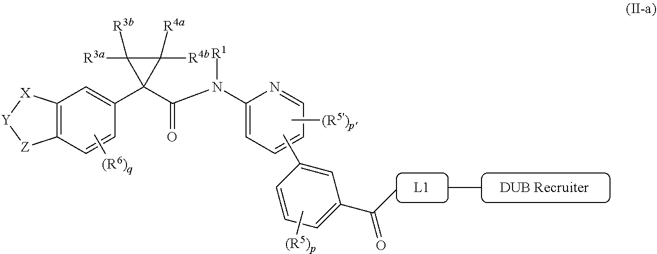

or a pharmaceutically acceptable salt, hydrate, solvate, prodrug, stereoisomer, or tautomer thereof, wherein X and Z are each independently O, S, or C(R^{7a})(R^{7b}); Y is C(R⁷ª)(R^{7b}) or NR⁷c; R¹ is H or C₁₋₆ alkyl; R^{3a}, R^{3b}, R+a, R^{3d} are each independently H, C₁₋₆ alkyl, C₁₋₆ haloalkyl, C₁₋₆ heteroalkyl, halo, cyano, or —OR^{A}; each R⁵, R^{5′}, and R⁶ is independently C₁₋₆ alkyl, C₁₋₆ haloalkyl, C₁₋₆ heteroalkyl, halo, cyano, —OR^{A}, —C(O)N(R^{B})(R^{C}), or —N(R^{B})CO(R^{D}); R^{7a} and R^{7b} are each independently H, C₁₋₆ alkyl, C₁₋₆ haloalkyl, C₁₋₆ heteroalkyl, or halo; R^{7c} is H or C₁₋₆ alkyl; R^{A}, R^{B}, R^{C}, and R^{D} are each independently H, C₁₋₆ alkyl, C₂₋₆ alkenyl, C₂₋₆ alkynyl, C₁₋₆ haloalkyl, C₁₋₆ heteroalkyl, cycloalkyl, heterocyclyl, aryl, or heteroaryl; p is 0, 1, 2, 3, or 4; p′ is 0, 1, 2, 3, or 4; q is 0, 1, 2, or 3; and L1 and DUB Recruiter are as defined for Formula (I).

In some embodiments, X is O. In some embodiments, Z is O. In some embodiments, each of X and Z is independently O. In some embodiments, Y is C(R^{7a})(R^{7b}). In some embodiments, R^{7a} and R^{7b} are each independently halo (e.g., fluoro). In some embodiments, X is O, Z is O, and Y is C(R^{7a})(R^{7b}). In some embodiments, X is O, Z is O, and Y is CF₂. In some embodiments, R^{3a} and R^{3b} are each independently H. In some embodiments, R^{3c} and R+D are each independently H. In some embodiments, each of R^{3a}, R^{3b}, R+a, R^{3d} is independently H. In some embodiments, R⁵ is C₁-6 alkyl (e.g., methyl). In some embodiments, R¹ is H. In some embodiments, p is 0. In some embodiments, p′ is 1. In some embodiments, q is 0. In some embodiments, each of p and q is independently 0. In some embodiments, p is 0, q is 0, p′ is 1, and R⁵ is C₁-6 alkyl. In some embodiments, p is 0, q is 0, p′ is 1, and R⁵ is methyl.

In some embodiments, the bifunctional compound of Formula (I) has the structure (II-b-i):

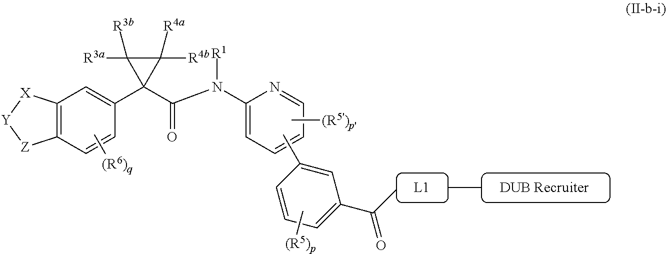

or a pharmaceutically acceptable salt, hydrate, solvate, prodrug, stereoisomer, or tautomer thereof, wherein X and Z are each independently O, S, or C(R^{7a})(R^{7b}); Y is C(R⁷ª)(R^{7b}) or NR⁷c; R¹ is H or C₁₋₆ alkyl; R² is H or C₁₋₆ alkyl; R^{3a}, R^{3b}, R+a, R^{3d} are each independently H, C₁₋₆ alkyl, C₁₋₆ haloalkyl, C₁₋₆ heteroalkyl, halo, cyano, or —OR^{A}; each R⁵, R^{5′}, and R⁶ is independently C₁₋₆ alkyl, C₁₋₆ haloalkyl, C₁₋₆ heteroalkyl, halo, cyano, —OR^{A}, —C(O)N(R^{B})(R^{C}), or —N(R^{B})CO(R^{D}); R^{7a} and R^{7b} are each independently H, C₁₋₆ alkyl, C₁₋₆ haloalkyl, C₁₋₆ heteroalkyl, or halo; R^{7c} is H or C₁₋₆ alkyl; R^{A}, R^{B}, R^{C}, and R^{D} are each independently H, C₁₋₆ alkyl, C₂₋₆ alkenyl, C₂₋₆ alkynyl, C₁₋₆ haloalkyl, C₁₋₆ heteroalkyl, cycloalkyl, heterocyclyl, aryl, or heteroaryl; p is 0, 1, 2, 3, or 4; p′ is 0, 1, 2, 3, or 4; q is 0, 1, 2, or 3; and L1 and DUB Recruiter are as defined for Formula (I).

In some embodiments, X is O. In some embodiments, Z is O. In some embodiments, each of X and Z is independently O. In some embodiments, Y is C(R^{7a})(R^{7b}). In some embodiments, R^{7a} and R^{7b} are each independently halo (e.g., fluoro). In some embodiments, X is O, Z is O, and Y is C(R^{7a})(R^{7b}). In some embodiments, X is O, Z is O, and Y is CF₂. In some embodiments, R^{3a} and R^{3b} are each independently H. In some embodiments, R^{3c} and R^{3d} are each independently H. In some embodiments, each of R^{3a}, R^{3b}, R^{3c}, R^{3d} is independently H. In some embodiments, R⁵ is C₁-6 alkyl (e.g., methyl). In some embodiments, R¹ is H. In some embodiments, R² is H. In some embodiments, each of R¹ and R² is independently H. In some embodiments, p is 0. In some embodiments, p′ is 1. In some embodiments, q is 0. In some embodiments, each of p and q is independently 0. In some embodiments, p is 0, q is 0, p′ is 1, and R⁵ is C₁-6 alkyl. In some embodiments, p is 0, q is 0, p′ is 1, and R⁵ is methyl. In some embodiments, the bifunctional compound of Formula (I) has the structure (II-b-ii):

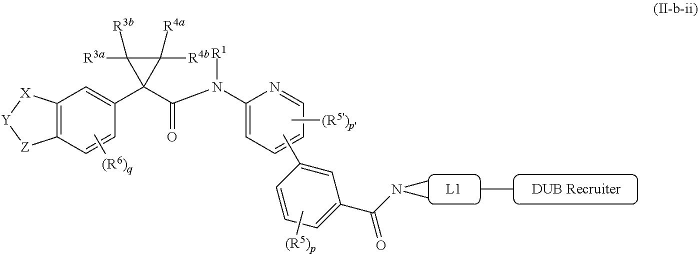

or a pharmaceutically acceptable salt, hydrate, solvate, prodrug, stereoisomer, or tautomer thereof, wherein X and Z are each independently O, S, or C(R^{7a})(R^{7b}); Y is C(R⁷ª)(R^{7b}) or NR⁷c; R¹ is H or C₁₋₆ alkyl; R^{3a}, R^{3b}, R+a, R+b are each independently H, C₁₋₆ alkyl, C₁₋₆ haloalkyl, C₁₋₆ heteroalkyl, halo, cyano, or —OR^{A}; each R⁵, R^{5′}, and R⁶ is independently C₁₋₆ alkyl, C₁₋₆ haloalkyl, C₁₋₆ heteroalkyl, halo, cyano, —OR^{A}, —C(O)N(R^{B})(R^{C}), or —N(R^{B})CO(R^{D}); R^{7a} and R^{7b} are each independently H, C₁₋₆ alkyl, C₁₋₆ haloalkyl, C₁₋₆ heteroalkyl, or halo; R^{7c} is H or C₁₋₆ alkyl; R^{A}, R^{B}, R^{C}, and R^{D} are each independently H, C₁₋₆ alkyl, C₂₋₆ alkenyl, C₂₋₆ alkynyl, C₁₋₆ haloalkyl, C₁₋₆ heteroalkyl, cycloalkyl, heterocyclyl, aryl, or heteroaryl; p is 0, 1, 2, 3, or 4; p′ is 0, 1, 2, 3, or 4; q is 0, 1, 2, or 3; and L1 and DUB Recruiter are as defined for Formula (I).

In some embodiments, X is O. In some embodiments, Z is O. In some embodiments, each of X and Z is independently O. In some embodiments, Y is C(R⁷ª)(R^{7b}). In some embodiments, R^{7a} and R^{7b} are each independently halo (e.g., fluoro). In some embodiments, X is O, Z is O, and Y is C(R^{7a})(R^{7b}). In some embodiments, X is O, Z is O, and Y is CF₂. In some embodiments, R^{3a} and R^{3b} are each independently H. In some embodiments, R+a and R^{3d} are each independently H. In some embodiments, each of R^{3a}, R^{3b}, R+a, R^{4b} is independently H. In some embodiments, R⁵ is C₁-6 alkyl (e.g., methyl). In some embodiments, R¹ is H. In some embodiments, R² is H. In some embodiments, each of R¹ and R² is independently H. In some embodiments, p is 0. In some embodiments, p′ is 1. In some embodiments, q is 0. In some embodiments, each of p and q is independently 0. In some embodiments, p is 0, q is 0, p′ is 1, and R^{5′} is C₁₋₆ alkyl. In some embodiments, p is 0, q is 0, p′ is 1, and R⁵ is methyl.

In some embodiments, the bifunctional compound of Formula (I) has the structure (II-c):

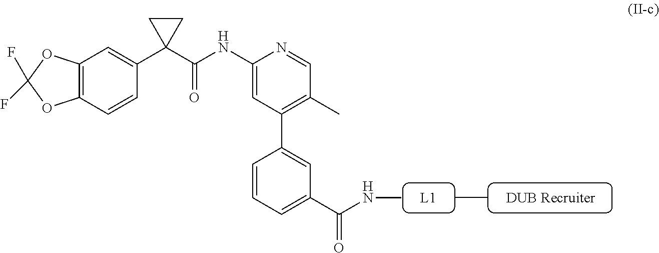

or a pharmaceutically acceptable salt, hydrate, solvate, prodrug, stereoisomer, or tautomer thereof, wherein L1 and DUB Recruiter are as defined for Formula (I).

In some embodiments, the bifunctional compound of Formula (I) has the structure (II-d):

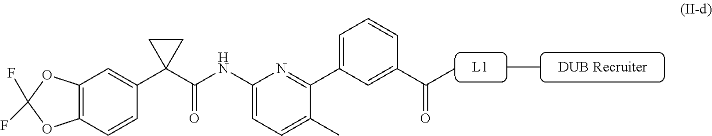

or a pharmaceutically acceptable salt, hydrate, solvate, prodrug, stereoisomer, or tautomer thereof, wherein L1 and DUB Recruiter are as defined for Formula (I).

In some embodiments, the bifunctional compound of Formula (I) has the structure (II-ei):

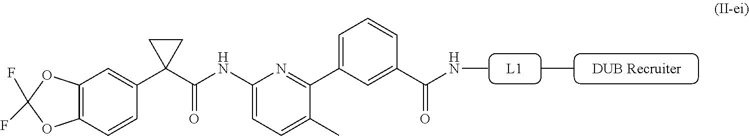

or a pharmaceutically acceptable salt, hydrate, solvate, prodrug, stereoisomer, or tautomer thereof, wherein L1 and DUB Recruiter are as defined for Formula (I).

In some embodiments, the bifunctional compound of Formula (I) has the structure (II-e-ii):

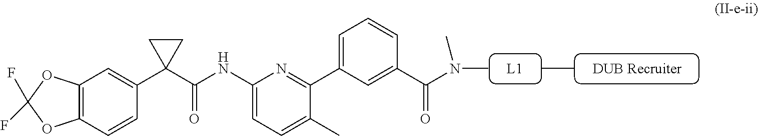

or a pharmaceutically acceptable salt, hydrate, solvate, prodrug, stereoisomer, or tautomer thereof, wherein L1 and DUB Recruiter are as defined for Formula (I).

In some embodiments, the bifunctional compound of Formula (I) has the structure (II-e-iii):

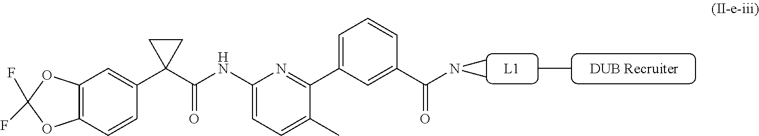

or a pharmaceutically acceptable salt, hydrate, solvate, prodrug, stereoisomer, or tautomer thereof, wherein L1 and DUB Recruiter are as defined for Formula (I).

In some embodiments, the Target Ligand is a derivative of ivacaftor. In some embodiments, the bifunctional compound of Formula (I) has the structure (II-f):

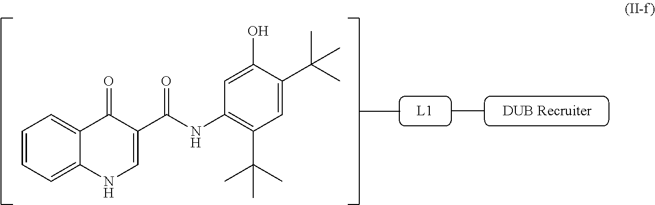

or a pharmaceutically acceptable salt, hydrate, solvate, prodrug, stereoisomer, or tautomer thereof, wherein L1 and DUB Recruiter are as defined for Formula (I).

In some embodiments, the Target Ligand is a derivative of tezacaftor. In some embodiments, the bifunctional compound of Formula (I) has the structure (II-g):

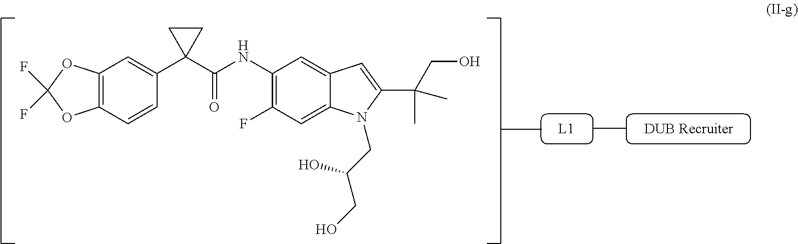

or a pharmaceutically acceptable salt, hydrate, solvate, prodrug, stereoisomer, or tautomer thereof, wherein L1 and DUB Recruiter are as defined for Formula (I).

In some embodiments, the Target Ligand is a derivative of elexacaftor. In some embodiments, the bifunctional compound of Formula (I) has the structure (II-h):

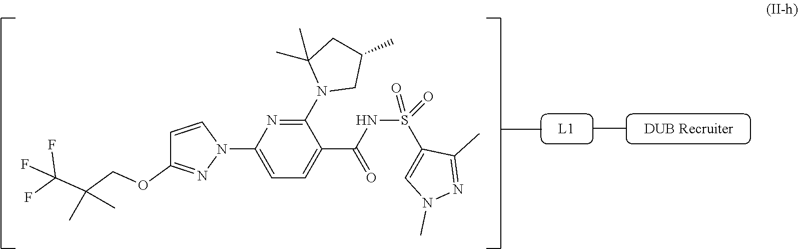

or a pharmaceutically acceptable salt, hydrate, solvate, prodrug, stereoisomer, or tautomer thereof, wherein L1 and DUB Recruiter are as defined for Formula (I).

In some embodiments, the Target Ligand is a derivative of icenticaftor. In some embodiments, the bifunctional compound of Formula (I) has the structure (II-i):

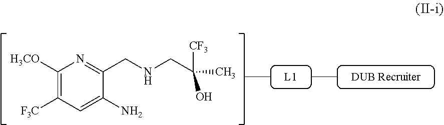

or a pharmaceutically acceptable salt, hydrate, solvate, prodrug, stereoisomer, or tautomer thereof, wherein L1 and DUB Recruiter are as defined for Formula (I).

The Target Ligand may or may not modulate an activity of the target protein (e.g., decrease or inhibit activity). In some embodiments, the Target Ligand is a CFTR inhibitor, wherein binding of the Target Ligand to CFTR decreases its activity, e.g., by about 1, 2, 2, 3, 4, 5, 6, 7, 8, 9, 10, 15, 20, 25, 30, 35, 40, 45, 50%, or more. In some embodiments, the Target Ligand is a CFTR inhibitor described in any of WO 2014/097147; WO 2014/097148; Verkman et al (2009) J Med Chem 6447; and Verkman et al (2013) ACS Med Chem Lett 456, each of which is incorporated herein by reference in its entirety. In some embodiments, the Targeting Ligand is a tricyclic CFTR inhibitor. In some embodiments, the Target Ligand is a structure of Formula (IV-a):

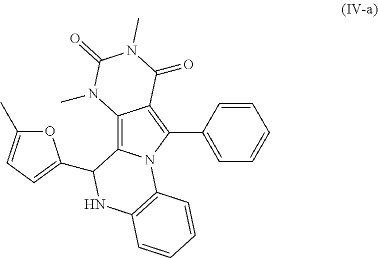 [SMILES: Cc1ccc(C2Nc3ccccc3-n3c(-c4ccccc4)c4c(=O)n(C)c(=O)n(C)c4c32)o1]

or a pharmaceutically acceptable salt, hydrate, solvate, prodrug, stereoisomer, or tautomer thereof. In some embodiments, the Target Ligand is PPQ-102 or a derivative thereof. In some embodiments, the Target Ligand is a structure of Formula (IV-b):

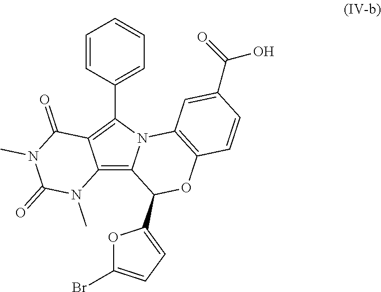 [SMILES: Cn1c(=O)c2c(-c3ccccc3)n3c(c2n(C)c1=O)[C@H](c1ccc(Br)o1)Oc1ccc(C(=O)O)cc1-3]

or a pharmaceutically acceptable salt, hydrate, solvate, prodrug, stereoisomer, or tautomer thereof. In some embodiments, the Target Ligand is BPO-27 of a derivative thereof.

In some embodiments, the Target Ligand is a kinase inhibitor. In some embodiments, the Target Ligand is a tumor suppressor kinase inhibitor, e.g., a WEE1 inhibitor. Exemplary WEE1 inhibitors include AZD1775 (i.e., MK1775, adavosertib), MK-3652, or related derivatives thereof. In some embodiments, the Target Ligand is AZD1775 or a related derivative thereof. In some embodiments, the Target Ligand is a compound disclosed in one or more of WO 2007/126122, WO 2011/035743, WO 2008/153207, WO 2009/151997, and US 2011/1035601, each of which is incorporated by reference in its entirety.

In some embodiments, the Target Ligand has the structure of Formula (I-h):

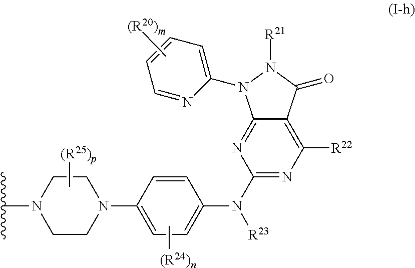

or a pharmaceutically acceptable salt, hydrate, solvate, prodrug, stereoisomer, or tautomer thereof, wherein each R²⁰, R²⁴, and R²⁵ is independently C₁₋₆ alkyl, C₂₋₆ alkenyl, C₂₋₆ alkynyl, C₁₋₆ haloalkyl, C₁₋₆ heteroalkyl, halo, cyano, —OR^{A}, —C(O)N(R^{B})(R^{C}), or —N(R^{B})CO(R^{D}); R²¹ and R²³ are each independently H or C₁₋₆ alkyl; R²² is C₁-6 alkyl, C₂₋₆ alkenyl, C₂₋₆ alkynyl, C₁₋₆ haloalkyl, C₁₋₆ heteroalkyl, halo, cyano, —OR^{A}, —C(O)N(R^{B})(R^{C}), or —N(R^{B})CO(R^{D}); R^{A}, R^{B}, R^{C}, and R^{D} are each independently H, C₁₋₆ alkyl, C₂₋₆ alkenyl, C₂₋₆ alkynyl, C₁₋₆ haloalkyl, C₁₋₆ heteroalkyl, cycloalkyl, heterocyclyl, aryl, or heteroaryl; m and n are each independently 0, 1, 2, 3, or 4; p is 0, 1, 2, 3, 4, 5, 6, 7, or 8; and [symbol] denotes the point of attachment to L1 in Formula (I).

In some embodiments, R²⁰ is C₁₋₆ heteroalkyl (e.g., C(CH₃)₂OH). In some embodiments, R²¹ is C₁₋₆ alkenyl (e.g., CH₂CH—CH₂). In some embodiments, R²² is H. In some embodiments, R²³ is H. In some embodiments, m is 1. In some embodiments, each of n and p is independently 0.

In some embodiments, the Target Ligand is AZD1775 or a derivative thereof. In some embodiments, the Target Ligand has the structure of Formula (I-i):

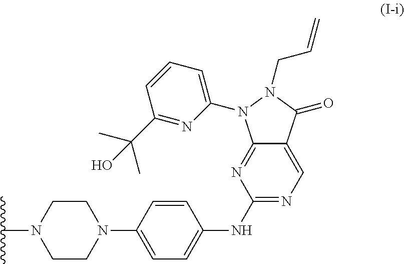 [SMILES: *N1CCN(c2ccc(Nc3ncc4c(=O)n(CC=C)n(-c5cccc(C(C)(C)O)n5)c4n3)cc2)CC1]

or a pharmaceutically acceptable salt, hydrate, solvate, prodrug, stereoisomer, or tautomer thereof, wherein [symbol] denotes the point of attachment to L1 in Formula (I).
or a pharmaceutically acceptable salt, hydrate, solvate, prodrug, stereoisomer, or tautomer thereof, wherein [symbol] denotes the point of attachment to L1 in Formula (I).

In some embodiments, the bifunctional compound of Formula (I) has the structure (II-j):

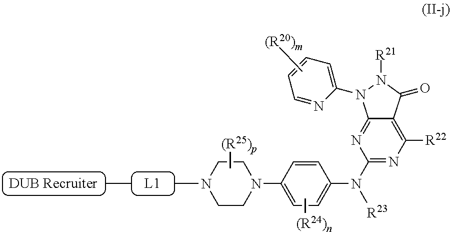

or a pharmaceutically acceptable salt, hydrate, solvate, prodrug, stereoisomer, or tautomer thereof, wherein each R²⁰, R²⁴, and R²⁵ is independently C₁₋₆ alkyl, C₂₋₆ alkenyl, C₂₋₆ alkynyl, C₁₋₆ haloalkyl, C₁₋₆ heteroalkyl, halo, cyano, —OR^{A}, —C(O)N(R^{B})(R^{C}), or —N(R^{B})CO(R^{D}); R²¹ and R²³ are each independently H or C₁₋₆ alkyl; R²² is C₁₋₆ alkyl, C₂₋₆ alkenyl, C₂₋₆ alkynyl, C₁₋₆ haloalkyl, C₁₋₆ heteroalkyl, halo, cyano, —OR^{A}, —C(O)N(R^{B})(R^{C}), or —N(R^{B})CO(R^{D}); R^{A}, R^{B}, R^{C}, and R^{D} are each independently H, C₁₋₆ alkyl, C₂₋₆ alkenyl, C₂₋₆ alkynyl, C₁₋₆ haloalkyl, C₁₋₆ heteroalkyl, cycloalkyl, heterocyclyl, aryl, or heteroaryl; m and n are each independently 0, 1, 2, 3, or 4; p is 0, 1, 2, 3, 4, 5, 6, 7, or 8; and L1 and DUB Recruiter are as defined for Formula (I).

In some embodiments, R²⁰ is C₁₋₆ heteroalkyl (e.g., C(CH₃)₂₀H). In some embodiments, R²¹ is C₁₋₆ alkenyl (e.g., CH₂CH═CH₂). In some embodiments, R²² is H. In some embodiments, R²³ is H. In some embodiments, m is 1. In some embodiments, each of n and p is independently 0.

In some embodiments, the bifunctional compound of Formula (I) has the structure (II-k):

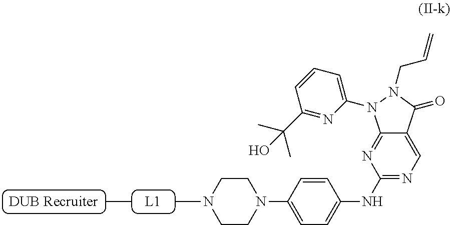

or a pharmaceutically acceptable salt, hydrate, solvate, prodrug, stereoisomer, or tautomer thereof, wherein L1 and DUB Recruiter are as defined for Formula (I).

### Linkers

The present disclosure features bifunctional compounds comprising a Target Ligand and a DUB Recruiter, separated by a linker (i.e., L1). In some embodiments, the linker is covalently bound to the Target Ligand. In some embodiments, the linker is covalently bound to the DUB Recruiter. In some embodiments, the linker is covalently bound to both the Target Ligand and the DUB Recruiter.

The linker may be a cleavable linker or a non-cleavable linker. In some embodiments, the linker is a non-cleavable linker. In some embodiments, the linker is not degraded or hydrolyzed at physiological conditions. In some embodiments, the linker comprises a bond that is not cleavable in a cell (e.g, a cell organelle) or the serum, e.g., of a sample or subject. In some embodiments, the linker comprises an alkyl, alkenyl, alkynyl, heteroalkyl, haloalkyl, ether, amine, alkoxy, aryl, heteroaryl, cycloalkyl, or heterocyclyl. In some embodiments, the linker comprises an alkylene or heteroalkylene.

In some embodiments, the linker (e.g., L1) has the structure of Formula (III-a):

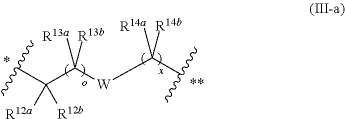

or a pharmaceutically acceptable salt, hydrate, solvate, prodrug, stereoisomer, or tautomer thereof, wherein R^{12a}, R^{12b}, R^{13a}, R^{13b}, R^{14a}, and R^{14b} are each independently H, C₁₋₄ alkyl, C₁₋₆ haloalkyl, C₁₋₆ heteroalkyl, halo, cyano, or —OR^{A}; or each of R^{12a} and R^{12b}, R^{13a} and R^{13b}, and R^{14a} and R^{14b} independently may be taken together with the carbon atom to which they are attached to form an oxo group; W is C(R^{15a})(R^{15b}), O, N(R¹⁶), or S; R^{15a} and R^{15b} are each independently H, C₁₋₆ alkyl, C₁₋₆ haloalkyl, C₁₋₆ heteroalkyl, halo, cyano, or —OR^{A}; or R^{15a} and R^{15b} may be taken together with the carbon atom to which they are attached to form an oxo group; R¹⁶ is H or C₁₋₆ alkyl; R^{A} is H, C₁₋₆ alkyl, C₂₋₆ alkenyl, C₂₋₆ alkynyl, C₁₋₆ haloalkyl, C₁₋₆ heteroalkyl, cycloalkyl, heterocyclyl, aryl, or heteroaryl; o and x are each independently an integer between 0 and 10; [symbol] denotes the point of attachment to the Target Ligand in Formula (I); and [symbol] denotes the point of attachment to the DUB Recruiter in Formula (I).

In some embodiments, each of R^{12a}, R^{12b}, R^{13a}, and R^{13b} is independently H. In some embodiments, each of R^{14a} and R^{14b} are taken together with the carbon atom to which they are attached form an oxo group. In some embodiments, W is N(R¹⁶) (e.g., NH). In some embodiments, o is selected from 2, 3, 4, 5, and 6. In some embodiments, p is selected from 1, 2, and 3.

In some embodiments, L1 has the structure of Formula (III-b):

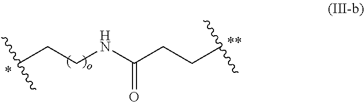

or a pharmaceutically acceptable salt, hydrate, solvate, prodrug, stereoisomer, or tautomer thereof, wherein o is an integer between 0 and 10; [symbol] denotes the point of attachment to the Target Ligand in Formula (I); and [symbol] denotes the point of attachment to the DUB Recruiter in Formula (I).

In some embodiments, L1 has the structure of Formula (III-c):

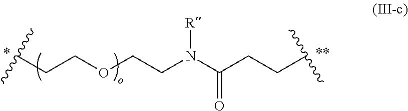

or a pharmaceutically acceptable salt, hydrate, solvate, prodrug, stereoisomer, or tautomer thereof, wherein R″ is H or C₁₋₆ alkyl, and o is an integer between 0 and 10; [symbol] denotes the point of attachment to the Target Ligand in Formula (I); and [symbol] denotes the point of attachment to the DUB Recruiter in Formula (I). In some embodiments, o is 1. In some embodiments, o is 2. In some embodiments, o is 3.

In some embodiments, the linker (e.g., L1) is selected from the group consisting of:

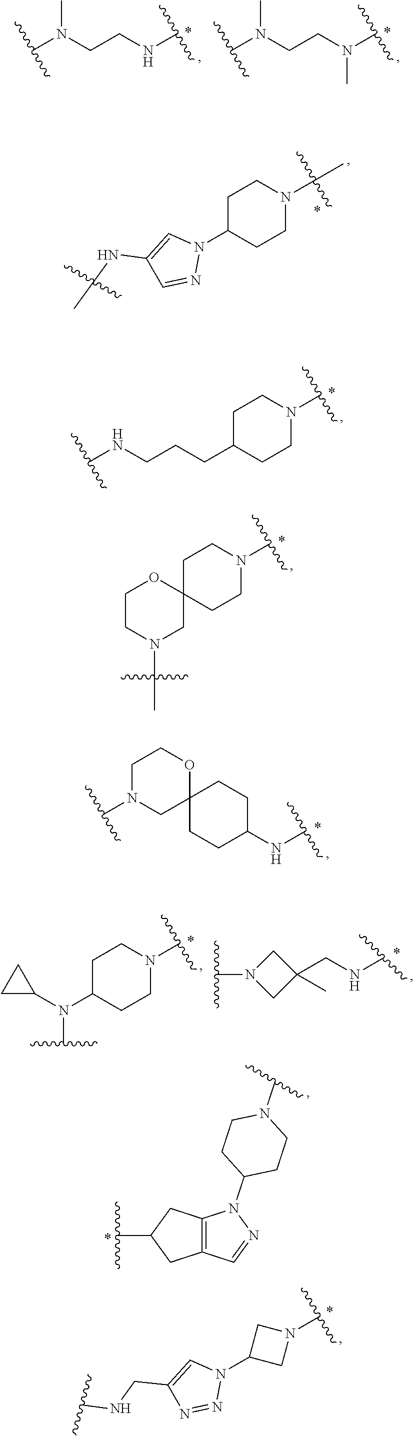

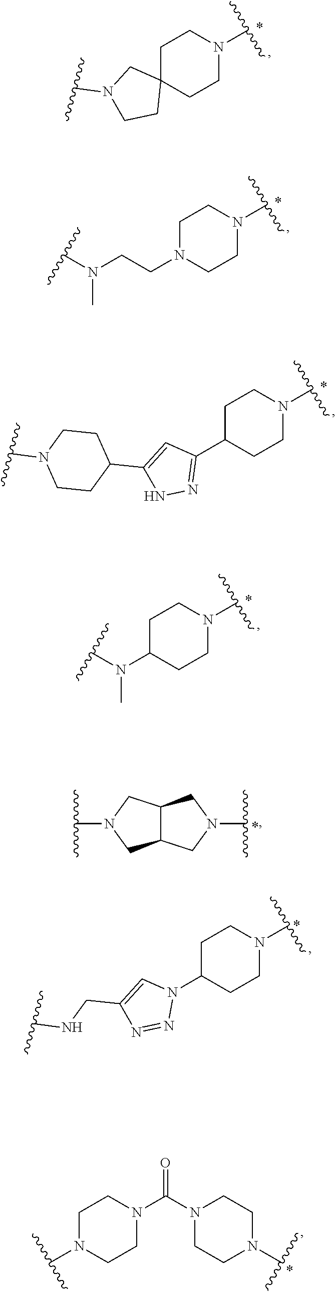

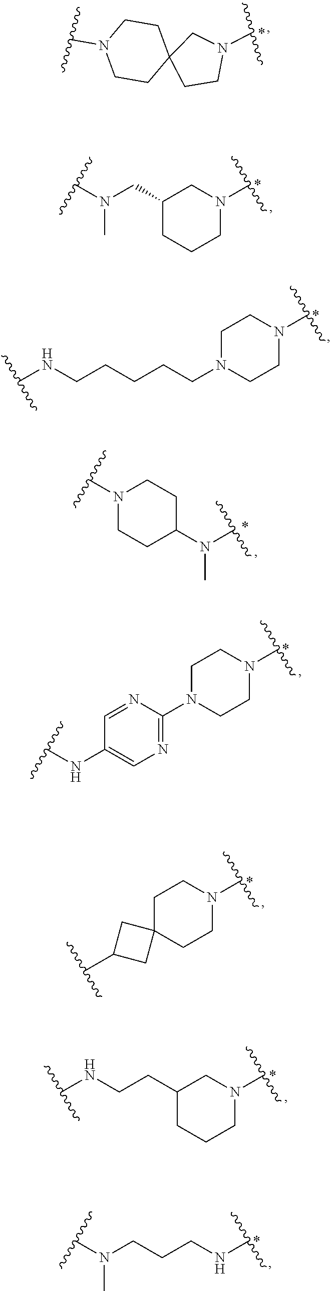

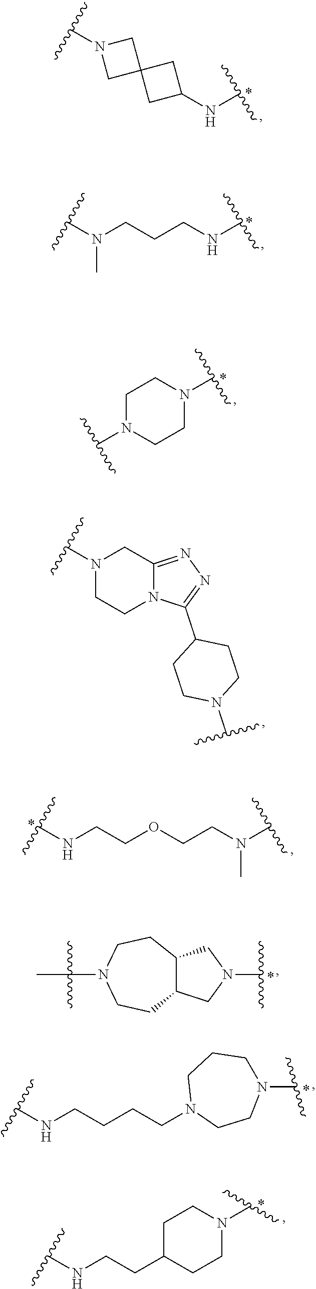

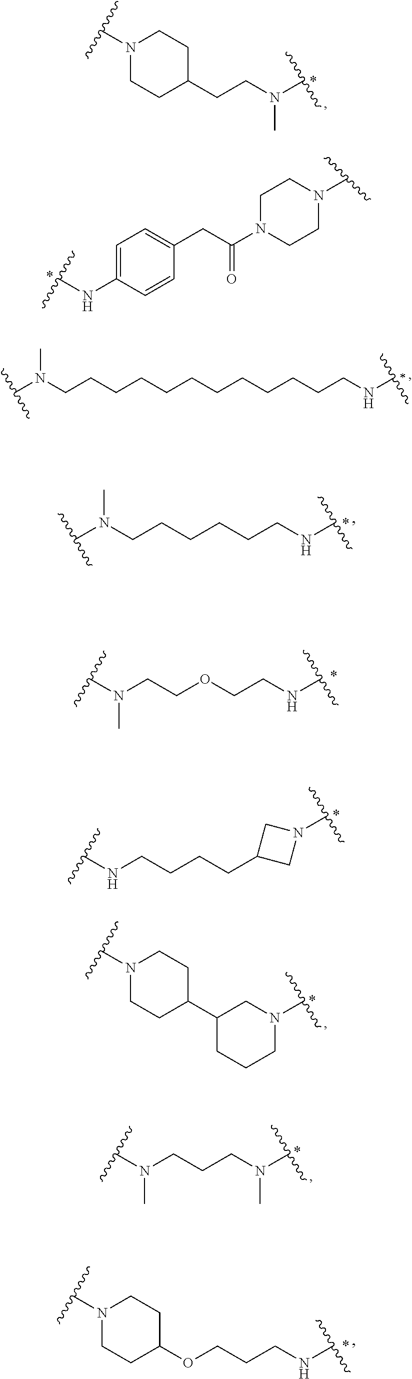

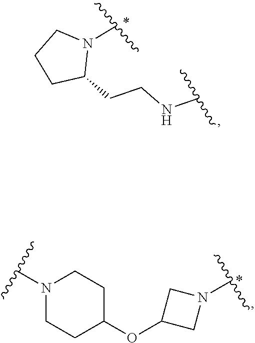 [SMILES: *NCC[C@@H]1CCCN1* ; *N1CCC(OC2CN(*)C2)CC1]

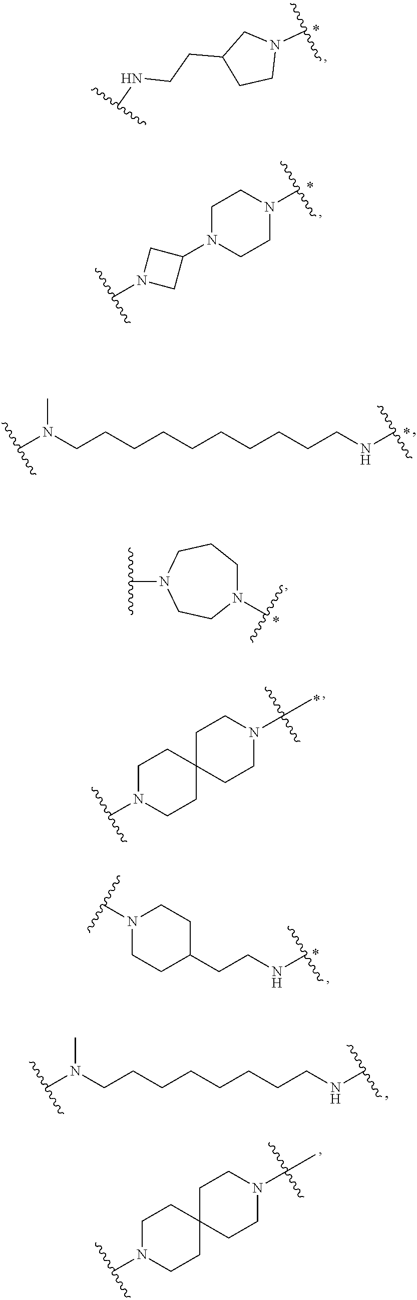

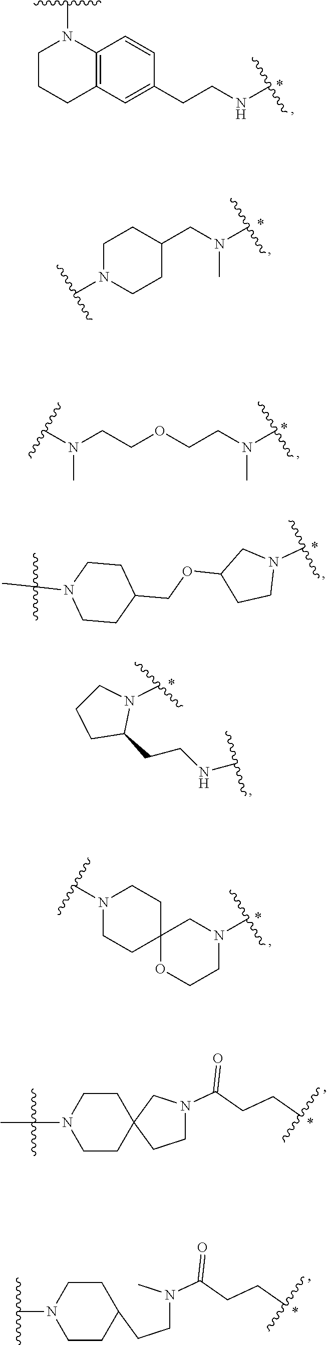

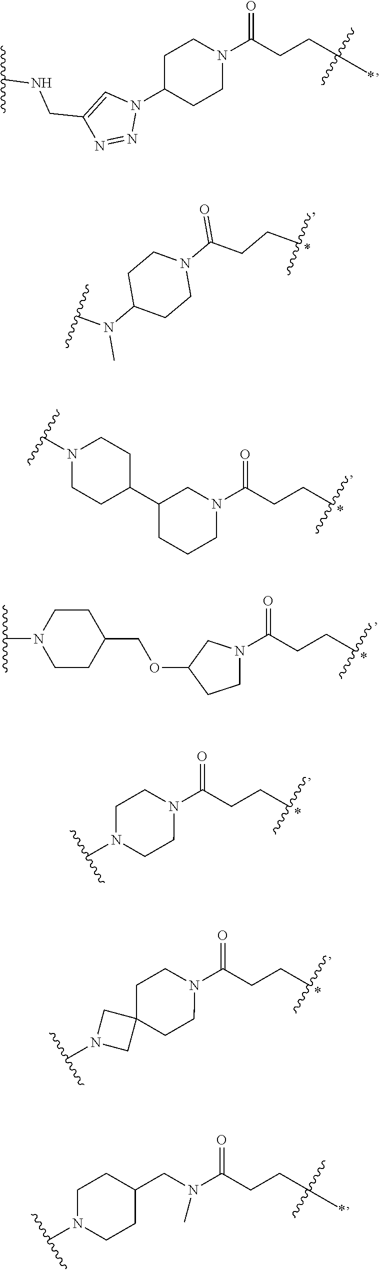

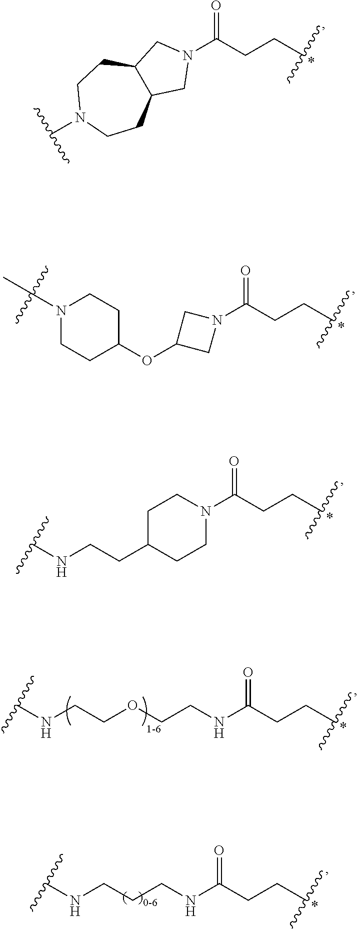

or a pharmaceutically acceptable salt, hydrate, solvate, prodrug, stereoisomer, or tautomer thereof, wherein “*” denotes the point of attachment to the Target Ligand or DUB Recruiter. In some embodiments, the linker (e.g., L1) is a variant of a linker described herein, e.g., wherein the linker (e.g., L1) comprises an additional C₁-C₆ alkyl or C₁-C₆ heteroalkyl moiety at a point of attachment, e.g., to the Target Ligand or the DUB Recruiter.

In some embodiments, the linker is a cleavable linker, e.g., a linker that is degraded or hydrolyzed at physiological conditions. In some embodiments, the linker comprises a bond cleavable in a cell (e.g, a cell organelle) or the serum, e.g., of a sample or subject. For example, the linker may be pH sensitive (e.g., acid labile or base labile) or cleaved through the action of an enzyme. In an embodiment, the rate of hydrolysis of the linker is increased by at least 0.5 times (e.g., at least 1, 1.5, 2, 2.5, 3, 4, 5, 7.5, 10, 12.5, 15, 20, 25, 50, 75, 100, 250, 500, 750, 1000 or more) compared with the rate of hydrolysis of the linker in the absence of an enzyme. In some embodiments, the enzyme is an esterase. In an embodiment, the linker comprises an ester, disulfide, thiol, hydrazone, ether, or amide.

In an embodiment, the linker (e.g., L1) is selected from the group consisting of:

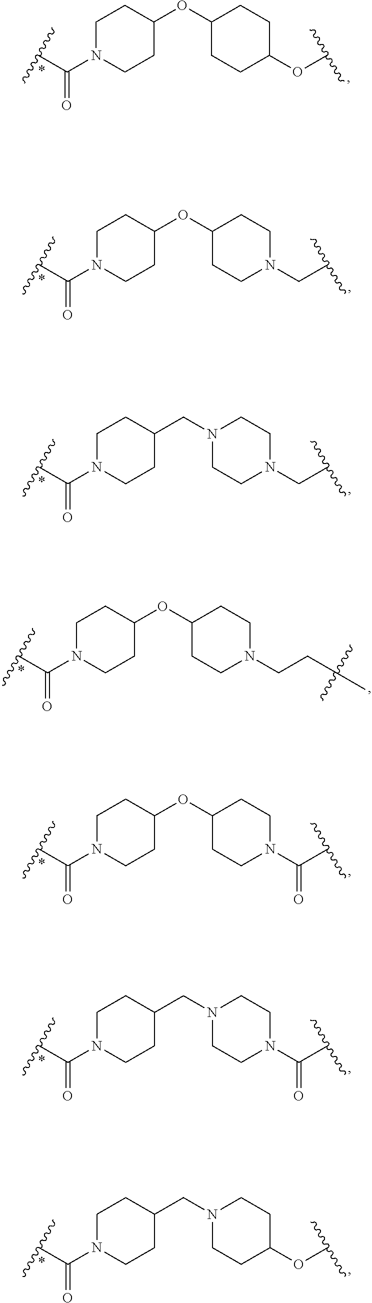

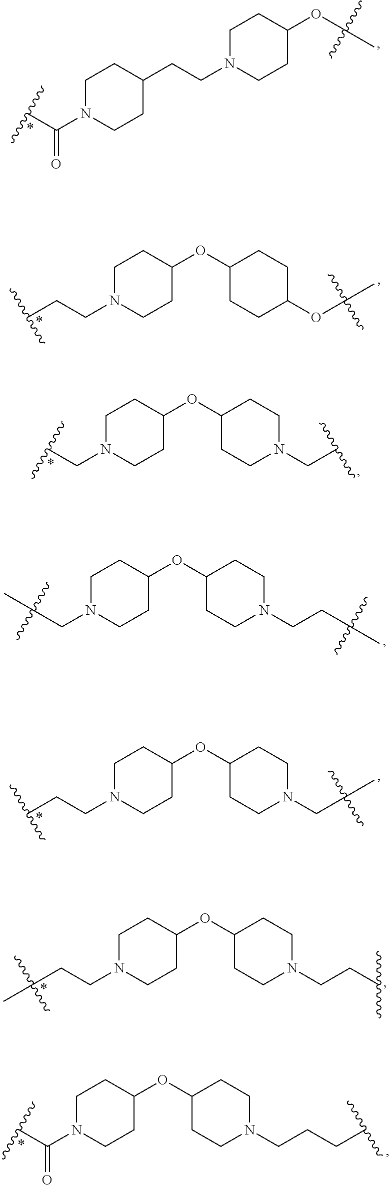

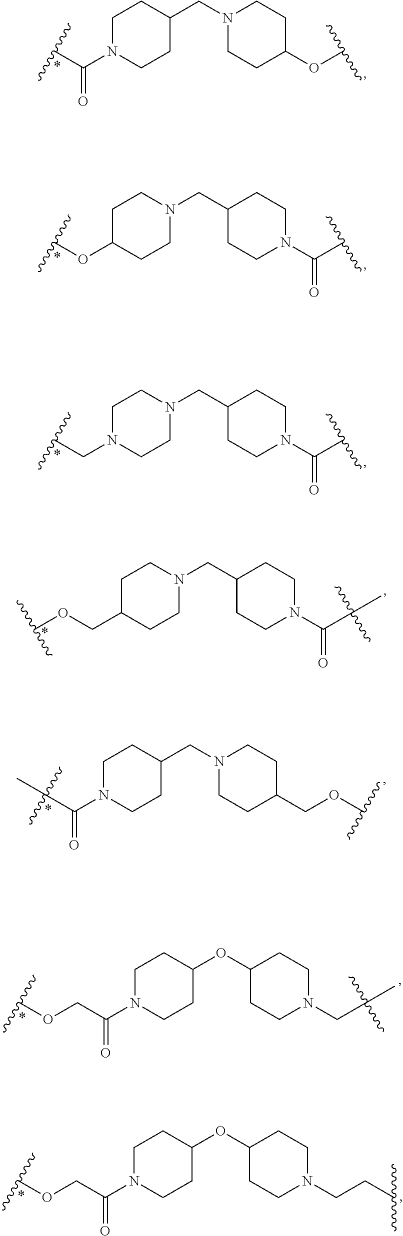

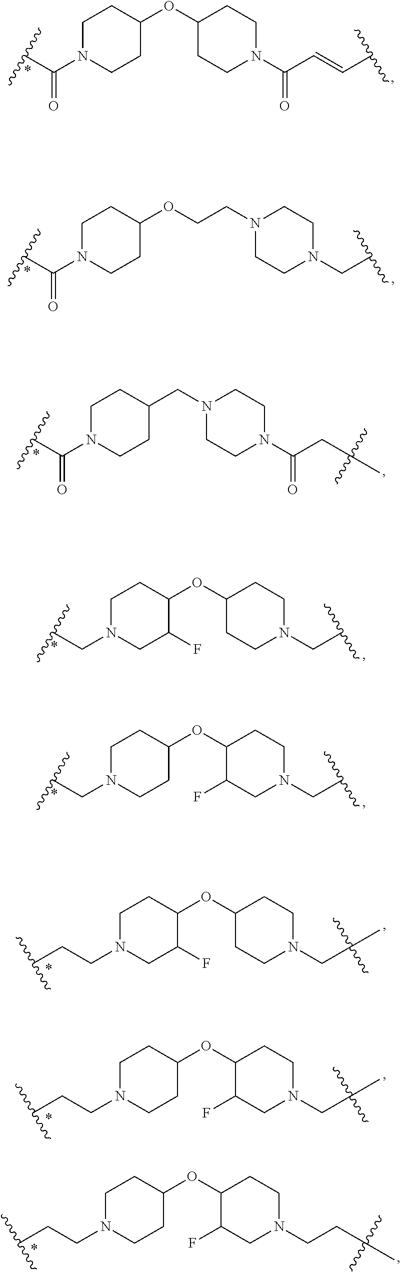

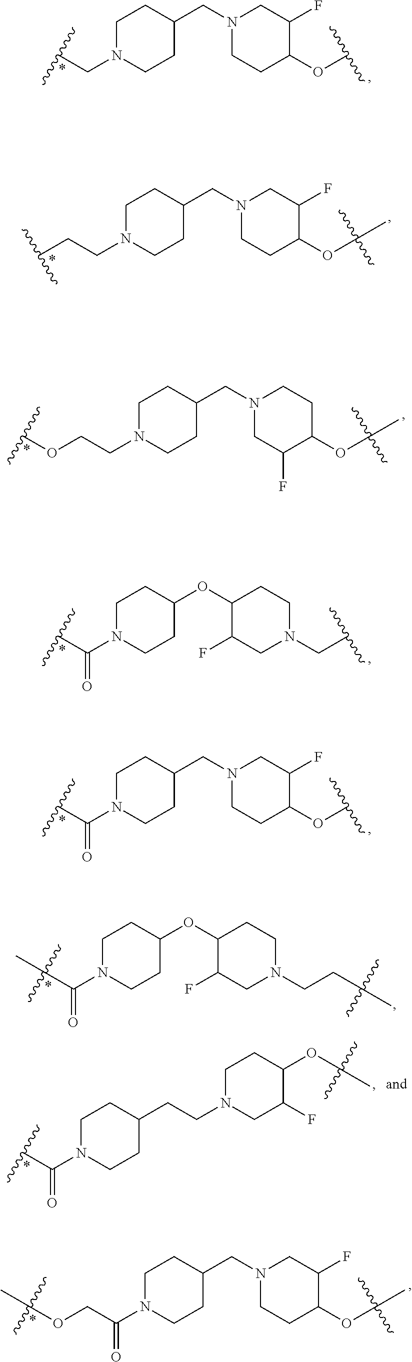

or a pharmaceutically acceptable salt, hydrate, solvate, prodrug, stereoisomer, or tautomer thereof, wherein “*” denotes the point of attachment to the Target Ligand or DUB Recruiter. In some embodiments, the linker (e.g., L1) is a variant of a linker described herein, e.g., wherein the linker (e.g., L1) comprises an additional C₁-C₆ alkyl or C₁-C₆ heteroalkyl moiety at a point of attachment, e.g., to the Target Ligand or the DUB Recruiter.

In an embodiment, the linker (e.g., L1) is selected from the group consisting of:

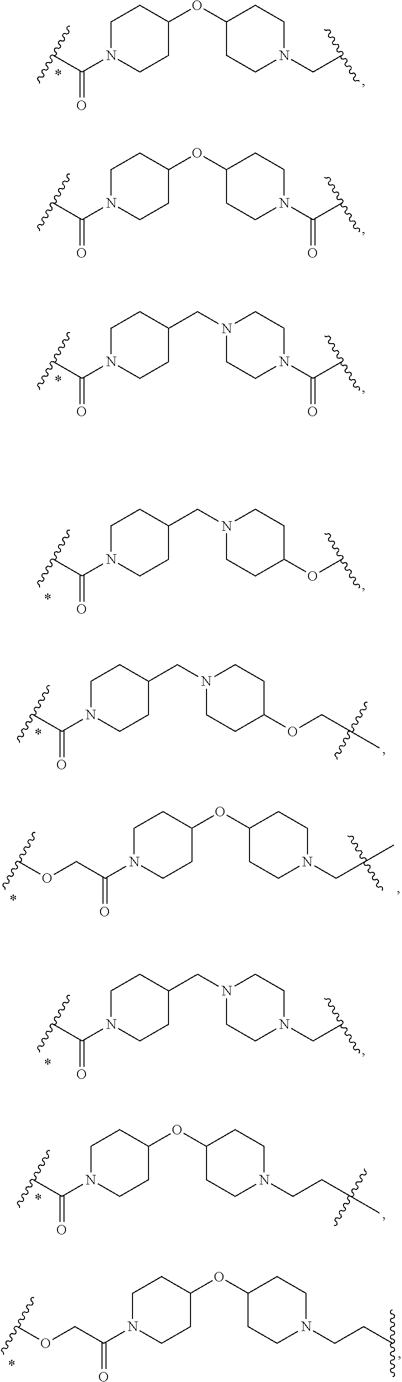

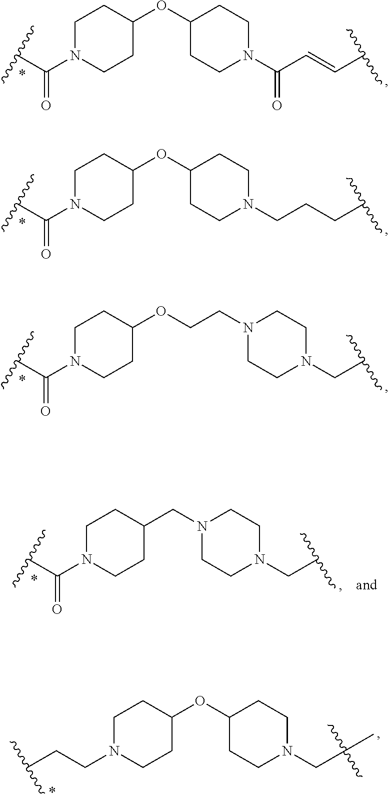 [SMILES: */C=C/C(=O)N1CCC(OC2CCN(C(*)=O)CC2)CC1 ; *CCCN1CCC(OC2CCN(C(*)=O)CC2)CC1 ; *CN1CCN(CCOC2CCN(C(*)=O)CC2)CC1 ; *CN1CCN(CC2CCN(C(*)=O)CC2)CC1 ; *CCN1CCC(OC2CCN(C*)CC2)CC1]

or a pharmaceutically acceptable salt, hydrate, solvate, prodrug, stereoisomer, or tautomer thereof, wherein “*” denotes the point of attachment to the Target Ligand or the DUB Recruiter. In some embodiments, the linker (e.g., L1) is a variant of a linker described herein, e.g., wherein the linker (e.g., L1) comprises an additional C₁-C₆ alkyl or C₁-C₆ heteroalkyl moiety at a point of attachment, e.g., to the Target Ligand or the DUB Recruiter.

In an embodiment, L1 has the structure of Formula (L1-I):

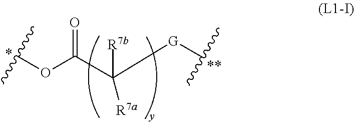

or a pharmaceutically acceptable salt, hydrate, solvate, prodrug, stereoisomer, or tautomer thereof, wherein each of R^{7a} and R^{7b} is independently H, C₁₋₆ alkyl, C₁₋₆ haloalkyl, C₁₋₆ heteroalkyl, cycloalkyl, and halo; G is absent, C₁₋₆ alkyl, C₁₋₆ heteroalkyl, cycloalkyl, heterocyclyl, aryl, heteroaryl, aryl-(C₁₋₆)alkylene, heteroaryl-(C₁₋₆)alkylene, aryl-(C₁₋₆)heteroalkylene, heteroaryl-(C₁₋₆)heteroalkylene, or —NR′—, wherein R′ is H, C₁₋₆ alkyl, or —(CH₂)₁₋₂—C(O)₂H, wherein each alkyl, heteroalkyl, cycloalkyl, heterocyclyl, aryl, or heteroaryl is substituted with 0-6 occurrences of R^{c}, wherein R^{c} is selected from the group consisting of halo, —C(O)OCH₂-aryl, and —C(O)OCH₂-heteroaryl; y is 0, 1, 2, 3, 4, or 5; and each “*” and “**” independently denote the point of attachment to the Target Ligand or DUB Recruiter in Formula (I).

In an embodiment, L1 is selected from the group consisting of:

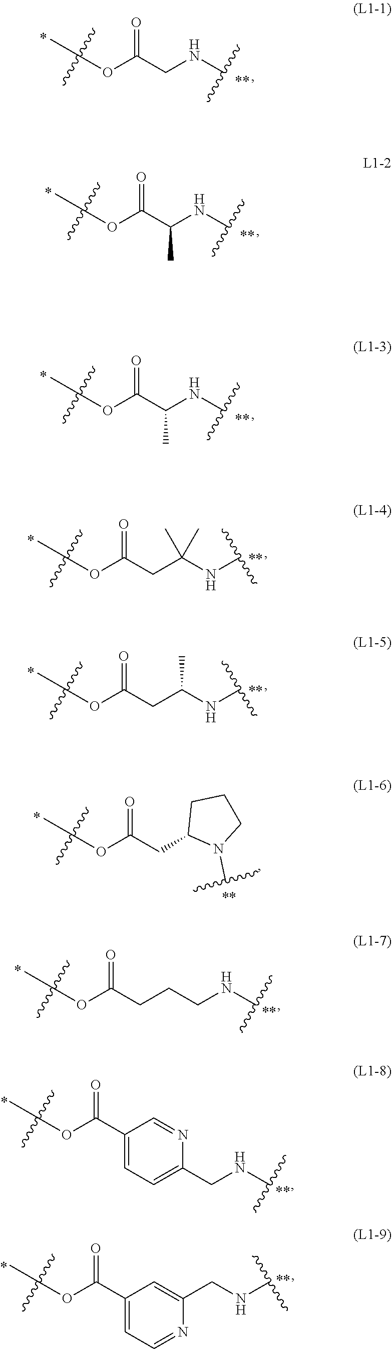

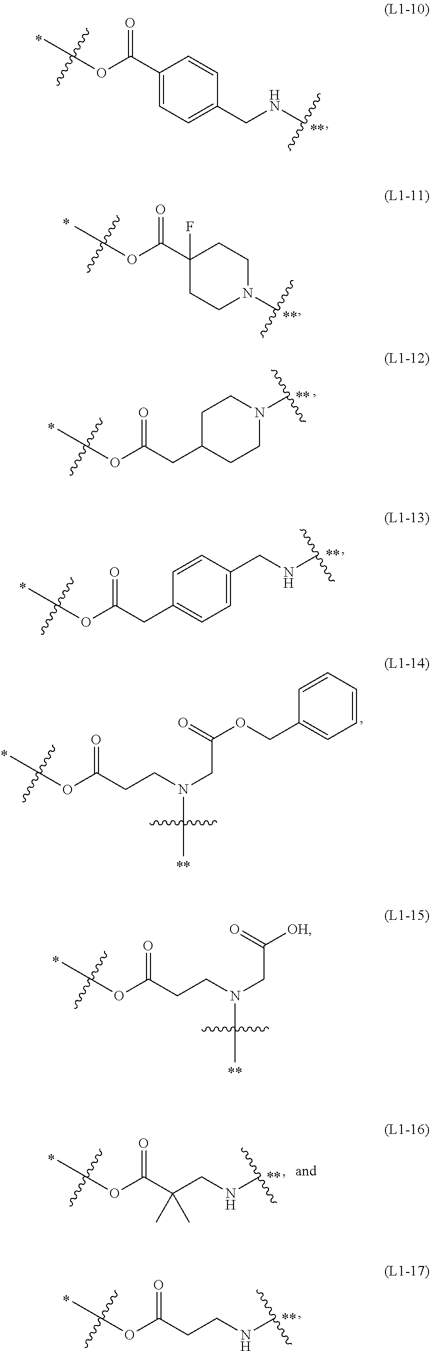

or a pharmaceutically acceptable salt, hydrate, solvate, prodrug, stereoisomer, or tautomer thereof, wherein “*” and “**” each independently denote the point of attachment to the Target Ligand or the DUB Recruiter. In some embodiments, the linker (e.g., L1) is a variant of a linker described herein, e.g., wherein the linker (e.g., L1) comprises an additional C₁-C₆ alkyl or C₁-C₆ heteroalkyl moiety at a point of attachment, e.g., to the Target Ligand or the DUB Recruiter.

### DUB Recruiter

The DUB Recruiter within the bifunctional compound is a small molecule moiety capable of binding to a cysteine amino acid residue within a deubiquitinase. The DUB Recruiter may bind to the deubiquitinase covalently or non-covalently. In some embodiments, the DUB Recruiter binds to the deubiquitinase covalently, e.g., through a thiol or thioester bond. In some embodiments, the DUB Recruiter binds to the deubiquitinase non-covalently, e.g., ionically.

In some embodiments, the DUB Recruiter binds to any deubiquitinase, e.g., in a cell, including cysteine protease deubiquitinases and metalloprotease deubiquitinases. In some embodiments, the DUB Recruiter binds to a cysteine protease deubiquitinase, e.g., comprising a catalytic site cysteine amino acid residue. The DUB Recruiter may bind to a full-length deubiquitinase or a fragment thereof. In some embodiments, the DUB Recruiter binds to a surface of deubiquitinase. In some embodiments, the DUB Recruiter binds to an internal cavity of the deubiquitinase. In some embodiments, the DUB Recruiter binds to a deubiquitinase selected from the group consisting of BAP1, CYLD, OTUB1, OTUB2, OTUD3, OTUD5, OTUD7A, OTUD7B, TNFAIP3, UCHL1, UCHL3, UCHL5, USP10, USP11, USP12, USP13, USP14, USP15, USP16, USP17L1, USP17L2, USP17L24, USP17L3, USP17L5, USP18, USP19, USP2, USP20, USP21, USP22, USP24, USP25, USP26, USP27X, USP28, USP3, USP30, USP31, USP33, USP34, USP35, USP36, USP37, USP38, USP4, USP40, USP41, USP42, USP43, USP44, USP45, USP46, USP47, USP48, USP49, USP5, USP50, USP51, USP54, USP7, USP8, USP9X, VCPIP1, WDR48, YOD1, ZRANB1, and ZUP1, or a fragment or variant thereof. In some embodiments, the DUB Recruiter binds to a deubiquitinase selected from the group consisting of WDR48, YOD1, OYUD3, OTUB1, USP8, USP5, USP16, UCHL3, UCHL1, and USP14, or a fragment thereof. In some embodiments, the DUB Recruiter binds to a deubiquitinase selected from the group consisting of WDR48, YOD1, OYUD3, OTUB1, OTUD5, USP8, USP5, USP14, USP15, USP16, UCHL3, and UCHL1, or a fragment thereof. In some embodiments, the DUB Recruiter binds to OTUB1 or a fragment or variant thereof. In some embodiments, the DUB Recruiter binds to OTUD5 or a fragment or variant thereof. In some embodiments, the DUB Recruiter binds to USP15 or a fragment or variant thereof. In some embodiments, the DUB Recruiter binds to a deubiquitinase listed in Table 1.

In some embodiments, the DUB Recruiter binds to a site other than a catalytic site within the deubiquitinase. In some embodiments, the DUB Recruiter binds to an allosteric site within the deubiquitinase. In some embodiments, binding of the DUB Recruiter to the deubiquitinase does not modulate the activity of the deubiquitinase more than 0.1, 0.5, 1, 1.5, 2, 2.5, 3, 4, 5, 6, 7, 8, 9, 10, 15, 20, 25, 30, 35, 40, 45, 50, 55, 60, 65, 70, 75, 80, 85, 90, 95, or 99%, relative to the activity of the deubiquitinase in the absence of the DUB Recruiter. In some embodiments, binding of the DUB Recruiter to the deubiquitinase does not modulate the activity of the deubiquitinase more than 0.1-50%, 1-50%, 1-25%, 1-10%, 0.1-10%, 1-5%, or 0.1-2%, relative to the activity of the deubiquitinase in the absence of the DUB Recruiter. In some embodiments, the binding of the DUB Recruiter to the deubiquitinase does not substantially modulate (e.g., inhibit) the activity (e.g., deubiquitinase activity) of the deubiquitinase.

In some embodiments, the DUB Recruiter binds to a site other than a catalytic site within the deubiquitinase. In some embodiments, the DUB Recruiter binds to an allosteric site within the deubiquitinase. In some embodiments, the DUB Recruiter binds to a cysteine amino acid residue within the deubiquitinase. In some embodiments, the DUB Recruiter preferentially binds to an allosteric amino acid residue (e.g., an allosteric cysteine amino acid residue) over a catalytic amino acid residue (e.g., a catalytic cysteine amino acid residue). In some embodiments, the DUB Recruiter does not substantially bind to a cysteine amino acid residue in the catalytic site of the deubiquitinase (e.g., a catalytic cysteine).

In some embodiments, the DUB Recruiter comprises a functional group selected from the group consisting of an amide, heterocyclyl, cycloalkyl, heterocyclyl, cycloalkyl, carbonyl, ester, alkyl, alkenyl, alkynyl, acyl, or acrylamide. In some embodiments, the DUB Recruiter comprises a heterocyclyl (e.g., a piperazinonyl). In some embodiments, the DUB Recruiter comprises an acrylamide moiety. In some embodiments, the DUB Recruiter comprises a heteroaryl (e.g., a furan moiety).

In some embodiments, the DUB Recruiter has the structure of Formula (V-a):

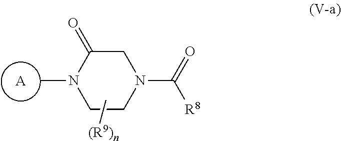

or a pharmaceutically acceptable salt, hydrate, solvate, prodrug, stereoisomer, or tautomer thereof, wherein Ring A is cycloalkyl, heterocyclyl, aryl, or heteroaryl, each of which is substituted with 0-12 R¹⁰; R⁸ is H, C₁₋₄ alkyl, or an electrophilic moiety; each R⁹ is independently C₁₋₆ alkyl, C₁₋₆ haloalkyl, C₁₋₆ heteroalkyl, halo, or —OR^{A}; each R¹⁰ is independently C₁₋₆ alkyl, C₁₋₆ haloalkyl, C₁₋₆ heteroalkyl, or halo; R^{A} is H, C₁₋₆ alkyl, C₂₋₆ alkenyl, C₂₋₆ alkynyl, C₁₋₆ haloalkyl, C₁₋₆ heteroalkyl, halo, cycloalkyl, heterocyclyl, aryl, or heteroaryl; and n is 0, 1, 2, 3, 4, 5, or 6.

In some embodiments, the DUB Recruiter has the structure of Formula (V-b):

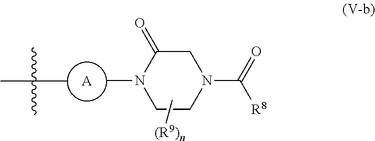

or a pharmaceutically acceptable salt, hydrate, solvate, prodrug, stereoisomer, or tautomer thereof, wherein Ring A is cycloalkyl, heterocyclyl, aryl, or heteroaryl, each of which is substituted with 0-12 R¹⁰; R⁸ is H, C₁₋₄ alkyl, or an electrophilic moiety; each R⁹ is independently C₁₋₆ alkyl, C₁₋₆ haloalkyl, C₁₋₆ heteroalkyl, halo, or —OR^{A}; each R¹⁰ is independently C₁₋₆ alkyl, C₁₋₆ haloalkyl, C₁₋₆ heteroalkyl, or halo; R^{A} is H, C₁₋₆ alkyl, C₂₋₆ alkenyl, C₂₋₆ alkynyl, C₁₋₆ haloalkyl, C₁₋₆ heteroalkyl, halo, cycloalkyl, heterocyclyl, aryl, or heteroaryl; and n is 0, 1, 2, 3, 4, 5, or 6, wherein [symbol] denotes the point of attachment to L1 in Formula (I).

In some embodiments, the DUB Recruiter has the structure of Formula (V-d):

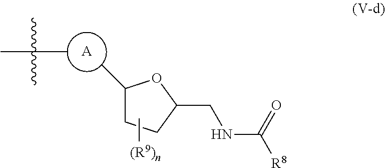

or a pharmaceutically acceptable salt, hydrate, solvate, prodrug, stereoisomer, or tautomer thereof, wherein Ring A is cycloalkyl, heterocyclyl, aryl, or heteroaryl, each of which is substituted with 0-12 R¹⁰; R⁸ is H, C₁₋₄ alkyl, or an electrophilic moiety; each R⁹ is independently C₁₋₆ alkyl, C₁₋₆ haloalkyl, C₁₋₆ heteroalkyl, halo, or —OR^{A}; each R¹⁰ is independently C₁₋₆ alkyl, C₁₋₆ haloalkyl, C₁₋₆ heteroalkyl, or halo; R^{A} is H, C₁₋₆ alkyl, C₂₋₆ alkenyl, C₂₋₆ alkynyl, C₁₋₆ haloalkyl, C₁₋₆ heteroalkyl, halo, cycloalkyl, heterocyclyl, aryl, or heteroaryl; and n is 0, 1, 2, 3, 4, 5, or 6, wherein denotes the point of attachment to L1 in [symbol] Formula (I).

In some embodiments, the DUB Recruiter has the structure of Formula (V-e):

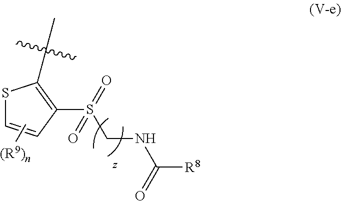

or a pharmaceutically acceptable salt, hydrate, solvate, prodrug, stereoisomer, or tautomer thereof, wherein R⁸ is H, C₁₋₆ alkyl, or an electrophilic moiety; each R⁹ is independently C₁₋₆ alkyl, C₁₋₆ haloalkyl, C₁₋₆ heteroalkyl, halo, or —OR^{A}; and n is 0, 1, or 2, wherein [symbol] denotes the point of attachment to L1 in Formula (I).

In some embodiments, the DUB Recruiter has the structure of Formula (V-f):

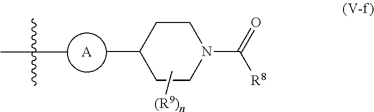

or a pharmaceutically acceptable salt, hydrate, solvate, prodrug, stereoisomer, or tautomer thereof, wherein Ring A is cycloalkyl, heterocyclyl, aryl, or heteroaryl, each of which is substituted with 0-12 R¹⁰; R⁸ is H, C₁₋₄ alkyl, or an electrophilic moiety; each R⁹ is independently C₁₋₆ alkyl, C₁₋₆ haloalkyl, C₁₋₆ heteroalkyl, halo, or —OR^{A}; each R¹⁰ is independently C₁₋₆ alkyl, C₁₋₆ haloalkyl, C₁₋₆ heteroalkyl, or halo; R^{A} is H, C₁₋₆ alkyl, C₂₋₆ alkenyl, C₂₋₆ alkynyl, C₁₋₆ haloalkyl, C₁₋₆ heteroalkyl, halo, cycloalkyl, heterocyclyl, aryl, or heteroaryl; and n is 0, 1, 2, 3, 4, 5, 6, 7, 8, or 9 wherein [symbol] denotes the point of attachment to L1 in Formula (I).

In some embodiments, Ring A is heteroaryl (e.g., a monocyclic heteroaryl). In some embodiments, Ring A is a 5-membered heteroaryl (e.g., furanyl). In some embodiments, R⁸ is an electrophilic moiety. In some embodiments, R⁸ is H, C₁₋₆ alkyl, C₂₋₆ alkenyl, C₂₋₆ alkynyl, C₁₋₆ haloalkyl, C₁₋₆ heteroalkyl, halo, cyano, azido, cycloalkyl, heterocyclyl, aryl, or heteroaryl, wherein alkyl, alkenyl, alkynyl, haloalkyl, heteroalkyl, cycloalkyl, heterocyclyl, aryl, and heteroaryl is substituted with 0-12 R¹⁰. In some embodiments, R⁸ is C₂₋₆ alkenyl (e.g., CH═CH₂). In some embodiments, n is 0.

In some embodiments, R⁸ is an electrophilic moiety. In some embodiments, R⁸ is a structure selected from one of:

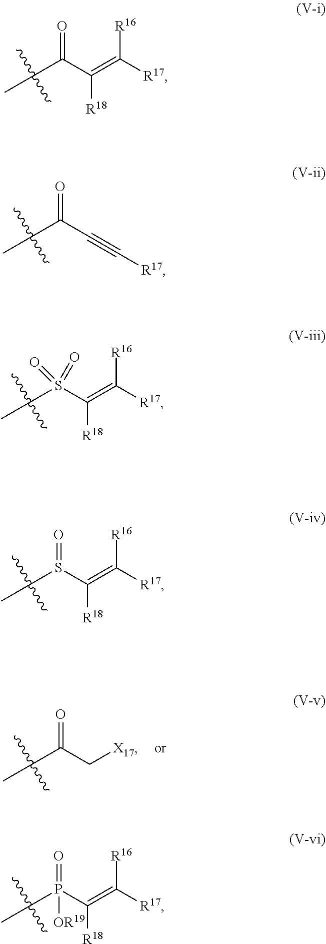

or a pharmaceutically acceptable salt, hydrate, solvate, prodrug, stereoisomer, or tautomer thereof, wherein:
R¹⁶ is H, halogen, —CX¹⁶₃, —CHX¹⁶₂, —CH₂X¹⁶, —CN, —SOₙ₁₆R^{16A}SOᵥ₁₆NR^{16A}R^{16B}, —NHNR^{16A}R^{16B}, —ONR^{16A}R^{16B}, —NHC(O)NHNR^{16A}R^{16B}, —N(O)ₘ₁₆, —NR^{16A}R^{16B}, —C(O)R^{16A}, —C(O)—OR^{16A}, —C(O)NR^{16A}R^{16B}, —OR^{16A}, NHC(O)NR^{16A}R^{16B}, —NR^{16A}SO₂R^{16B}, —NR^{16A}C(O)R^{16B}, —NR^{16A}C(O)OR^{16B}, —NR^{16A}OR^{16B}, —OCX¹⁶₃, —OCHX¹⁶², —OCH₂X¹⁶, C₁₋₆ alkyl, C₁₋₆ heteroalkyl, cycloalkyl, heterocyclyl, aryl, heteroaryl, wherein each alkyl, heteroalkyl, cycloalkyl, heterocyclyl, aryl, and heteroaryl is substituted with 0-12 R²⁵;
R¹⁷ is H, halogen, —CX¹⁷³, —CHX¹⁷², —CH₂X¹⁷, —CN, —SOₙ₁₇R^{17A}SOᵥ₁₇NR^{17A}R^{17B}, —NHNR^{17A}R^{17B}, —ONR^{17A}R^{17B}, —NHC(O)NHNR^{17A}R^{17B}, —NHC(O)NR^{17A}R^{17B}, —N(O)ₘ₁₇, —NR^{17A}R^{17B}, —C(O)R^{17A}, —C(O)—OR^{17A}, —C(O)NR^{17A}R^{17B}, —OR^{17A}, —NR^{17A}SO₂R^{17B}, —NR^{17A}C(O)R^{17B}, —NR^{17A}C(O)OR^{17B}, —NR^{17A}OR^{17B}, —OCX¹⁷₃, —OCHX¹⁷₂, —OC H₂X¹⁷, C₁₋₆ alkyl, C₁₋₆ heteroalkyl, cycloalkyl, heterocyclyl, aryl, or heteroaryl, wherein each alkyl, heteroalkyl, cycloalkyl, heterocyclyl, aryl, and heteroaryl is substituted with 0-12 R²⁵;
R¹⁸ is H, halogen, —CX¹⁸³, —CHX¹⁸², —CH₂X¹⁸, —CN, —SOₙ₁₈R^{18A}, —SOᵥ₁₈NR^{18A}R^{18B}, —NHNR^{18A}R^{18B}, —ONR^{18A}R^{18B}, —NHC(O)NHNR^{18A}R^{18B}, —NHC(O)NR^{18A}R^{18B}, —N(O)ₘ₁₈, —NR^{18A}R^{18B}, —C(O)R^{18A}, —C(O)—OR^{18A}, —C(O)NR^{18A}R^{18B}, —OR^{18A}, —NR^{18A}SO₂R^{18B}, —NR¹⁸AC(O)R^{18B}, —NR¹⁸AC(O)OR^{18B}, —NR^{18A}OR^{18B}, —OCX¹⁸³, —OCHX¹⁸², —OCH₂X¹⁸, C₁₋₆ alkyl, C₁₋₆ heteroalkyl, cycloalkyl, heterocyclyl, aryl, or heteroaryl, wherein each alkyl, heteroalkyl, cycloalkyl, heterocyclyl, aryl, and heteroaryl is substituted with 0-12 R²⁵;
R¹⁹ is H, halogen, —CX¹⁹₃, —CHX¹⁹₂, —CH₂X¹⁹, —CN, —SOₙ₁₉R^{19A}, —SOᵥ₁₉NR^{19A}R^{19B}, —NHNR^{19A}R^{19B}, —ONR^{19A}R^{19B}, —NHC(O)NHNR^{19A}R^{19B}, —NHC(O)NR^{19A}R^{19B}, —N(O)ₘ₁₉, —NR^{19A}R^{19B}, —C(O)R^{19A}, —C(O)—OR^{19A}, —C(O)NR^{19A}R^{19B}, —OR¹⁹A —NR^{19A}SO₂R^{19B}, —NR¹⁹AC(O)R^{19B}, —NR¹⁹AC(O)OR^{19B}, —NR^{19A}OR^{19B}, —OCX¹⁹³, —OCHX¹⁹², —OC H₂X¹⁹, C₁₋₆ alkyl, C₁₋₆ heteroalkyl, cycloalkyl, heterocyclyl, aryl, or heteroaryl, wherein each alkyl, heteroalkyl, cycloalkyl, heterocyclyl, aryl, and heteroaryl is substituted with 0-12 R²⁵;
R^{16A}, R^{16B}, R^{17A}, R^{17B}, R^{18A}, R^{18B}, R^{19A}, and R^{19B} are each independently H, —CX₃, —CHX₂, —CH₂X, —CN, —OH, —COOH, —CONH₂, C₁₋₆ alkyl, C₁₋₆ heteroalkyl, cycloalkyl, heterocyclyl, aryl; or
R^{16A} and R^{16B} substituents bonded to the same nitrogen atom may optionally be joined to form a heterocyclyl or heteroaryl;
R^{17A} and R^{17B} substituents bonded to the same nitrogen atom may optionally be joined to form a heterocyclyl or heteroaryl;
R^{18A} and R^{18B} substituents bonded to the same nitrogen atom may optionally be joined to form a heterocyclyl or heteroaryl;
R^{19A} and R^{19B} substituents bonded to the same nitrogen atom may optionally be joined to form a heterocyclyl or heteroaryl;
each X, X¹⁶, X¹⁷, X¹⁸, and X¹⁹ is independently —F, —Cl, —Br, or —I;
n16, n17, n18, and n19 are independently an integer from 0 to 4; and
m16, m17, m18, m19, v16, v17, v18, and v19 are independently 1 or 2.

In some embodiments, R⁸ is selected from the group consisting of:

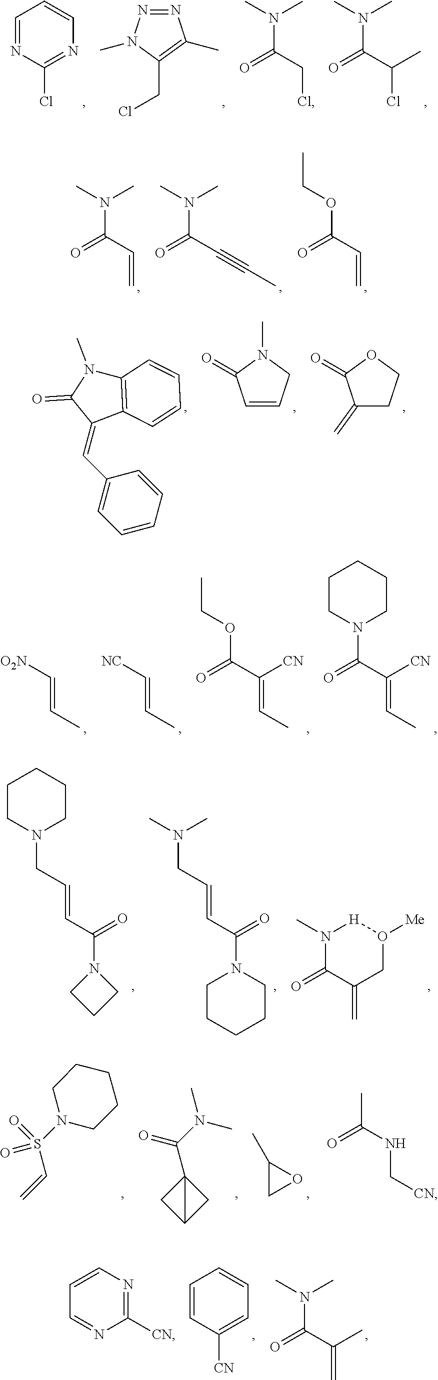

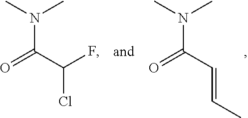

wherein the electrophilic moiety is bound to the structure of Formula (V-a) at any position.

In some embodiments, the DUB Recruiter is selected from the group consisting of:

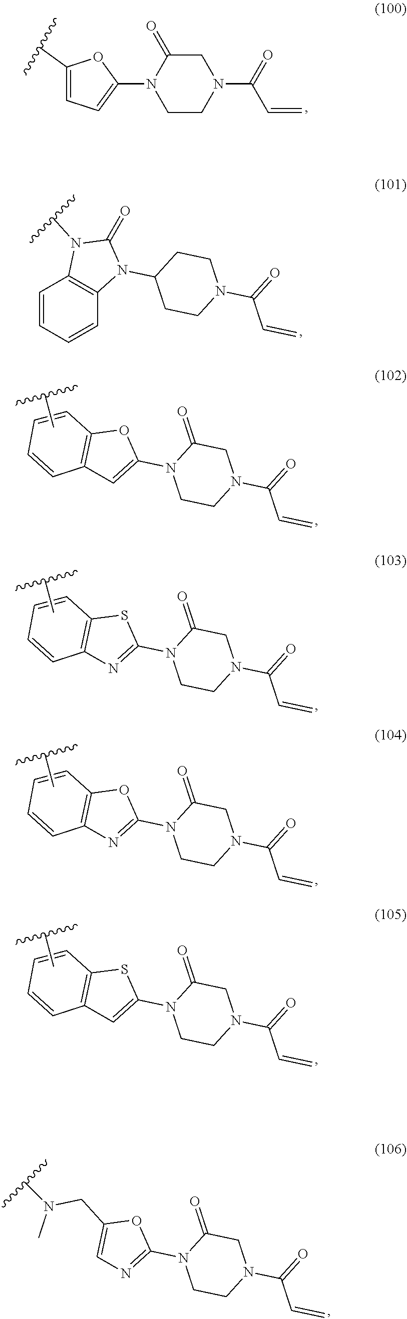

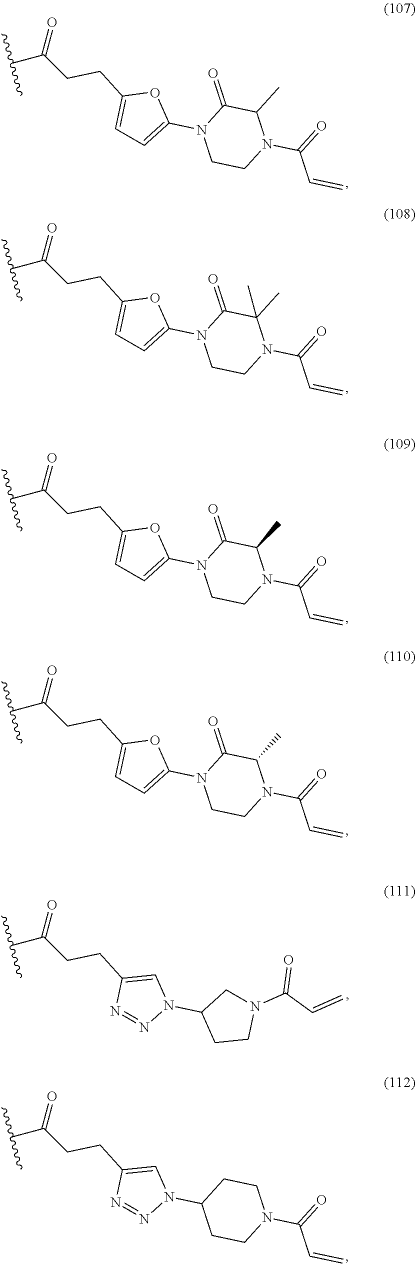 [SMILES: *C(=O)CCc1ccc(N2CCN(C(=O)C=C)C(C)C2=O)o1 ; *C(=O)CCc1ccc(N2CCN(C(=O)C=C)C(C)(C)C2=O)o1 ; *C(=O)CCc1ccc(N2CCN(C(=O)C=C)[C@H](C)C2=O)o1 ; *C(=O)CCc1ccc(N2CCN(C(=O)C=C)[C@@H](C)C2=O)o1 ; *C(=O)CCc1cn(C2CCN(C(=O)C=C)C2)nn1 ; *C(=O)CCc1cn(C2CCN(C(=O)C=C)CC2)nn1]

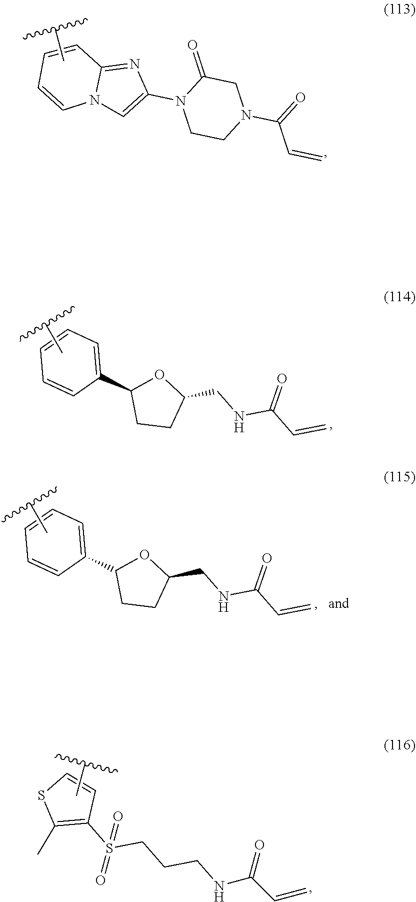

or a pharmaceutically acceptable salt, hydrate, solvate, prodrug, stereoisomer, or tautomer thereof, wherein [symbol] denotes the point of attachment to L1 in Formula (I).

In some embodiments, the DUB Recruiter is selected from the group consisting of:

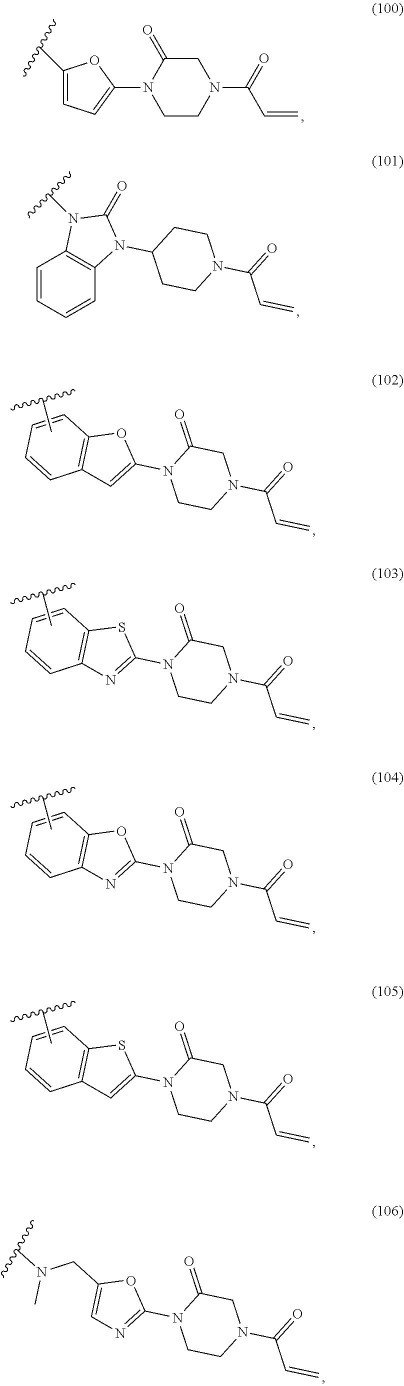

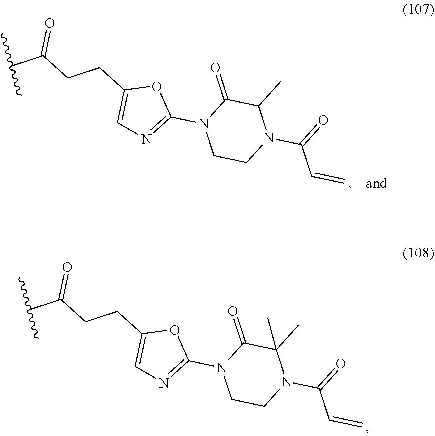 [SMILES: *C(=O)CCc1cnc(N2CCN(C(=O)C=C)C(C)C2=O)o1 ; *C(=O)CCc1cnc(N2CCN(C(=O)C=C)C(C)(C)C2=O)o1]

or a pharmaceutically acceptable salt, hydrate, solvate, prodrug, stereoisomer, or tautomer thereof, wherein [symbol] denotes the point of attachment to L1 in Formula (I).

In some embodiments, the DUB Recruiter is Compound 100:

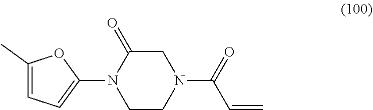

or a pharmaceutically acceptable salt, hydrate, solvate, prodrug, stereoisomer, or tautomer thereof, wherein [symbol] denotes the point of attachment to L1 in Formula (I).

In some embodiments, the DUB Recruiter is Compound 114:

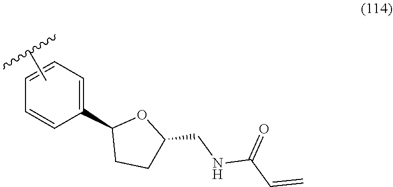

or a pharmaceutically acceptable salt, hydrate, solvate, prodrug, stereoisomer, or tautomer thereof, wherein [symbol] denotes the point of attachment to L1 in Formula (I).

In some embodiments, the DUB Recruiter is Compound 116:

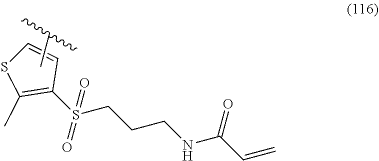

or a pharmaceutically acceptable salt, hydrate, solvate, prodrug, stereoisomer, or tautomer thereof, wherein [symbol] denotes the point of attachment to L1 in Formula (I).

In some embodiments, the bifunctional compound of Formula (I) has the structure (II-k):

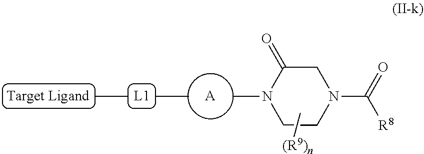

or a pharmaceutically acceptable salt, hydrate, solvate, prodrug, stereoisomer, or tautomer thereof, wherein Ring A is cycloalkyl, heterocyclyl, aryl, or heteroaryl, each of which is substituted with 0-12 R¹⁰; R⁸ is H, C₁₋₄ alkyl, or an electrophilic moiety; each R⁹ is independently C₁₋₆ alkyl, C₁₋₆ haloalkyl, C₁₋₆ heteroalkyl, halo, or —OR^{A}; each R¹⁰ is independently C₁₋₆ alkyl, C₁₋₆ haloalkyl, C₁₋₆ heteroalkyl, or halo; R^{A} is H, C₁₋₆ alkyl, C₂₋₆ alkenyl, C₂₋₆ alkynyl, C₁₋₆ haloalkyl, C₁₋₆ heteroalkyl, halo, cycloalkyl, heterocyclyl, aryl, or heteroaryl; n is 0, 1, 2, 3, 4, 5, or 6; wherein the Target Ligand and L1 are as defined as for Formula (I).

In some embodiments, Ring A is heteroaryl (e.g., a monocyclic heteroaryl). In some embodiments, Ring A is a 5-membered heteroaryl (e.g., furanyl). In some embodiments, R⁸ is an electrophilic moiety. In some embodiments, R⁸ is H, C₁₋₆ alkyl, C₂₋₆ alkenyl, C₂₋₆ alkynyl, C₁₋₆ haloalkyl, C₁₋₆ heteroalkyl, halo, cyano, azido, cycloalkyl, heterocyclyl, aryl, or heteroaryl, wherein alkyl, alkenyl, alkynyl, haloalkyl, heteroalkyl, cycloalkyl, heterocyclyl, aryl, and heteroaryl is substituted with 0-12 R¹⁰. In some embodiments, R⁸ is C₂₋₆ alkenyl (e.g., CH═CH₂). In some embodiments, n is 0.

In some embodiments, the bifunctional compound of Formula (I) has the structure (II-1):

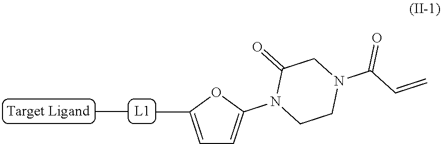

or a pharmaceutically acceptable salt, hydrate, solvate, prodrug, stereoisomer, or tautomer thereof, wherein the Target Ligand and L1 are as defined as for Formula (I).

In some embodiments, the bifunctional compound of Formula (I) has the structure (II-m):

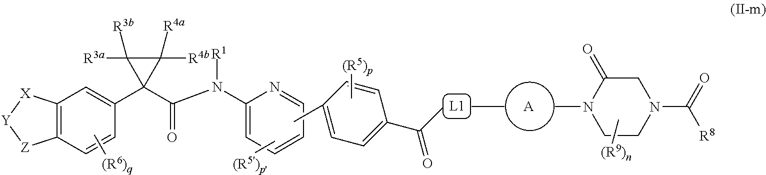

or a pharmaceutically acceptable salt, hydrate, solvate, prodrug, stereoisomer, or tautomer thereof, wherein X and Z are each independently O, S, or C(R^{7a})(R^{7b}); Y is C(R^{7a})(R^{7b}) or NR⁷⁰; Ring A is cycloalkyl, heterocyclyl, aryl, or heteroaryl, each of which is substituted with 0-12 R¹⁰; R¹ is H or C₁₋₆ alkyl; R^{3a}, R^{3b}, R^{4a}, R^{4b} are each independently H, C₁₋₆ alkyl, C₁₋₆ haloalkyl, C₁₋₆ heteroalkyl, halo, cyano, or —OR^{A}; each R⁵, R^{5′}, and R⁶ is independently C₁₋₄ alkyl, C₁₋₆ haloalkyl, C₁₋₆ heteroalkyl, halo, cyano, —OR^{A}, —C(O)N(R^{B})(R^{C}), or —N(R^{B})CO(R^{D}); R^{7a} and R^{7b} are each independently H, C₁₋₆ alkyl, C₁₋₄ haloalkyl, C₁₋₆ heteroalkyl, or halo; R⁷, is H or C₁₋₆ alkyl; R⁸ is H, C₁₋₆ alkyl, or an electrophilic moiety; R⁸ is H, C₁₋₆ s alkyl, or an electrophilic moiety; each R⁹ is independently C₁₋₆ alkyl, C₁₋₆ haloalkyl, C₁₋₆ heteroalkyl, halo, or —OR^{A}; each R¹⁰ is independently C₁₋₆ alkyl, C₁₋₆ haloalkyl, C₁₋₄ heteroalkyl, or halo; R^{A}, R^{B}, R^{C}, and R^{D} are each independently H, C₁₋₆ alkyl, C₂₋₆ alkenyl, C₂₋₆ alkynyl, C₁₋₆ haloalkyl, C₁₋₆ heteroalkyl, cycloalkyl, heterocyclyl, aryl, or heteroaryl; n is 0, 1, 2, 3, 4, 5, or 6; p is 0, 1, 2, 3, or 4; p′ is 0, 1, 2, 3, or 4; q is 0, 1, 2, or 3; and L1 is as defined as in Formula (I).

In some embodiments, the bifunctional compound of Formula (I) has the structure (II-n):

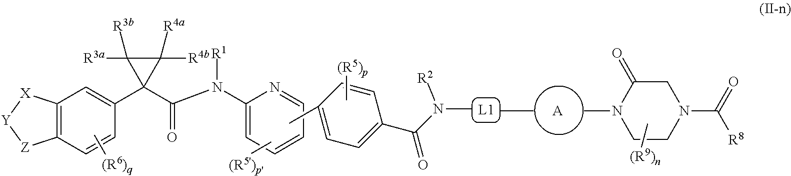

or a pharmaceutically acceptable salt, hydrate, solvate, prodrug, stereoisomer, or tautomer thereof, wherein X and Z are each independently O, S, or C(R^{7a})(R^{7b}); Y is C(R^{7a})(R^{7b}) or NR^{7c}; Ring A is cycloalkyl, heterocyclyl, aryl, or heteroaryl, each of which is substituted with 0-12 R¹⁰; R¹ is H or C₁₋₆ alkyl; R² is H or C₁₋₆ alkyl; R^{3a}, R^{3b}, R^{4a}, R^{4b} are each independently H, C₁₋₆ alkyl, C₁₋₆ haloalkyl, C₁₋₆ heteroalkyl, halo, cyano, or —OR^{A}; each R⁵, R^{5′}, and R⁶ is independently C₁₋₆ alkyl, C₁₋₆ haloalkyl, C₁₋₆ heteroalkyl, halo, cyano, —OR^{A}, —C(O)N(R^{B})(R^{C}) or —N(R^{B})CO(R^{D}); R^{7a} and R^{7b} are each independently H, C₁₋₆ alkyl, C₁₋₆ haloalkyl, C₁₋₆ heteroalkyl, or halo; R^{7c} is H or C₁₋₆ alkyl; R⁸ is H, C₁₋₆ alkyl, or an electrophilic moiety; R⁸ is H, C₁₋₆ alkyl, or an electrophilic moiety; each R⁹ is independently C₁₋₆ alkyl, C₁₋₆ haloalkyl, C₁₋₆ heteroalkyl, halo, or —OR^{A}; each R¹⁰ is independently C₁₋₆ alkyl, C₁₋₆ haloalkyl, C₁₋₆ heteroalkyl, or halo; R^{A}, R^{B}, R^{C}, and R^{D} are each independently H, C₁₋₆ alkyl, C₂₋₆ alkenyl, C₂₋₆ alkynyl, C₁₋₆-haloalkyl, C₁₋₆ heteroalkyl, cycloalkyl, heterocyclyl, aryl, or heteroaryl; n is 0, 1, 2, 3, 4, 5, or 6; p is 0, 1, 2, 3, or 4; p′ is 0, 1, 2, 3, or 4; q is 0, 1, 2, or 3; and L1 is as defined as in Formula (I).

In some embodiments, the bifunctional compound of Formula (I) has the structure (II-o):

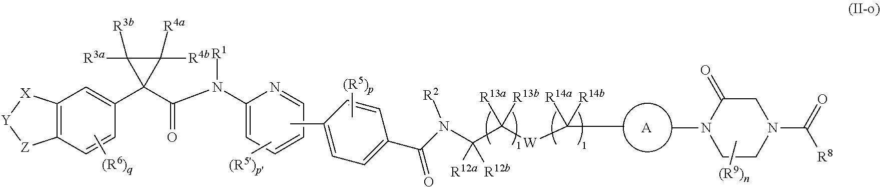

or a pharmaceutically acceptable salt, hydrate, solvate, prodrug, stereoisomer, or tautomer thereof, wherein X and Z are each independently O, S, or C(R^{7a})(R^{7b}); Y is C(R^{7a})(R^{7b}) or NR^{7c}; Ring A is cycloalkyl, heterocyclyl, aryl, or heteroaryl, each of which is substituted with 0-12 R¹⁰; R¹ is H or C₁₋₆ alkyl; R² is H or C₁₋₆ alkyl; R^{3a}, R^{3b}, R^{4a}, R^{4b} are each independently H, C₁₋₆ alkyl, C₁₋₆ haloalkyl, C₁₋₆ heteroalkyl, halo, cyano, or —OR^{A}; each R⁵, R^{5′}, and R⁶ is independently C₁₋₆ alkyl, C₁₋₆ haloalkyl, C₁₋₆ heteroalkyl, halo, cyano, —OR^{A}, —C(O)N(R^{B})(R^{C}), or —N(R^{B})CO(R^{D}); R^{7a} and R^{7b} are each independently H, C₁₋₆ alkyl, C₁₋₆ haloalkyl, C₁₋₆ heteroalkyl, or halo; R^{7c} is H or C₁₋₆ alkyl; R⁸ is H, C₁₋₆ alkyl, or an electrophilic moiety; each R⁹ is independently C₁₋₆ alkyl, C₁₋₆ haloalkyl, C₁₋₆ heteroalkyl, halo, or —OR^{A}; each R¹⁰ is independently C₁₋₆ alkyl, C₁₋₆ haloalkyl, C₁₋₆ heteroalkyl, or halo; R^{12a}, R^{12b}, R^{13a}, R^{13b}, R^{14a}, and R^{14b} are each independently H, C₁₋₆ alkyl, C₁₋₆ haloalkyl, C₁₋₆ heteroalkyl, halo, cyano, or —OR^{A}; or each of R^{12a} and R^{12b}, R^{13a} and R^{13b}, and R^{14a} and R^{14b} independently may be taken together with the carbon atom to which they are attached to form an oxo group; W is C(R^{15a})(R^{15b}), O, N(R¹⁶), or S; R^{15a} and R^{15b} are each independently H, C₁₋₆ alkyl, C₁₋₆ haloalkyl, C₁₋₆ heteroalkyl, halo, cyano, or —OR^{A}; or R^{15a} and R^{15b} may be taken together with the carbon atom to which they are attached to form an oxo group; R¹⁶ is H or C₁₋₆ alkyl; R^{A}, R^{B}, R^{C}, and R^{D} are each independently H, C₁₋₆ alkyl, C₂₋₆ alkenyl, C₂₋₆ alkynyl, C₁₋₆ haloalkyl, C₁₋₆ heteroalkyl, cycloalkyl, heterocyclyl, aryl, or heteroaryl; n is 0, 1, 2, 3, 4, 5, or 6; o and x are each independently an integer between 0 and 10; p is 0, 1, 2, 3, or 4; p′ is 0, 1, 2, 3, or 4; and q is 0, 1, 2, or 3.

In some embodiments, the bifunctional compound of Formula (I) has the structure (II-v):

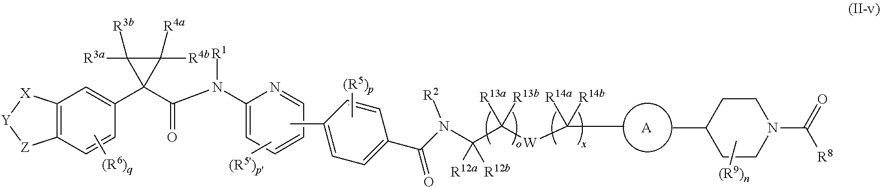

or a pharmaceutically acceptable salt, hydrate, solvate, prodrug, stereoisomer, or tautomer thereof, wherein X and Z are each independently O, S, or C(R^{7a})(R^{7b}); Y is C(R^{7a})(R^{7b}) or NR^{7c}; Ring A is cycloalkyl, heterocyclyl, aryl, or heteroaryl, each of which is substituted with 0-12 R¹⁰; R¹ is H or C₁₋₆ alkyl; R² is H or C₁₋₆ alkyl; R^{3a}, R^{3b}, R^{4a}, R^{4b} are each independently H, C₁₋₆ alkyl, C₁₋₆ haloalkyl, C₁₋₆ heteroalkyl, halo, cyano, or —OR^{A}; each R⁵, R^{5′}, and R⁶ is independently C₁₋₆ alkyl, C₁₋₆ haloalkyl, C₁₋₆ heteroalkyl, halo, cyano, —OR^{A}, —C(O)N(R^{B})(R_{C}), or —N(R^{B})CO(R^{D}); R^{7a} and R^{7b} are each independently H, C₁₋₆ alkyl, C₁₋₆ haloalkyl, C₁₋₆ heteroalkyl, or halo; R^{7c} is H or C₁₋₆ alkyl; R⁸ is H, C₁₋₆ alkyl, or an electrophilic moiety; each R⁹ is independently C₁₋₆ alkyl, C₁₋₆ haloalkyl, C₁₋₆ heteroalkyl, halo, or —OR^{A}; each R¹⁰ is independently C₁₋₆ alkyl, C₁₋₆ haloalkyl, C₁₋₆ heteroalkyl, or halo; R^{12a}, R^{12b}, R^{13a}, R^{13b}, R^{14a}, and R^{14b} are each independently H, C₁₋₆ alkyl, C₁₋₆ haloalkyl, C₁₋₆ heteroalkyl, halo, cyano, or —OR^{A}; or each of R^{12a} and R^{12b}, R^{13a} and R^{13b}, and R^{14a} and R^{14b} independently may be taken together with the carbon atom to which they are attached to form an oxo group; W is C(R^{15a})(R^{15b}), O, N(R¹⁶), or S; R^{15a} and R^{15b} are each independently H, C₁₋₆ alkyl, C₁₋₆ haloalkyl, C₁₋₆ heteroalkyl, halo, cyano, or —OR^{A}; or R^{15a} and R^{15b} may be taken together with the carbon atom to which they are attached to form an oxo group; R¹⁶ is H or C₁₋₆ alkyl; R^{A}, R^{B}, R^{C}, and R^{D} are each independently H, C₁₋₆ alkyl, C₂₋₆ alkenyl, C₂₋₆ alkynyl, C₁₋₆ haloalkyl, C₁₋₆ heteroalkyl, cycloalkyl, heterocyclyl, aryl, or heteroaryl; n is 0, 1, 2, 3, 4, 5, or 6; o and x are each independently an integer between 0 and 10; p is 0, 1, 2, 3, or 4; p′ is 0, 1, 2, 3, or 4; and q is 0, 1, 2, or 3.

In some embodiments, the bifunctional compound of Formula (I) has the structure (II-p):

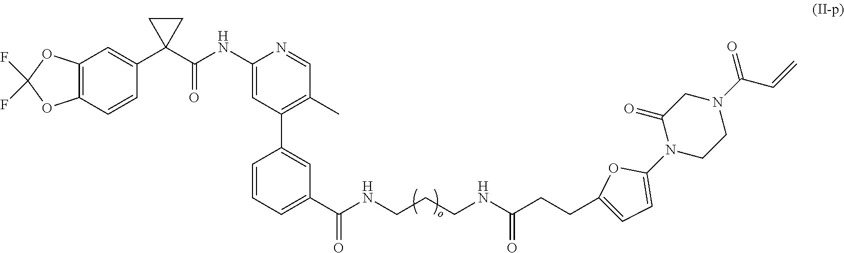

or a pharmaceutically acceptable salt, hydrate, solvate, prodrug, stereoisomer, or tautomer thereof, wherein o is selected from 0, 1, 2, 3, 4, 5, and 6. In some embodiments, o is 0. In some embodiments, o is 1. In some embodiments, o is 2. In some embodiments, o is 3. In some embodiments, o is 4.

In some embodiments, the bifunctional compound of Formula (I) has the structure (II-q):

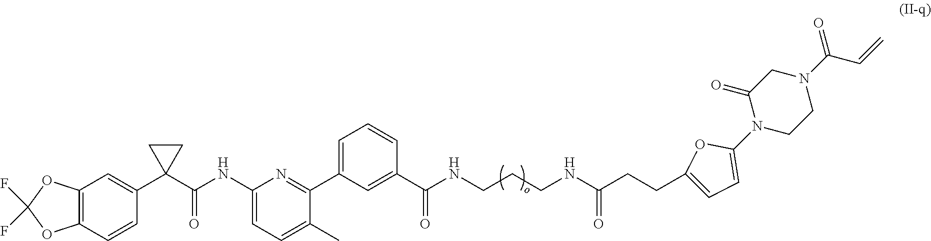

or a pharmaceutically acceptable salt, hydrate, solvate, prodrug, stereoisomer, or tautomer thereof, wherein o is selected from 0, 1, 2, 3, 4, 5, and 6. In some embodiments, o is 0. In some embodiments, o is 1. In some embodiments, o is 2. In some embodiments, o is 3. In some embodiments, o is 4.

In some embodiments, the bifunctional compound of Formula (I) has the structure (II-r):

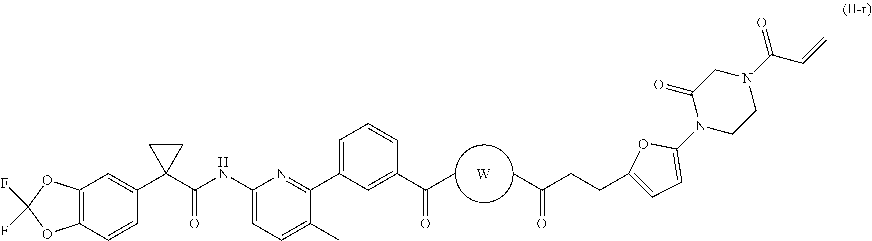

or a pharmaceutically acceptable salt, hydrate, solvate, prodrug, stereoisomer, or tautomer thereof, wherein W is heterocyclyl (e.g., monocyclic heterocyclyl or bicyclic heterocyclyl). In some embodiments, W is a nitrogen-containing heterocyclyl.

In some embodiments, the bifunctional compound of Formula (I) has the structure (II-s):

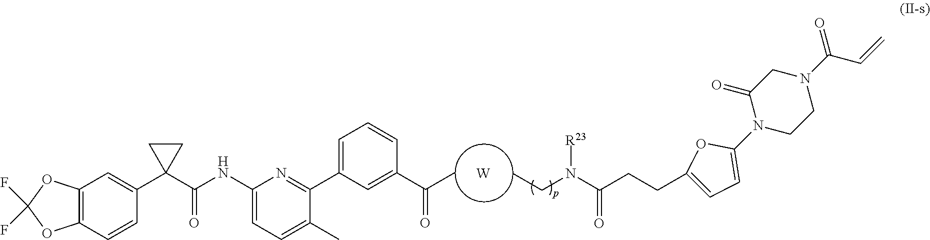

or a pharmaceutically acceptable salt, hydrate, solvate, prodrug, stereoisomer, or tautomer thereof, wherein W is heterocyclyl (e.g., monocyclic heterocyclyl or bicyclic heterocyclyl); R²³ is H or C₁₋₆ alkyl; and p is selected from 0, 1, 2, 3 or 4. In some embodiments, W is a nitrogen-containing heterocyclyl. In some embodiments, R²³ is C₁₋₄ alkyl. In some embodiments, and p is 1 or 2.

In some embodiments, the bifunctional compound of Formula (I) has the structure (II-t):

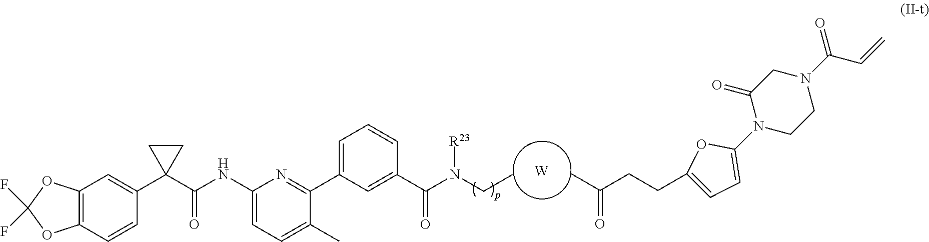

or a pharmaceutically acceptable salt, hydrate, solvate, prodrug, stereoisomer, or tautomer thereof, wherein W is heterocyclyl (e.g., monocyclic heterocyclyl or bicyclic heterocyclyl); R²³ is H or C₁₋₆ alkyl; and p is selected from 0, 1, 2, 3 or 4. In some embodiments, W is a nitrogen-containing heterocyclyl. In some embodiments, R²³ is H. In some embodiments, and p is 1 or 2.

In some embodiments, the bifunctional compound of Formula (I) has the structure (II-u):

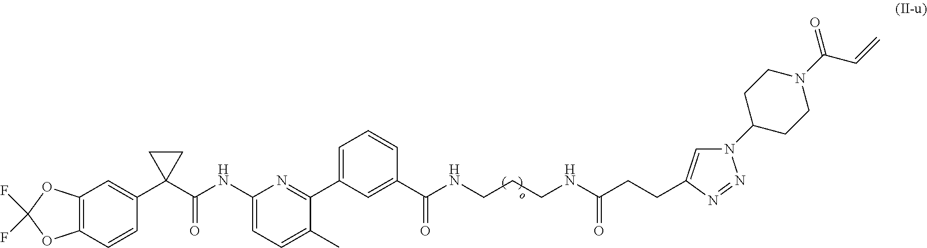

or a pharmaceutically acceptable salt, hydrate, solvate, prodrug, stereoisomer, or tautomer thereof, wherein o is selected from 0, 1, 2, 3, 4, 5, and 6. In some embodiments, o is 0. In some embodiments, o is 1. In some embodiments, o is 2. In some embodiments, o is 3. In some embodiments, o is 4.

In some embodiments, the bifunctional compound of Formula (I) has the structure (II-j):

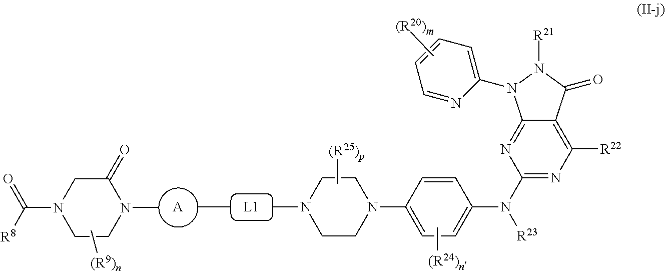

or a pharmaceutically acceptable salt, hydrate, solvate, prodrug, stereoisomer, or tautomer thereof, wherein each R²⁰, R²⁴, and R²⁵ is independently C₁₋₄ alkyl, C₂₋₆ alkenyl, C₂₋₆ alkynyl, C₁₋₆ haloalkyl, C₁₋₆ heteroalkyl, halo, cyano, —OR^{A}, —C(O)N(R^{B})(R^{C}), or —N(R^{B})CO(R^{D}R²¹ and R²³ are each independently H or C₁₋₆ alkyl; R²² is C₁₋₆ alkyl, C₂₋₆ alkenyl, C₂₋₆ alkynyl, C₁₋₆ haloalkyl, C₁₋₆ heteroalkyl, halo, cyano, —OR^{A}, —C(O)N(R^{B})(R^{C}), or —N(R^{B})CO(R^{D}); R^{A}, R^{B}R^{C}, and R^{D} are each independently H, C₁₋₆ alkyl, C₂₋₆ alkenyl, C₂₋₆ alkynyl, C₁₋₆ haloalkyl, C₁₋₆ heteroalkyl, cycloalkyl, heterocyclyl, aryl, or heteroaryl; m and n′ are each independently 0, 1, 2, 3, or 4; p is 0, 1, 2, 3, 4, 5, 6, 7, or 8; L1 is as defined as in Formula (I).

In some embodiments, the bifunctional compound is selected from a bifunctional compound listed in Table 2, or a pharmaceutically acceptable salt, hydrate, solvate, prodrug, stereoisomer, or tautomer thereof.

TABLE 2
Exemplary bifunctional compounds

| Compound | |
|---|---|
| No. | Structure |
| 200 | 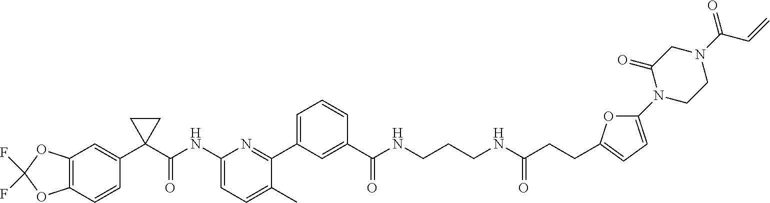 [SMILES: C=CC(=O)N1CCN(c2ccc(CCC(=O)NCCCNC(=O)c3cccc(-c4nc(NC(=O)C5(c6ccc7c(c6)OC(F)(F)O7)CC5)ccc4C)c3)o2)C(=O)C1] |
| 202 | 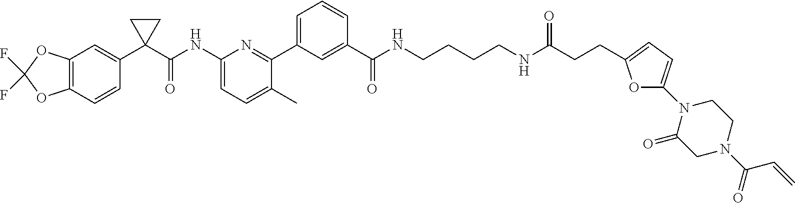 [SMILES: C=CC(=O)N1CCN(c2ccc(CCC(=O)NCCCCNC(=O)c3cccc(-c4nc(NC(=O)C5(c6ccc7c(c6)OC(F)(F)O7)CC5)ccc4C)c3)o2)C(=O)C1] |
| 201 | 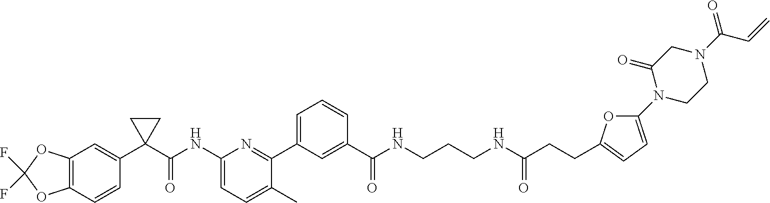 [SMILES: C=CC(=O)N1CCN(c2ccc(CCC(=O)NCCCNC(=O)c3cccc(-c4nc(NC(=O)C5(c6ccc7c(c6)OC(F)(F)O7)CC5)ccc4C)c3)o2)C(=O)C1] |
| 203 | 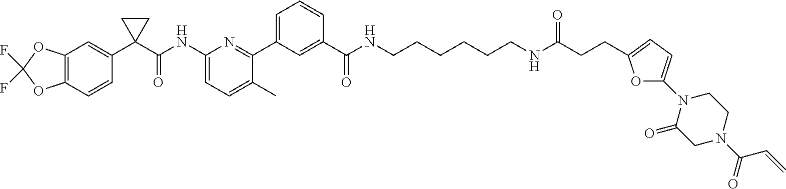 [SMILES: C=CC(=O)N1CCN(c2ccc(CCC(=O)NCCCCCCNC(=O)c3cccc(-c4nc(NC(=O)C5(c6ccc7c(c6)OC(F)(F)O7)CC5)ccc4C)c3)o2)C(=O)C1] |
| 204 | 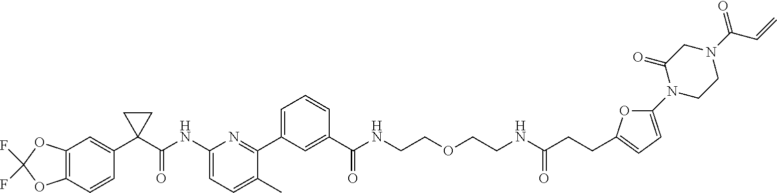 [SMILES: C=CC(=O)N1CCN(c2ccc(CCC(=O)NCCOCCNC(=O)c3cccc(-c4nc(NC(=O)C5(c6ccc7c(c6)OC(F)(F)O7)CC5)ccc4C)c3)o2)C(=O)C1] |
| 205 | 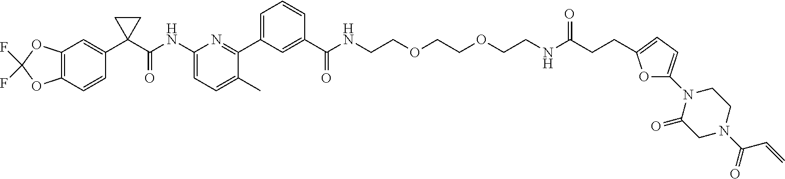 [SMILES: C=CC(=O)N1CCN(c2ccc(CCC(=O)NCCOCCOCCNC(=O)c3cccc(-c4nc(NC(=O)C5(c6ccc7c(c6)OC(F)(F)O7)CC5)ccc4C)c3)o2)C(=O)C1] |
| 206 | 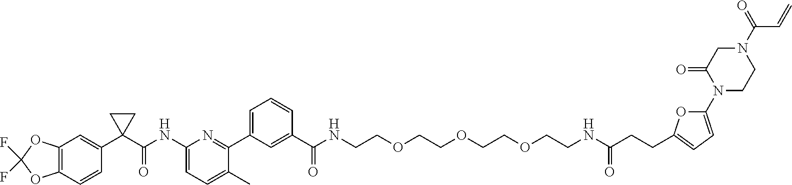 [SMILES: C=CC(=O)N1CCN(c2ccc(CCC(=O)NCCOCCOCCOCCNC(=O)c3cccc(-c4nc(NC(=O)C5(c6ccc7c(c6)OC(F)(F)O7)CC5)ccc4C)c3)o2)C(=O)C1] |
| 207 | 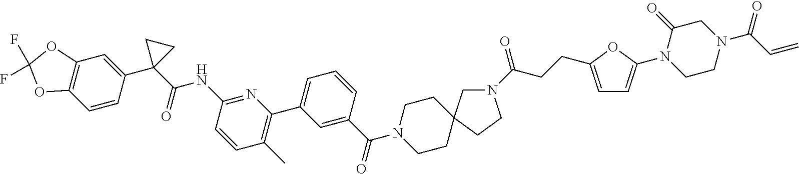 [SMILES: C=CC(=O)N1CCN(c2ccc(CCC(=O)N3CCC4(CCN(C(=O)c5cccc(-c6nc(NC(=O)C7(c8ccc9c(c8)OC(F)(F)O9)CC7)ccc6C)c5)CC4)C3)o2)C(=O)C1] |
| 208 | 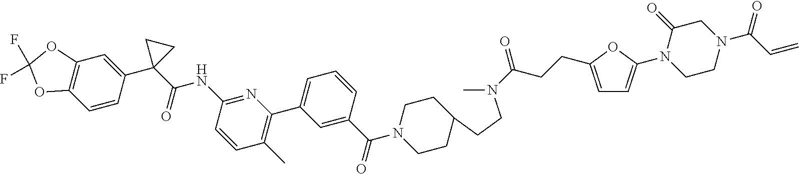 [SMILES: C=CC(=O)N1CCN(c2ccc(CCC(=O)N(C)CCC3CCN(C(=O)c4cccc(-c5nc(NC(=O)C6(c7ccc8c(c7)OC(F)(F)O8)CC6)ccc5C)c4)CC3)o2)C(=O)C1] |
| 209 | 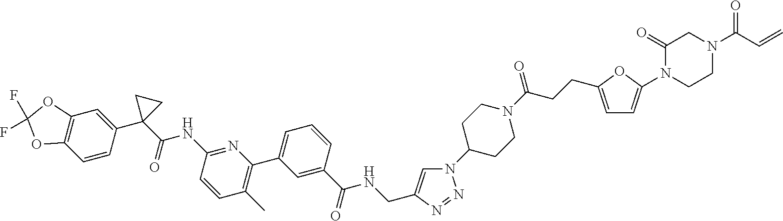 [SMILES: C=CC(=O)N1CCN(c2ccc(CCC(=O)N3CCC(n4cc(CNC(=O)c5cccc(-c6nc(NC(=O)C7(c8ccc9c(c8)OC(F)(F)O9)CC7)ccc6C)c5)nn4)CC3)o2)C(=O)C1] |
| 210 | 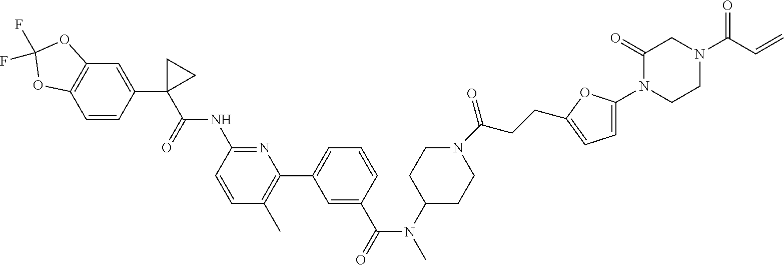 [SMILES: C=CC(=O)N1CCN(c2ccc(CCC(=O)N3CCC(N(C)C(=O)c4cccc(-c5nc(NC(=O)C6(c7ccc8c(c7)OC(F)(F)O8)CC6)ccc5C)c4)CC3)o2)C(=O)C1] |
| 211 | 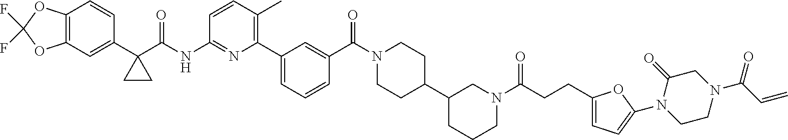 [SMILES: C=CC(=O)N1CCN(c2ccc(CCC(=O)N3CCCC(C4CCN(C(=O)c5cccc(-c6nc(NC(=O)C7(c8ccc9c(c8)OC(F)(F)O9)CC7)ccc6C)c5)CC4)C3)o2)C(=O)C1] |
| 212 | 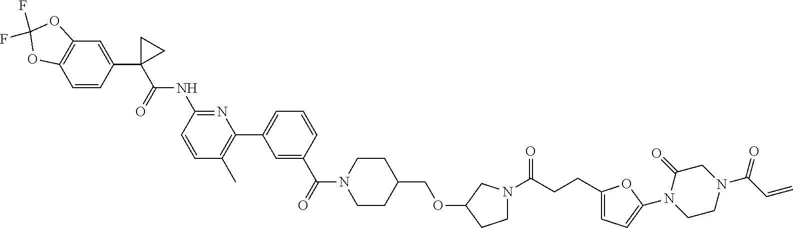 [SMILES: C=CC(=O)N1CCN(c2ccc(CCC(=O)N3CCC(OCC4CCN(C(=O)c5cccc(-c6nc(NC(=O)C7(c8ccc9c(c8)OC(F)(F)O9)CC7)ccc6C)c5)CC4)C3)o2)C(=O)C1] |
| 213 | 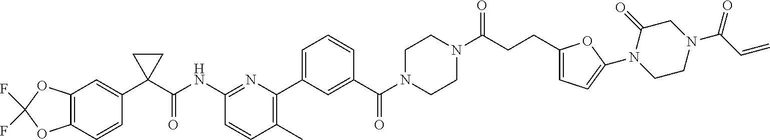 [SMILES: C=CC(=O)N1CCN(c2ccc(CCC(=O)N3CCN(C(=O)c4cccc(-c5nc(NC(=O)C6(c7ccc8c(c7)OC(F)(F)O8)CC6)ccc5C)c4)CC3)o2)C(=O)C1] |
| 214 | 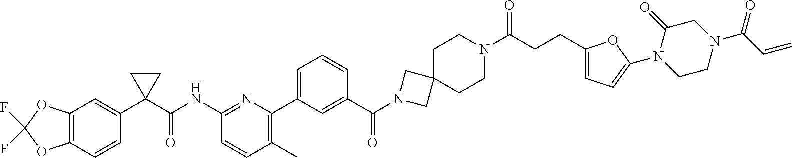 [SMILES: C=CC(=O)N1CCN(c2ccc(CCC(=O)N3CCC4(CC3)CN(C(=O)c3cccc(-c5nc(NC(=O)C6(c7ccc8c(c7)OC(F)(F)O8)CC6)ccc5C)c3)C4)o2)C(=O)C1] |
| 215 | 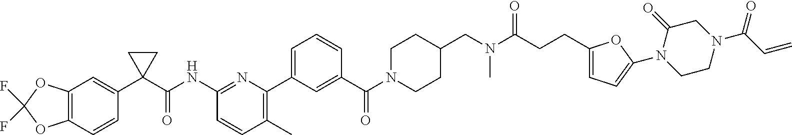 [SMILES: C=CC(=O)N1CCN(c2ccc(CCC(=O)N(C)CC3CCN(C(=O)c4cccc(-c5nc(NC(=O)C6(c7ccc8c(c7)OC(F)(F)O8)CC6)ccc5C)c4)CC3)o2)C(=O)C1] |
| 216 | 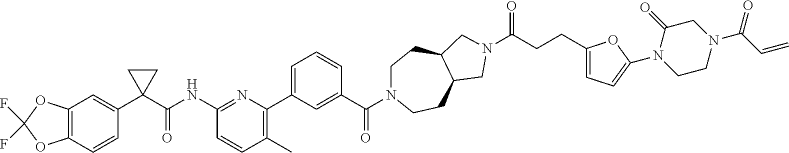 [SMILES: C=CC(=O)N1CCN(c2ccc(CCC(=O)N3C[C@H]4CCN(C(=O)c5cccc(-c6nc(NC(=O)C7(c8ccc9c(c8)OC(F)(F)O9)CC7)ccc6C)c5)CC[C@H]4C3)o2)C(=O)C1] |
| 217 | 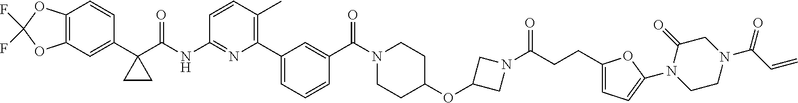 [SMILES: C=CC(=O)N1CCN(c2ccc(CCC(=O)N3CC(OC4CCN(C(=O)c5cccc(-c6nc(NC(=O)C7(c8ccc9c(c8)OC(F)(F)O9)CC7)ccc6C)c5)CC4)C3)o2)C(=O)C1] |
| 218 | 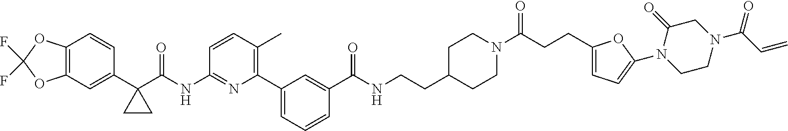 [SMILES: C=CC(=O)N1CCN(c2ccc(CCC(=O)N3CCC(CCNC(=O)c4cccc(-c5nc(NC(=O)C6(c7ccc8c(c7)OC(F)(F)O8)CC6)ccc5C)c4)CC3)o2)C(=O)C1] |
| 219 | 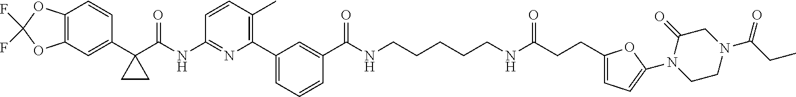 [SMILES: CCC(=O)N1CCN(c2ccc(CCC(=O)NCCCCCNC(=O)c3cccc(-c4nc(NC(=O)C5(c6ccc7c(c6)OC(F)(F)O7)CC5)ccc4C)c3)o2)C(=O)C1] |
| 220 | 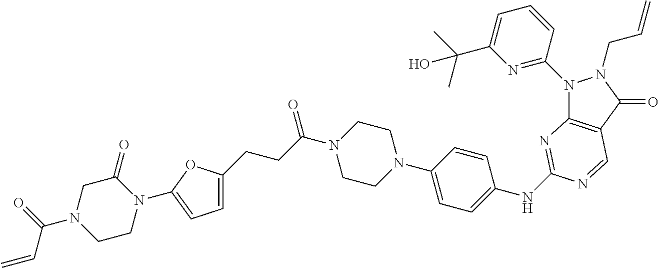 [SMILES: C=CCn1c(=O)c2cnc(Nc3ccc(N4CCN(C(=O)CCc5ccc(N6CCN(C(=O)C=C)CC6=O)o5)CC4)cc3)nc2n1-c1cccc(C(C)(C)O)n1] |
| 221 | 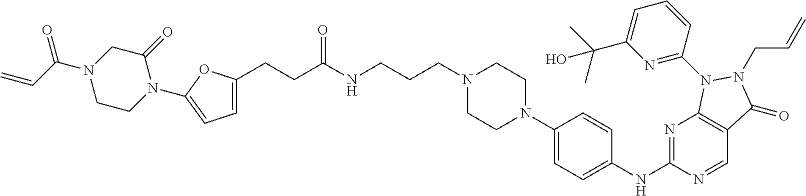 [SMILES: C=CCn1c(=O)c2cnc(Nc3ccc(N4CCN(CCCNC(=O)CCc5ccc(N6CCN(C(=O)C=C)CC6=O)o5)CC4)cc3)nc2n1-c1cccc(C(C)(C)O)n1] |
| 222 | 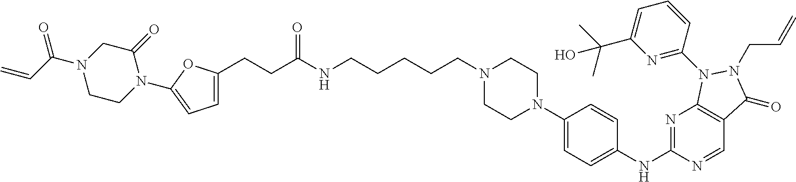 [SMILES: C=CCn1c(=O)c2cnc(Nc3ccc(N4CCN(CCCCCNC(=O)CCc5ccc(N6CCN(C(=O)C=C)CC6=O)o5)CC4)cc3)nc2n1-c1cccc(C(C)(C)O)n1] |
| 223 | 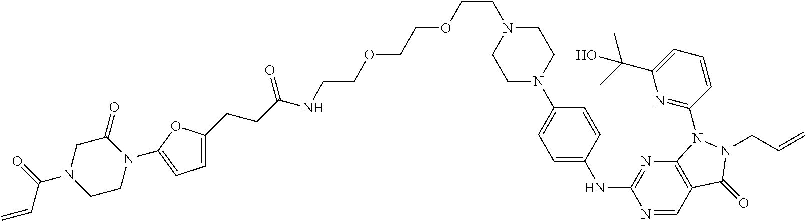 [SMILES: C=CCn1c(=O)c2cnc(Nc3ccc(N4CCN(CCOCCOCCNC(=O)CCc5ccc(N6CCN(C(=O)C=C)CC6=O)o5)CC4)cc3)nc2n1-c1cccc(C(C)(C)O)n1] |
| 224 | 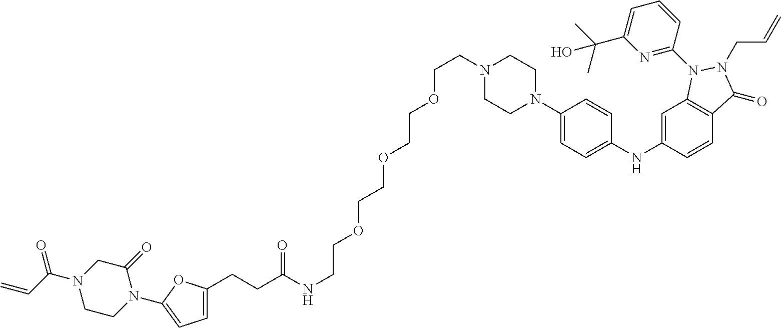 [SMILES: C=CCn1c(=O)c2ccc(Nc3ccc(N4CCN(CCOCCOCCOCCNC(=O)CCc5ccc(N6CCN(C(=O)C=C)CC6=O)o5)CC4)cc3)cc2n1-c1cccc(C(C)(C)O)n1] |
| 225 | 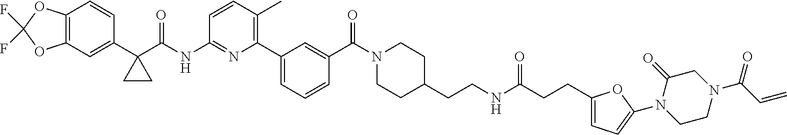 [SMILES: C=CC(=O)N1CCN(c2ccc(CCC(=O)NCCC3CCN(C(=O)c4cccc(-c5nc(NC(=O)C6(c7ccc8c(c7)OC(F)(F)O8)CC6)ccc5C)c4)CC3)o2)C(=O)C1] |
| 226 | 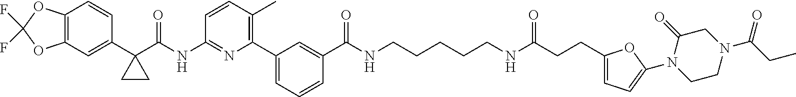 [SMILES: CCC(=O)N1CCN(c2ccc(CCC(=O)NCCCCCNC(=O)c3cccc(-c4nc(NC(=O)C5(c6ccc7c(c6)OC(F)(F)O7)CC5)ccc4C)c3)o2)C(=O)C1] |
| 227 | 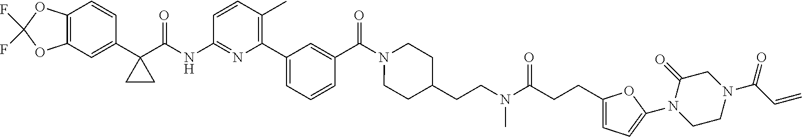 [SMILES: C=CC(=O)N1CCN(c2ccc(CCC(=O)N(C)CCC3CCN(C(=O)c4cccc(-c5nc(NC(=O)C6(c7ccc8c(c7)OC(F)(F)O8)CC6)ccc5C)c4)CC3)o2)C(=O)C1] |
| 228 | 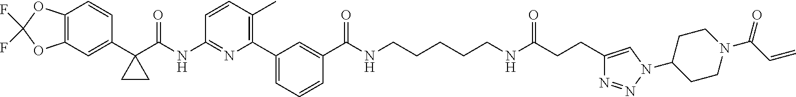 [SMILES: C=CC(=O)N1CCC(n2cc(CCC(=O)NCCCCCNC(=O)c3cccc(-c4nc(NC(=O)C5(c6ccc7c(c6)OC(F)(F)O7)CC5)ccc4C)c3)nn2)CC1] |
| 229 | 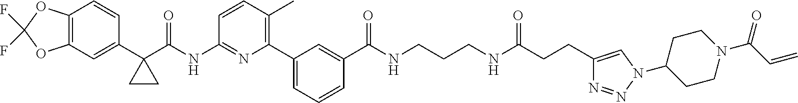 [SMILES: C=CC(=O)N1CCC(n2cc(CCC(=O)NCCCNC(=O)c3cccc(-c4nc(NC(=O)C5(c6ccc7c(c6)OC(F)(F)O7)CC5)ccc4C)c3)nn2)CC1] |
| 230 | 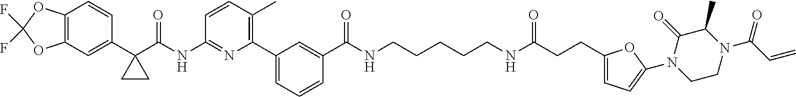 [SMILES: C=CC(=O)N1CCN(c2ccc(CCC(=O)NCCCCCNC(=O)c3cccc(-c4nc(NC(=O)C5(c6ccc7c(c6)OC(F)(F)O7)CC5)ccc4C)c3)o2)C(=O)[C@H]1C] |
| 231 | 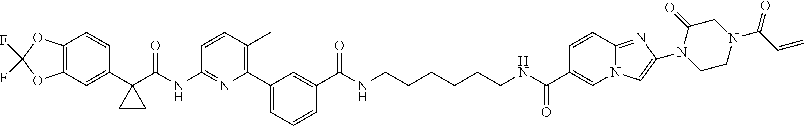 [SMILES: C=CC(=O)N1CCN(c2cn3cc(C(=O)NCCCCCCNC(=O)c4cccc(-c5nc(NC(=O)C6(c7ccc8c(c7)OC(F)(F)O8)CC6)ccc5C)c4)ccc3n2)C(=O)C1] |
| 232 | 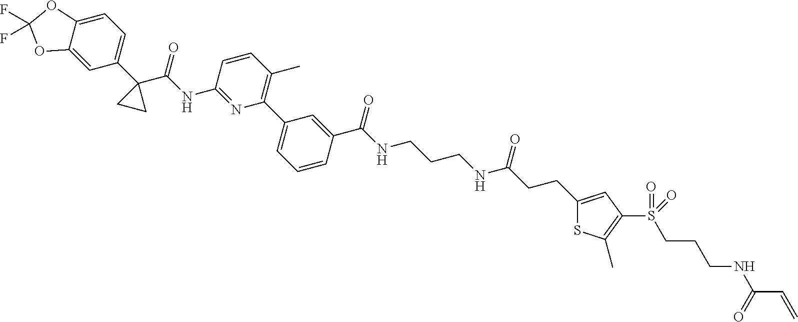 [SMILES: C=CC(=O)NCCCS(=O)(=O)c1cc(CCC(=O)NCCCNC(=O)c2cccc(-c3nc(NC(=O)C4(c5ccc6c(c5)OC(F)(F)O6)CC4)ccc3C)c2)sc1C] |
| 233 | 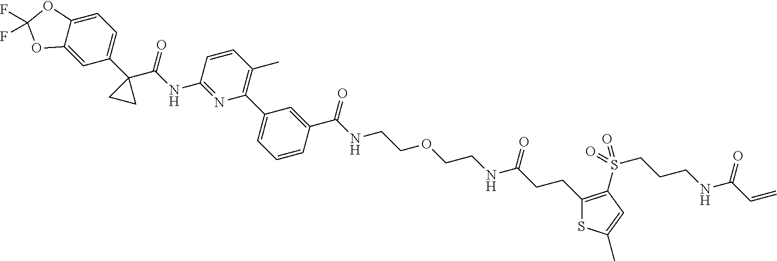 [SMILES: C=CC(=O)NCCCS(=O)(=O)c1cc(C)sc1CCC(=O)NCCOCCNC(=O)c1cccc(-c2nc(NC(=O)C3(c4ccc5c(c4)OC(F)(F)O5)CC3)ccc2C)c1] |
| 234 | 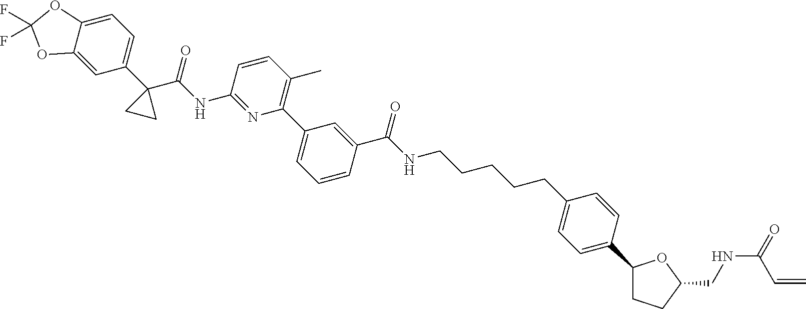 [SMILES: C=CC(=O)NC[C@@H]1CC[C@@H](c2ccc(CCCCCNC(=O)c3cccc(-c4nc(NC(=O)C5(c6ccc7c(c6)OC(F)(F)O7)CC5)ccc4C)c3)cc2)O1] |
| 235 | 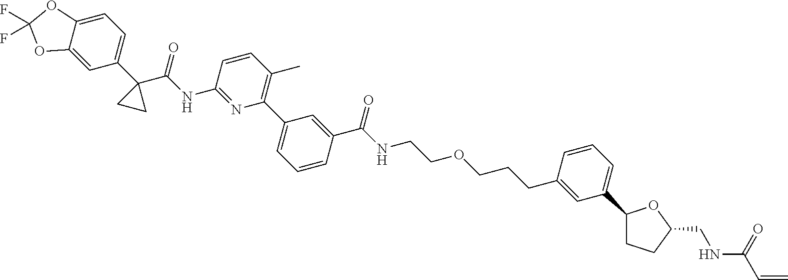 [SMILES: C=CC(=O)NC[C@@H]1CC[C@@H](c2cccc(CCCOCCNC(=O)c3cccc(-c4nc(NC(=O)C5(c6ccc7c(c6)OC(F)(F)O7)CC5)ccc4C)c3)c2)O1] |
| 236 | 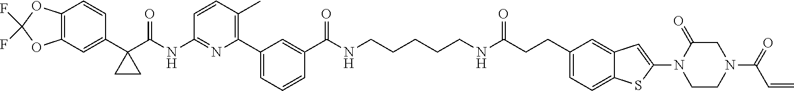 [SMILES: C=CC(=O)N1CCN(c2cc3cc(CCC(=O)NCCCCCNC(=O)c4cccc(-c5nc(NC(=O)C6(c7ccc8c(c7)OC(F)(F)O8)CC6)ccc5C)c4)ccc3s2)C(=O)C1] |

In some embodiments, the bifunctional compound is selected from the group consisting of:

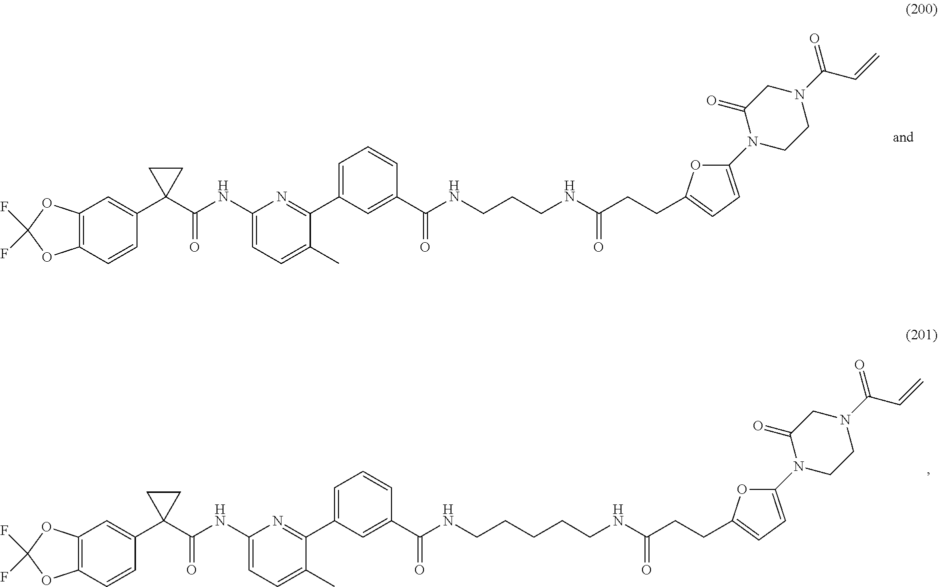 [SMILES: C=CC(=O)N1CCN(c2ccc(CCC(=O)NCCCNC(=O)c3cccc(-c4nc(NC(=O)C5(c6ccc7c(c6)OC(F)(F)O7)CC5)ccc4C)c3)o2)C(=O)C1 ; C=CC(=O)N1CCN(c2ccc(CCC(=O)NCCCCCNC(=O)c3cccc(-c4nc(NC(=O)C5(c6ccc7c(c6)OC(F)(F)O7)CC5)ccc4C)c3)o2)C(=O)C1]

or a pharmaceutically acceptable salt, hydrate, solvate, prodrug, stereoisomer, or tautomer thereof.

In some embodiments, the bifunctional compound is Compound 200 or a pharmaceutically acceptable salt, hydrate, solvate, prodrug, stereoisomer, or tautomer thereof. In some embodiments, the bifunctional compound is Compound 201 or a pharmaceutically acceptable salt, hydrate, solvate, prodrug, stereoisomer, or tautomer thereof. In some embodiments, the bifunctional compound is Compound 202 or a pharmaceutically acceptable salt, hydrate, solvate, prodrug, stereoisomer, or tautomer thereof. In some embodiments, the bifunctional compound is Compound 203 or a pharmaceutically acceptable salt, hydrate, solvate, prodrug, stereoisomer, or tautomer thereof. In some embodiments, the bifunctional compound is Compound 204 or a pharmaceutically acceptable salt, hydrate, solvate, prodrug, stereoisomer, or tautomer thereof. In some embodiments, the bifunctional compound is Compound 205 or a pharmaceutically acceptable salt, hydrate, solvate, prodrug, stereoisomer, or tautomer thereof. In some embodiments, the bifunctional compound is Compound 206 or a pharmaceutically acceptable salt, hydrate, solvate, prodrug, stereoisomer, or tautomer thereof. In some embodiments, the bifunctional compound is Compound 207 or a pharmaceutically acceptable salt, hydrate, solvate, prodrug, stereoisomer, or tautomer thereof. In some embodiments, the bifunctional compound is Compound 208 or a pharmaceutically acceptable salt, hydrate, solvate, prodrug, stereoisomer, or tautomer thereof. In some embodiments, the bifunctional compound is Compound 209 or a pharmaceutically acceptable salt, hydrate, solvate, prodrug, stereoisomer, or tautomer thereof. In some embodiments, the bifunctional compound is Compound 210 or a pharmaceutically acceptable salt, hydrate, solvate, prodrug, stereoisomer, or tautomer thereof. In some embodiments, the bifunctional compound is Compound 211 or a pharmaceutically acceptable salt, hydrate, solvate, prodrug, stereoisomer, or tautomer thereof. In some embodiments, the bifunctional compound is Compound 212 or a pharmaceutically acceptable salt, hydrate, solvate, prodrug, stereoisomer, or tautomer thereof. In some embodiments, the bifunctional compound is Compound 213 or a pharmaceutically acceptable salt, hydrate, solvate, prodrug, stereoisomer, or tautomer thereof. In some embodiments, the bifunctional compound is Compound 214 or a pharmaceutically acceptable salt, hydrate, solvate, prodrug, stereoisomer, or tautomer thereof. In some embodiments, the bifunctional compound is Compound 215 or a pharmaceutically acceptable salt, hydrate, solvate, prodrug, stereoisomer, or tautomer thereof. In some embodiments, the bifunctional compound is Compound 216 or a pharmaceutically acceptable salt, hydrate, solvate, prodrug, stereoisomer, or tautomer thereof. In some embodiments, the bifunctional compound is Compound 217 or a pharmaceutically acceptable salt, hydrate, solvate, prodrug, stereoisomer, or tautomer thereof. In some embodiments, the bifunctional compound is Compound 218 or a pharmaceutically acceptable salt, hydrate, solvate, prodrug, stereoisomer, or tautomer thereof. In some embodiments, the bifunctional compound is Compound 219 or a pharmaceutically acceptable salt, hydrate, solvate, prodrug, stereoisomer, or tautomer thereof. In some embodiments, the bifunctional compound is Compound 220 or a pharmaceutically acceptable salt, hydrate, solvate, prodrug, stereoisomer, or tautomer thereof. In some embodiments, the bifunctional compound is Compound 221 or a pharmaceutically acceptable salt, hydrate, solvate, prodrug, stereoisomer, or tautomer thereof. In some embodiments, the bifunctional compound is Compound 222 or a pharmaceutically acceptable salt, hydrate, solvate, prodrug, stereoisomer, or tautomer thereof. In some embodiments, the bifunctional compound is Compound 223 or a pharmaceutically acceptable salt, hydrate, solvate, prodrug, stereoisomer, or tautomer thereof. In some embodiments, the bifunctional compound is Compound 224 or a pharmaceutically acceptable salt, hydrate, solvate, prodrug, stereoisomer, or tautomer thereof. In some embodiments, the bifunctional compound is Compound 225 or a pharmaceutically acceptable salt, hydrate, solvate, prodrug, stereoisomer, or tautomer thereof. In some embodiments, the bifunctional compound is Compound 226 or a pharmaceutically acceptable salt, hydrate, solvate, prodrug, stereoisomer, or tautomer thereof. In some embodiments, the bifunctional compound is Compound 227 or a pharmaceutically acceptable salt, hydrate, solvate, prodrug, stereoisomer, or tautomer thereof. In some embodiments, the bifunctional compound is Compound 228 or a pharmaceutically acceptable salt, hydrate, solvate, prodrug, stereoisomer, or tautomer thereof. In some embodiments, the bifunctional compound is Compound 229 or a pharmaceutically acceptable salt, hydrate, solvate, prodrug, stereoisomer, or tautomer thereof. In some embodiments, the bifunctional compound is Compound 230 or a pharmaceutically acceptable salt, hydrate, solvate, prodrug, stereoisomer, or tautomer thereof. In some embodiments, the bifunctional compound is Compound 231 or a pharmaceutically acceptable salt, hydrate, solvate, prodrug, stereoisomer, or tautomer thereof. In some embodiments, the bifunctional compound is Compound 232 or a pharmaceutically acceptable salt, hydrate, solvate, prodrug, stereoisomer, or tautomer thereof. In some embodiments, the bifunctional compound is Compound 233 or a pharmaceutically acceptable salt, hydrate, solvate, prodrug, stereoisomer, or tautomer thereof. In some embodiments, the bifunctional compound is Compound 234 or a pharmaceutically acceptable salt, hydrate, solvate, prodrug, stereoisomer, or tautomer thereof. In some embodiments, the bifunctional compound is Compound 235 or a pharmaceutically acceptable salt, hydrate, solvate, prodrug, stereoisomer, or tautomer thereof. In some embodiments, the bifunctional compound is Compound 236 or a pharmaceutically acceptable salt, hydrate, solvate, prodrug, stereoisomer, or tautomer thereof.

### Definitions

### Selected Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

When a range of values is listed, it is intended to encompass each value and sub-range within the range. For example “C₁-C₆ alkyl” or “C₁₋₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The following terms are intended to have the meanings presented therewith below and are useful in understanding the description and intended scope of the present invention.

The term “alkyl” refers to a radical of a straight-chain or branched saturated hydrocarbon group having from 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tertiary amyl), and hexyl (C₆) (e.g., n-hexyl).

“Alkylene” refers to a divalent radical of an alkyl group, e.g., —CH₂—, —CH₂CH₂—, and —CH₂CH₂CH₂—.

“Heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁-6 alkyl”). In some embodiments, a heteroalkyl group is a saturated group having 1 to 5 carbon atoms and 1 or 2 heteroatoms within the parent chain (“heteroC₁_s alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC1 alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC1-10 alkyl. In certain embodiments, the heteroalkyl group is a substituted heteroC1-10 alkyl.

“Heteroalkylene” refers to a divalent radical of a heteroalkyl group.

“Alkoxy” or “alkoxyl” refers to an —O-alkyl radical. In some embodiments, the alkoxy groups are methoxy, ethoxy, n-propoxy, isopropoxy, n-butoxy, tert-butoxy, sec-butoxy, n-pentoxy, n-hexoxy, and 1,2-dimethylbutoxy. In some embodiments, alkoxy groups are lower alkoxy, i.e., with between 1 and 6 carbon atoms. In some embodiments, alkoxy groups have between 1 and 4 carbon atoms.

As used herein, the term “aryl” refers to a stable, aromatic, mono- or bicyclic ring radical having the specified number of ring carbon atoms. Examples of aryl groups include, but are not limited to, phenyl, 1-naphthyl, 2-naphthyl, and the like. The related term “aryl ring” likewise refers to a stable, aromatic, mono- or bicyclic ring having the specified number of ring carbon atoms.

As used herein, the term “heteroaryl” refers to a stable, aromatic, mono- or bicyclic ring radical having the specified number of ring atoms and comprising one or more heteroatoms individually selected from nitrogen, oxygen and sulfur. The heteroaryl radical may be bonded via a carbon atom or heteroatom. Examples of heteroaryl groups include, but are not limited to, furyl, pyrrolyl, thienyl, pyrazolyl, imidazolyl, thiazolyl, isothiazolyl, oxazolyl, isoxazolyl, triazolyl, tetrazolyl, pyrazinyl, pyridazinyl, pyrimidyl, pyridyl, quinolinyl, isoquinolinyl, indolyl, indazolyl, oxadiazolyl, benzothiazolyl, quinoxalinyl, and the like. The related term “heteroaryl ring” likewise refers to a stable, aromatic, mono- or bicyclic ring having the specified number of ring atoms and comprising one or more heteroatoms individually selected from nitrogen, oxygen and sulfur.

As used herein, the term “cycloalkyl” refers to a stable, saturated or unsaturated, non-aromatic, mono- or bicyclic (fused, bridged, or spiro) ring radical having the specified number of ring carbon atoms. Examples of cycloalkyl groups include, but are not limited to, the cycloalkyl groups identified above, cyclobutenyl, cyclopentenyl, cyclohexenyl, and the like. In an embodiment, the specified number is C₃-C₁₂ carbons. The related term “carbocyclic ring” likewise refers to a stable, saturated or unsaturated, non-aromatic, mono- or bicyclic (fused, bridged, or spiro) ring having the specified number of ring carbon atoms. In an embodiment, the cycloalkyl can be substituted or unsubstituted. In an embodiment, the cycloalkyl can be substituted with 0-4 occurrences of Ra, wherein each Ra is independently selected from the group consisting of C₁₋₆ alkyl, C₁₋₆ alkoxyl, and halogen.

As used herein, the term “heterocyclyl” refers to a stable, saturated or unsaturated, non-aromatic, mono- or bicyclic (fused, bridged, or spiro) ring radical having the specified number of ring atoms and comprising one or more heteroatoms individually selected from nitrogen, oxygen and sulfur. The heterocyclyl radical may be bonded via a carbon atom or heteroatom. In an embodiment, the specified number is C₃-C₁₂ carbons. Examples of heterocyclyl groups include, but are not limited to, azetidinyl, oxetanyl, pyrrolinyl, pyrrolidinyl, tetrahydrofuryl, tetrahydrothienyl, piperidyl, piperazinyl, tetrahydropyranyl, morpholinyl, perhydroazepinyl, tetrahydropyridinyl, tetrahydroazepinyl, octahydropyrrolopyrrolyl, and the like. The related term “heterocyclic ring” likewise refers to a stable, saturated or unsaturated, non-aromatic, mono- or bicyclic (fused, bridged, or spiro) ring having the specified number of ring atoms and comprising one or more heteroatoms individually selected from nitrogen, oxygen and sulfur. In an embodiment, the heterocyclyl can be substituted or unsubstituted. In an embodiment, the heterocyclyl can be substituted with 0-4 occurrences of Ra, wherein each Ra is independently selected from the group consisting of C₁₋₆ alkyl, C₁₋₆ alkoxyl, and halogen.

As used herein, “spirocycloalkyl” or “spirocyclyl” means carbogenic bicyclic ring systems with both rings connected through a single atom. The rings can be different in size and nature, or identical in size and nature. Examples include spiropentane, spriohexane, spiroheptane, spirooctane, spirononane, or spirodecane. One or both of the rings in a spirocycle can be fused to another ring carbocyclic, heterocyclic, aromatic, or heteroaromatic ring. For example, a (C₃-C₁₂)spirocycloalkyl is a spirocycle containing between 3 and 12 carbon atoms.

As used herein, “spiroheterocycloalkyl” or “spiroheterocyclyl” means a spirocycle wherein at least one of the rings is a heterocycle wherein one or more of the carbon atoms can be substituted with a heteroatom (e.g., one or more of the carbon atoms can be substituted with a heteroatom in at least one of the rings). One or both of the rings in a spiroheterocycle can be fused to another ring carbocyclic, heterocyclic, aromatic, or heteroaromatic ring.

As used herein, “halo” or “halogen” refers to fluorine (fluoro, —F), chlorine (chloro, —Cl), bromine (bromo, —Br), or iodine (iodo, —I).

As used herein, “haloalkyl” means an alkyl group substituted with one or more halogens. Examples of haloalkyl groups include, but are not limited to, trifluoromethyl, difluoromethyl, pentafluoroethyl, and trichloromethyl.

As used herein, “substituted”, whether preceded by the term “optionally” or not, means that one or more hydrogens of the designated moiety are replaced with a suitable substituent.

As used herein, the definition of each expression, e.g., alkyl, m, n, etc., when it occurs more than once in any structure, is intended to be independent of its definition elsewhere in the same structure.

Various embodiments of the disclosure are described herein. It will be recognized that features specified in each embodiment may be combined with other specified features, including as indicated in the embodiments below, to provide further embodiments of the present disclosure.

It is understood that in the following embodiments, combinations of substituents or variables of the depicted formulae are permissible only if such combinations result in stable compounds.

Certain compounds described herein may exist in particular geometric or stereoisomeric forms. If, for instance, a particular enantiomer of a compound described herein is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Unless otherwise stated, structures depicted herein are also meant to include geometric (or conformational) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the disclosed compounds are within the scope of the disclosure. Unless otherwise stated, all tautomeric forms of the compounds described herein are within the scope of the disclosure. Additionally, unless otherwise stated, structures depicted herein are also meant to include compounds that differ only in the presence of one or more isotopically enriched atoms. For example, compounds having the disclosed structures including the replacement of hydrogen by deuterium or tritium, or the replacement of a carbon by a ¹³C or ¹⁴C enriched carbon are within the scope of this disclosure. Such compounds are useful, for example, as analytical tools, as probes in biological assays, or as therapeutic agents in accordance with the disclosure.

The “enantiomeric excess” or “% enantiomeric excess” of a composition can be calculated using the equation shown below. In the example shown below a composition contains 90% of one enantiomer, e.g., the S enantiomer, and 10% of the other enantiomer, i.e., the R enantiomer. ee=(90−10)/100×100=80%.

Thus, a composition containing 90% of one enantiomer and 10% of the other enantiomer is said to have an enantiomeric excess of 80%. The compounds or compositions described herein may contain an enantiomeric excess of at least 50%, 75%, 90%, 95%, or 99% of one form of the compound, e.g., the S-enantiomer. In other words such compounds or compositions contain an enantiomeric excess of the S enantiomer over the R enantiomer.

Where a particular enantiomer is preferred, it may, in some embodiments be provided substantially free of the corresponding enantiomer, and may also be referred to as “optically enriched.” “Optically enriched,” as used herein, means that the compound is made up of a significantly greater proportion of one enantiomer. In certain embodiments, the compound is made up of at least about 90% by weight of a preferred enantiomer. In other embodiments, the compound is made up of at least about 95%, 98%, or 99% by weight of a preferred enantiomer.

Preferred enantiomers may be isolated from racemic mixtures by any method known to those skilled in the art, including chiral high pressure liquid chromatography (HPLC) and the formation and crystallization of chiral salts or prepared by asymmetric syntheses. See e.g., Jacques et al., Enantiomers, Racemates and Resolutions (Wiley Interscience, New York, 1981); Wilen, et al., Tetrahedron 33:2725 (1977); Eliel, E. L. Stereochemistry of Carbon Compounds (McGraw Hill, N Y, 1962); Wilen, S. H. Tables of Resolving Agents and Optical Resolutions p. 268 (E. L. Eliel, Ed., Univ. of Notre Dame Press, Notre Dame, IN 1972).

All methods described herein can be performed in any suitable order unless otherwise indicated herein or otherwise clearly contradicted by context. The use of any and all examples, or exemplary language (e.g. “such as”) provided herein is intended merely to better illuminate the disclosure and does not pose a limitation on the scope of the disclosure otherwise claimed.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds described herein into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

### Other Definitions

The following definitions are more general terms used throughout the present disclosure.

As used herein, the term “a,” “an,” “the” and similar terms used in the context of the disclosure (especially in the context of the claims) are to be construed to cover both the singular and plural unless otherwise indicated herein or clearly contradicted by the context.

As used herein, the term “about” means within the typical ranges of tolerances in the art. For example, “about” can be understood as about 2 standard deviations from the mean. In certain embodiments, about means±10%. In certain embodiments, about means±5%. When about is present before a series of numbers or a range, it is understood that “about” can modify each of the numbers in the series or range.

“Acquire” or “acquiring” as used herein, refer to obtaining possession of a value, e.g., a numerical value, or image, or a physical entity (e.g., a sample), by “directly acquiring” or “indirectly acquiring” the value or physical entity. “Directly acquiring” means performing a process (e.g., performing an analytical method or protocol) to obtain the value or physical entity. “Indirectly acquiring” refers to receiving the value or physical entity from another party or source (e.g., a third-party laboratory that directly acquired the physical entity or value). Directly acquiring a value or physical entity includes performing a process that includes a physical change in a physical substance or the use of a machine or device. Examples of directly acquiring a value include obtaining a sample from a human subject. Directly acquiring a value includes performing a process that uses a machine or device, e.g., mass spectrometer to acquire mass spectrometry data.

The terms “administer,” “administering,” or “administration,” as used herein refers to implanting, absorbing, ingesting, injecting, inhaling, or otherwise introducing an inventive compound, or a pharmaceutical composition thereof.

As used herein, the terms “condition,” “disease,” and “disorder” are used interchangeably.

As used herein, the terms “degrades”, “degrading”, or “degradation” refers to the partial or full breakdown of a target protein by the cellular proteasome system to an extent that reduces or eliminates the biological activity (especially aberrant activity) of target protein.

As used herein, the terms “inhibit”, “inhibition”, or “inhibiting” refer to the reduction or suppression of a given condition, symptom, or disorder, or disease, or a significant decrease in the baseline activity of a biological activity or process.

As used herein, the term “modulating a target protein” or “modulating target protein activity” means the alteration of at least one feature of a target protein. For example, modulation may comprise one or more of: (i) modulating the folding of the target protein; (ii) modulating the half-life of the target protein; (iii) modulating trafficking of the target protein to the proteasome; (iv) modulating the level of ubiquitination of the target protein; (v) modulating degradation (e.g., proteasomal degradation) of the target protein; (vi) modulating target protein signaling; (vii) modulating target protein localization; (viii) modulating trafficking of the target protein to the lysosome; and (ix) modulating target protein interactions with another protein. In an embodiment, modulating a target protein refers to one or more of: improving the folding of a protein, increasing the half-life of a protein, preventing the trafficking of the target protein to the proteasome, decreasing the level of ubiquitination of the target protein, preventing degradation of the target protein, improving target protein signaling, improving target protein signaling, preventing trafficking of the target protein to the lysosome, and improving target protein interactions with another protein.

Modulating a target protein may be achieved by stabilizing the level the target protein in vivo or in vitro. The amount of target protein stabilized can be measured by comparing the amount of target protein remaining after treatment with a bifunctional compound described herein as compared to the initial amount or level of target protein present as measured prior to treatment with a bifunctional compound described herein. In an embodiment, at least about 30% of the target protein is modulated (e.g., stabilized) compared to initial levels. In an embodiment, at least about 40% of the target protein is modulated (e.g., stabilized) compared to initial levels. In an embodiment, at least about 50% of the target protein is modulated (e.g., stabilized) compared to initial levels. In an embodiment, at least about 60% of the target protein is modulated (e.g., stabilized) compared to initial levels. In an embodiment, at least about 70% of the target protein is modulated (e.g., stabilized) compared to initial levels. In an embodiment, at least about 80% of the target protein is modulated (e.g., stabilized) compared to initial levels. In an embodiment, at least about 90% of the target protein is modulated (e.g., stabilized) compared to initial levels. In an embodiment, at least about 95% of the target protein is modulated (e.g., stabilized) compared to initial levels. In an embodiment, over 95% of the target protein is modulated (e.g., stabilized) compared to initial levels. In an embodiment, at least about 99% of the target protein is modulated (e.g., stabilized) compared to initial levels.

In an embodiment, the target protein is modulated (e.g., stabilized) in an amount of from about 30% to about 99% compared to initial levels. In an embodiment, the target protein is modulated (e.g., stabilized) in an amount of from about 40% to about 99% compared to initial levels. In an embodiment, the target protein is modulated (e.g., stabilized) in an amount of from about 50% to about 99% compared to initial levels. In an embodiment, the target protein is modulated (e.g., stabilized) in an amount of from about 60% to about 99% compared to initial levels. In an embodiment, the target protein is modulated (e.g., stabilized) in an amount of from about 70% to about 99% compared to initial levels. In an embodiment, the target protein is modulated (e.g., stabilized) in an amount of from about 80% to about 99% compared to initial levels. In an embodiment, the target protein is modulated (e.g., stabilized) in an amount of from about 90% to about 99% compared to initial levels. In an embodiment, the target protein is modulated (e.g., stabilized) in an amount of from about 95% to about 99% compared to initial levels. In an embodiment, the target protein is modulated (e.g., stabilized) in an amount of from about 90% to about 95% compared to initial levels.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprised therein. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types.

As used herein, the term “selectivity for the target protein” means, for example, a bifunctional compound described herein binds to the target protein in preference to, or to a greater extent than, another protein or proteins.

As used herein, the term “subject” refers to an animal. Typically, the animal is a mammal. A subject also refers to, for example, primates (e.g., humans, male or female), cows, sheep, goats, horses, dogs, cats, rabbits, rats, mice, fish, birds, and the like. In an embodiment, the subject is a primate. In a preferred embodiment, the subject is a human.

As used herein, the term “a therapeutically effective amount” of a compound described herein refers to an amount of the compound described herein that will elicit the biological or medical response of a subject, for example, reduction or inhibition of an enzyme or a protein activity, or ameliorate symptoms, alleviate conditions, slow or delay disease progression, or prevent a disease, etc. In one embodiment, the term “a therapeutically effective amount” refers to the amount of the compound described herein that, when administered to a subject, is effective to (1) at least partially alleviate, prevent and/or ameliorate a condition, or a disorder or a disease (i) mediated by a target protein, (ii) associated with activity of a target protein, or (iii) characterized by activity (normal or abnormal) of a target protein; or (2) reduce or inhibit the activity of a target protein; or (3) reduce or inhibit the expression of a target protein. These effects may be achieved for example by increasing the amount of a target protein by stabilizing the target protein or preventing degradation of the target protein. In one embodiment, the term “a therapeutically effective amount” refers to the amount of the compound described herein that, when administered to a cell, or a tissue, or a non-cellular biological material, or a medium, is effective to at least prevent or partially prevent reduction of the level of a target protein; or at least maintain or partially increase the activity of a target protein, for example by removing a Ubl covalent bound to the target protein.

As used herein, the terms “treat”, “treating”, or “treatment” of any disease or disorder refer in an embodiment, to ameliorating the disease or disorder (i.e., slowing or arresting or reducing the development of the disease or at least one of the clinical symptoms thereof). In an embodiment, “treat”, “treating”, or “treatment” refers to alleviating or ameliorating at least one physical parameter including those which may not be discernible by the patient.

As used herein, the term “preventing” refers to a reduction in the frequency of, or delay in the onset of, symptoms of the condition or disease.

As used herein, a subject is “in need of” a treatment if such subject would benefit biologically, medically, or in quality of life from such treatment.

### Pharmaceutically Acceptable Salts

Pharmaceutically acceptable salts of the compounds described herein are also contemplated for the uses described herein. As used herein, the terms “salt” or “salts” refer to an acid addition or base addition salt of a compound described herein. “Salts” include in particular “pharmaceutical acceptable salts.” The term “pharmaceutically acceptable salts” refers to salts that retain the biological effectiveness and properties of the compounds disclosed herein and, which typically are not biologically or otherwise undesirable. In many cases, the compounds disclosed herein are capable of forming acid and/or base salts by virtue of the presence of amino and/or carboxyl groups or groups similar thereto.

Pharmaceutically acceptable acid addition salts can be formed with inorganic acids and organic acids.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium, and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine, and tromethamine.

In some embodiments, the bifunctional compound of Formula (I) is provided as an acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate, mandelate, mesylate, methylsulphate, mucate, naphthoate, napsylate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate trifenatate, trifluoroacetate, or xinafoate salt form.

### Pharmaceutical Compositions

Another embodiment is a pharmaceutical composition comprising one or more compounds described herein or a pharmaceutically acceptable salt, hydrate, solvate, prodrug, stereoisomer, or tautomer thereof, and one or more pharmaceutically acceptable carrier(s). The term “pharmaceutically acceptable carrier” refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The compositions described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions of the disclosure are administered orally, intraperitoneally or intravenously. Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tween®, Spans and other emulsifying agents or bioavailability enhancers which are commonly used in the manufacture of pharmaceutically acceptable solid, liquid, or other dosage forms may also be used for the purposes of formulation.

The pharmaceutically acceptable compositions described herein may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutically acceptable compositions of this disclosure may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax, and polyethylene glycols.

The pharmaceutically acceptable compositions of this disclosure may also be administered topically, especially when the target of treatment includes areas or organs readily accessible by topical application, including diseases of the eye, the skin, or the lower intestinal tract. Suitable topical formulations are readily prepared for each of these areas or organs. Topical application for the lower intestinal tract can be effected in a rectal suppository formulation (see above) or in a suitable enema formulation. Topically-transdermal patches may also be used.

For topical applications, the pharmaceutically acceptable compositions may be formulated in a suitable ointment containing the active component suspended or dissolved in one or more carriers. Carriers for topical administration of the compounds of this disclosure include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol, and water.

The pharmaceutically acceptable compositions of this disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. The amount of the compounds of the present disclosure that may be combined with the carrier materials to produce a composition in a single dosage form will vary depending upon the host treated, the particular mode of administration. Preferably, the compositions should be formulated so that a dosage of between 0.01-100 mg/kg body weight/day of the inhibitor can be administered to a patient receiving these compositions.

### Isotopically Labelled Compounds

A compound described herein or a pharmaceutically acceptable salt, hydrate, solvate, prodrug, stereoisomer, or tautomer thereof, is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds described herein include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F, ³¹P, ³²P ³⁵S, ³⁶Cl, ¹²³I, ¹²⁴, ¹²⁵I, respectively. The disclosure includes various isotopically labeled compounds as defined herein, for example, those into which radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically-labeled compounds described herein or a pharmaceutically acceptable salt, hydrate, solvate, prodrug, stereoisomer, or tautomer thereof, can generally be prepared by conventional techniques known to those skilled in the art or by processes analogous to those described in the accompanying Examples and Preparations using an appropriate isotopically-labeled reagents in place of the non-labeled reagent previously employed.

Further, substitution with heavier isotopes, particularly deuterium (i.e., ²H or D) may afford certain therapeutic advantages resulting from greater metabolic stability, for example, increased in vivo half-life or reduced dosage requirements or an improvement in therapeutic index. It is understood that deuterium in this context is regarded as a substituent of a compound described herein or a pharmaceutically acceptable salt, hydrate, solvate, prodrug, stereoisomer, or tautomer thereof. The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound described herein is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

### Dosages

Toxicity and therapeutic efficacy of compounds described herein, including pharmaceutically acceptable salts and deuterated variants, can be determined by standard pharmaceutical procedures in cell cultures or experimental animals. The LD₅₀ is the dose lethal to 50% of the population. The ED₅₀ is the dose therapeutically effective in 50% of the population. The dose ratio between toxic and therapeutic effects (LD₅₀/ED₅₀) is the therapeutic index. Compounds that exhibit large therapeutic indexes are preferred. While compounds that exhibit toxic side effects may be used, care should be taken to design a delivery system that targets such compounds to the site of affected tissue in order to minimize potential damage to uninfected cells and thereby reduce side effects.

Data obtained from the cell culture assays and animal studies can be used in formulating a range of dosage for use in humans. The dosage of such compounds may lie within a range of circulating concentrations that include the ED₅₀ with little or no toxicity. The dosage may vary within this range depending upon the dosage form employed and the route of administration utilized. For any compound, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the composition.

### Methods of Use

In one aspect, the present disclosure features a method of modulating a target protein, e.g., a target protein described herein, in a subject in need thereof, the method comprising administering to the subject a therapeutically effective amount of a compound of Formula (I), or a pharmaceutically acceptable salt, hydrate, solvate, prodrug, stereoisomer, or tautomer thereof. In some embodiments, the modulating comprises one or more of: (i) modulating the folding of the target protein; (ii) modulating the half-life of the target protein; (iii) modulating trafficking of the target protein to the proteasome; (iv) modulating the level of ubiquitination of the target protein; (v) modulating degradation (e.g., proteasomal degradation) of the target protein; (vi) modulating target protein signaling; (vii) modulating target protein localization; (viii) modulating trafficking of the target protein to the lysosome; and (ix) modulating target protein interactions with another protein.

In another aspect, the present disclosure features a method of stabilizing a target protein, e.g., a target protein described herein, in a subject in need thereof, the method comprising administering to the subject a therapeutically effective amount of a compound of Formula (I), or a pharmaceutically acceptable salt, hydrate, solvate, prodrug, stereoisomer, or tautomer thereof. In some embodiments, the stabilizing comprises increasing the half-life of a target protein or removal of a Ubl from a target protein, e.g., compared to a reference standard. In some embodiments, the stabilizing improves the function of a target protein.

In another aspect, the present disclosure features a method of forming a protein complex comprising a deubiquitinase, e.g., a deubiquitinase described herein, and a target protein, upon administration of a compound of Formula (I) or a pharmaceutically acceptable salt, hydrate, solvate, prodrug, stereoisomer, or tautomer thereof. In some embodiments, the protein complex is formed in vitro (e.g., in a sample) or in vivo (e.g., in a cell or tissue, e.g., in a subject).

Formulation of the protein complex may be observed and characterized by any method known in the art, e.g., mass spectrometry (native mass spectrometry) or SDS PAGE. In some embodiments, forming the protein complex modulates the level of a target protein, e.g., increases the half-life of the target protein, e.g., compared to a reference standard. In some embodiments, forming the protein enhances removal of a Ubl from the target protein, e.g., compared to a reference standard. In some embodiments, the deubiquitinase is OTUB1. In some embodiments, the target protein comprises CFTR.

Another embodiment is a method for removing a Ubl (e.g., a ubiquitin or ubiquitin-like protein) from a target protein, e.g., a target protein described herein, in a subject in need thereof, the method comprising administering to the subject a therapeutically effective amount of a compound of Formula (I), or a pharmaceutically acceptable salt, hydrate, solvate, prodrug, stereoisomer, or tautomer thereof.

In another aspect, the present disclosure provides a method of maintaining, improving, or increasing the activity of a target protein, e.g., a target protein described herein, the method comprising administering to the subject a therapeutically effective amount of a compound of Formula (I), or a pharmaceutically acceptable salt, hydrate, solvate, prodrug, stereoisomer, or tautomer thereof.

In an embodiment, maintaining, improving, or increasing the activity of a target protein comprises recruiting a deubiquitinase (e.g., a deubiquitinase of Table 1) with the bifunctional compound described herein (e.g., the DUB Recruiter within the bifunctional compound), e.g., a compound of Formula (I), forming a ternary complex of the target protein, the bifunctional compound, and the deubiquitinase, to thereby maintain, improve, or increase the activity of the target protein.

In another aspect, the present disclosure features a method of treating or preventing a disease, disorder or condition mediated by a target protein, e.g., a target protein described herein, the method comprising administering to the subject a therapeutically effective amount of a compound of Formula (I), or a pharmaceutically acceptable salt, hydrate, solvate, prodrug, stereoisomer, or tautomer thereof. In some embodiments, the disease, disorder, or condition is selected from the group consisting of a respiratory disorder, a proliferative disorder, an autoimmune disorder, an autoinflammatory disorder, an inflammatory disorder, a metabolic disorder, a neurological disorder, and an infectious disease. In some embodiments, the disease, disorder, or condition is selected from the group consisting of a respiratory disorder, a proliferative disorder, an autoimmune disorder, an autoinflammatory disorder, an inflammatory disorder, a neurological disorder, and an infectious disease. In some embodiments, the disease, disorder, or condition comprises a respiratory disorder. In some embodiments, the disease, disorder, or condition comprises a proliferative disorder. In some embodiments, the disease, disorder, or condition comprises an autoinflammatory disorder. In some embodiments, the disease, disorder, or condition comprises an inflammatory disorder. In some embodiments, the disease, disorder, or condition comprises a metabolic disorder. In some embodiments, the disease, disorder, or condition comprises a neurological disorder. In some embodiments, the disease, disorder, or condition comprises an infectious disease. In some embodiments, the disease, disorder, or condition is cancer. In some embodiments, the disease, disorder, or condition is cystic fibrosis. In some embodiments, the disease, disorder, or condition is diabetes (e.g., maturity-onset diabetes of the young type 2, MODY2).

In another aspect, the disclosure provides a compound of Formula (I) or a pharmaceutically acceptable salt, hydrate, solvate, prodrug, stereoisomer, or tautomer thereof, for use in inhibiting or modulating a target protein in a subject in need thereof.

Another embodiment is a use of a compound of Formula (I), or a pharmaceutically acceptable salt, hydrate, solvate, prodrug, stereoisomer, or tautomer thereof, in the manufacture of a medicament for treating or preventing a respiratory disorder, a proliferative disorder, an autoimmune disorder, an autoinflammatory disorder, an inflammatory disorder, a neurological disorder, and an infectious disease or disorder in a subject in need thereof.

### Examples

The disclosure is further illustrated by the following examples and synthesis schemes, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

Compounds of the present disclosure may be prepared by methods known in the art of organic synthesis. In all of the methods it is understood that protecting groups for sensitive or reactive groups may be employed where necessary in accordance with general principles of chemistry. Protecting groups are manipulated according to standard methods of organic synthesis (T. W. Green and P. G. M. Wuts (1999) Protective Groups in Organic Synthesis, 3rd edition, John Wiley & Sons). These groups are removed at a convenient stage of the compound synthesis using methods that are readily apparent to those skilled in the art.

### General Methods

Cysteine-reactive covalent ligand libraries were either previously synthesized and described or purchased from Enamine. Lumacaftor was purchased from Medchemexpress.

### Cell Culture

CFBE41o-4.7 ΔF508-CFTR Human CF Bronchial Epithelial cells were purchased from Millipore Sigma (SCC159). CFBE41o-4.7 ΔF508-CFTR Human CF Bronchial Epithelial cells were cultured in MEM (Gibco) containing 10% (v/v) fetal bovine serum (FBS) and maintained at 37° C. with 5% CO₂.

### Gel-Based Activity-Based Protein Profiling (ABPP)

Recombinant OTUB1 (0.1 g/sample) was pre-treated with either DMSO vehicle or covalent ligand or bifunctional compounds at 37° C. for 30 min in 25 μL of PBS, and subsequently treated with of IA-Rhodamine (Setareh Biotech) at room temperature for 1 h. The reaction was stopped by addition of 4× reducing Laemmli SDS sample loading buffer (Alfa Aesar). After boiling at 95° C. for 5 min, the samples were separated on precast 4-20% Criterion TGX gels (Bio-Rad). Probe-labeled proteins were analyzed by in-gel fluorescence using a ChemiDoc MP (Bio-Rad).

### Deubiquitinase Activity Assay

Previously described methods were used to assess DUB Recruiters effects on OTUB1 activity. Recombinant OTUB1 (500 nM) was pre-incubated with DMSO or Compound 100 (50 mM) for 1 hr. To initiate assay pre-treated OTUB1 enzyme was mixed 1:1 with di-Ub reaction mix for final concentrations of 250 nM OTUB1, 1.5 μM di-Ub, 12.5 μM UBE2D1 and 5 mM DTT. The appearance of mono-Ub was monitored by Western blotting over time by removing a portion of the reaction mix and adding Laemmli's buffer to terminate the reaction. Blot shown is a representative gel from n=3 biologically independent experiments/group.

### Western Blotting

Proteins were resolved by SDS/PAGE and transferred to nitrocellulose membranes using the Trans-Blot Turbo transfer system (Bio-Rad). Membranes were blocked with 5% BSA in Tris-buffered saline containing Tween 20 (TBS-T) solution for 30 min at RT, washed in TBS-T, and probed with primary antibody diluted in recommended diluent per manufacturer overnight at 4° C. After 3 washes with TBS-T, the membranes were incubated in the dark with IR680- or IR800-conjugated secondary antibodies at 1:10,000 dilution in 5% BSA in TBS-T at room temperature for 1 h. After 3 additional washes with TBST, blots were visualized using an Odyssey Li-Cor fluorescent scanner. The membranes were stripped using ReBlot Plus Strong Antibody Stripping Solution (EMD Millipore) when additional primary antibody incubations were performed. Antibodies used in this study were CFTR (Cell Signaling Technologies, Rb mAb #78335), CFTR (R&D Systems, Ms mAb, #MAB25031), CFTR (Millipore, Ms mAb, #MAB3484), CFTR (Prestige, Rb pAb, #HPA021939), GAPDH (Proteintech, Ms mAb, #60004-1-Ig), OTUB1 (Abcam, Rb mAb, #ab175200, [EPR13028(B)]), CTNNB1 (Cell Signaling Technologies, Rb mAb, #8480), and WEE1 (Cell Signaling Technologies, #4936).

### IsoTOP-ABPP Chemoproteomic Experiments

IsoTOP-ABPP studies were done as previously reported. Our aggregate chemoproteomic data analysis of DUBs was obtained from 455 distinct isoTOP-ABPP experiments previously evaluated. These data are aggregated from various human cell lines, including 231MFP, A549, HeLa, HEK293T, HEK293A, UM-Chor1, PaCa2, PC3, HUH7, NCI-H460, THP1, SKOV3, U20S, and K562 cells. All of the isoTOP-ABPP datasets were prepared as previously described using the IA-alkyne probe. Cells were lysed by probe sonication in PBS and protein concentrations were measured by BCA assay. Cells were treated for 4 h with either DMSO vehicle or a covalent ligand (from 1,000×DMSO stock) before cell collection and lysis. Proteomes were subsequently labeled with IA-alkyne labeling (100 μM for DUB ligandability analysis and 200 mM for profiling cysteine-reactivity of Compound 201) for 1 h at room temperature. CuAAC was used by sequential addition of tris(2-carboxyethyl)phosphine (1 mM, Strem, 15-7400), tris[(1-benzyl-1H-1,2,3-triazol-4-yl)methyl]amine (34 μM, Sigma, 678937), copper(II) sulfate (1 mM, Sigma, 451657) and biotin-linker-azide—the linker functionalized with a tobacco etch virus (TEV) protease recognition sequence as well as an isotopically light or heavy valine for treatment of control or treated proteome, respectively. After CuAAC, proteomes were precipitated by centrifugation at 6,500 g, washed in ice-cold methanol, combined in a 1:1 control:treated ratio, washed again, then denatured and resolubilized by heating in 1.2% SDS-PBS to 80° C. for 5 min. Insoluble components were precipitated by centrifugation at 6,500 g and soluble proteome was diluted in 5 ml 0.2% SDS-PBS. Labeled proteins were bound to streptavidin-agarose beads (170 μl resuspended beads per sample, Thermo Fisher, 20349) while rotating overnight at 4° C. Bead-linked proteins were enriched by washing three times each in PBS and water, then resuspended in 6 M urea/PBS, and reduced in TCEP (1 mM, Strem, 15-7400), alkylated with iodoacetamide (18 mM, Sigma), before being washed and resuspended in 2 M urea/PBS and trypsinized overnight with 0.5 μg/L sequencing grade trypsin (Promega, V5111). Tryptic peptides were eluted off. Beads were washed three times each in PBS and water, washed in TEV buffer solution (water, TEV buffer, 100 μM dithiothreitol) and resuspended in buffer with Ac-TEV protease (Invitrogen, 12575-015) and incubated overnight. Peptides were diluted in water and acidified with formic acid (1.2 M, Fisher, A117-50) and prepared for analysis.

### IsoTOP-ABPP Mass Spectrometric Analysis

Peptides from all chemoproteomic experiments were pressure-loaded onto a 250 m inner diameter fused silica capillary tubing packed with 4 cm of Aqua C₁₈ reverse-phase resin (Phenomenex, 04A-4299), which was previously equilibrated on an Agilent 600 series high-performance liquid chromatograph using the gradient from 100% buffer A to 100% buffer B over 10 min, followed by a 5 min wash with 100% buffer B and a 5 min wash with 100% buffer A. The samples were then attached using a MicroTee PEEK 360 m fitting (Thermo Fisher Scientific p-888) to a 13 cm laser pulled column packed with 10 cm Aqua C18 reverse-phase resin and 3 cm of strong-cation exchange resin for isoTOP-ABPP studies. Samples were analyzed using an Q Exactive Plus mass spectrometer (Thermo Fisher Scientific) using a five-step Multidimensional Protein Identification Technology (MudPIT) program, using 0, 25, 50, 80 and 100% salt bumps of 500 mM aqueous ammonium acetate and using a gradient of 5-55% buffer B in buffer A (buffer A: 95:5 water:acetonitrile, 0.1% formic acid; buffer B 80:20 acetonitrile:water, 0.1% formic acid). Data were collected in data-dependent acquisition mode with dynamic exclusion enabled (60 s). One full mass spectrometry (MS1) scan (400-1,800 mass-to-charge ratio (m z)) was followed by 15 MS2 scans of the nth most abundant ions. Heated capillary temperature was set to 200° C. and the nanospray voltage was set to 2.75 kV. Data were extracted in the form of MS1 and MS2 files using Raw Extractor v.1.9.9.2 (Scripps Research Institute) and searched against the Uniprot human database using ProLuCID search methodology in IP2 v.3 (Integrated Proteomics Applications, Inc.). Cysteine residues were searched with a static modification for carboxyaminomethylation (+57.02146) and up to two differential modifications for methionine oxidation and either the light or heavy TEV tags (+464.28596 or +470.29977, respectively). Peptides were required to be fully tryptic peptides and to contain the TEV modification. ProLUCID data were filtered through DTASelect to achieve a peptide false-positive rate below 5%. Only those probe-modified peptides that were evident across two out of three biological replicates were interpreted for their isotopic light to heavy ratios. For those probe-modified peptides that showed ratios greater than two, we only interpreted those targets that were present across all three biological replicates, were statistically significant and showed good quality MS1 peak shapes across all biological replicates. Light versus heavy isotopic probe-modified peptide ratios are calculated by taking the mean of the ratios of each replicate paired light versus heavy precursor abundance for all peptide-spectral matches associated with a peptide. The paired abundances were also used to calculate a paired sample t-test P value in an effort to estimate constancy in paired abundances and significance in change between treatment and control. P values were corrected using the Benjamini-Hochberg method.

### Knockdown Studies

RNA interference was performed using siRNA purchased from Dharmacon. CFBE41o-4.7 cells were seeded at 400,000 cells per 6 cm plate and allowed to adhere overnight. Cells were transfected with 33 nM of either nontargeting (ON-TARGETplus Non-targeting Control Pool, Dharmacon #D-001810-10-20) or anti-CFTR siRNA (Dharmacon, custom) using 8 mL of transfection reagent: either DharmaFECT 1 (Dharmacon #T-2001-02), DharmaFECT 4 (Dharmacon, T-2004-02) or Lipofectamine 2000 (ThermoFisher #11668027). Transfection reagent was added to OPTIMEM (ThermoFisher #31985070) media, allowed to incubate for 5 minutes at room temperature. Meanwhile siRNA was added to an equal amount of OPTIMEM. Solutions of transfection reagent and siRNA in OPTIMEM were then combined and allowed to incubate for 30 minutes at room temperature. These combined solutions were diluted with complete MEM to provide 33 nM siRNA and 8 mL of transfection reagent per 4 mL MEM, and the media exchanged. Cells were incubated with transfection reagents for 24h, at which point the media replaced with media containing DMSO or 10 mM Compound 201 and incubated for another 24h. Cells were then harvested, and protein abundance analyzed by Western blotting.

### Quantitative TMT Proteomics Analysis

Quantitative TMT-based proteomic analysis was performed as previously described. Acquired MS data was processed using Proteome Discoverer v. 2.2.0.388 software (Thermo) utilizing Mascot v 2.5.1 search engine (Matrix Science, London, UK) together with Percolator validation node for peptide-spectral match filtering. Data was searched against Uniprot protein database (canonical human and mouse sequences, EBI, Cambridge, UK) supplemented with sequences of common contaminants. Peptide search tolerances were set to 10 ppm for precursors, and 0.8 Da for fragments. Trypsin cleavage specificity (cleavage at K, R except if followed by P) allowed for up to 2 missed cleavages. Carbamidomethylation of cysteine was set as a fixed modification, methionine oxidation, and TMT-modification of N-termini and lysine residues were set as variable modifications. Data validation of peptide and protein identifications was done at the level of the complete dataset consisting of combined Mascot search results for all individual samples per experiment via the Percolator validation node in Proteome Discoverer. Reporter ion ratio calculations were performed using summed abundances with most confident centroid selected from 20 ppm window. Only peptide-to-spectrum matches that are unique assignments to a given identified protein within the total dataset are considered for protein quantitation. High confidence protein identifications were reported using a Percolator estimated <1% false discovery rate (FDR) cut-off. Differential abundance significance was estimated using a background-based ANOVA with Benjamini-Hochberg correction to determine adjusted p-values.

### Example 1: Identification of Deubiquitinases with Ligandable Cysteine Residues

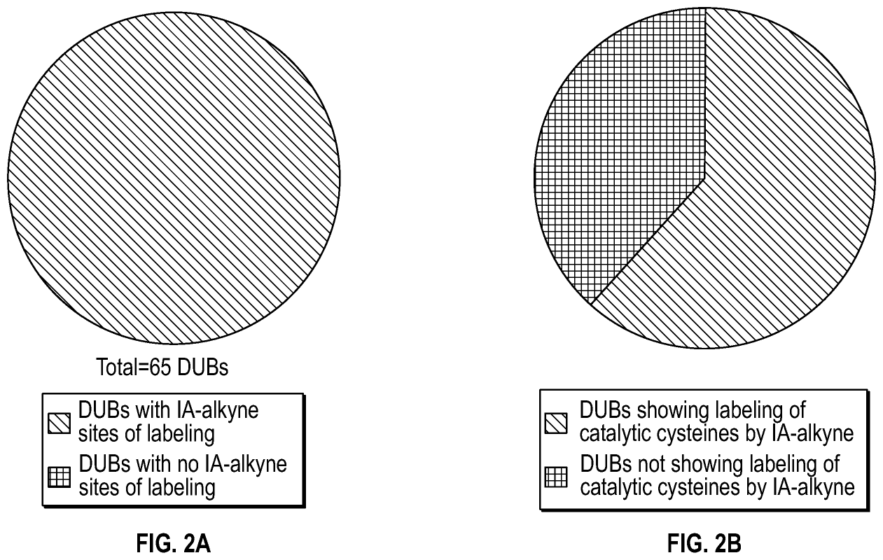

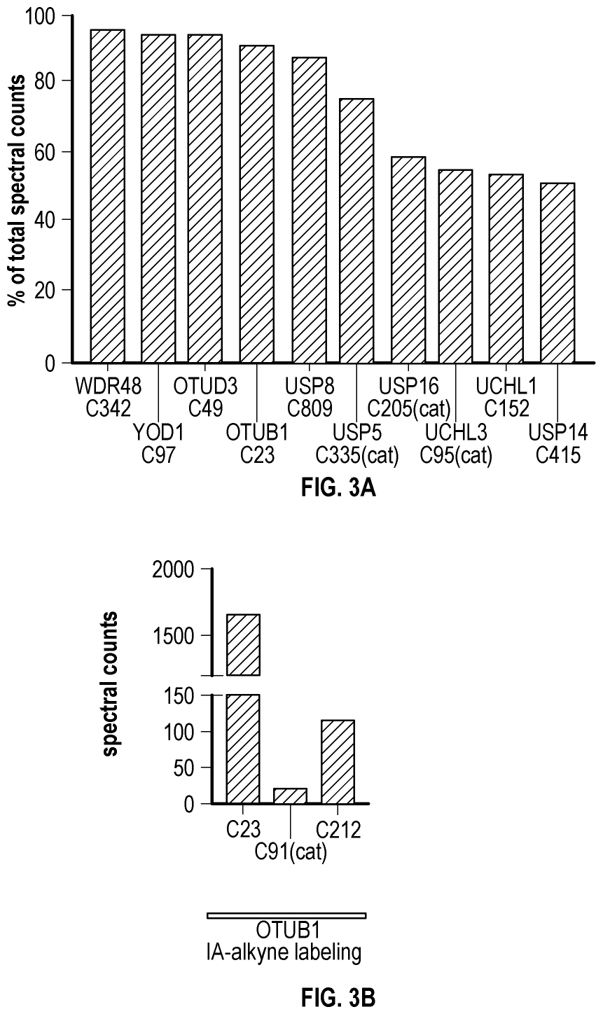

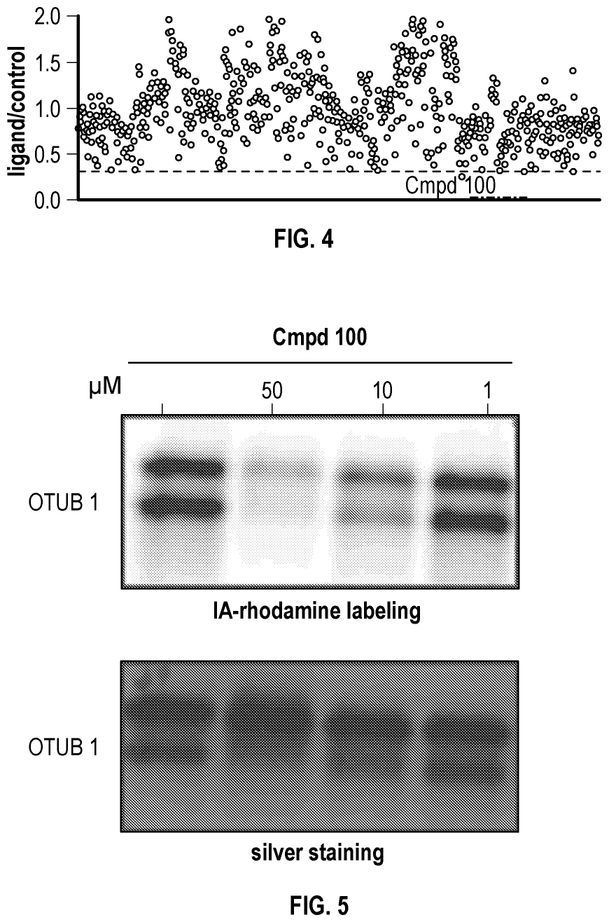

Out of 65 DUBs mined in chemoproteomic datasets of cysteine-reactive probe labeling with IA-alkyne in various complex proteomes, probe-modified cysteines were identified across all 100% of the 65 DUBs (FIG. 2A). Among the 65 DUBs that showed probe-modified cysteines, 39 of these DUBs showed >10 aggregate spectral counts across our chemoproteomic datasets (FIG. 2B). 24 DUBs, or 62%, of these 39 DUBs showed labeling of the DUB catalytic or active site cysteines. 10 DUBs were identified in which there was one probe-modified cysteine that represented >50% of the total aggregate spectral counts for probe-modified cysteine peptides for the particular DUB. 7 of those 10 DUBs do not target a known catalytic cysteine, and 3 do target the catalytic cysteine (abbreviated by cat, FIG. 3A). Analysis of aggregate chemoproteomic data for OTUB1 IA-alkyne labeling showing that C₂₃ is the dominant site labeled by IA-alkyne compared to the catalytic (cat) C91 (FIG. 3B).

### Example 2: Identification of Cysteine-Labeling Agents that Target an Exemplary Deubiquitinase (OTUB1)

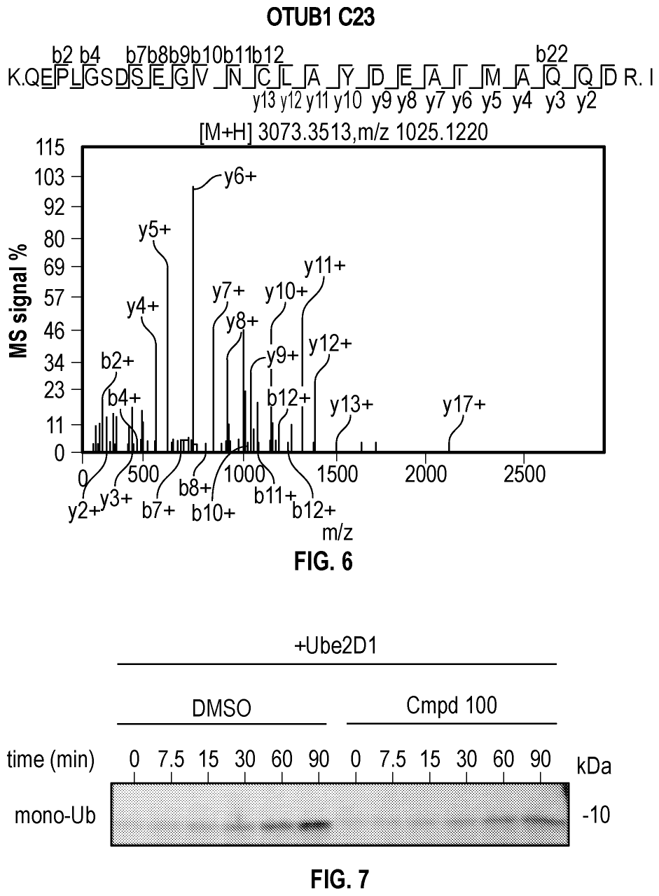

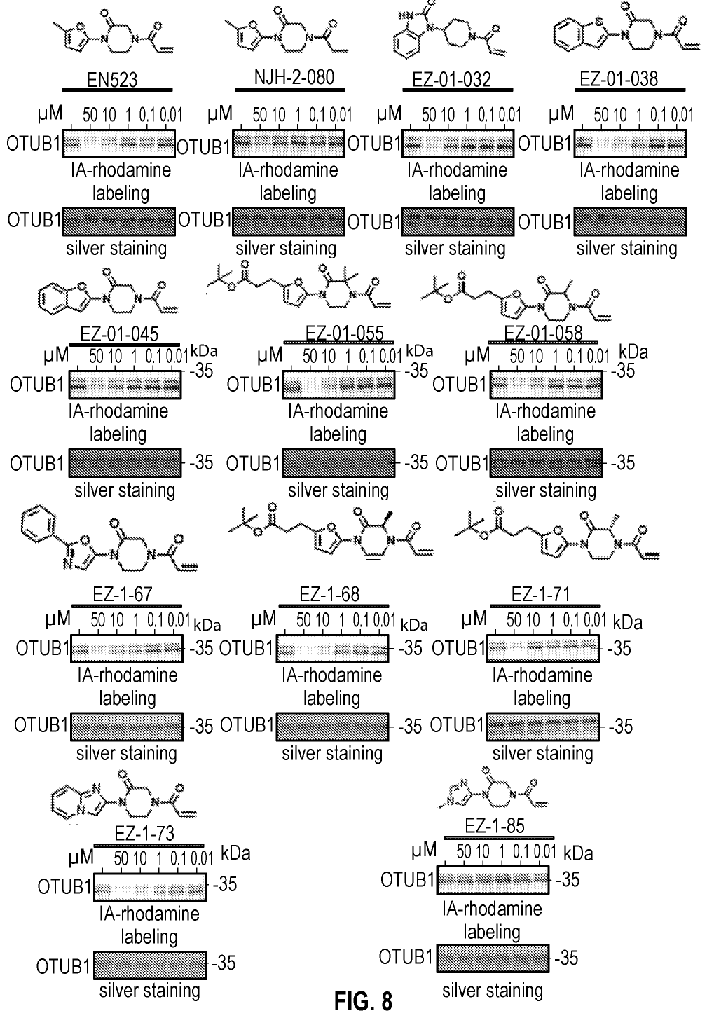

A covalent ligand screen of cysteine-reactive libraries competed against IA-rhodamine labeling of a recombinant exemplary deubiquitinase OTUB1 was carried out to identify small molecule binders to OTUB1 by gel-based activity-based protein profiling (ABPP). Vehicle DMSO or cysteine-reactive covalent ligands (50 mM) were pre-incubated with OTUB1 for 30 min at room temperature prior to IA-rhodamine labeling (500 nM, 30 min room temperature); see FIG. 4. OTUB1 was then separated by SDS/PAGE and in-gel fluorescence was assessed and quantified. Gel-based ABPP data of in-gel fluorescence is shown in FIG. 5.

### Example 3: Synthesis of Exemplary Bifunctional Compounds

### Chemical Synthesis and Characterization

Starting materials, reagents and solvents were purchased from commercial suppliers and were used without further purification unless otherwise noted. All reactions were monitored by thin layer chromatography (TLC; TLC Silica gel 60 F₂₅₄, Sepulco Millipore Sigma). Reaction products were purified by flash column chromatography using a Biotage Isolera with Biotage Sfar® or Silicycle normal-phase silica flash columns (5 g, 10 g, 25 g, or 40 g). 1H NMR and 13C NMR spectra were recorded on a 400 MHz Bruker Avance I spectrometer or a 600 MHz Bruker Avance II spectrometer equipped with a 5 mm 1H-1/BB Prodigy crypo-probe. Chemical shifts are reported in parts per million (ppm, δ) downfield from tetramethylsilane (TMS). Coupling constants (J) are reported in Hz. Spin multiplicities are described as br (broad), s (singlet), d (doublet), t (triplet), q (quartet) and m (multiplet).

### General Procedure A:

Carboxylic acid (1.0 eq.) was dissolved in dichloromethane (DCM; 0.1 M). An amine (1.25 eq.) was added, followed by diisopropylethylamine (DIEA; 4.0 eq.), hydrobenzotriazyle (HOBt; 0.2 eq.) and 1-ethyl-3-(3-dimethyaminopropyl) carbodiimide hydrochloride (EDCI; 2.0 eq.). The reaction mixture was stirred overnight at room temperature, water was added, and the mixture extracted three times with DCM. Combined organic extracts were washed with 1M HCl, washed with brine, dried over sodium sulfate, and concentrated. The crude product was purified by silica gel chromatography to provide the amide.

### General Procedure B:

Boc-protected amine was dissolved in DCM (0.1 M), and trifluoroacetic acid (TFA) was added to give a 1:2 TFA:DCM ratio. The solution was allowed to stir for 1h. The volatiles were then evaporated, and the resulting oil redissolved in DCM and treated with aqueous saturated NaHCO₃. The resulting mixture was then extracted with DCM three times, then the combined organic extracts dried over Na₂SO₄ and concentrated to provide the amine without further purification.

### General Procedure C:

Tert-butyl ester such as Intermediate 3 (30 mg, 0.086 mmol, 3.0 eq) was dissolved in DCM (600 mL). TFA (300 mL) was added and the solution stirred for 1h. Volatiles were evaporated under vacuum, and DCM (1 mL) was added and evaporated to give the carboxylic acid intermediate, though some excess TFA remained. This intermediate was dissolved in dimethylformamide (DMF; 500 mL) and DIEA (150 mL, 30 eq.) and the appropriate amine (0.029 mmol, 1.0 eq) were added, followed by 1-(bis(dimethylamino)methylene-1H-1,2,3-triazolo(4,5-b)pyridinium 3-oxide hexafluorophosphate (HATU; 30 mg, 0.079 mmol, 2.7 eq.). The reaction mixture was allowed to stir for 1h at rt. Water was added, and the mixture extracted three times with EtOAc or 4:1 CHCl₃:IPA. Organic extracts were combined, washed with brine, dried over sodium sulfate, and concentrated. Crude residues were purified by silica gel chromatography to provide the final compounds.

### General Procedure D:

To a solution of the appropriate bromide dissolved in dioxane, N,N′-dimethylethylenediamine (0.25 eq), K₂CO₃ (3.0 eq), CuI (0.1 eq), and the appropriate amide coupling partner (1.0 eq) were added. The reaction mixture was degassed, the atmosphere exchanged for nitrogen, and stirred at 100° C. overnight. Saturated NH₄Cl was added to the completed reaction mixture once cooled, which was stirred for 20 minutes, then filtered through celite and the celite pad was washed with ethyl acetate (EtOAc). The mixture was extracted with EtOAc three times, washed with brine twice, and dried by NaSO₄, before concentration in vacuo. Resulting crude mixtures were purified via silica gel column chromatography.

### General Procedure E:

The appropriate amine was dissolved in tetrahydrofuran (THF) and water (2:1 THF:H₂O) with potassium carbonate (3.0 eq). Benzyl chloroformate (1-2 eq) was added dropwise to the reaction mixture, which was then stirred vigorously overnight at room temperature. Water was added and the mixture extracted with EtOAc three times. Organic extracts were combined, washed with brine twice, concentrated and the resulting crude purified using flash column chromatography.

### General Procedure F:

The coupled product was dissolved in DCM, followed by a dropwise addition of TFA (1:2 TFA:DCM) until consumption of starting material was observed via TLC (15-30 min). The mixture was then washed with DCM twice and immediately used without further purification.

### General Procedure G:

Pd/C (10% wt.) was added to a mixture of the Cbz-protected compound in ethanol (EtOH; 0.2 M), and the atmosphere was exchanged for H₂ (balloon). The reaction mixture was stirred vigorously overnight, before being diluted with DCM, filtered through a syringe filter (0.45 m), concentrated, and purified using silica gel column chromatography.

### General Procedure H.

The amine starting material was dissolved in DCM on ice. Triethylamine (TEA; 3.0 eq) and acryloyl chloride (1.5 eq) were then added to the reaction mixture until consumption of the starting material was observed by TLC (0.5-2 hrs). Water was added, and the reaction mixture was extracted with DCM three times. Organic extracts were combined, washed with H₂O then brine, concentrated, and purified via silica gel column chromatography.

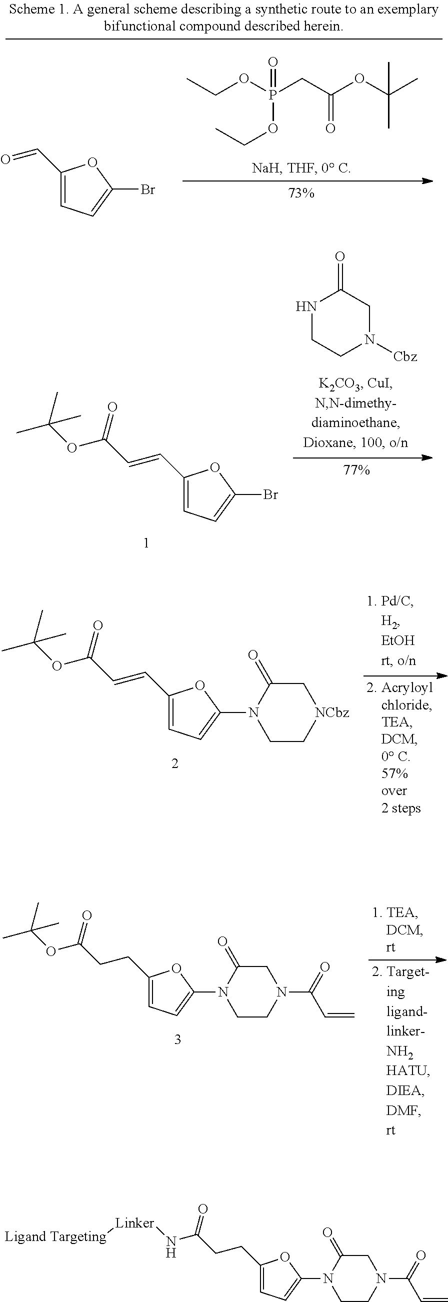

### Synthesis of Compound 200

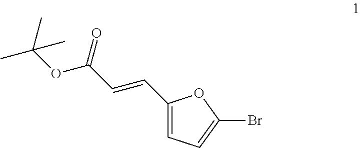

tert-butyl (E)-3-(5-bromofuran-2-yl)acrylate (1): tert-butyl diethylphosphonoacetate (971 mg, 0.908 mL, 3.85 mmol) was dissolved in THF (22 mL) and the solution cooled to 0° C. Then, 2-bromofuran-2carbaldehyde (613 mg, 3.50 mmol) was added portion-wise over 5 minutes. The reaction was stirred for 20 minutes at 0° C. as a gummy solid precipitated and then water was added. The resulting mixture was extracted with EtOAc three times, combined organic extracts were washed with brine, dried over Na₂SO₄, and concentrated. The crude residue was purified by silica gel chromatography (0-15% EtOAc/Hex) to provide the title compound as an oil (782 mg, 2.86 mmol, 82%). 1H NMR (400 MHz, CDCl₃) δ 7.26 (d, J=15.7 Hz, 1H), 6.55 (d, J=3.5 Hz, 1H), 6.42 (d, J=3.4 Hz, 1H), 6.29 (d, J=15.7 Hz, 1H), 1.55 (s, 9H).

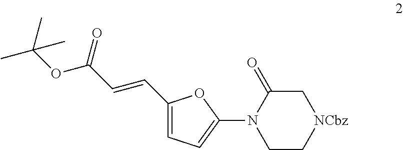 [SMILES: CC(C)(C)OC(=O)/C=C/c1ccc(N2CCN(C(=O)OCc3ccccc3)CC2=O)o1]

benzyl (E)-4-(5-(3-(tert-butoxy)-3-oxoprop-1-en-1-yl)furan-2-yl)-3-oxopiperazine-1-carboxylate (2): tert-butyl (E)-3-(5-bromofuran-2-yl)acrylate (1.62 g, 5.94 mmol) was dissolved in dioxane (30 mL) and benzyl 3-oxopiperazine-1-carboxylate (1.4 g, 5.94 mmol), K₂CO₃ (2.46 g, 17.8 mmol), N,N′-dimethyldiaminoethane (0.167 mL, 1.49 mmol), and CuI (114 mg, 0.59 mmol) were added. The mixture was stirred under nitrogen at reflux for 40 h, then cooled to rt. 5 mL saturated aq. NH₄Cl was added and the mixture stirred for 30 min. Then the mixture was diluted in EtOAc, filtered through celite, water was added, the mixture partitioned, and the aqueous layer extracted with EtOAc. The extracts were combined, washed with brine, dried over Na₂SO₄, concentrated, and purified by silica gel chromatography (0-35% EtOAc/Hex) to provide the title compound as an oil (1.95 g, 4.59 mmol, 77%). LC/MS [M+2H-tBu]⁺ m/z calc. 371.18, found 373.1. 1H NMR (400 MHz, DMSO-d6) δ 7.45-7.24 (m, 6H), 6.98 (s, 1H), 6.57 (s, 1H), 6.08 (dd, J=15.7, 3.4 Hz, 1H), 5.14 (dd, J=4.4, 2.3 Hz, 2H), 4.22 (s, 2H), 4.01 (s, 2H), 3.77 (s, 2H), 1.47 (s, 9H).

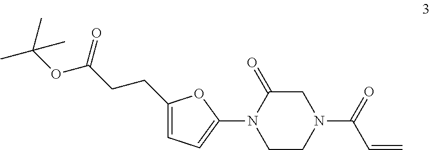 [SMILES: C=CC(=O)N1CCN(c2ccc(CCC(=O)OC(C)(C)C)o2)C(=O)C1]

tert-butyl 3-(5-(4-acryloyl-2-oxopiperazin-1-yl)furan-2-yl)propanoate (3, or Intermediate 1): Benzyl (E)-4-(5-(3-(tert-butoxy)-3-oxoprop-1-en-1-yl)furan-2-yl)-3-oxopiperazine-1-carboxylate (1.95 g, 4.59 mmol) was dissolved in EtOH (25 mL) and Pd/C (200 mg, 10% wt. Pd) was added. The reaction was placed under an atmosphere of H₂ and stirred vigorously overnight, before being filtered through celite twice and concentrated. The crude product was then redissolved in DCM (25 mL), cooled to O ° C. and treated with TEA (1.28 mL, 9.18 mmol) before a solution of acryloyl chloride (445 mL, 5.51 mmol) in DCM (5 mL) was added over 2 minutes. After stirring for 20 min, water was added and the mixture extracted with DCM three times. Combined organic extracts were washed with brine, dried over Na₂SO₄, concentrated, and the resulting crude oil was purified by silica gel chromatography (0-75% EtOAc/Hex) to obtain the title compound (3) as an oil (846 mg, 2.43 mmol, 53% over two steps). The title compound (3) was stored at −20° C. to avoid decomposition. LC/MS [M+2H-tBu]⁺ m/z calc. 293.1, found 293.1. 1H NMR (400 MHz, CDCl₃) δ 6.64-6.46 (m, 1H), 6.41 (dd, J=16.7, 2.0 Hz, 1H), 6.29 (d, J=3.2 Hz, 1H), 6.04 (d, J=3.3 Hz, 1H), 5.82 (dd, J=10.2, 2.0 Hz, 1H), 4.42 (d, J=24.9 Hz, 2H), 4.06-3.82 (m, 4H), 2.88 (t, J=7.8 Hz, 2H), 2.54 (d, J=7.6 Hz, 2H), 1.44 (s, 9H).

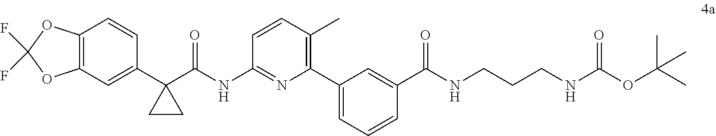 [SMILES: Cc1ccc(NC(=O)C2(c3ccc4c(c3)OC(F)(F)O4)CC2)nc1-c1cccc(C(=O)NCCCNC(=O)OC(C)(C)C)c1]

tert-butyl (3-(3-(6-(1-(2,2-difluorobenzo[d][1,3]dioxol-5-yl)cyclopropane-1-carboxamido)-3-methylpyridin-2-yl)benzamido)propyl)carbamate (4a): Lumacaftor (3-(6-(1-(2,2-difluorobenzo[d][1,3]dioxol-5-yl)cyclopropane-1-carboxamido)-3-methylpyridin-2-yl)benzoic acid) (18 mg, 0.04 mmol), tert-butyl (3-aminopropyl)carbamate (14 mg, 0.08 mmol), DIEA (35 mL, 0.20 mmol), and HOBt (5.4 mg, 0.04 mmol) were dissolved in DCM (1 mL), followed by the addition of EDCI HCl (15 mg, 0.05 mmol). The reaction was stirred at rt for 2 days before water was added, the mixture partitioned, and the aqueous layer extracted with DCM twice. The combined organic extracts were washed with brine, dried over Na₂SO₄, concentrated, and the resulting crude oil was purified by silica gel chromatography (0-60% EtOAc/Hex) to obtain the title compound (4a) as a clear oil (23 mg, 0.038 mmol, 94%). LC/MS [M+H]⁺ m/z calc. 609.24, found 609.3. 1H NMR (400 MHz, CDCl₃) δ 8.13 (d, J=8.4 Hz, 1H), 7.95 (s, 1H), 7.88 (d, J=7.6 Hz, 1H), 7.74 (s, 1H), 7.62 (d, J=8.5 Hz, 1H), 7.60-7.49 (m, 2H), 7.34 (s, 1H), 7.30-7.18 (m, 2H), 7.11 (d, J=8.2 Hz, 1H), 4.96 (s, 1H), 3.54 (q, J=6.2 Hz, 2H), 3.27 (q, J=6.3 Hz, 2H), 2.31 (s, 3H), 1.78 (q, J=3.9 Hz, 2H), 1.76-1.70 (m, 2H), 1.47 (s, 9H), 1.19 (q, J=3.9 Hz, 2H).

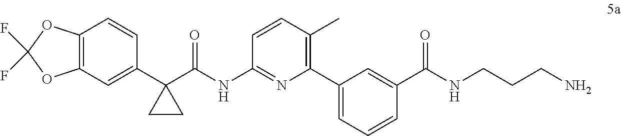 [SMILES: Cc1ccc(NC(=O)C2(c3ccc4c(c3)OC(F)(F)O4)CC2)nc1-c1cccc(C(=O)NCCCN)c1]

N-(3-aminopropyl)-3-(6-(1-(2,2-difluorobenzo[d][1,3]dioxol-5-yl)cyclopropane-1-carboxamido)-3-methylpyridin-2-yl)benzamide (5a): The Boc-protected amine 4a (23 mg, 0.038 mmol) was dissolved in DCM (1 mL) and TFA (1 mL) was added and the solution stirred for 2 hours. The volatiles were then evaporated and the resulting oil redissolved in DCM and treated with aqueous saturated NaHCO₃. The resulting mixture was then extracted with DCM three times, combined organic extracts dried over Na₂SO₄, concentrated to provide the title compound 5a (15 mg, 0.029 mmol, 78%) as a colorless oil which was used in the next step without further purification. LC/MS [M+H]⁺ m/z calc. 509.19, found 509.2. 1H NMR (400 MHz, CDCl₃) δ 10.73 (s, 1H), 8.96 (s, 1H), 8.66 (t, J=5.7 Hz, 1H), 7.95-7.85 (m, 3H), 7.79-7.66 (m, 2H), 7.60 (d, J=7.6 Hz, 1H), 7.56-7.49 (m, 2H), 7.41-7.30 (m, 2H), 3.33 (q, J=6.4 Hz, 2H), 2.88-2.77 (m, 2H), 2.21 (s, 3H), 1.79 (p, J=6.9 Hz, 2H), 1.52 (dd, J=4.9, 2.5 Hz, 2H), 1.19-1.15 (m, 2H).

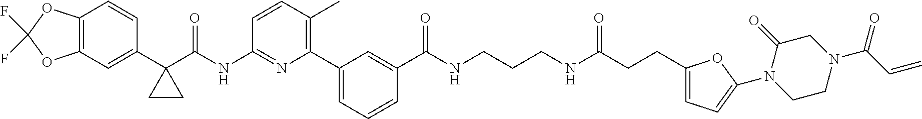 [SMILES: C=CC(=O)N1CCN(c2ccc(CCC(=O)NCCCNC(=O)c3cccc(-c4nc(NC(=O)C5(c6ccc7c(c6)OC(F)(F)O7)CC5)ccc4C)c3)o2)C(=O)C1]

N-(3-(3-(5-(4-acryloyl-2-oxopiperazin-1-yl)furan-2-yl)propanamido)propyl)-3-(6-(1-(2,2-difluorobenzo[d][1,3]dioxol-5-yl)cyclopropane-1-carboxamido)-3-methylpyridin-2-yl)benzamide (Compound 200): Intermediate 1 (tert-butyl 3-(5-(4-acryloyl-2-oxopiperazin-1-yl)furan-2-yl)propanoate) (14 mg, 0.04 mmol) was dissolved in DCM (0.6 mL) and TFA (0.3 mL) was added and the solution was stirred for 1 h at rt until starting material was consumed as monitored by TLC. Volatiles were evaporated, DCM was added and evaporated again. The residue was dissolved in DCM (1.5 mL) and DIEA (140 mL, 0.80 mmol) was added followed by N-(3-aminopropyl)-3-(6-(1-(2,2-difluorobenzo[d][1,3]dioxol-5-yl)cyclopropane-1-carboxamido)-3-methylpyridin-2-yl)benzamide (5.4 mg, 0.1 mmol). EDCI HCl (15 mg, 0.08 mmol) was then added and the mixture stirred for 16h. Water was added and the resulting suspension was extracted with DCM three times. The combined organic extracts were washed with brine and dried over Na₂SO₄ before being concentrated. The crude residue was purified by silica gel chromatography (0-5% MeOH/DCM) to obtain the title compound (Compound 200, 9.5 mg, 0.012 mmol, 30%) as a powder following lyophilization from 1:1 water:acetonitrile (2 mL). HRMS [M+H]⁺ m/z calc. 783.2949, found 783.2954. 1H NMR (400 MHz, CDCl₃) δ 8.09 (d, J=8.4 Hz, 1H), 7.93-7.87 (m, 1H), 7.83 (dt, J=7.5, 1.6 Hz, 1H), 7.72 (s, 1H), 7.59 (d, J=8.5 Hz, 1H), 7.57-7.45 (m, 2H), 7.29 (s, 1H), 7.23 (dd, J=8.2, 1.8 Hz, 1H), 7.19 (d, J=1.7 Hz, 1H), 7.07 (d, J=8.1 Hz, 1H), 6.50 (s, 1H), 6.43-6.33 (m, 2H), 6.19 (d, J=3.2 Hz, 1H), 6.07 (d, J=3.3 Hz, 1H), 5.81 (d, J=10.1 Hz, 1H), 4.47-4.31 (m, 2H), 4.04-3.78 (m, 4H), 3.36 (q, J=6.2 Hz, 2H), 3.32-3.23 (m, 2H), 2.96 (t, J=7.2 Hz, 2H), 2.55 (t, J=7.2 Hz, 2H), 2.26 (s, 3H), 1.74 (q, J=3.9 Hz, 2H), 1.69-1.58 (m, 2H), 1.16 (q, J=3.9 Hz, 2H). 13C NMR (151 MHz, CDCl₃) δ 172.5, 171.8, 167.4, 165.0, 155.5, 149.8, 148.9, 145.0, 144.1, 143.6, 141.0, 140.2, 134.9, 134.6, 131.8, 131.7, 130.0, 128.5, 127.8, 127.0, 126.6, 126.5, 126.3, 112.9, 112.4, 110.2, 107.6, 101.3, 36.0, 35.9, 35.2, 31.2, 29.5, 24.4, 19.2, 17.2 Synthesis of Compound 202

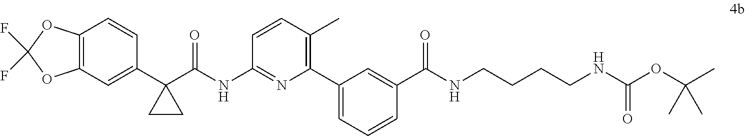 [SMILES: Cc1ccc(NC(=O)C2(c3ccc4c(c3)OC(F)(F)O4)CC2)nc1-c1cccc(C(=O)NCCCCNC(=O)OC(C)(C)C)c1]

tert-butyl (4-(3-(6-(1-(2,2-difluorobenzo[d][1,3]dioxol-5-yl)cyclopropane-1-carboxamido)-3-methylpyridin-2-yl)benzamido)butyl)carbamate (4b): Lumacaftor (3-(6-(1-(2,2-difluorobenzo[d][1,3]dioxol-5-yl)cyclopropane-1-carboxamido)-3-methylpyridin-2-yl)benzoic acid) (181 mg, 0.40 mmol), tert-butyl (5-aminopentyl)carbamate (121 mg, 0.60 mmol), DIEA (350 mL, 2.00 mmol), and HOBt (54 mg, 0.4 mmol) were reacted according to General Procedure A and purified by silica gel chromatography to obtain the title compound 4b as a clear oil (240 mg, 0.38 mmol, 95%). LC/MS [M+H]⁺ m/z calc. 637.28, found 637.3. 1H NMR (400 MHz, CDCl₃) δ 8.14 (d, J=8.4 Hz, 1H), 7.84 (s, 1H), 7.80 (dt, J=7.6, 1.6 Hz, 1H), 7.73 (s, 1H), 7.63 (d, J=8.5 Hz, 1H), 7.57 (dt, J=7.7, 1.5 Hz, 1H), 7.51 (t, J=7.6 Hz, 1H), 7.27 (dd, J=8.1, 1.8 Hz, 1H), 7.23 (d, J=1.7 Hz, 1H), 7.12 (d, J=8.2 Hz, 1H), 6.25 (s, 1H), 3.17 (d, J=6.8 Hz, 2H), 4.61 (s, 1H), 3.49 (q, J=7.0, 6.8, 6.3 Hz, 2H), 2.29 (s, 3H), 1.79 (q, J=3.9 Hz, 2H), 1.56 (q, J=7.2 Hz, 2H), 1.46 (s, 11H), 1.36-1.27 (m, 2H), 1.20 (q, J=3.9 Hz, 2H), 0.97-0.89 (m, 2H).

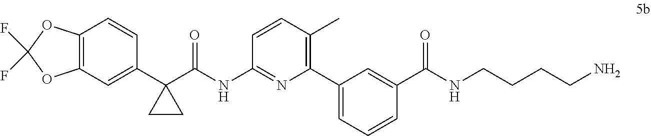 [SMILES: Cc1ccc(NC(=O)C2(c3ccc4c(c3)OC(F)(F)O4)CC2)nc1-c1cccc(C(=O)NCCCCN)c1]

N-(4-aminobutyl)-3-(6-(1-(2,2-difluorobenzo[d][1,3]dioxol-5-yl)cyclopropane-1-carboxamido)-3-methylpyridin-2-yl)benzamide (5b): The Boc-protected amine 4b (240 mg, 0.038 mmol) was deprotected according to General Procedure B to provide the amine 5b (104 mg, 0.20 mmol, quant.) as a colorless oil. LC/MS: [M+H]⁺ m/z calc. 523.2, found 523.2. 1H NMR (400 MHz, CDCl₃) δ 8.13 (dd, J=8.4, 1.7 Hz, 1H), 7.85 (tt, J=8.5, 1.8 Hz, 1H), 7.81 (dt, J=7.6, 1.6 Hz, 1H), 7.73 (s, 1H), 7.62 (dd, J=8.5, 2.1 Hz, 1H), 7.56 (ddt, J=7.7, 2.9, 1.5 Hz, 1H), 7.50 (td, J=7.6, 3.0 Hz, 1H), 7.27 (dd, J=8.2, 1.8 Hz, 1H), 7.22 (t, J=1.8 Hz, 1H), 7.11 (d, J=8.1 Hz, 1H), 7.03 (d, J=5.3 Hz, 1H), 3.57-3.46 (m, 2H), 3.27 (d, J=6.7 Hz, 1H), 2.80 (t, J=6.7 Hz, 1H), 2.28 (d, J=2.5 Hz, 3H), 1.98 (d, J=1.4 Hz, 1H), 1.86 (s, 1H), 1.79 (q, J=3.9 Hz, 2H), 1.72 (dd, J=8.1, 6.3 Hz, 1H), 1.63-1.53 (m, 1H), 1.20 (qd, J=4.0, 1.1 Hz, 2H)).

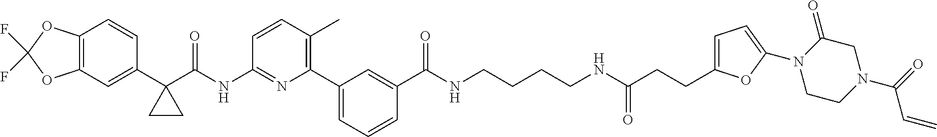 [SMILES: C=CC(=O)N1CCN(c2ccc(CCC(=O)NCCCCNC(=O)c3cccc(-c4nc(NC(=O)C5(c6ccc7c(c6)OC(F)(F)O7)CC5)ccc4C)c3)o2)C(=O)C1]

N-(5-(3-(5-(4-acryloyl-2-oxopiperazin-1-yl)furan-2-yl)propanamido)pentyl)-3-(6-(1-(2,2-difluorobenzo[d][1,3]dioxol-5-yl)cyclopropane-1-carboxamido)-3-methylpyridin-2-yl)benzamide (Compound 202): Intermediate 1 (tert-butyl 3-(5-(4-acryloyl-2-oxopiperazin-1-yl)furan-2-yl)propanoate) (30 mg, 0.086 mmol) was dissolved in DCM (0.6 mL) and TFA (0.3 mL) was added and the solution stirred for 1 h until starting material was consumed. Volatiles were evaporated, DCM was added and evaporated again. The residue was dissolved in DCM (1.5 mL) and DIEA (150 mL, 0.86 mmol) was added followed by N-(4-aminobutyl)-3-(6-(1-(2,2-difluorobenzo[d][1,3]dioxol-5-yl)cyclopropane-1-carboxamido)-3-methylpyridin-2-yl)benzamide (5b) (15 mg, 0.029 mmol). HATU (30 mg, 0.079 mmol) was then added and the mixture stirred for 16h. Water was added and the resulting suspension was extracted with DCM three times. Combined organic extracts were washed brine and dried over sodium sulfate, concentrated, then the crude residue was purified by silica gel chromatography (0-5% MeOH/DCM) to obtain Compound 202 (9.5 mg, 0.012 mmol, 30%) as a solid. HRMS (ESI): m z calc. 797.3032, found 797.3109. 1H NMR (400 MHz, CDCl₃) δ 8.12 (d, J=8.4 Hz, 1H), 7.88 (t, J=1.8 Hz, 1H), 7.84 (dt, J=7.6, 1.6 Hz, 1H), 7.74 (s, 1H), 7.62 (d, J=8.5 Hz, 1H), 7.56 (dt, J=7.7, 1.5 Hz, 1H), 7.51 (t, J=7.6 Hz, 1H), 7.26 (dd, J=8.2, 1.7 Hz, 1H), 7.22 (d, J=1.7 Hz, 1H), 7.11 (d, J=8.2 Hz, 1H), 6.80 (s, 1H), 6.53 (d, J=24.7 Hz, 1H), 6.41 (dd, J=16.7, 2.0 Hz, 1H), 6.20 (d, J=3.2 Hz, 1H), 6.07 (d, J=3.3 Hz, 2H), 5.83 (dd, J=10.2, 2.0 Hz, 1H), 4.38 (d, J=28.2 Hz, 2H), 4.07-3.79 (m, 4H), 3.73 (tt, J=9.8, 4.9 Hz, 1H), 3.45 (q, J=6.4 Hz, 2H), 3.27 (q, J=6.2 Hz, 2H), 3.20 (qd, J=7.4, 3.4 Hz, 1H), 2.94 (q, J=6.1, 5.0 Hz, 2H), 2.52 (t, J=7.2 Hz, 2H), 2.28 (s, 3H), 1.77 (q, J=3.9 Hz, 2H), 1.63-1.51 (m, 2H), 1.19 (q, J=3.9 Hz, 2H). 13C NMR (151 MHz, CDCl3) δ 171.8, 167.4, 165.0, 155.5, 149.9, 148.9, 144.7, 144.1, 143.6, 141.0, 140.2, 134.9, 134.7, 133.4, 131.8, 131.7, 128.5, 127.6, 127.0, 126.6, 126.4, 112.9, 112.4, 110.2, 107.4, 101.2, 55.5, 43.5, 39.6, 39.0, 34.9, 31.2, 26.8, 26.7, 24.3, 19.2, 18.6, 17.2, 17.2, 12.5.

### Synthesis of Compound 201

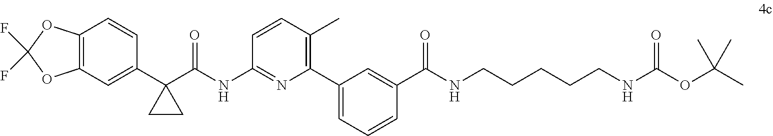 [SMILES: Cc1ccc(NC(=O)C2(c3ccc4c(c3)OC(F)(F)O4)CC2)nc1-c1cccc(C(=O)NCCCCCNC(=O)OC(C)(C)C)c1]

tert-butyl (5-(3-(6-(1-(2,2-difluorobenzo[d][1,3]dioxol-5-yl)cyclopropane-1-carboxamido)-3-methylpyridin-2-yl)benzamido)pentyl)carbamate (4c): Lumacaftor (3-(6-(1-(2,2-difluorobenzo[d][1,3]dioxol-5-yl)cyclopropane-1-carboxamido)-3-methylpyridin-2-yl)benzoic acid) (181 mg, 0.40 mmol), tert-butyl (5-aminopentyl)carbamate (121 mg, 0.60 mmol), DIEA (350 μL, 2.00 mmol), and HOBt (54 mg, 0.4 mmol) were dissolved in DCM (6 mL), followed by addition of EDCI HCl (153 mg, 0.50 mmol). The reaction was stirred at rt for 16 hours before water was added, the mixture partitioned, and the aqueous layer extracted with DCM twice. The combined organic extracts were washed with brine, dried over Na₂SO₄, concentrated, and the resulting crude oil was purified by silica gel chromatography (0-50% EtOAc/Hex) to obtain 4c as an oil (240 mg, 0.38 mmol, 95%). LC/MS [M+H]⁺ m/z calc. 637.28, found 637.3. 1H NMR (400 MHz, CDCl₃) δ 8.14 (d, J=8.4 Hz, 1H), 7.84 (s, 1H), 7.80 (dt, J=7.6, 1.6 Hz, 1H), 7.73 (s, 1H), 7.63 (d, J=8.5 Hz, 1H), 7.57 (dt, J=7.7, 1.5 Hz, 1H), 7.51 (t, J=7.6 Hz, 1H), 7.27 (dd, J=8.1, 1.8 Hz, 1H), 7.23 (d, J=1.7 Hz, 1H), 7.12 (d, J=8.2 Hz, 1H), 6.25 (s, 1H), 3.17 (d, J=6.8 Hz, 2H), 4.61 (s, 1H), 3.49 (q, J=7.0, 6.8, 6.3 Hz, 2H), 2.29 (s, 3H), 1.79 (q, J=3.9 Hz, 2H), 1.56 (q, J=7.2 Hz, 2H), 1.46 (s, 11H), 1.36-1.27 (m, 2H), 1.20 (q, J=3.9 Hz, 2H), 0.97-0.89 (m, 2H).

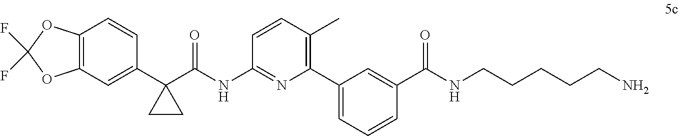 [SMILES: Cc1ccc(NC(=O)C2(c3ccc4c(c3)OC(F)(F)O4)CC2)nc1-c1cccc(C(=O)NCCCCCN)c1]

N-(5-aminopentyl)-3-(6-(1-(2,2-difluorobenzo[d][1,3]dioxol-5-yl)cyclopropane-1-carboxamido)-3-methylpyridin-2-yl)benzamide (5c): 4c (240 mg, 0.038 mmol) was dissolved in DCM (2 mL), TFA (2 mL) was added, and the solution stirred for 2 hours. The volatiles were then evaporated and the resulting oil redissolved in DCM and treated with aqueous saturated NaHCO₃. The layers were separated and the aqueous layer was then extracted with DCM three times. The combined organic extracts were dried over Na₂SO₄, and concentrated to provide the title compound 5c (184 mg, 0.34 mmol, 85% over two steps) as an oil which was used in the next step without further purification. LC/MS [M+H]⁺ m/z calc. 537.22, found 537.2. 1H NMR (400 MHz, CDCl₃) δ 8.09 (d, J=8.4 Hz, 1H), 7.80 (t, J=1.8 Hz, 1H), 7.76 (dd, J=7.7, 1.5 Hz, 1H), 7.69 (s, 1H), 7.59 (d, J=8.5 Hz, 1H), 7.57-7.50 (m, 1H), 7.47 (t, J=7.6 Hz, 1H), 7.23 (dd, J=8.2, 1.7 Hz, 1H), 7.19 (d, J=1.8 Hz, 1H), 7.08 (d, J=8.2 Hz, 1H), 6.30 (s, 1H), 3.45 (q, J=6.7 Hz, 2H), 2.74 (t, J=6.8 Hz, 2H), 2.25 (s, 3H), 1.65-1.59 (m, 2H), 1.57-1.47 (m, 2H), 1.48-1.40 (m, 2H), 1.33-1.23 (m, 2H), 1.20-1.12 (m, 2H), 0.91-0.85 (m, 2H).

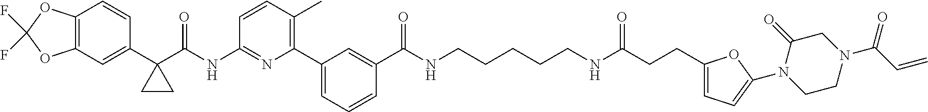 [SMILES: C=CC(=O)N1CCN(c2ccc(CCC(=O)NCCCCCNC(=O)c3cccc(-c4nc(NC(=O)C5(c6ccc7c(c6)OC(F)(F)O7)CC5)ccc4C)c3)o2)C(=O)C1]

N-(5-(3-(5-(4-acryloyl-2-oxopiperazin-1-yl)furan-2-yl)propanamido)pentyl)-3-(6-(1-(2,2-difluorobenzo[d][1,3]dioxol-5-yl)cyclopropane-1-carboxamido)-3-methylpyridin-2-yl)benzamide (Compound 201): 3 (tert-butyl 3-(5-(4-acryloyl-2-oxopiperazin-1-yl)furan-2-yl)propanoate) (70 mg, 0.20 mmol) was dissolved in DCM (1.0 mL) and TFA (0.8 mL) was added and the solution stirred for 1 h until starting material was consumed as monitored by TLC. The volatiles were evaporated, DCM was added and evaporated again. The residue was dissolved in DMF (1.5 mL) and DIEA (150 μL, 0.86 mmol) was added followed by intermediate 5c (N-(5-aminopentyl)-3-(6-(1-(2,2-difluorobenzo[d][1,3]dioxol-5-yl)cyclopropane-1-carboxamido)-3-methylpyridin-2-yl)benzamide) (54 mg, 0.1 mmol). HATU (152 mg, 0.4 mmol) was then added and the mixture stirred for 1 h. Water was added, and the resulting suspension was extracted with DCM three times. Combined organic extracts were washed twice with 1M HCl twice, saturated NaHCO₃, twice with 5% LiCl, brine, and dried over Na₂SO₄ before being concentrated. The crude residue was purified by silica gel chromatography (0-4% MeOH/DCM) to obtain the Compound 202 (35 mg, 0.043 mmol, 43%) as a powder following lyophilization from 1:1 water:acetonitrile (2 mL). HRMS [M+H]⁺ m/z calc. 811.3262, found 811.3267. 1H NMR (600 MHz, CDCl₃) δ 8.11 (d, J=8.4 Hz, 1H), 7.85 (t, J=1.8 Hz, 1H), 7.81 (dt, J=7.8, 1.5 Hz, 1H), 7.71 (s, 1H), 7.61 (d, J=8.5 Hz, 1H), 7.55 (dt, J=7.7, 1.4 Hz, 1H), 7.49 (t, J=7.6 Hz, 1H), 7.25 (dd, J=8.2, 1.8 Hz, 1H), 7.21 (d, J=1.8 Hz, 1H), 7.10 (d, J=8.2 Hz, 1H), 6.53 (s, 1H), 6.41 (dd, J=16.7, 1.8 Hz, 2H), 6.22 (d, J=3.3 Hz, 1H), 6.03 (d, J=3.3 Hz, 1H), 5.82 (dd, J=10.4, 1.8 Hz, 2H), 4.54-4.32 (m, 2H), 4.07-3.79 (m, 4H), 3.45 (q, J=6.6 Hz, 2H), 3.24 (q, J=6.6 Hz, 2H), 2.91 (t, J=7.3 Hz, 2H), 2.46 (t, J=7.3 Hz, 2H), 2.27 (s, 3H), 1.77 (q, J=3.9 Hz, 2H), 1.65-1.59 (m, 2H), 1.52 (p, J=7.0 Hz, 2H), 1.40-1.32 (m, 2H), 1.18 (q, J=3.9 Hz, 2H). 13C NMR (151 MHz, CDCl₃) δ 171.7, 167.4, 165.0, 155.5, 148.9, 144.8, 144.1, 143.6, 141.0, 140.2, 134.9, 134.8, 131.8, 128.4, 127.5, 127.0, 126.6, 126.6, 126.3, 112.9, 112.4, 110.2, 107.4, 100.9, 39.7, 39.1, 31.2, 29.0, 24.2, 23.7, 19.2, 17.2.

### Synthesis of Compound 203

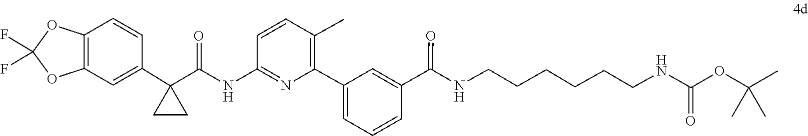 [SMILES: Cc1ccc(NC(=O)C2(c3ccc4c(c3)OC(F)(F)O4)CC2)nc1-c1cccc(C(=O)NCCCCCCNC(=O)OC(C)(C)C)c1]

tert-butyl (6-(3-(6-(1-(2,2-difluorobenzo[d][1,3]dioxol-5-yl)cyclopropane-1-carboxamido)-3-methylpyridin-2-yl)benzamido)hexyl)carbamate (4d): Lumacaftor (100 mg, 0.22 mmol) and tert-butyl (6-aminohexyl)carbamate were reacted according to General Procedure A and purified by silica gel chromatography (0-60% EtOAc/Hex) to obtain intermediate 4d (114 mg, 0.18 mmol, 80%) as an oil. LC/MS [M+H]⁺ m/z calc. 651.3, found 651.2. 1H NMR (400 MHz, CDCl3) δ 8.14 (d, J=8.4 Hz, 1H), 7.86 (s, 1H), 7.82 (d, J=7.7 Hz, 1H), 7.72 (s, 1H), 7.63 (d, J=8.5 Hz, 1H), 7.57 (dt, J=7.6, 1.5 Hz, 1H), 7.51 (t, J=7.6 Hz, 1H), 7.27 (dd, J=8.2, 1.8 Hz, 1H), 7.23 (d, J=1.7 Hz, 1H), 7.12 (d, J=8.1 Hz, 1H), 6.37 (s, 1H), 4.58 (s, 1H), 3.48 (q, J=6.7 Hz, 2H), 3.17 (q, J=6.7 Hz, 2H), 2.29 (s, 3H), 1.79 (q, J=3.9 Hz, 2H), 1.69-1.64 (m, 1H), 1.58-1.49 (m, 1H), 1.46 (s, 9H), 1.45-1.38 (m, 6H), 1.20 (q, J=3.9 Hz, 2H).

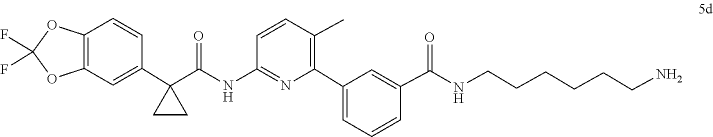 [SMILES: Cc1ccc(NC(=O)C2(c3ccc4c(c3)OC(F)(F)O4)CC2)nc1-c1cccc(C(=O)NCCCCCCN)c1]

N-(6-aminohexyl)-3-(6-(1-(2,2-difluorobenzo[d][1,3]dioxol-5-yl)cyclopropane-1-carboxamido)-3-methylpyridin-2-yl)benzamide) (5d): 4d (114 mg, 0.18 mmol) was deprotected according to General Procedure B to provide the amine 5d (99 mg, 0.18 mmol, quant.) as an oil. LC/MS [M+H]⁺ m/z calc. 551.2, found 551.2. 1H NMR (400 MHz, CDCl3) δ 8.10 (d, J=8.4 Hz, 1H), 7.78 (s, 1H), 7.74 (dt, J=7.5, 1.6 Hz, 1H), 7.69 (s, 1H), 7.59 (d, J=8.4 Hz, 1H), 7.53 (dt, J=7.7, 1.5 Hz, 1H), 7.47 (t, J=7.6 Hz, 1H), 7.22 (dd, J=8.2, 1.8 Hz, 1H), 7.18 (d, J=1.6 Hz, 1H), 7.07 (d, J=8.2 Hz, 1H), 6.17 (s, 1H), 3.44 (td, J=7.2, 5.8 Hz, 2H), 2.68 (t, J=6.8 Hz, 2H), 2.24 (s, 3H), 1.99 (s, 1H), 1.81 (s, 1H), 1.74 (q, J=3.9 Hz, 2H), 1.67-1.55 (m, 3H), 1.51-1.33 (m, 5H), 1.16 (q, J=3.9 Hz, 2H).

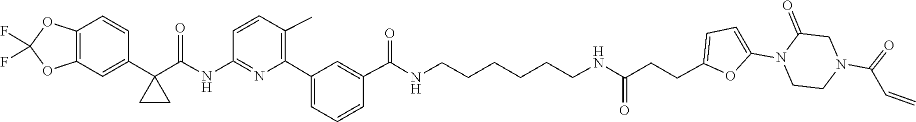 [SMILES: C=CC(=O)N1CCN(c2ccc(CCC(=O)NCCCCCCNC(=O)c3cccc(-c4nc(NC(=O)C5(c6ccc7c(c6)OC(F)(F)O7)CC5)ccc4C)c3)o2)C(=O)C1]

N-(6-(3-(5-(4-acryloyl-2-oxopiperazin-1-yl)furan-2-yl)propanamido)hexyl)-3-(6-(1-(2,2-difluorobenzo[d][1,3]dioxol-5-yl)cyclopropane-1-carboxamido)-3-methylpyridin-2-yl)benzamide (Compound 203): 3 (30 mg, 0.086 mmol) was deprotected and coupled to 5d (16 mg, 0.029 mmol) following General Procedure C to provide Compound 203 (17.4 mg, 0.021 mmol, 73%) as a clear colorless oil. HRMS (ESI): [M+H]⁺ m/z calc. 825.3345, found 825.3425. 1H NMR (400 MHz, CDCl₃) δ 8.12 (d, J=8.4 Hz, 1H), 7.89-7.79 (m, 2H), 7.73 (s, 1H), 7.62 (d, J=8.5 Hz, 1H), 7.56 (dt, J=7.7, 1.5 Hz, 1H), 7.51 (t, J=7.6 Hz, 1H), 7.27 (dd, J=8.2, 1.8 Hz, 1H), 7.22 (d, J=1.7 Hz, 1H), 7.11 (d, J=8.2 Hz, 1H), 6.54 (d, J=31.0 Hz, 2H), 6.41 (dd, J=16.8, 1.9 Hz, 1H), 6.25 (d, J=3.3 Hz, 1H), 6.07 (d, J=3.3 Hz, 1H), 5.98 (d, J=39.7 Hz, 1H), 5.83 (dd, J=10.3, 2.0 Hz, 1H), 4.42 (d, J=21.6 Hz, 2H), 4.05-3.81 (m, 4H), 3.74 (p, J=6.7 Hz, 2H), 3.45 (q, J=6.7 Hz, 2H), 3.22 (dq, J=13.2, 6.9 Hz, 3H), 2.94 (q, J=6.4, 5.5 Hz, 2H), 2.52 (t, J=7.4 Hz, 2H), 2.28 (s, 3H), 1.78 (q, J=3.9 Hz, 2H), 1.61 (p, J=6.9 Hz, 2H), 1.42-1.30 (m, 3H), 1.20 (q, J=3.9 Hz, 2H). 13C NMR (151 MHz, CDCl₃) δ 171.8, 167.3, 155.5, 149.7, 148.9, 144.7, 144.1, 143.6, 141.0, 140.2, 134.9, 134.9, 133.4, 131.7, 131.7, 130.0, 128.5, 127.5, 127.0, 126.6, 126.6, 126.4, 112.9, 112.4, 110.2, 107.3, 100.8, 55.6, 43.6, 39.6, 39.1, 34.8, 31.2, 29.4, 29.3, 26.0, 25.9, 24.2, 19.1, 18.6, 17.2, 12.5.

Synthesis of Compound 204

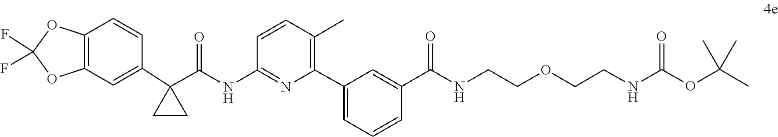 [SMILES: Cc1ccc(NC(=O)C2(c3ccc4c(c3)OC(F)(F)O4)CC2)nc1-c1cccc(C(=O)NCCOCCNC(=O)OC(C)(C)C)c1]

tert-butyl (2-(2-(3-(6-(1-(2,2-difluorobenzo[d][1,3]dioxol-5-yl)cyclopropane-1-carboxamido)-3-methylpyridin-2-yl)benzamido)ethoxy)ethyl)carbamate (4e): Lumacaftor (100 mg, 0.22 mmol) and tert-butyl (2-(2-aminoethoxy)ethyl)carbamate (57 mg, 0.28 mmol) were reacted according to General Procedure A and purified by silica gel chromatography (0-60% EtOAc/Hex) to obtain 4e (122 mg, 0.19 mmol, 87%) as a clear colorless oil. LC/MS [M+H]+ m/z calc. 639.3, found 639.2. 1H NMR (400 MHz, Chloroform-d) δ 8.14 (d, J=8.4 Hz, 1H), 7.88 (t, J=1.8 Hz, 1H), 7.81 (dt, J=7.5, 1.6 Hz, 1H), 7.72 (s, 1H), 7.63 (d, J=8.5 Hz, 1H), 7.59 (dt, J=7.7, 1.5 Hz, 1H), 7.52 (t, J=7.6 Hz, 1H), 7.27 (dd, J=8.2, 1.8 Hz, 1H), 7.23 (d, J=1.7 Hz, 1H), 7.12 (d, J=8.2 Hz, 1H), 6.60 (s, 1H), 4.87 (s, 1H), 3.74-3.62 (m, 4H), 3.58 (t, J=5.2 Hz, 2H), 3.41-3.31 (m, 2H), 2.29 (s, 3H), 1.79 (q, J=3.9 Hz, 2H), 1.46 (s, 9H), 1.20 (q, J=3.9 Hz, 2H).

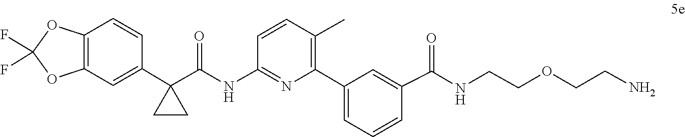 [SMILES: Cc1ccc(NC(=O)C2(c3ccc4c(c3)OC(F)(F)O4)CC2)nc1-c1cccc(C(=O)NCCOCCN)c1]

N-(2-(2-aminoethoxy)ethyl)-3-(6-(1-(2,2-difluorobenzo[d][1,3]dioxol-5-yl)cyclopropane-1-carboxamido)-3-methylpyridin-2-yl)benzamide (5e): 4e (122 mg, 0.19 mmol) was deprotected according to General Procedure B to provide the amine 5e (102 mg, 0.19 mmol, quant.) as an oil. LC/MS: [M+H]⁺ m/z calc. 539.2 found 639.2. 1H NMR (400 MHz, Chloroform-d) δ 8.14 (d, J=8.4 Hz, 1H), 7.88 (t, J=1.7 Hz, 1H), 7.85 (s, 1H), 7.77 (s, 1H), 7.63 (d, J=8.5 Hz, 1H), 7.57 (dt, J=7.7, 1.5 Hz, 1H), 7.51 (t, J=7.6 Hz, 1H), 7.27 (dd, J=8.1, 1.8 Hz, 1H), 7.23 (d, J=1.7 Hz, 1H), 7.12 (d, J=8.2 Hz, 1H), 6.91 (s, OH), 3.70 (tdd, J=7.9, 4.0, 1.2 Hz, 4H), 3.55 (t, J=5.2 Hz, 2H), 2.91 (t, J=5.2 Hz, 2H), 2.29 (s, 3H), 1.79 (q, J=3.9 Hz, 2H), 1.20 (q, J=3.9 Hz, 2H).

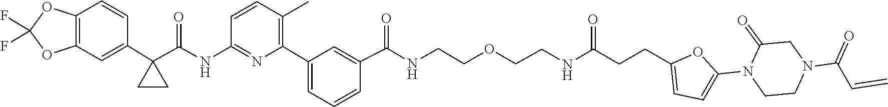 [SMILES: C=CC(=O)N1CCN(c2ccc(CCC(=O)NCCOCCNC(=O)c3cccc(-c4nc(NC(=O)C5(c6ccc7c(c6)OC(F)(F)O7)CC5)ccc4C)c3)o2)C(=O)C1]

N-(2-(2-(3-(5-(4-acryloyl-2-oxopiperazin-1-yl)furan-2-yl)propanamido)ethoxy)ethyl)-3-(6-(1-(2,2-difluorobenzo[d][1,3]dioxol-5-yl)cyclopropane-1-carboxamido)-3-methylpyridin-2-yl)benzamide (Compound 204). 3 (30 mg, 0.086 mmol) was deprotected and coupled to intermediate 5e (23 mg, 0.043 mmol) following General Procedure C to provide Compound 204 (10.9 mg, 0.0134 mmol, 31% yield) as a foam. HRMS (ESI): [M+H]⁺ m/z calc. 813.31, found 813.3055. ¹H NMR (600 MHz, Chloroform-d) δ 8.10 (d, J=8.4 Hz, 1H), 7.88 (t, J=1.8 Hz, 1H), 7.82 (dt, J=7.7, 1.5 Hz, 1H), 7.72 (s, 1H), 7.60 (d, J=8.5 Hz, 1H), 7.55 (dt, J=7.6, 1.4 Hz, 1H), 7.48 (t, J=7.7 Hz, 1H), 7.25 (dd, J=8.2, 1.8 Hz, 1H), 7.21 (d, J=1.7 Hz, 1H), 7.09 (d, J=8.2 Hz, 1H), 6.86 (s, 1H), 6.39 (dd, J=16.7, 1.8 Hz, 1H), 6.20 (d, J=3.3 Hz, 1H), 6.02 (d, J=3.2 Hz, 1H), 5.81 (dd, J=10.4, 1.8 Hz, 1H), 3.82 (s, 2H), 3.73 (hept, J=6.6 Hz, 2H), 3.63 (d, J=4.1 Hz, 4H), 3.53 (t, J=5.1 Hz, 2H), 3.41 (q, J=5.3 Hz, 2H), 3.19 (q, J=7.4 Hz, 2H), 2.89 (t, J=7.5 Hz, 2H), 2.47 (t, J=7.3 Hz, 2H), 2.26 (s, 3H), 1.48 (t, J=7.4 Hz, 3H), 1.18 (q, J=3.9 Hz, 2H), 0.12-0.06 (m, 1H). ¹³C NMR (151 MHz, CDCl₃) δ 171.77, 167.53, 165.03, 155.44, 149.75, 148.91, 144.71, 144.11, 143.59, 140.95, 140.22, 134.94, 134.55, 131.91, 131.68, 128.46, 127.72, 126.98, 126.64, 126.36, 112.96, 112.39, 110.21, 107.27, 100.91, 69.63, 69.50, 55.72, 53.43, 43.65, 39.83, 39.18, 34.69, 31.20, 24.08, 19.14, 17.18, 12.52.

### Synthesis of Compound 205

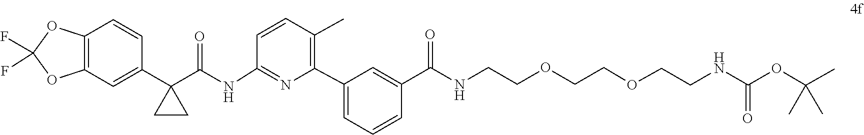 [SMILES: Cc1ccc(NC(=O)C2(c3ccc4c(c3)OC(F)(F)O4)CC2)nc1-c1cccc(C(=O)NCCOCCOCCNC(=O)OC(C)(C)C)c1]

tert-butyl (2-(2-(2-(3-(6-(1-(2,2-difluorobenzo[d][1,3]dioxol-5-yl)cyclopropane-1-carboxamido)-3-methylpyridin-2-yl)benzamido)ethoxy)ethoxy)ethyl)carbamate (4f): Lumacaftor (100 mg, 0.22 mmol) and tert-butyl (2-(2-(2-aminoethoxy)ethoxy)ethyl)carbamate (70 mg, 0.28 mmol) were reacted according to General Procedure A and purified by silica gel chromatography (0-80% EtOAc/Hex) to obtain 4f (127 mg, 0.19 mmol, 85%) as an oil. LC/MS: [M+H]⁺ m/z calc. 683.3, found 683.3. 1H NMR (400 MHz, Chloroform-d) δ 8.14 (d, J=8.4 Hz, 1H), 7.88 (s, 1H), 7.80 (d, J=7.5 Hz, 1H), 7.77-7.72 (m, 1H), 7.63 (d, J=8.4 Hz, 1H), 7.57 (d, J=7.5 Hz, 1H), 7.52 (t, J=7.6 Hz, 1H), 7.27 (dd, J=8.2, 1.8 Hz, 1H), 7.23 (d, J=1.7 Hz, 1H), 7.11 (d, J=8.2 Hz, 1H), 6.74 (s, 1H), 5.02 (s, 1H), 3.75-3.61 (m, 8H), 3.56 (t, J=5.4 Hz, 2H), 3.31 (d, J=5.8 Hz, 2H), 2.28 (s, 3H), 1.79 (q, J=3.9 Hz, 2H), 1.45 (s, 9H), 1.20 (q, J=3.9 Hz, 2H).

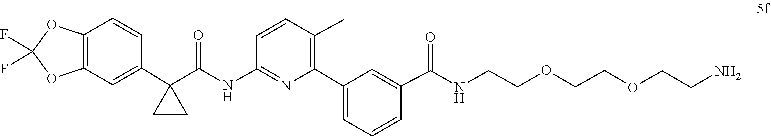 [SMILES: Cc1ccc(NC(=O)C2(c3ccc4c(c3)OC(F)(F)O4)CC2)nc1-c1cccc(C(=O)NCCOCCOCCN)c1]

N-(2-(2-(2-aminoethoxy)ethoxy)ethyl)-3-(6-(1-(2,2-difluorobenzo[d][1,3]dioxol-5-yl)cyclopropane-1-carboxamido)-3-methylpyridin-2-yl)benzamide (5f): 4f (127 mg, 0.19 mmol) was deprotected according to General Procedure B to provide the amine 5f (111 mg, 0.19 mmol, quant.) as an oil. LC/MS: [M+H]⁺ m/z calc. 583.2, found 583.3. 1H NMR (400 MHz, Chloroform-d) δ 8.13 (d, J=8.4 Hz, 1H), 7.89 (t, J=1.7 Hz, 1H), 7.83 (dt, J=7.7, 1.5 Hz, 1H), 7.78 (s, 1H), 7.63 (d, J=8.5 Hz, 1H), 7.56 (dt, J=7.7, 1.5 Hz, 1H), 7.51 (t, J=7.6 Hz, 1H), 7.27 (dd, J=8.2, 1.7 Hz, 1H), 7.23 (d, J=1.7 Hz, 1H), 7.12 (d, J=8.2 Hz, 1H), 7.08 (s, 1H), 3.73-3.62 (m, 9H), 3.51 (t, J=5.2 Hz, 2H), 2.82 (t, J=5.1 Hz, 2H), 2.28 (s, 3H), 1.79 (q, J=3.9 Hz, 2H), 1.20 (q, J=3.9 Hz, 2H).

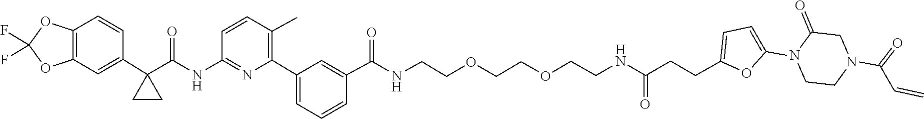 [SMILES: C=CC(=O)N1CCN(c2ccc(CCC(=O)NCCOCCOCCNC(=O)c3cccc(-c4nc(NC(=O)C5(c6ccc7c(c6)OC(F)(F)O7)CC5)ccc4C)c3)o2)C(=O)C1]

N-(2-(2-(2-(3-(5-(4-acryloyl-2-oxopiperazin-1-yl)furan-2-yl)propanamido)ethoxy)ethoxy)ethyl)-3-(6-(1-(2,2-difluorobenzo[d][1,3]dioxol-5-yl)cyclopropane-1-carboxamido)-3-methylpyridin-2-yl)benzamide (Compound 205). Intermediate 3 (30 mg, 0.086 mmol) was deprotected and coupled to intermediate 5f (25 mg, 0.043 mmol) following General Procedure C to provide Compound 205 (11.6 mg, 0.0134 mmol, 31% yield) as an oil. HRMS (ESI): [M+H]⁺ m/z calc. 857.33, found 857.3319. ¹H NMR (600 MHz, Chloroform-d) δ 8.11 (d, J=8.4 Hz, 1H), 7.86 (tt, J=1.8, 1.2 Hz, 1H), 7.79 (ddd, J=7.7, 1.8, 1.2 Hz, 1H), 7.72 (s, 1H), 7.62-7.58 (m, 1H), 7.55 (ddd, J=7.6, 1.7, 1.2 Hz, 1H), 7.48 (td, J=7.7, 0.6 Hz, 1H), 7.25 (dd, J=8.2, 1.8 Hz, 1H), 7.21 (d, J=1.7 Hz, 1H), 7.09 (d, J=8.2 Hz, 1H), 6.83 (d, J=5.8 Hz, 1H), 6.41 (dd, J=16.7, 1.8 Hz, 1H), 6.24 (d, J=3.2 Hz, 1H), 6.18 (s, 1H), 6.05 (dd, J=3.3, 1.0 Hz, 1H), 5.82 (dd, J=10.5, 1.8 Hz, 1H), 5.32 (s, 1H), 4.40 (d, J=39.8 Hz, 2H), 3.94 (d, J=47.9 Hz, 1H), 3.85 (s, 2H), 3.70-3.58 (m, 7H), 3.50 (dd, J=5.6, 4.8 Hz, 2H), 3.39 (q, J=5.4 Hz, 2H), 2.93 (t, J=7.5 Hz, 2H), 2.47 (t, J=7.5 Hz, 2H), 2.25 (s, 3H), 2.19 (s, 1H), 1.76 (q, J=3.8 Hz, 2H), 1.47 (d, J=12.2 Hz, 1H), 1.18 (p, J=3.8 Hz, 2H). ¹³C NMR 151 MHz, CDCl₃) δ 171.78, 171.54, 167.31, 164.98, 155.46, 148.91, 144.68, 144.12, 143.60, 140.94, 140.25, 134.93, 134.64, 131.88, 131.68, 128.46, 127.72, 126.98, 126.63, 126.51, 126.34, 113.00, 112.39, 110.19, 107.18, 100.77, 70.23, 70.18, 69.80, 55.62, 53.43, 43.58, 39.81, 39.16, 34.67, 31.20, 30.92, 23.97, 19.13, 17.19, 12.47, 1.02.

### Synthesis of Compound 206

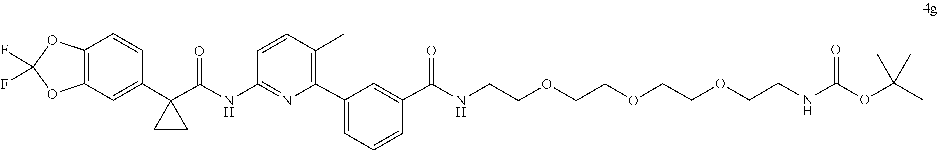 [SMILES: Cc1ccc(NC(=O)C2(c3ccc4c(c3)OC(F)(F)O4)CC2)nc1-c1cccc(C(=O)NCCOCCOCCOCCNC(=O)OC(C)(C)C)c1]

tert-butyl (1-(3-(6-(1-(2,2-difluorobenzo[d][1,3]dioxol-5-yl)cyclopropane-1-carboxamido)-3-methylpyridin-2-yl)phenyl)-1-oxo-5,8,11-trioxa-2-azatridecan-13-yl)carbamate (4g): Lumacaftor (100 mg, 0.22 mmol) and tert-butyl (2-(2-(2-(2-aminoethoxy)ethoxy)ethoxy)-ethyl)carbamate (82 mg, 0.28 mmol) were reacted according to General Procedure A and purified by silica gel chromatography (0-100% EtOAc/Hex) to obtain 4g (139 mg, 0.19 mmol, 87%) as an oil. LC/MS: [M+H]⁺ m/z calc. 727.3, found 727.2. 1H NMR (400 MHz, Chloroform-d) δ 8.14 (d, J=8.4 Hz, 1H), 7.89 (s, 1H), 7.82 (d, J=7.5 Hz, 1H), 7.73 (s, 1H), 7.63 (d, J=8.5 Hz, 1H), 7.57 (d, J=7.5 Hz, 1H), 7.51 (t, J=7.6 Hz, 1H), 7.27 (dd, J=8.2, 1.8 Hz, 1H), 7.23 (d, J=1.7 Hz, 1H), 7.12 (d, J=8.2 Hz, 1H), 6.80 (s, 1H), 3.73-3.66 (m, 9H), 3.64 (dd, J=6.1, 3.2 Hz, 2H), 3.59 (dd, J=6.1, 3.2 Hz, 2H), 3.50 (t, J=5.1 Hz, 2H), 3.30 (d, J=5.7 Hz, 2H), 2.28 (s, 3H), 1.79 (q, J=3.9 Hz, 2H), 1.46 (s, 9H), 1.20 (q, J=3.9 Hz, 2H).

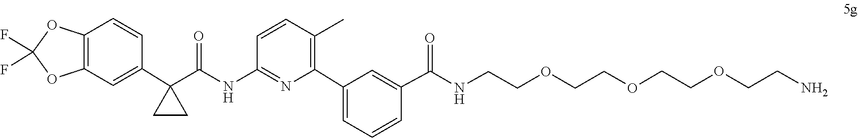 [SMILES: Cc1ccc(NC(=O)C2(c3ccc4c(c3)OC(F)(F)O4)CC2)nc1-c1cccc(C(=O)NCCOCCOCCOCCN)c1]

N-(2-(2-(2-(2-aminoethoxy)ethoxy)ethoxy)ethyl)-3-(6-(1-(2,2-difluorobenzo[d][1,3]dioxol-5-yl)cyclopropane-1-carboxamido)-3-methylpyridin-2-yl)benzamide (5g): 4g (139 mg, 0.19 mmol) was deprotected according to General Procedure B to provide the amine 5g (119 mg, 0.19 mmol, quant.) as an oil. LC/MS: [M+H]⁺ m/z calc. 627.3, found 627.3. 1H NMR (400 MHz, Chloroform-d) δ 8.13 (d, J=8.4 Hz, 1H), 7.93 (t, J=1.8 Hz, 1H), 7.87 (dt, J=7.6, 1.6 Hz, 1H), 7.76 (s, 1H), 7.62 (d, J=8.5 Hz, 1H), 7.60 (s, 1H), 7.55 (dt, J=7.7, 1.5 Hz, 1H), 7.50 (t, J=7.6 Hz, 1H), 7.27 (dd, J=8.2, 1.7 Hz, 1H), 7.23 (d, J=1.7 Hz, 1H), 7.12 (d, J=8.2 Hz, 1H), 3.73-3.63 (m, 9H), 3.61 (dt, J=6.0, 1.8 Hz, 4H), 3.48-3.43 (m, 2H), 2.82-2.75 (m, 2H), 2.29 (s, 3H), 1.79 (q, J=3.9 Hz, 2H), 1.20 (q, J=3.9 Hz, 2H).

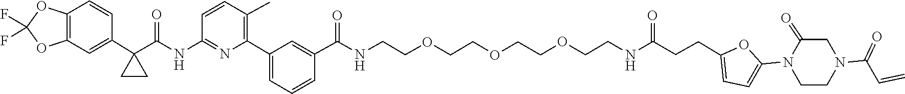 [SMILES: C=CC(=O)N1CCN(c2ccc(CCC(=O)NCCOCCOCCOCCNC(=O)c3cccc(-c4nc(NC(=O)C5(c6ccc7c(c6)OC(F)(F)O7)CC5)ccc4C)c3)o2)C(=O)C1]

N-(15-(5-(4-acryloyl-2-oxopiperazin-1-yl)furan-2-yl)-13-oxo-3,6,9-trioxa-12-azapentadecyl)-3-(6-(1-(2,2-difluorobenzo[d][1,3]dioxol-5-yl)cyclopropane-1-carboxamido)-3-methylpyridin-2-yl)benzamide (Compound 206). 3 (30 mg, 0.086 mmol) was deprotected and coupled to intermediate 5g (27 mg, 0.043 mmol) following General Procedure C to provide Compound 206 (13.7 mg, 0.0152 mmol, 35% yield) as an oil. HRMS (ESI): [M+H]⁺ m/z calc. 901.36, found 901.3584. ¹H NMR 1H NMR (600 MHz, Chloroform-d) δ 8.10 (d, J=8.5 Hz, 1H), 7.87 (t, J=1.8 Hz, 1H), 7.80 (dt, J=7.8, 1.5 Hz, 1H), 7.73 (s, 1H), 7.60 (d, J=8.5 Hz, 1H), 7.54 (dt, J=7.7, 1.4 Hz, 1H), 7.48 (t, J=7.7 Hz, 1H), 7.25 (dd, J=8.2, 1.8 Hz, 1H), 7.21 (d, J=1.7 Hz, 1H), 7.09 (d, J=8.2 Hz, 1H), 6.93 (d, J=6.0 Hz, 1H), 6.41 (dd, J=16.7, 1.8 Hz, 1H), 6.25 (d, J=3.2 Hz, 1H), 6.05 (d, J=3.3 Hz, 1H), 5.82 (dd, J=10.4, 1.8 Hz, 1H), 4.41 (d, J=35.7 Hz, 2H), 3.95 (d, J=50.4 Hz, 3H), 3.85 (s, 2H), 3.70-3.62 (m, 8H), 3.62-3.57 (m, 2H), 3.57-3.52 (m, 2H), 3.47 (dd, J=5.6, 4.6 Hz, 2H), 3.39 (q, J=5.3 Hz, 2H), 2.93 (t, J=7.6 Hz, 2H), 2.47 (t, J=7.6 Hz, 2H), 2.25 (s, 3H), 2.19 (s, 1H), 1.76 (q, J=3.9 Hz, 2H), 1.18 (q, J=3.9 Hz, 2H). ¹³C NMR (151 MHz, CDCl₃) δ 171.78, 171.50, 167.25, 164.97, 155.52, 148.90, 144.64, 144.13, 143.60, 140.92, 140.21, 134.93, 134.65, 133.37, 131.81, 131.68, 129.98, 128.40, 127.78, 126.98, 126.62, 126.59, 126.35, 112.96, 112.38, 110.20, 107.12, 100.70, 70.43, 70.38, 70.18, 70.07, 69.85, 69.82, 53.43, 39.81, 39.19, 34.59, 31.20, 30.92, 23.93, 19.13, 17.18.

### Synthesis of Alkyne-Linker-Compound 100

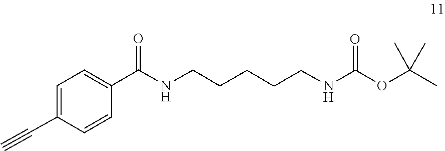 [SMILES: C#Cc1ccc(C(=O)NCCCCCNC(=O)OC(C)(C)C)cc1]

N-(5-aminopentyl)-4-ethynylbenzamide (11): 4-ethynylbenzoic acid (27 mg, 0.19 mmol), N-Boc-1,5-diaminopentane (47 mg, 0.23 mmol), HOBt (26 mg, 0.19 mmol), and DIEA (165 mL, 0.95 mmol) were dissolved in DCM (1.5 mL) and EDCI—HCl (73 mg, 0.38 mmol) was added. After stirring the mixture for 16h at rt, water was added, the mixture partitioned, and the aqueous phase extracted with DCM. Combined organic extracts were washed with brine and dried over Na₂SO₄, concentrated, and the crude residue was purified by silica gel chromatography (0-50% EtOAc/Hex) to obtain the Boc-protected amine 11 (27 mg, 0.082 mmol, 43%) as a solid. LC/MS [M+H]⁺ m/z calc. 331.19, found 331.1. 1H NMR (300 MHz, CDCl₃) δ 7.78 (d, J=8.3 Hz, 2H), 7.59 (d, J=8.7 Hz, 2H), 6.32 (s, 1H), 4.63 (s, 1H), 3.50 (td, J=7.0, 5.7 Hz, 2H), 3.23 (s, 1H), 3.18 (q, J=6.5 Hz, 2H), 1.70 (d, J=7.5 Hz, 2H), 1.62-1.52 (m, 2H), 1.46 (s, 11H).

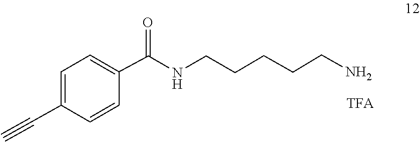 [SMILES: C#Cc1ccc(C(=O)NCCCCCN)cc1]

N-(5-aminopentyl)-4-ethynylbenzamide (12): tert-butyl (5-(4-ethynylbenzamido)pentyl)-carbamate 11 (27 mg, 0.082 mmol) was dissolved in DCM (1 mL) and TFA (0.5 mL) was added. After stirring at rt for 2h, the mixture was diluted in DCM and evaporated repeatedly to remove volatiles and provide the amine as a TFA salt and an oil (32 mg, 0.096 mmol, 117%), which was used without further purification. LC/MS [M+H]⁺ m/z calc. 231.14, found 231.1. 1H NMR (400 MHz, DMSO-d6) δ 8.55 (t, J=5.7 Hz, 1H), 7.84 (d, J=8.2 Hz, 2H), 7.63 (s, 2H), 7.57 (d, J=8.1 Hz, 2H), 4.39 (s, 1H), 3.26 (q, J=6.6 Hz, 2H), 2.83-2.74 (m, 2H), 1.62-1.48 (m, 4H), 1.40-1.32 (m, 2H).

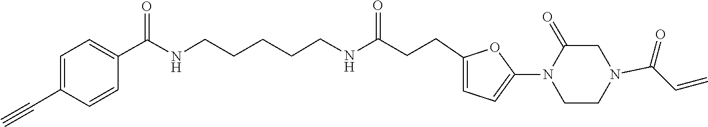 [SMILES: C#Cc1ccc(C(=O)NCCCCCNC(=O)CCc2ccc(N3CCN(C(=O)C=C)CC3=O)o2)cc1]

N-(5-(3-(5-(4-acryloyl-2-oxopiperazin-1-yl)furan-2-yl)propanamido)pentyl)-4-ethynylbenzamide (Compound 100): Intermediate 1, tert-butyl 3-(5-(4-acryloyl-2-oxopiperazin-1-yl)furan-2-yl)propanoate (20 mg, 0.057 mmol) was dissolved in DCM (0.5 mL) and treated with TFA (0.25 mL). The mixture was stirred at rt for 45 minutes until the starting material was consumed, followed by dilution with DCM and evaporation to remove volatiles. The carboxylic acid was then dissolved in DMF, and 12 (N-(5-aminopentyl)-4-ethynylbenzamide TFA; 22 mg, 0.062 mmol), DIEA (50 mL, 0.29 mmol), and HATU (43 mg, 0.11 mmol) were added. After stirring the mixture at rt for 1 h, water was added. The resulting suspension was extracted three times with DCM. Combined organic extracts were washed brine and dried over Na₂SO₄, concentrated, and the crude residue was purified by silica gel chromatography (0-4% MeOH/DCM) to obtain Compound 100 (7.6 mg, 0.016 mmol, 27%) as an oil. HRMS [M+H]⁺ m/z calc. 380.1586, found 380.1581. 1H NMR (300 MHz, CDCl₃) δ 7.82 (d, J=8.3 Hz, 2H), 7.58 (d, J=8.3 Hz, 2H), 6.77-6.50 (m, 2H), 6.43 (dd, J=16.7, 2.1 Hz, 1H), 6.24 (d, J=3.2 Hz, 1H), 6.06 (d, J=3.3 Hz, 1H), 5.93 (s, 1H), 5.86 (dd, J=10.1, 2.1 Hz, 1H), 4.44 (d, J=17.4 Hz, 2H), 4.01 (s, 2H), 3.91-3.84 (m, 2H), 3.46 (q, J=6.6 Hz, 2H), 3.32-3.19 (m, 3H), 2.93 (t, J=7.2 Hz, 2H), 2.50 (t, J=7.3 Hz, 2H), 1.72-1.61 (m, 2H), 1.60-1.46 (m, 2H), 1.44-1.35 (m, 2H). 13C NMR (151 MHz, DMSO) δ 171.0, 165.7, 164.6, 150.1, 135.2, 132.1, 128.8, 127.9, 124.7, 106.9, 100.5, 83.4, 83.1, 38.9, 33.8, 29.3, 29.2, 24.3, 24.0.

### Synthesis of Compound 226

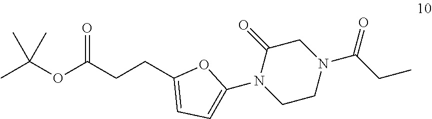 [SMILES: CCC(=O)N1CCN(c2ccc(CCC(=O)OC(C)(C)C)o2)C(=O)C1]

tert-butyl 3-(5-(2-oxo-4-propionylpiperazin-1-yl)furan-2-yl)propanoate (10): Intermediate 2 (benzyl (E)-4-(5-(3-(tert-butoxy)-3-oxoprop-1-en-1-yl)furan-2-yl)-3-oxopiperazine-1-carboxylate) (85 mg, 0.20 mmol) was dissolved in EtOH (5 mL) and Pd/C (10 mg, 10% wt.) was added. The atmosphere was exchanged for hydrogen (balloon) and the mixture was stirred vigorously overnight. After 16h, the suspension was diluted with DCM and filtered through Celite to remove Pd/C, then concentrated. The crude residue was redissolved in DCM (2 mL), and TEA (83 μL, 0.60 mmol) was added. The solution was then cooled to 0° C. and propionyl chloride (25 μL, 0.31 mmol) was added and the mixture stirred for 30 min at 0° C. Water was added and the mixture was extracted with DCM three times. Organic extracts were combined, washed with brine, dried over sodium sulfate, and concentrated. The crude residue was purified by silica gel chromatography to provide Intermediate 10 (48 mg, 0.14 mmol, 69% yield over two steps) as a solid. 1H NMR (600 MHz, CDCl₃) δ 6.28 (d, J=3.2 Hz, 1H), 6.04 (d, J=3.2 Hz, 1H), 4.40 (s, 1H), 4.29 (s, 1H), 3.91 (dt, J=30.8, 5.3 Hz, 2H), 3.85-3.78 (m, 2H), 2.88 (t, J=7.6 Hz, 2H), 2.54 (t, J=7.5 Hz, 2H), 2.43-2.34 (m, 2H), 1.44 (s, 9H), 1.19 (q, J=6.9 Hz, 3H).LC/MS: [M+H]⁺ m/z calc. 351.2, found 351.2.

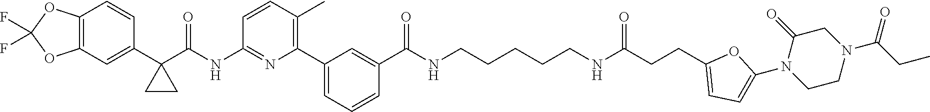 [SMILES: CCC(=O)N1CCN(c2ccc(CCC(=O)NCCCCCNC(=O)c3cccc(-c4nc(NC(=O)C5(c6ccc7c(c6)OC(F)(F)O7)CC5)ccc4C)c3)o2)C(=O)C1]

3-(6-(1-(2,2-difluorobenzo[d][1,3]dioxol-5-yl)cyclopropane-1-carboxamido)-3-methylpyridin-2-yl)-N-(5-(3-(5-(2-oxo-4-propionylpiperazin-1-yl)furan-2-yl)propanamido)pentyl)benzamide (Compound 226): Intermediate 10 (15 mg, 0.043 mmol) and Intermediate 5c (15 mg, 0.029 mmol) were reacted according to General Procedure C to provide Compound 226 (18 mg, 0.022 mmol, 76%) as an oil. 1H NMR (400 MHz, CDCl₃) δ 8.10 (d, J=8.4 Hz, 1H), 7.82 (s, 1H), 7.79 (d, J=7.6 Hz, 1H), 7.72 (s, 1H), 7.59 (d, J=8.5 Hz, 1H), 7.52 (d, J=7.7 Hz, 1H), 7.46 (t, J=7.6 Hz, 1H), 7.22 (dd, J=8.1, 1.8 Hz, 1H), 7.19 (d, J=1.7 Hz, 1H), 7.07 (d, J=8.1 Hz, 1H), 6.51-6.39 (m, 1H), 6.22-6.15 (m, 1H), 6.02-5.97 (m, 1H), 5.88-5.76 (m, 1H), 4.31 (d, J=50.6 Hz, 2H), 3.96-3.71 (m, 4H), 3.42 (q, J=6.6 Hz, 2H), 3.21 (q, J=6.5 Hz, 2H), 2.92-2.82 (m, 2H), 2.44 (t, J=7.4 Hz, 2H), 2.41-2.29 (m, 2H), 2.24 (s, 3H), 1.74 (q, J=3.9 Hz, 2H), 1.64-1.56 (m, 2H), 1.53-1.43 (m, 2H), 1.38-1.26 (m, 2H), 1.21-1.09 (m, 5H). 13C NMR (151 MHz, CDCl₃) δ 206.9, 172.2, 171.8, 167.3, 155.4, 149.8, 148.9, 144.9, 144.1, 143.6, 141.0, 140.1, 134.9, 134.8, 131.7, 131.7, 128.4, 127.5, 127.0, 126.6, 113.0, 112.4, 110.2, 107.3, 100.9, 53.4, 49.3, 47.2, 42.4, 39.7, 39.1, 38.7, 34.9, 31.2, 29.0, 26.5, 24.2, 23.8, 19.1, 17.2, 9.0. HRMS (ESI): [M+H]⁺ m/z calc. 813.3345, found 813.3422.

### Synthesis of Compound 101

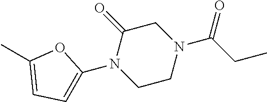 [SMILES: CCC(=O)N1CCN(c2ccc(C)o2)C(=O)C1]

1-(5-methylfuran-2-yl)-4-propionylpiperazin-2-one: 1-(5-methylfuran-2-yl)piperazin-2-one (30 mg, 0.17 mmol) was dissolved in DCM (2 mL). The solution was cooled to 0° C. and TEA (69 μL, 0.50 mmol) and propionyl chloride (21 μL, 0.25 mmol) were added. After stirring at 0° C. for 30 min, water was added, and the reaction extracted three times with DCM. Organic extracts were combined, washed with brine, dried over sodium sulfate, and concentrated. The crude residue was purified by silica gel chromatography (0-100% EtOAc/Hex) to provide 1-(5-methylfuran-2-yl)-4-propionylpiperazin-2-one (17.3 mg, 0.073 mmol, 43%) as a solid. 1H NMR (600 MHz, CDCl₃) δ 6.25 (d, J=3.2 Hz, 1H), 6.00 (d, J=2.2 Hz, 1H), 4.41 (s, 1H), 4.29 (s, 1H), 3.97-3.86 (m, 2H), 3.82 (t, J=5.5 Hz, 2H), 2.45-2.34 (m, 2H), 2.27 (s, 3H), 1.23-1.16 (m, 3H). 13C NMR (151 MHz, CDCl3) δ 172.2, 163.4, 147.5, 144.4, 107.2, 101.0, 49.3, 46.9, 38.8, 26.5, 13.4, 9.0.13C NMR (151 MHz, CDCl₃) δ 172.2, 163.4, 147.5, 144.4, 107.2, 101.0, 49.3, 46.9, 38.8, 26.5, 13.4, 9.0. HRMS (ESI): [M+H]⁺ m/z calc. 259.1160, found 259.1053.

### Synthesis of Compound 220

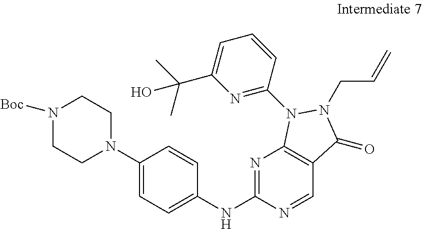 [SMILES: C=CCn1c(=O)c2cnc(Nc3ccc(N4CCN(C(=O)OC(C)(C)C)CC4)cc3)nc2n1-c1cccc(C(C)(C)O)n1]

tert-butyl 4-(4-((2-allyl-1-(6-(2-hydroxypropan-2-yl)pyridin-2-yl)-3-oxo-2,3-dihydro-1H-pyrazolo[3,4-d]pyrimidin-6-yl)amino)phenyl)piperazine-1-carboxylate (Intermediate 7). Commercially available Intermediate 6 2-allyl-1-(6-(2-hydroxypropan-2-yl)pyridin-2-yl)-6-(methylthio)-1,2-dihydro-3H-pyrazolo[3,4-d]pyrimidin-3-one (250 mg, 0.7 mmol) was dissolved in 7 mL of toluene and cooled to 0° C. meta-Chloroperoxybenzoic acid (190 mg, 0.77 mmol) was added to the reaction mixture on ice, and the reaction mixture was warmed to room temperature and stirred for 1 hour. N,N-Diisopropylethylamine (365 mL, 2.1 mmol) and 1-Piperazine-carboxylic acid, 4-(4-aminophenyl)-, 1,1-dimethylethyl ester (232 mg, 0.84 mmol) were then added slowly and the reaction mixture was stirred overnight. The reaction mixture was extracted in EtOAc, washed 3× with brine, and dried on silica. Purification by flash column chromatography (DCM/Hexane 5:95) yielded Intermediate 7 (0.445 mmol, 64% yield). ¹H NMR (400 MHz, Chloroform-d) δ 8.99 (s, 1H), 7.95 (t, J=7.9 Hz, 1H), 7.80 (dd, J=8.1, 0.8 Hz, 1H), 7.44 (dd, J=7.7, 0.8 Hz, 1H), 5.83-5.65 (m, 1H), 5.13-5.04 (m, 1H), 4.97 (dq, J=17.1, 1.4 Hz, 1H), 4.85 (dt, J=6.2, 1.4 Hz, 2H), 3.80 (s, 1H), 2.63 (s, 3H), 1.63 (s, 6H).

LC/MS: [M+H]⁺ m/z calc.

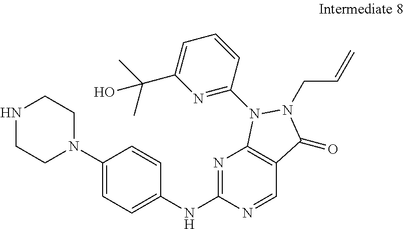 [SMILES: C=CCn1c(=O)c2cnc(Nc3ccc(N4CCNCC4)cc3)nc2n1-c1cccc(C(C)(C)O)n1]

2-allyl-1-(6-(2-hydroxypropan-2-yl)pyridin-2-yl)-6-((4-(piperazin-1-yl)phenyl)amino)-1,2-dihydro-3H-pyrazolo[3,4-d]pyrimidin-3-one (Intermediate 8). Intermediate 7 (261 mg, 0.445 mmol) was dissolved in 4 mL of DCM and cooled to 0° C. 1 mL of trifluoroacetic acid was added dropwise on ice. The reaction mixture was stirred at room temperature for 1 hour, then extracted in DCM, washed 3× with brine, and dried on silica. Purification by flash column chromatography (DCM/Hexane 5:95) yielded Intermediate 8 (0.398 mmol, 89% yield). ¹H NMR (400 MHz, Chloroform-d) δ 8.84 (s, 1H), 7.86 (t, J=7.9 Hz, 1H), 7.75 (d, J=8.1 Hz, 1H), 7.48 (d, J=8.5 Hz, 2H), 7.34 (d, J=7.6 Hz, 1H), 6.93 (d, J=9.0 Hz, 2H), 5.78-5.59 (m, 1H), 5.04 (d, J=10.2 Hz, 1H), 4.94 (d, J=17.0 Hz, 1H), 4.74 (d, J=6.2 Hz, 2H), 3.94 (s, 1H), 3.60 (t, J=5.1 Hz, 4H), 3.11 (t, J=5.1 Hz, 4H), 2.05 (s, 1H), 1.59 (s, 6H), 1.49 (s, 9H).LC/MS: [M+H]⁺ m/z calc.

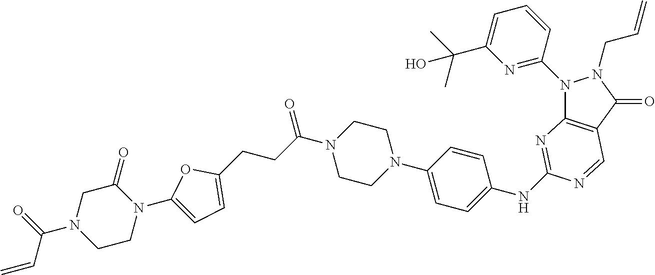 [SMILES: C=CCn1c(=O)c2cnc(Nc3ccc(N4CCN(C(=O)CCc5ccc(N6CCN(C(=O)C=C)CC6=O)o5)CC4)cc3)nc2n1-c1cccc(C(C)(C)O)n1]

6-((4-(4-(3-(5-(4-acryloyl-2-oxopiperazin-1-yl)furan-2-yl)propanoyl)piperazin-1-yl)phenyl)amino)-2-allyl-1-(6-(2-hydroxypropan-2-yl)pyridin-2-yl)-1,2-dihydro-3H-pyrazolo[3,4-d]pyrimidin-3-one (Compound 220). LEB-03-139 (0.0449 mmol) was dissolved in 3 mL of DCM and the reaction mixture was cooled on ice. 1 mL of trifluoroacetic acid was added dropwise and the solution was warmed to room temperature and stirred for 1 hour. The deprotected amine salt was washed twice with DCM and dried under vacuum. Immediately following deprotection, the crude product was dissolved in 0.5 mL DMF and deprotected intermediate 3 (0.0898 mmol) was added to the mixture, followed by DIPEA (0.449 mmol) and HATU (0.0898 mmol). The reaction was stirred for 30 minutes before water was added. The mixture was extracted three times with EtOAc, and combined organic extracts were washed with brine, dried over sodium sulfate, and concentrated. Purification by flash column chromatography (MeOH:DCM 8:92) yielded Compound 220 as a solid (12.9 mg, 0.0169 mmol, 38% yield). ¹H NMR (600 MHz, Chloroform-d) δ 8.76 (s, 1H), 7.80 (t, J=7.9 Hz, 1H), 7.66 (d, J=8.0 Hz, 1H), 7.43 (d, J=8.4 Hz, 2H), 7.29 (d, J=7.6 Hz, 1H), 7.19 (s, 1H), 6.87-6.82 (m, 2H), 6.45 (s, 1H), 6.34 (dd, J=16.7, 1.8 Hz, 1H), 6.20 (d, J=3.2 Hz, 1H), 6.01 (d, J=3.2 Hz, 1H), 5.74 (t, J=11.1, 10.6 Hz, 1H), 5.67-5.59 (m, 1H), 5.23 (s, 1H), 4.97 (dd, J=9.8, 0.8 Hz, 1H), 4.87 (dd, J=17.4, 0.8 Hz, 1H), 4.67 (d, J=6.2 Hz, 2H), 4.38 (s, 1H), 4.31 (s, 1H), 3.73 (t, J=5.2 Hz, 2H), 3.55 (t, J=5.1 Hz, 2H), 3.06 (t, J=5.2 Hz, 4H), 2.92 (t, J=7.8 Hz, 2H), 2.62 (d, J=8.4 Hz, 2H), 1.59 (s, 4H), 1.52 (s, 6H). ¹³C NMR (151 MHz, Chloroform-d) δ 169.96, 165.90, 165.00, 162.18, 161.36, 161.26, 156.36, 150.16, 147.68, 147.51, 144.67, 138.85, 131.56, 131.29, 126.30, 119.07, 117.20, 116.21, 116.12, 107.23, 101.12, 72.46, 50.15, 49.85, 49.49, 47.67, 45.40, 41.64, 31.55, 30.56, 23.71. HRMS (ESI): [M+H]⁺ m/z calc. 761.35, found 761.3522.

### Synthesis of Compound 221

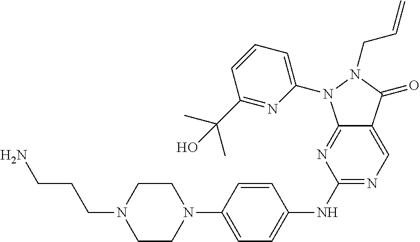 [SMILES: C=CCn1c(=O)c2cnc(Nc3ccc(N4CCN(CCCN)CC4)cc3)nc2n1-c1cccc(C(C)(C)O)n1]

2-allyl-6-((4-(4-(3-aminopropyl)piperazin-1-yl)phenyl)amino)-1-(6-(2-hydroxypropan-2-yl)pyridin-2-yl)-1,2-dihydro-3H-pyrazolo[3,4-d]pyrimidin-3-one (Intermediate 12). Intermediate 8 (40 mg, 0.0823 mmol) was dissolved in 0.5 mL of DMF. tert-butyl (3-bromopropyl)carbamate (24 mg, 1.2 eq, 0.0987 mmol) and potassium carbonate (34 mg, 3.0 eq, 0.247 mmol) were added to the mixture, and the reaction was warmed to 50° C. and stirred overnight. Water was added, the mixture extracted three times with EtOAc, combined organic extracts were washed with brine, and dried over sodium sulfate, and concentrated. Purification by flash column chromatography (EtOAc:Hexanes 50:50) yielded the boc-protected intermediate. This was immediately dissolved in 3 mL of DCM and the reaction mixture was cooled on ice. 1 mL of trifluoroacetic acid was added dropwise and the solution was warmed to room temperature and stirred for 1 hour. The deprotected amine TFA salt was washed twice with DCM and dried under vacuum to yield Intermediate 12 (33 mg, 0.0497 mmol, 60% yield over two steps) as an oil. ¹H NMR (300 MHz, Chloroform-d) δ 8.80 (s, 1H), 7.92 (t, J=7.9 Hz, 1H), 7.63 (d, J=8.0 Hz, 1H), 7.54 (d, J=8.4 Hz, 3H), 6.90 (d, J=8.9 Hz, 2H), 5.75-5.54 (m, 1H), 5.05 (d, J=10.2 Hz, 1H), 4.89 (d, J=17.1 Hz, 1H), 4.75 (d, J=6.2 Hz, 2H), 3.66 (s, 1H), 3.43 (s, 9H), 3.28 (q, J=9.4, 8.5 Hz, 2H), 3.19 (s, 1H), 3.06 (t, J=7.1 Hz, 2H), 2.23 (d, J=8.2 Hz, 3H), 1.59 (s, 6H). LC/MS: [M+H]⁺ m/z calc. 544.3, found 544.3.

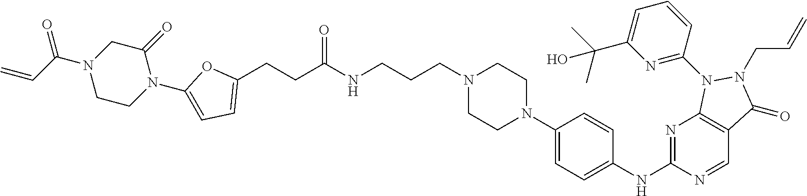 [SMILES: C=CCn1c(=O)c2cnc(Nc3ccc(N4CCN(CCCNC(=O)CCc5ccc(N6CCN(C(=O)C=C)CC6=O)o5)CC4)cc3)nc2n1-c1cccc(C(C)(C)O)n1]

3-(5-(4-acryloyl-2-oxopiperazin-1-yl)furan-2-yl)-N-(3-(4-(4-((2-allyl-1-(6-(2-hydroxypropan-2-yl)pyridin-2-yl)-3-oxo-2,3-dihydro-1H-pyrazolo[3,4-d]pyrimidin-6-yl)amino)phenyl)piperazin-1-yl)propyl)propenamide (Compound 221). Intermediate 3 (19 mg, 0.0558 mmol) and Intermediate 12 (0.0497 mmol) were reacted according to general procedure C. After hydrolysis, deprotected 3 and Intermediate 12 were dissolved in DMF (0.5 mL), followed by DIPEA (43 mL, 0.249 mmol) and HATU (23 mg, 0.0596 mmol). The reaction was stirred for 30 minutes. Water was added and the mixture extracted three times with 4:1 CHCl₃:IPA. Combined organic extracts were washed with brine, and dried over sodium sulfate, and concentrated. Purification by prep TLC (10% MeOH in DCM) yielded Compound 221 as a solid (8.1 mg, 0.0099 mmol, 20% yield). ¹H NMR (600 MHz, DMSO-d6) δ 10.07 (s, 1H), 8.75 (s, 1H), 7.97 (s, 1H), 7.83 (t, J=5.6 Hz, 1H), 7.68 (d, J=8.1 Hz, 1H), 7.54 (d, J=7.7 Hz, 1H), 7.51 (s, 2H), 6.85 (d, J=8.6 Hz, 2H), 6.80-6.72 (m, 1H), 6.16-6.08 (m, 2H), 6.04 (d, J=3.2 Hz, 1H), 5.71-5.66 (m, 1H), 5.64-5.55 (m, 1H), 5.24 (s, 1H), 4.92 (d, J=10.2 Hz, 1H), 4.76 (d, J=17.0 Hz, 1H), 4.61 (d, J=6.0 Hz, 2H), 4.27 (d, J=93.6 Hz, 2H), 3.95-3.63 (m, 4H), 3.02 (q, J=6.4 Hz, 6H), 2.73 (t, J=7.5 Hz, 2H), 2.31 (t, J=7.5 Hz, 2H), 2.24 (t, J=7.2 Hz, 2H), 1.55-1.47 (m, 2H), 1.39 (s, 2H), 1.17 (s, 6H), 0.80-0.74 (m, 2H). ¹³C NMR (151 MHz, DMSO) δ 171.0, 168.0, 164.6, 161.6, 156.5, 150.1, 139.3, 132.7, 131.3, 128.8, 118.7, 116.8, 115.9, 106.9, 100.5, 72.8, 55.9, 53.2, 49.2, 47.6, 47.1, 46.9, 42.5, 37.4, 34.7, 33.8, 31.4, 30.9, 29.5, 26.9, 25.3, 24.0, 22.6, 22.5, 14.4. HRMS (ESI): [M+H]⁺ m/z calc. 818.41, found 818.4101.

### Synthesis of Compound 222

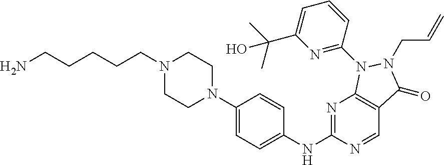 [SMILES: C=CCn1c(=O)c2cnc(Nc3ccc(N4CCN(CCCCCN)CC4)cc3)nc2n1-c1cccc(C(C)(C)O)n1]

2-allyl-6-((4-(4-(5-aminopentyl)piperazin-1-yl)phenyl)amino)-1-(6-(2-hydroxypropan-2-yl)pyridin-2-yl)-1,2-dihydro-3H-pyrazolo[3,4-d]pyrimidin-3-one (Intermediate 13). Intermediate 8 (40 mg, 0.0823 mmol) was dissolved in 0.5 mL of DMF. tert-butyl (5-bromopentyl)carbamate (26 mg, 1.2 eq, 0.0987 mmol) and potassium carbonate (34 mg, 3.0 eq, 0.247 mmol) were added to the mixture, and the reaction was warmed to 50° C. and stirred overnight. Water was added, the mixture extracted three times with EtOAc, combined organic extracts were washed with brine, and dried over sodium sulfate, and concentrated. Purification by flash column chromatography (EtOAc:Hexanes 50:50) yielded boc-protected intermediate. This was immediately dissolved in 3 mL of DCM and the reaction mixture was cooled on ice. 1 mL of trifluoroacetic acid was added dropwise and the solution was warmed to room temperature and stirred for 1 hour. The deprotected amine TFA salt was washed twice with DCM and dried under vacuum to yield Intermediate 13 (21 mg, 0.0307 mmol, 37% yield over two steps) as an oil. ¹H NMR (300 MHz, Chloroform-d) δ 8.80 (s, 1H), 8.12 (s, 1H), 7.94 (t, J=7.9 Hz, 1H), 7.64 (d, J=8.0 Hz, 1H), 7.56 (d, J=8.2 Hz, 3H), 6.92 (d, J=8.8 Hz, 2H), 5.67 (dd, J=16.8, 10.4 Hz, 1H), 5.07 (d, J=10.2 Hz, 1H), 4.90 (d, J=17.1 Hz, 1H), 4.76 (d, J=6.2 Hz, 2H), 3.69 (s, 1H), 3.55-3.47 (m, 8H), 3.22 (s, 1H), 3.19-2.89 (m, 4H), 1.91-1.66 (m, 4H), 1.61 (s, 6H), 1.51 (s, 2H), 1.27 (s, 1H). LC/MS: [M+H]⁺ m/z calc. 572.3, found 572.3.

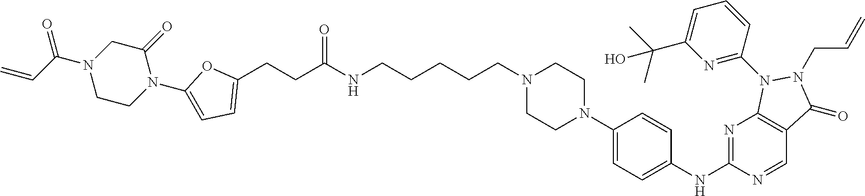 [SMILES: C=CCn1c(=O)c2cnc(Nc3ccc(N4CCN(CCCCCNC(=O)CCc5ccc(N6CCN(C(=O)C=C)CC6=O)o5)CC4)cc3)nc2n1-c1cccc(C(C)(C)O)n1]

3-(5-(4-acryloyl-2-oxopiperazin-1-yl)furan-2-yl)-N-(5-(4-(4-((2-allyl-1-(6-(2-hydroxypropan-2-yl)pyridin-2-yl)-3-oxo-2,3-dihydro-1H-pyrazolo[3,4-d]pyrimidin-6-yl)amino)phenyl)piperazin-1-yl)pentyl)propanamide (Compound 222). Intermediate 3 (19 mg, 0.0558 mmol) and Intermediate 13 (21 mg, 0.0307 mmol) were coupled according to general procedure C. After hydrolysis, deprotected 3 and Intermediate 13 were dissolved in DMF (0.5 mL), followed by DIPEA (27 mL, 0.153 mmol) and HATU (14 mg, 0.0368 mmol). Water was added and the mixture extracted three times with 4:1 CHCl₃:IPA. Combined organic extracts were washed with brine, and dried over sodium sulfate, and concentrated. Purification by prep TLC (8% MeOH in DCM) yielded Compound 222 as a solid (10.1 mg, 0.0119 mmol, 39% yield). ¹H NMR (600 MHz, DMSO-d6) δ 10.15 (s, 1H), 8.83 (s, 1H), 8.05 (s, 1H), 7.86 (t, J=5.6 Hz, 1H), 7.78-7.72 (m, 1H), 7.61 (d, J=7.7 Hz, 2H), 7.58 (s, 2H), 6.92 (d, J=8.7 Hz, 2H), 6.88-6.76 (m, 1H), 6.24-6.19 (m, 1H), 6.10 (d, J=3.2 Hz, 1H), 5.76 (q, J=9.8, 8.3 Hz, 1H), 5.72-5.61 (m, 1H), 5.36-5.26 (m, 1H), 5.00 (dq, J=10.3, 1.3 Hz, 1H), 4.84 (dq, J=17.2, 1.5 Hz, 1H), 4.69 (d, J=6.0 Hz, 2H), 4.43 (s, 1H), 4.27 (s, 1H), 3.95 (d, J=5.8 Hz, 1H), 3.86 (s, 1H), 3.82-3.73 (m, 2H), 3.13-3.08 (m, 4H), 3.08-3.01 (m, 2H), 2.80 (t, J=7.5 Hz, 2H), 2.38 (t, J=7.5 Hz, 2H), 2.30 (t, J=7.4 Hz, 2H), 1.47 (s, 6H), 1.45-1.37 (m, 2H), 1.26-1.21 (m, 6H), 0.89-0.81 (m, 2H). ¹³C NMR (151 MHz, DMSO) δ 170.97, 168.04, 161.64, 156.46, 150.07, 139.28, 132.67, 128.77, 118.72, 115.93, 106.92, 100.44, 72.78, 58.33, 53.28, 49.17, 47.57, 47.06, 46.88, 42.46, 38.88, 33.80, 30.92, 29.54, 29.48, 29.16, 26.48, 24.81, 24.00, 22.56, 14.42. HRMS (ESI): [M+H]⁺ m/z calc. 846.44, found 846.4395.

### Synthesis of Compound 223

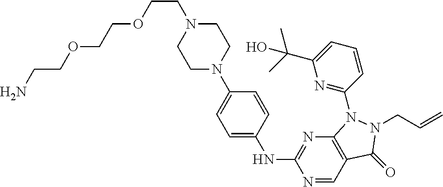 [SMILES: C=CCn1c(=O)c2cnc(Nc3ccc(N4CCN(CCOCCOCCN)CC4)cc3)nc2n1-c1cccc(C(C)(C)O)n1]

2-allyl-6-((4-(4-(2-(2-(2-aminoethoxy)ethoxy)ethyl)piperazin-1-yl)phenyl)amino)-1-(6-(2-hydroxypropan-2-yl)pyridin-2-yl)-1,2-dihydro-3H-pyrazolo[3,4-d]pyrimidin-3-one (Intermediate 14). Intermediate 8 (40 mg, 0.0823 mmol) was dissolved in 0.5 mL of DMF. tert-butyl (2-(2-(bromomethoxy)ethoxy)ethyl)carbamate (31 mg, 1.2 eq, 0.0987 mmol) and potassium carbonate (34 mg, 3.0 eq, 0.247 mmol) were added to the mixture, and the reaction was warmed to 50° C. and stirred overnight. Water was added, the mixture extracted three times with EtOAc, combined organic extracts were washed with brine, and dried over sodium sulfate, and concentrated. Purification by flash column chromatography (EtOAc:Hexanes 50:50) yielded boc-protected intermediate. This was immediately dissolved in 3 mL of DCM and the reaction mixture was cooled on ice. 1 mL of trifluoroacetic acid was added dropwise and the solution was warmed to room temperature and stirred for 1 hour. The deprotected amine TFA salt was washed twice with DCM and dried under vacuum to yield Intermediate 14 (28 mg, 0.0389 mmol, 47% yield). ¹H NMR (300 MHz, Chloroform-d) δ 10.99 (s, 1H), 8.74 (s, 1H), 8.25 (s, 1H), 7.97 (t, J=7.9 Hz, 1H), 7.60 (t, J=8.9 Hz, 2H), 7.50 (d, J=8.5 Hz, 2H), 6.87 (d, J=8.7 Hz, 2H), 5.66 (ddd, J=16.5, 10.3, 5.6 Hz, 1H), 5.07 (d, J=10.2 Hz, 1H), 4.90 (d, J=17.1 Hz, 1H), 4.75 (d, J=6.3 Hz, 4H), 3.87 (d, J=4.6 Hz, 4H), 3.76-3.69 (m, 4H), 3.65 (s, 4H), 3.39-3.10 (m, 8H), 1.61 (s, 6H). LC/MS: [M+H]⁺ m/z calc. 618.3, found 618.3.

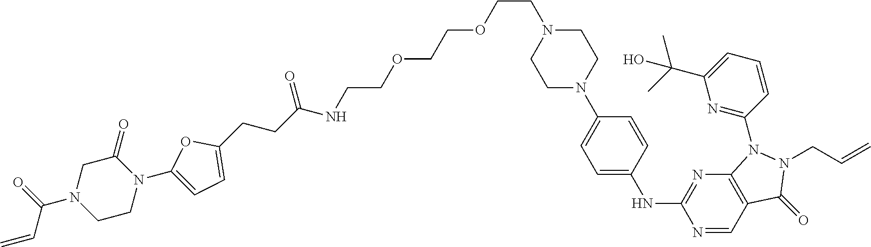 [SMILES: C=CCn1c(=O)c2cnc(Nc3ccc(N4CCN(CCOCCOCCNC(=O)CCc5ccc(N6CCN(C(=O)C=C)CC6=O)o5)CC4)cc3)nc2n1-c1cccc(C(C)(C)O)n1]

3-(5-(4-acryloyl-2-oxopiperazin-1-yl)furan-2-yl)-N-(2-(2-(2-(4-(4-((2-allyl-1-(6-(2-hydroxypropan-2-yl)pyridin-2-yl)-3-oxo-2,3-dihydro-1H-pyrazolo[3,4-d]pyrimidin-6-yl)amino)phenyl)piperazin-1-yl)ethoxy)ethoxy)ethyl)propanamide (Compound 223). Intermediate 3 (19 mg, 0.0558 mmol) and Intermediate 14 (28 mg, 0.0389 mmol) were coupled according to general procedure C. After hydrolysis, deprotected 3 and Intermediate 14 were dissolved in DMF (0.5 mL), followed by DIPEA (34 mL, 0.195 mmol) and HATU (18 mg, 0.0466 mmol). The reaction was stirred for 30 minutes. Water was added and the mixture extracted three times with 4:1 CHCl₃:IPA. Combined organic extracts were washed with brine, and dried over sodium sulfate, and concentrated. Purification by prep TLC (8% MeOH in DCM) yielded Compound 223 as a solid (8.3 mg, 0.0093 mmol, 17% yield). ¹H NMR (600 MHz, DMSO-d6) δ 8.83 (s, 1H), 8.05 (s, 1H), 7.97 (t, J=5.8 Hz, 1H), 7.76 (s, 1H), 7.61 (d, J=7.8 Hz, 1H), 7.58 (s, 3H), 6.92 (d, J=8.5 Hz, 2H), 6.81 (d, J=12.8 Hz, 1H), 6.23-6.15 (m, 2H), 6.10 (d, J=3.2 Hz, 1H), 5.76 (d, J=7.0 Hz, 2H), 5.67 (ddt, J=16.5, 10.8, 6.0 Hz, 1H), 5.31 (s, 1H), 5.04-4.97 (m, 1H), 4.87-4.80 (m, 1H), 4.69 (s, 2H), 4.42 (s, 1H), 4.26 (s, 1H), 3.94 (s, 1H), 3.85 (s, 1H), 3.77 (d, J=24.8 Hz, 2H), 3.56 (t, J=5.8 Hz, 2H), 3.54-3.49 (m, 6H), 3.42 (t, J=5.9 Hz, 2H), 3.22 (q, J=5.8 Hz, 2H), 3.09 (d, J=5.8 Hz, 4H), 2.79 (t, J=7.6 Hz, 2H), 2.58 (t, J=4.8 Hz, 4H), 2.44-2.36 (m, 4H), 1.47 (s, 6H), 0.86 (d, J=7.4 Hz, 1H). ¹³C NMR (151 MHz, DMSO) δ 171.31, 168.04, 161.64, 156.46, 150.02, 139.28, 132.68, 128.76, 118.72, 115.93, 106.93, 100.44, 72.78, 70.12, 70.04, 69.58, 68.89, 57.72, 53.63, 49.14, 47.07, 46.88, 42.46, 39.07, 33.65, 30.92, 29.49, 23.89, 14.42. HRMS (ESI): [M+H]⁺ m/z calc. 892.45, found 892.4454.

### Synthesis of Compound 224

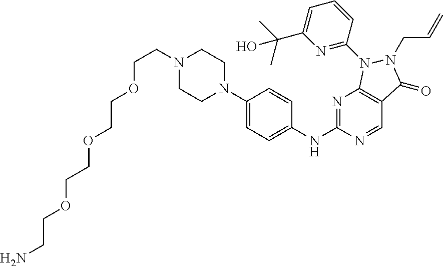 [SMILES: C=CCn1c(=O)c2cnc(Nc3ccc(N4CCN(CCOCCOCCOCCN)CC4)cc3)nc2n1-c1cccc(C(C)(C)O)n1]

2-allyl-6-((4-(4-(2-(2-(2-(2-aminoethoxy)ethoxy)ethoxy)ethyl)piperazin-1-yl)phenyl)amino)-1-(6-(2-hydroxypropan-2-yl)pyridin-2-yl)-1,2-dihydro-3H-pyrazolo[3,4-d]pyrimidin-3-one (Intermediate 15). Intermediate 8 (40 mg, 0.0823 mmol) was dissolved in 0.5 mL of DMF. tert-butyl (2-(2-(2-(bromomethoxy)ethoxy)ethoxy)ethyl)carbamate (35 mg, 1.2 eq, 0.0987 mmol) and potassium carbonate (34 mg, 3.0 eq, 0.247 mmol) were added to the mixture, and the reaction was warmed to 50° C. and stirred overnight. Water was added, the mixture extracted three times with EtOAc, combined organic extracts were washed with brine, and dried over sodium sulfate, and concentrated. Purification by flash column chromatography (EtOAc:Hexanes 50:50) yielded boc-protected intermediate. This was immediately dissolved in 3 mL of DCM and the reaction mixture was cooled on ice. 1 mL of trifluoroacetic acid was added dropwise and the solution was warmed to room temperature and stirred for 1 hour. The deprotected amine TFA salt was washed twice with DCM and dried under vacuum to yield Intermediate 15 (22 mg, 0.0279 mmol, 34% yield) as an oil. ¹H NMR (300 MHz, Chloroform-d) δ 11.44 (s, 1H), 8.71 (s, 1H), 8.10 (s, 4H), 8.00 (t, J=7.9 Hz, 1H), 7.65 (d, J=8.0 Hz, 1H), 7.57 (t, J=7.4 Hz, 3H), 6.91 (d, J=8.6 Hz, 2H), 5.69 (ddt, J=16.5, 10.1, 6.2 Hz, 1H), 5.11 (d, J=10.1 Hz, 1H), 4.92 (d, J=17.1 Hz, 1H), 4.78 (d, J=6.3 Hz, 2H), 3.98-3.77 (m, 5H), 3.77-3.63 (m, 9H), 3.33 (d, J=59.7 Hz, 8H), 1.64 (s, 6H), 1.29 (s, 1H). LC/MS: [M+H]⁺ m/z calc. 662.3, found 662.4

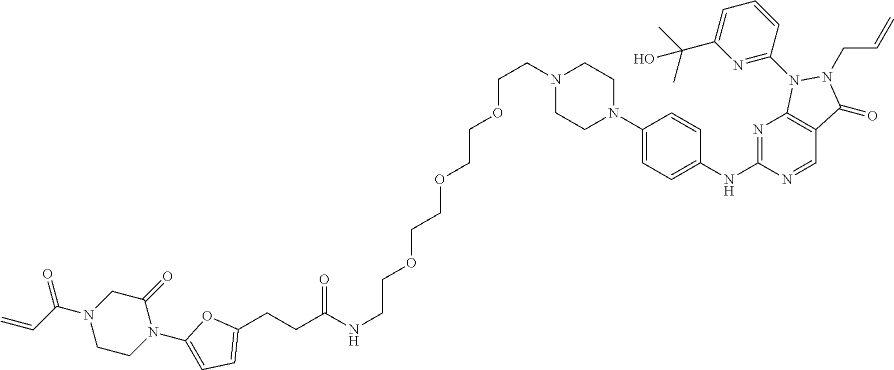 [SMILES: C=CCn1c(=O)c2cnc(Nc3ccc(N4CCN(CCOCCOCCOCCNC(=O)CCc5ccc(N6CCN(C(=O)C=C)CC6=O)o5)CC4)cc3)nc2n1-c1cccc(C(C)(C)O)n1]

3-(5-(4-acryloyl-2-oxopiperazin-1-yl)furan-2-yl)-N-(2-(2-(2-(2-(4-(4-((2-allyl-1-(6-(2-hydroxypropan-2-yl)pyridin-2-yl)-3-oxo-2,3-dihydro-1H-pyrazolo[3,4-d]pyrimidin-6-yl)amino)phenyl)piperazin-1-yl)ethoxy)ethoxy)ethoxy)ethyl)propanamide (Compound 224). Intermediate 3 (19 mg, 0.0558 mmol) and Intermediate 15 (22 mg, 0.0279 mmol) were coupled according to general procedure C. After hydrolysis, deprotected 3 and Intermediate 14 were dissolved in DMF (0.5 mL), followed by DIPEA (49 mL, 0.279 mmol) and HATU (21 mg, 0.0558 mmol). The reaction was stirred for 30 minutes. Water was added and the mixture extracted three times with 4:1 CHCl₃:IPA. Combined organic extracts were washed with brine, and dried over sodium sulfate, and concentrated. Purification by prep TLC (8% MeOH in DCM) yielded Compound 224 as a solid (10.0 mg, 0.0107 mmol, 19% yield). ¹H NMR (600 MHz, DMSO-d6) δ 10.14 (s, 1H), 8.83 (s, 1H), 8.05 (s, 1H), 7.97 (t, J=5.6 Hz, 1H), 7.76 (d, J=8.1 Hz, 1H), 7.61 (d, J=7.4 Hz, 1H), 6.92 (d, J=8.8 Hz, 2H), 6.87-6.75 (m, 1H), 6.21 (d, J=3.2 Hz, 1H), 6.11-6.06 (m, 1H), 5.76 (s, 2H), 5.67 (ddt, J=16.3, 10.2, 6.0 Hz, 1H), 5.32 (s, 1H), 5.00 (dq, J=10.2, 1.4 Hz, 1H), 4.84 (dq, J=17.1, 1.5 Hz, 1H), 4.69 (d, J=6.0 Hz, 2H), 4.26 (s, 1H), 3.95 (s, 1H), 3.77 (d, J=24.9 Hz, 2H), 3.59-3.48 (m, 9H), 3.41 (t, J=5.9 Hz, 2H), 3.21 (q, J=5.8 Hz, 2H), 3.09 (d, J=5.3 Hz, 4H), 2.83-2.76 (m, 2H), 2.57 (t, J=5.0 Hz, 4H), 2.51 (p, J=1.9 Hz, 9H), 1.47 (s, 6H). ¹³C NMR (151 MHz, DMSO-d₆) δ 171.30, 164.62, 156.47, 150.02, 147.70, 139.28, 132.68, 128.76, 118.72, 116.75, 115.93, 106.92, 100.44, 72.78, 70.26, 70.23, 70.17, 70.08, 69.59, 68.87, 57.72, 55.38, 53.62, 49.15, 47.54, 47.07, 46.87, 42.45, 39.05, 33.64, 30.92, 23.88. HRMS (ESI): [M+H]⁺ m/z calc. 936.47, found 936.4723.

Additional bifunctional compounds were prepared according to the procedures described herein. Characterization of these compounds is provided below.

### N-(6-(3-(2-(3-(5-(4-acryloyl-2-oxopiperazin-1-yl)furan-2-yl)propanoyl)-2,8 diazaspiro[4.5]-decane-8-carbonyl)phenyl)-5-methylpyridin-2-yl)-1-(2,2-difluorobenzo[d][1,3]dioxol-5-yl)cyclopropane-1-carboxamide (Compound 207)

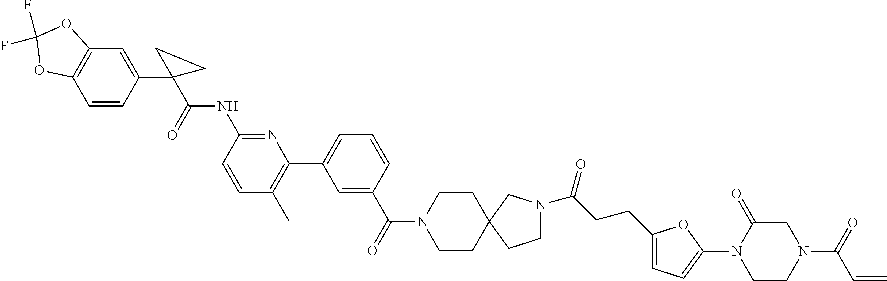 [SMILES: C=CC(=O)N1CCN(c2ccc(CCC(=O)N3CCC4(CCN(C(=O)c5cccc(-c6nc(NC(=O)C7(c8ccc9c(c8)OC(F)(F)O9)CC7)ccc6C)c5)CC4)C3)o2)C(=O)C1]

1H NMR (400 MHz, CDCl3) δ 8.11 (d, J=8.4 Hz, 1H), 7.83 (s, 1H), 7.61 (d, J=8.5 Hz, 1H), 7.49-7.43 (m, 3H), 7.42-7.38 (m, 1H), 7.25-7.20 (m, 1H), 7.19 (t, J=1.7 Hz, 1H), 7.08 (dd, J=8.2, 3.9 Hz, 1H), 6.52 (s, 1H), 6.44-6.36 (m, 1H), 6.25 (dd, J=3.2, 2.2 Hz, 1H), 6.05 (t, J=2.5 Hz, 1H), 5.82 (d, J=1.5 Hz, 1H), 4.49-4.36 (m, 2H), 4.06-3.76 (m, 5H), 3.62-3.17 (m, 7H), 2.96 (t, J=7.6 Hz, 2H), 2.57 (dd, J=8.7, 6.4 Hz, 2H), 2.26 (s, 3H), 2.11 (d, J=15.1 Hz, 2H), 1.91-1.78 (m, 2H), 1.75 (q, J=3.8 Hz, 2H), 1.54-1.39 (m, 2H), 1.17 (q, J=4.1 Hz, 2H)

13C NMR (101 MHz, CDCl3) δ 171.83, 170.42, 170.19, 169.97, 169.92, 164.98, 154.97, 150.26, 148.76, 144.60, 144.12, 143.62, 141.43, 139.60, 135.93, 135.85, 134.85, 131.69, 130.31, 130.23, 128.45, 127.68, 127.12, 126.82, 126.68, 126.29, 113.10, 112.42, 110.20, 107.11, 101.12, 56.64, 54.66, 44.71, 44.04, 41.63, 39.62, 36.60, 33.97, 33.08, 32.74, 31.23, 29.72, 23.40, 19.18, 17.27. 19F: (376 MHz, CDCl3) δ −49.52.

HRMS (TOF, ES+): m/z calcd for C46H47F2N6O8 (M+H)+ 849.3423; found 849.3419.

### N-(6-(3-(4-(2-(3-(5-(4-acryloyl-2-oxopiperazin-1-yl)furan-2-yl)-N-methylpropanamido)-ethyl)-piperidine-1-carbonyl)phenyl)-5-methylpyridin-2-yl)-1-(2,2-difluorobenzo[d][1,3]dioxol-5-yl)cyclopropane-1-carboxamide (Compound 208)

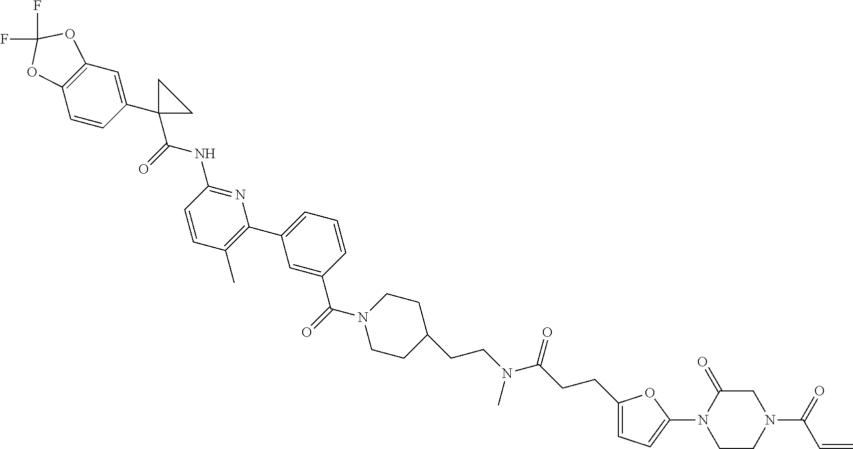 [SMILES: C=CC(=O)N1CCN(c2ccc(CCC(=O)N(C)CCC3CCN(C(=O)c4cccc(-c5nc(NC(=O)C6(c7ccc8c(c7)OC(F)(F)O8)CC6)ccc5C)c4)CC3)o2)C(=O)C1]

1H NMR (400 MHz, CDCl₃) δ 8.14 (s, 1H), 7.82-7.52 (m, 2H), 7.50-7.43 (m, 3H), 7.40 (s, 1H), 7.23 (dt, J=8.3, 2.2 Hz, 1H), 7.19 (d, J=1.6 Hz, 1H), 7.08 (dd, J=8.2, 5.6 Hz, 1H), 6.51 (d, J=14.1 Hz, 1H), 6.45-6.33 (m, 1H), 6.26 (d, J=3.2 Hz, 1H), 6.05 (d, J=3.3 Hz, 1H), 5.84-5.76 (m, 1H), 4.68 (s, 1H), 4.50-4.33 (m, 2H), 4.05-3.69 (m, 5H), 3.54-3.18 (m, 2H), 3.05-2.86 (m, 6H), 2.76 (d, J=17.3 Hz, 1H), 2.64-2.55 (m, 2H), 2.27 (s, 3H), 1.89-1.73 (m, 3H), 1.54-1.44 (m, 3H), 1.35-1.28 (m, 1H), 1.23-1.04 (m, 4H).

13C: (101 MHz, CDCl3) δ 171.10, 170.9, 164.97, 150.26, 144.52, 144.15, 143.67, 134.21, 131.70, 130.05, 128.40, 127.69, 126.69, 126.31, 112.46, 110.20, 107.09, 100.97, 47.46, 45.37, 35.07, 34.92, 33.91, 33.67, 33.54, 31.86, 31.32, 29.72, 23.78, 23.55, 19.15, 17.27. 19F: (376 MHz, CDCl3) δ −49.50, −49.52.

HRMS (TOF, ES+): m/z calcd for C46H49F2N6O8 (M+H)+ 851.3580; found 851.3572.

### N-((1-(1-(3-(5-(4-acryloyl-2-oxopiperazin-1-yl)furan-2-yl)propanoyl)piperidin-4-yl)-1H-1,2,3-triazol-4-yl)methyl)-3-(6-(1-(2,2-difluorobenzo[d][1,3]dioxol-5-yl)cyclopropane-1-carboxamido)-3-methylpyridin-2-yl)benzamide (Compound 209)

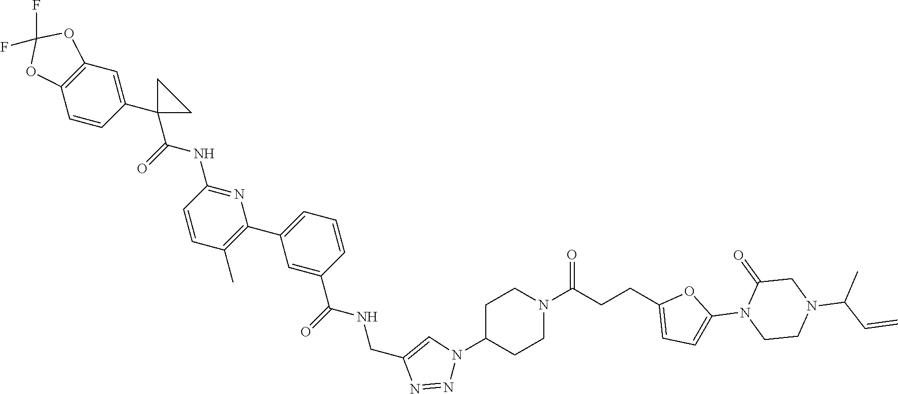 [SMILES: C=CC(C)N1CCN(c2ccc(CCC(=O)N3CCC(n4cc(CNC(=O)c5cccc(-c6nc(NC(=O)C7(c8ccc9c(c8)OC(F)(F)O9)CC7)ccc6C)c5)nn4)CC3)o2)C(=O)C1]

1H NMR (400 MHz, CDCl₃) δ 8.18 (br s, 1H), 8.01-7.59 (m, 5H), 7.58-7.44 (m, 3H), 7.23 (dd, J=8.2, 1.8 Hz, 1H), 7.18 (d, J=1.7 Hz, 1H), 7.08 (d, J=8.2 Hz, 1H), 6.50 (d, J=11.6 Hz, 1H), 6.39 (dd, J=16.7, 1.9 Hz, 1H), 6.25 (d, J=3.2 Hz, 1H), 6.07 (d, J=3.2 Hz, 1H), 5.80 (dd, J=10.3, 1.9 Hz, 1H), 4.78-4.67 (m, 3H), 4.63 (tt, J=11.3, 4.1 Hz, 1H), 4.47-4.33 (m, 2H), 4.07-3.79 (m, 5H), 3.22 (ddd, J=14.2, 11.9, 2.8 Hz, 1H), 2.97 (td, J=7.6, 2.8 Hz, 2H), 2.90-2.78 (m, 1H), 2.68 (q, J=7.4 Hz, 2H), 2.41-2.13 (m, 5H), 2.01-1.84 (m, 2H), 1.77 (q, J=4.0 Hz, 2H), 1.21 (s, 2H)

13C NMR (101 MHz, CDCl3) δ 169.91, 167.08, 164.98, 150.02, 144.71, 144.20, 143.75, 134.23, 131.97, 131.69, 129.94, 129.15, 128.68, 126.71, 126.33, 120.54, 112.46, 110.24, 107.36, 101.06, 57.82, 49.45, 46.82, 44.13, 40.51, 35.57, 32.74, 32.09, 31.98, 31.45, 23.78, 19.02, 17.43.

19F: (376 MHz, CDCl3) δ −49.46

HRMS (TOF, ES+): m/z calcd for C46H46F2N9O8 (M+H)+ 890.3437; found 890.3433.

### N-(1-(3-(5-(4-acryloyl-2-oxopiperazin-1-yl)furan-2-yl)propanoyl)piperidin-4-yl)-3-(6-(1-(2,2-difluorobenzo[d][1,3]dioxol-5-yl)cyclopropane-1-carboxamido)-3-methylpyridin-2-yl)-N-methylbenzamide (Compound 210)

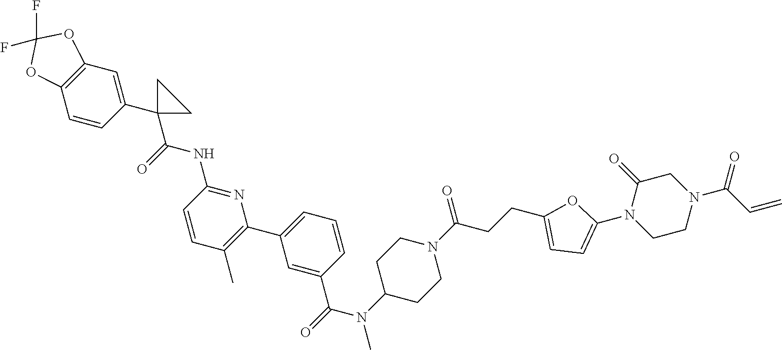 [SMILES: C=CC(=O)N1CCN(c2ccc(CCC(=O)N3CCC(N(C)C(=O)c4cccc(-c5nc(NC(=O)C6(c7ccc8c(c7)OC(F)(F)O8)CC6)ccc5C)c4)CC3)o2)C(=O)C1]

1H NMR (400 MHz, CDCl₃) δ 8.10 (s, 1H), 7.76-7.55 (m, 1H), 7.51-7.43 (m, 3H), 7.39 (d, J=6.9 Hz, 1H), 7.23 (dd, J=8.2, 1.7 Hz, 1H), 7.19 (d, J=1.7 Hz, 1H), 7.08 (d, J=8.2 Hz, 1H), 6.50 (s, 1H), 6.41 (dd, J=16.7, 2.0 Hz, 1H), 6.27 (d, J=3.2 Hz, 1H), 6.07 (d, J=3.2 Hz, 1H), 5.82 (dd, J=10.2, 2.0 Hz, 1H), 4.77 (s, 2H), 4.50-4.32 (m, 2H), 4.09-3.71 (m, 6H), 3.17 (s, 1H), 2.96 (t, J=7.7 Hz, 2H), 2.93-2.75 (m, 3H), 2.66 (s, 2H), 2.27 (s, 3H), 1.81-1.72 (m, 3H), 1.59 (s, 2H), 1.37-1.28 (m, 1H), 1.18 (s, 2H)

13C NMR (101 MHz, CDCl3) δ 169.70, 164.97, 150.17, 144.62, 144.16, 143.65, 131.69, 130.14, 128.39, 126.68, 126.30, 112.46, 110.21, 107.16, 100.94, 69.02, 49.37, 44.78, 39.08, 31.50, 29.72, 23.72, 19.21, 17.26.

19F: (376 MHz, CDCl3) δ −49.56

HRMS (TOF, ES+): m/z calcd for C44H45F2N6O8 (M+H)+ 823.3267; found 823.3247.

### N-(6-(3-(4-(3-(5-(4-acryloyl-2-oxopiperazin-1-yl)furan-2-yl)propanoyl)piperazine-1-carbonyl)phenyl)-5-methylpyridin-2-yl)-1-(2,2-difluorobenzo[d][1,3]dioxol-5-yl)cyclopropane-1-carboxamide (Compound 213)

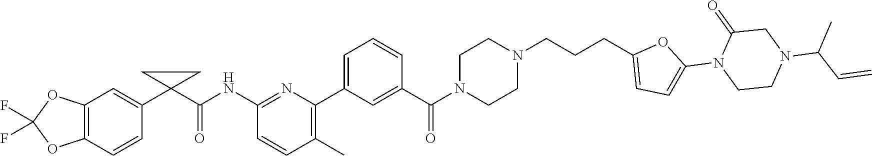 [SMILES: C=CC(C)N1CCN(c2ccc(CCCN3CCN(C(=O)c4cccc(-c5nc(NC(=O)C6(c7ccc8c(c7)OC(F)(F)O8)CC6)ccc5C)c4)CC3)o2)C(=O)C1]

1H NMR (400 MHz, CDCl₃) δ 8.08 (d, J=8.4 Hz, 1H), 7.65 (s, 1H), 7.59 (d, J=8.4 Hz, 1H), 7.54-7.45 (m, 3H), 7.40 (dt, J=7.4, 1.6 Hz, 1H), 7.23 (dd, J=8.2, 1.7 Hz, 1H), 7.19 (d, J=1.7 Hz, 1H), 7.10 (d, J=8.2 Hz, 1H), 6.52 (s, 1H), 6.41 (dd, J=16.7, 2.0 Hz, 1H), 6.26 (d, J=3.3 Hz, 1H), 6.07 (d, J=3.3 Hz, 1H), 5.82 (dd, J=10.2, 2.0 Hz, 1H), 4.48-4.36 (m, 2H), 4.05-3.83 (m, 4H), 3.80-3.37 (m, 8H), 2.97 (dd, J=8.8, 6.4 Hz, 2H), 2.65 (d, J=9.4 Hz, 2H), 2.26 (s, 3H), 1.74 (q, J=3.9 Hz, 2H), 1.16 (q, J=3.9 Hz, 2H)

13C NMR (101 MHz, CDCl3) δ 171.75, 170.24, 170.14, 164.97, 155.06, 149.93, 148.92, 144.72, 144.13, 143.62, 141.11, 140.28, 135.04, 134.94, 134.23, 131.68, 130.70, 129.99, 129.14, 128.49, 128.00, 126.94, 126.84, 126.62, 126.29, 112.94, 112.44, 110.26, 107.31, 101.08, 49.46, 46.83, 39.06, 31.56, 31.19, 23.60, 19.28, 17.23.

19F: (376 MHz, CDCl3) δ −49.54

HRMS (TOF, ES+): m/z calcd for C42H41F2N6O8 (M+H)+ 795.2954, found 795.2943.

### N-(6-(3-(7-(3-(5-(4-acryloyl-2-oxopiperazin-1-yl)furan-2-yl)propanoyl)-2,7-diazaspiro[3.5]-nonane-2-carbonyl)phenyl)-5-methylpyridin-2-yl)-1-(2,2-difluorobenzo[d][1,3]dioxol-5-yl)cyclopropane-1-carboxamide (Compound 214)

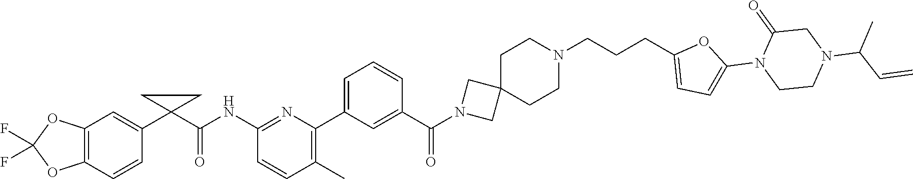 [SMILES: C=CC(C)N1CCN(c2ccc(CCCN3CCC4(CC3)CN(C(=O)c3cccc(-c5nc(NC(=O)C6(c7ccc8c(c7)OC(F)(F)O8)CC6)ccc5C)c3)C4)o2)C(=O)C1]

1H NMR (400 MHz, CDCl₃) δ 8.08 (d, J=8.4 Hz, 1H), 7.80-7.67 (m, 2H), 7.64-7.58 (m, 2H), 7.52 (dt, J=7.7, 1.5 Hz, 1H), 7.46 (t, J=7.6 Hz, 1H), 7.23 (dd, J=8.2, 1.8 Hz, 1H), 7.18 (d, J=1.7 Hz, 1H), 7.07 (d, J=8.2 Hz, 1H), 6.50 (d, J=13.4 Hz, 1H), 6.40 (dd, J=16.7, 2.0 Hz, 1H), 6.25 (d, J=3.2 Hz, 1H), 6.05 (d, J=3.3 Hz, 1H), 5.82 (dd, J=10.3, 2.0 Hz, 1H), 4.51-4.31 (m, 2H), 4.06-3.80 (m, 8H), 3.66-3.45 (m, 2H), 3.36 (t, J=5.6 Hz, 2H), 2.94 (dd, J=8.9, 6.4 Hz, 2H), 2.68-2.57 (m, 2H), 2.26 (s, 3H), 1.88-1.62 (m, 6H), 1.17 (q, J=3.8 Hz, 2H)

13C NMR (101 MHz, CDCl3) δ 170.11, 169.84, 164.98, 150.17, 148.82, 144.61, 144.13, 143.63, 134.78, 134.23, 133.15, 131.68, 131.57, 129.98, 129.14, 128.63, 128.32, 127.64, 127.05, 126.71, 126.29, 113.07, 112.43, 110.23, 107.17, 101.05, 62.93, 58.32, 49.46, 46.77, 42.44, 39.07, 38.84, 35.67, 34.91, 34.55, 31.54, 31.21, 29.71, 23.71, 19.17, 17.38.

19F: (376 MHz, CDCl3) δ −49.49

HRMS (TOF, ES+): m/z calcd for C45H45F2N6O8 (M+H)+ 835.3267; found 835.3298.

### N-(6-(3-(4-(2-(3-(5-(4-acryloyl-2-oxopiperazin-1-yl)furan-2-yl)propanamido)ethyl)piperidine-1-carbonyl)phenyl)-5-methylpyridin-2-yl)-1-(2,2-difluorobenzo[d][1,3]dioxol-5-yl)cyclopropane-1-carboxamide (Compound 225)

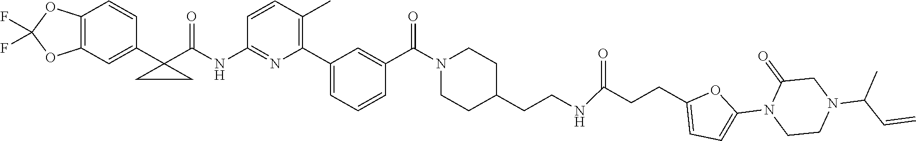 [SMILES: C=CC(C)N1CCN(c2ccc(CCC(=O)NCCC3CCN(C(=O)c4cccc(-c5nc(NC(=O)C6(c7ccc8c(c7)OC(F)(F)O8)CC6)ccc5C)c4)CC3)o2)C(=O)C1]

1H NMR (400 MHz, CDCl3) δ 8.10 (s, 1H), 7.82-7.54 (m, 2H), 7.49-7.42 (m, 3H), 7.39 (dd, J=5.4, 3.2 Hz, 1H), 7.23 (dd, J=8.2, 1.7 Hz, 1H), 7.19 (d, J=1.7 Hz, 1H), 7.09 (d, J=8.1 Hz, 1H), 6.52 (s, 1H), 6.41 (dd, J=16.7, 2.0 Hz, 1H), 6.20 (d, J=3.2 Hz, 1H), 6.05 (d, J=3.2 Hz, 1H), 5.82 (dd, J=10.2, 2.0 Hz, 1H), 5.61 (s, 1H), 4.67 (s, 1H), 4.51-4.32 (m, 2H), 4.05-3.72 (m, 5H), 3.25 (q, J=6.9 Hz, 2H), 2.99-2.87 (m, 3H), 2.73 (s, 1H), 2.48 (t, J=7.3 Hz, 2H), 2.26 (s, 3H), 1.83-1.72 (m, 3H), 1.57-1.47 (m, 2H), 1.42 (q, J=7.1 Hz, 2H), 1.23-1.00 (m, 4H)

13C NMR (101 MHz, CDCl3) δ 171.45, 169.80, 164.99, 149.89, 148.76, 144.92, 144.14, 143.64, 136.32, 134.87, 134.24, 131.69, 130.07, 129.17, 128.37, 127.68, 127.09, 126.69, 126.26, 112.93, 112.46, 110.21, 107.37, 101.10, 47.99, 42.42, 39.06, 36.98, 36.12, 35.02, 33.69, 32.67, 31.74, 31.26, 24.26, 19.18, 17.25.

19F: (376 MHz, CDCl3) δ −49.50

HRMS (TOF, ES+): m/z calcd for C45H47F2N6O8 (M+H)+ 837.3423, found 837.3448.

### N-(6-(3-(4-((3-(5-(4-acryloyl-2-oxopiperazin-1-yl)furan-2-yl)-N-methylpropanamido)methyl)piperidine-1-carbonyl)phenyl)-5-methylpyridin-2-yl)-1-(2,2-difluorobenzo[d][1,3]dioxol-5-yl)cyclopropane-1-carboxamide (Compound 215)

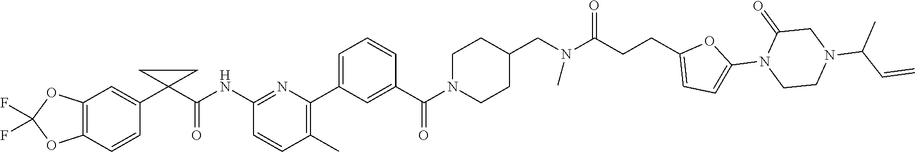 [SMILES: C=CC(C)N1CCN(c2ccc(CCC(=O)N(C)CC3CCN(C(=O)c4cccc(-c5nc(NC(=O)C6(c7ccc8c(c7)OC(F)(F)O8)CC6)ccc5C)c4)CC3)o2)C(=O)C1]

1H NMR (400 MHz, CDCl₃) δ 8.07 (d, J=8.4 Hz, 1H), 7.71 (s, 1H), 7.59 (d, J=8.5 Hz, 1H), 7.48-7.43 (m, 3H), 7.38 (dt, J=6.8, 1.9 Hz, 1H), 7.24 (dd, J=8.2, 1.7 Hz, 1H), 7.19 (d, J=1.7 Hz, 1H), 7.09 (d, J=8.2 Hz, 1H), 6.52 (s, 1H), 6.40 (dd, J=16.7, 2.0 Hz, 1H), 6.25 (d, J=3.3 Hz, 1H), 6.05 (t, J=3.8 Hz, 1H), 5.81 (dd, J=10.3, 2.0 Hz, 1H), 4.66 (s, 1H), 4.48-4.32 (m, 2H), 4.06-3.69 (m, 5H), 3.40 (s, 1H), 3.27-3.11 (m, 1H), 3.00 (s, 3H), 2.96-2.91 (m, 3H), 2.81-2.72 (m, 1H), 2.63 (t, J=6.5 Hz, 2H), 2.26 (s, 3H), 2.00-1.87 (m, 1H), 1.79-1.72 (m, 3H), 1.57-1.40 (m, 2H), 1.20-1.12 (m, 3H)

13C NMR (101 MHz, CDCl3) δ 171.85, 171.66, 169.94, 164.98, 150.27, 148.80, 144.57, 144.11, 143.60, 141.22, 136.21, 134.90, 134.23, 131.68, 130.07, 129.14, 128.44, 128.35, 127.67, 127.07, 126.71, 126.30, 112.88, 112.45, 110.22, 107.11, 100.95, 53.44, 49.46, 39.09, 36.61, 34.92, 31.88, 31.20, 30.53, 29.58, 23.79, 23.57, 19.24, 17.27.

19F: (376 MHz, CDCl3) δ −49.51

HRMS (TOF, ES+): m/z calcd for C45H47F2N6O8 (M+H)+ 837.3423; found 837.3439.

### N-(6-(3-((3aR,8aS)-2-(3-(5-(4-acryloyl-2-oxopiperazin-1-yl)furan-2-yl)propanoyl)decahydropyrrolo[3,4-d]azepine-6-carbonyl)phenyl)-5-methylpyridin-2-yl)-1-(2,2-difluorobenzo[d][1,3]dioxol-5-yl)cyclopropane-1-carboxamide (Compound 216)

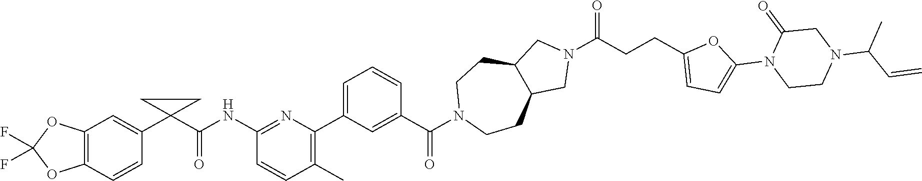 [SMILES: C=CC(C)N1CCN(c2ccc(CCC(=O)N3C[C@H]4CCN(C(=O)c5cccc(-c6nc(NC(=O)C7(c8ccc9c(c8)OC(F)(F)O9)CC7)ccc6C)c5)CC[C@H]4C3)o2)C(=O)C1]

1H NMR (400 MHz, CDCl₃) δ 8.09 (d, J=8.4 Hz, 1H), 7.66 (s, 1H), 7.59 (d, J=8.4 Hz, 1H), 7.48-7.42 (m, 3H), 7.38 (dt, J=6.3, 2.0 Hz, 1H), 7.23 (dd, J=8.2, 1.8 Hz, 1H), 7.19 (d, J=1.7 Hz, 1H), 7.08 (d, J=8.2 Hz, 1H), 6.52 (s, 1H), 6.40 (dd, J=16.7, 2.0 Hz, 1H), 6.25 (d, J=1.8 Hz, 1H), 6.05 (d, J=3.3 Hz, 1H), 5.81 (dd, J=10.2, 2.0 Hz, 1H), 4.52-4.28 (m, 2H), 4.06-3.79 (m, 5H), 3.74-3.45 (m, 4H), 3.37-3.25 (m, 2H), 3.22-3.11 (m, 1H), 2.95 (t, J=7.6 Hz, 2H), 2.56 (t, J=8.3 Hz, 3H), 2.53-2.38 (m, 2H), 2.25 (s, 3H), 2.11-1.97 (m, 1H), 1.85-1.72 (m, 5H), 1.16 (q, J=3.9 Hz, 2H)

13C NMR (101 MHz, CDCl3) δ 171.77, 170.94, 170.04, 164.97, 163.22, 155.29, 150.26, 148.85, 144.58, 144.11, 143.60, 141.07, 140.07, 136.55, 134.94, 134.23, 131.68, 130.08, 129.14, 128.43, 127.48, 126.99, 126.70, 126.53, 126.31, 112.89, 112.46, 110.20, 107.11, 100.98, 52.48, 51.83, 51.57, 51.14, 49.46, 47.76, 43.17, 42.94, 40.49, 39.10, 32.86, 31.20, 30.18, 23.39, 19.24, 17.26. 19F: (376 MHz, CDCl3) δ −49.55 HRMS (TOF, ES+): m/z calcd for C46H47F2N6O8 (M+H)+ 849.3423; found 849.3475.

### N-(6-(3-(4-((1-(3-(5-(4-acryloyl-2-oxopiperazin-1-yl)furan-2-yl)propanoyl)azetidin-3-yl)oxy)piperidine-1-carbonyl)phenyl)-5-methylpyridin-2-yl)-1-(2,2-difluorobenzo[d][1,3]dioxol-5-yl)cyclopropane-1-carboxamide (Compound 217)

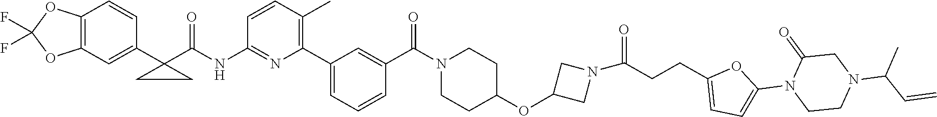 [SMILES: C=CC(C)N1CCN(c2ccc(CCC(=O)N3CC(OC4CCN(C(=O)c5cccc(-c6nc(NC(=O)C7(c8ccc9c(c8)OC(F)(F)O9)CC7)ccc6C)c5)CC4)C3)o2)C(=O)C1]

1H NMR (400 MHz, CDCl₃) δ 8.09 (d, J=8.4 Hz, 1H), 7.68 (s, 1H), 7.59 (d, J=8.4 Hz, 1H), 7.50-7.42 (m, 3H), 7.39 (dt, J=7.0, 1.8 Hz, 1H), 7.23 (dd, J=8.2, 1.8 Hz, 1H), 7.19 (d, J=1.7 Hz, 1H), 7.09 (d, J=8.2 Hz, 1H), 6.52 (s, 1H), 6.40 (dd, J=16.7, 2.0 Hz, 1H), 6.26 (d, J=3.3 Hz, 1H), 6.05 (d, J=3.3 Hz, 1H), 5.82 (dd, J=10.3, 2.0 Hz, 1H), 4.50-4.34 (m, 3H), 4.27-4.16 (m, 2H), 4.12-3.81 (m, 7H), 3.71-3.54 (m, 2H), 3.45 (d, J=21.9 Hz, 1H), 3.22 (s, 1H), 2.96-2.86 (m, 2H), 2.39 (t, J=7.6 Hz, 2H), 2.26 (s, 3H), 1.88 (s, 1H), 1.80-1.72 (m, 3H), 1.51 (s, 2H), 1.17 (q, J=3.9 Hz, 2H)

13C NMR (101 MHz, CDCl3) δ 171.78, 171.58, 169.98, 164.97, 163.22, 155.21, 149.95, 148.84, 144.63, 144.12, 143.61, 141.12, 135.90, 134.94, 134.24, 131.69, 130.25, 129.98, 129.15, 128.43, 127.63, 127.01, 126.69, 126.29, 112.89, 112.46, 110.20, 107.21, 101.04, 73.90, 65.51, 58.03, 55.96, 49.46, 46.72, 44.80, 39.10, 31.96, 31.21, 30.09, 23.25, 19.22, 17.25.

19F: (376 MHz, CDCl3) δ −49.52

HRMS (TOF, ES+): m/z calcd for C46H47F2N6O9 (M+H)+ 865.3373; found 865.3416.

### N-(6-(3-(1-(3-(5-(4-acryloyl-2-oxopiperazin-1-yl)furan-2-yl)propanoyl)-[3,4′-bipiperidine]-1′-carbonyl)phenyl)-5-methylpyridin-2-yl)-1-(2,2-difluorobenzo[d][1,3]dioxol-5-yl)cyclopropane-1-carboxamide (Compound 211)

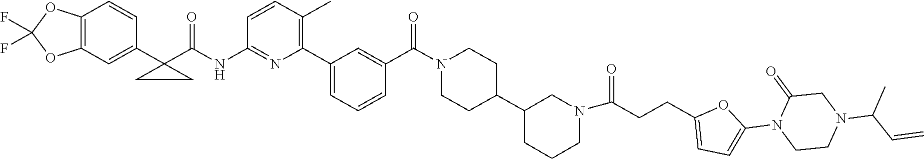 [SMILES: C=CC(C)N1CCN(c2ccc(CCC(=O)N3CCCC(C4CCN(C(=O)c5cccc(-c6nc(NC(=O)C7(c8ccc9c(c8)OC(F)(F)O9)CC7)ccc6C)c5)CC4)C3)o2)C(=O)C1]

1H NMR (400 MHz, CDCl₃) δ 8.09 (d, J=8.4 Hz, 1H), 7.70 (s, 1H), 7.61 (s, 1H), 7.49-7.43 (m, 3H), 7.40 (t, J=4.1 Hz, 1H), 7.25-7.22 (m, 1H), 7.20 (d, J=1.7 Hz, 1H), 7.09 (d, J=7.6 Hz, 1H), 6.52 (s, 1H), 6.41 (dd, J=16.7, 2.0 Hz, 1H), 6.26 (t, J=3.2 Hz, 1H), 6.05 (d, J=3.2 Hz, 1H), 5.82 (dd, J=10.2, 2.0 Hz, 1H), 4.74 (s, 1H), 4.53 (t, J=13.4 Hz, 1H), 4.48-4.31 (m, 2H), 4.07-3.70 (m, 6H), 3.02-2.87 (m, 4H), 2.77-2.68 (m, 1H), 2.63 (td, J=7.2, 2.1 Hz, 2H), 2.53-2.35 (m, 1H), 2.27 (s, 3H), 1.94-1.80 (m, 2H), 1.80-1.70 (m, 4H), 1.47-1.34 (m, 3H), 1.31-1.23 (m, 1H), 1.22-1.10 (m, 4H)

13C NMR (101 MHz, CDCl3) δ 169.67, 164.98, 148.74, 144.52, 144.14, 143.64, 134.81, 134.21, 131.69, 130.06, 128.41, 127.65, 126.71, 126.30, 113.01, 112.45, 110.22, 107.05, 100.95, 100.86, 49.41, 46.25, 42.69, 41.71, 40.51, 39.08, 31.56, 31.25, 27.87, 25.79, 24.97, 23.72, 19.20, 17.29.

19F: (376 MHz, CDCl3) δ −49.53

HRMS (TOF, ES+): m/z calcd for C48H51F2N6O8 (M+H)+ 877.3736; found 877.3794.

### N-(2-(1-(3-(5-(4-acryloyl-2-oxopiperazin-1-yl)furan-2-yl)propanoyl)piperidin-4-yl)ethyl)-3-(6-(1-(2,2-difluorobenzo[d][1,3]dioxol-5-yl)cyclopropane-1-carboxamido)-3-methylpyridin-2-yl)benzamide (218)

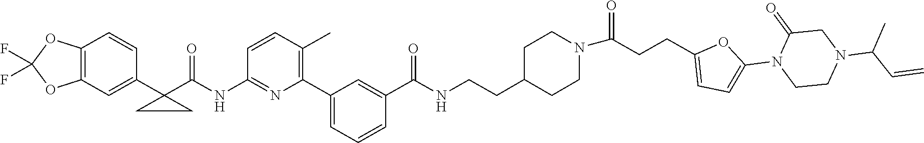 [SMILES: C=CC(C)N1CCN(c2ccc(CCC(=O)N3CCC(CCNC(=O)c4cccc(-c5nc(NC(=O)C6(c7ccc8c(c7)OC(F)(F)O8)CC6)ccc5C)c4)CC3)o2)C(=O)C1]

1H NMR (400 MHz, CDCl₃) δ 8.10 (d, J=8.4 Hz, 1H), 7.79 (s, 1H), 7.75 (d, J=7.5 Hz, 1H), 7.68 (s, 1H), 7.59 (d, J=8.4 Hz, 1H), 7.54 (dt, J=7.7, 1.5 Hz, 1H), 7.48 (t, J=7.6 Hz, 1H), 7.22 (dd, J=8.2, 1.8 Hz, 1H), 7.19 (d, J=1.7 Hz, 1H), 7.07 (d, J=8.2 Hz, 1H), 6.50 (s, 1H), 6.39 (dd, J=16.7, 2.0 Hz, 1H), 6.27 (d, J=3.2 Hz, 1H), 6.19 (s, 1H), 6.05 (d, J=3.2 Hz, 1H), 5.81 (dd, J=10.3, 2.0 Hz, 1H), 4.67-4.56 (m, 1H), 4.48-4.33 (m, 2H), 4.04-3.77 (m, 5H), 3.49 (q, J=6.6 Hz, 2H), 3.03-2.91 (m, 3H), 2.67-2.58 (m, 2H), 2.54 (td, J=12.9, 2.8 Hz, 1H), 2.25 (s, 3H), 1.85-1.72 (m, 4H), 1.61-1.52 (m, 3H), 1.21-1.08 (m, 4H)

13C NMR (101 MHz, CDCl3) δ 171.81, 169.64, 169.34, 167.25, 164.98, 155.36, 148.90, 144.49, 144.15, 143.64, 141.04, 140.19, 134.87, 134.23, 131.83, 131.68, 128.55, 127.46, 127.00, 126.66, 126.52, 126.32, 113.00, 112.44, 110.19, 107.10, 100.88, 49.45, 45.68, 42.16, 42.04, 39.07, 37.54, 36.29, 33.83, 32.54, 32.44, 31.84, 31.76, 31.53, 31.20, 23.79, 19.15, 17.28.

19F: (376 MHz, CDCl3) δ −49.52

HRMS (TOF, ES+): m/z calcd for C45H47F2N6O8 (M+H)+ 837.3423; found 837.3455.

### N-(6-(3-(4-(((1-(3-(5-(4-acryloyl-2-oxopiperazin-1-yl)furan-2-yl)propanoyl)pyrrolidin-3-yl)oxy)methyl)piperidine-1-carbonyl)phenyl)-5-methylpyridin-2-yl)-1-(2,2-difluorobenzo[d][1,3]dioxol-5-yl)cyclopropane-1-carboxamide (Compound 212)

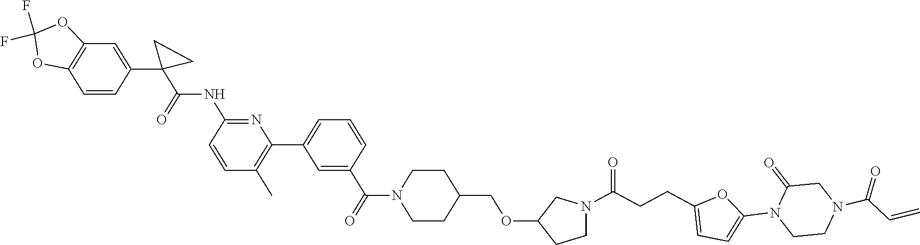 [SMILES: C=CC(=O)N1CCN(c2ccc(CCC(=O)N3CCC(OCC4CCN(C(=O)c5cccc(-c6nc(NC(=O)C7(c8ccc9c(c8)OC(F)(F)O9)CC7)ccc6C)c5)CC4)C3)o2)C(=O)C1]

1H NMR (400 MHz, CDCl₃) δ 8.09 (d, J=8.3 Hz, 1H), 7.68 (s, 1H), 7.59 (d, J=8.4 Hz, 1H), 7.47-7.42 (m, 3H), 7.39 (d, J=1.8 Hz, 1H), 7.23 (dd, J=8.2, 1.8 Hz, 1H), 7.19 (d, J=1.7 Hz, 1H), 7.08 (d, J=8.1 Hz, 1H), 6.52 (s, 1H), 6.40 (dd, J=16.7, 2.0 Hz, 1H), 6.26 (dd, J=3.2, 1.2 Hz, 1H), 6.05 (d, J=3.2 Hz, 1H), 5.81 (dd, J=10.2, 2.0 Hz, 1H), 4.72 (s, 1H), 4.48-4.34 (m, 2H), 4.10-3.75 (m, 6H), 3.66-3.58 (m, 1H), 3.54-3.39 (m, 3H), 3.34-3.22 (m, 2H), 3.05-2.87 (m, 3H), 2.81-2.70 (m, 1H), 2.60-2.50 (m, 2H), 2.26 (s, 3H), 2.12-1.95 (m, 2H), 1.94-1.71 (m, 5H), 1.21-1.04 (m, 4H)

13C NMR (101 MHz, CDCl3) δ 171.78, 170.23, 170.02, 169.92, 164.97, 150.29, 148.82, 144.50, 144.13, 143.61, 141.08, 136.23, 134.92, 134.24, 131.69, 130.09, 128.33, 127.73, 127.69, 127.02, 126.67, 126.31, 112.85, 112.44, 110.20, 107.03, 100.93, 78.63, 73.53, 73.39, 52.05, 50.93, 44.59, 43.69, 42.18, 39.06, 36.74, 36.70, 33.03, 32.80, 31.66, 31.21, 29.66, 23.29, 19.25, 17.23.

19F: (376 MHz, CDCl3) δ −49.51, −49.52

HRMS (TOF, ES+): m/z calcd for C48H51F2N6O9 (M+H)+ 893.3686; found 893.3688.

### Synthesis of Compound 231

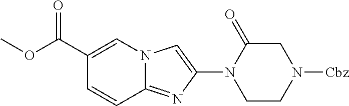 [SMILES: COC(=O)c1ccc2nc(N3CCN(C(=O)OCc4ccccc4)CC3=O)cn2c1]

methyl 2-(4-((benzyloxy)carbonyl)-2-oxopiperazin-1-yl)imidazo[1,2-a]pyridine-6-carboxylate: methyl 2-bromoimidazo[1,2-a]pyridine-6-carboxylate (100 mg, 0.39 mmol), benzyl 3-oxopiperazine-1-carboxylate (101 mg, 0.43 mmol), potassium carbonate (161 mg, 1.17 mmol), copper (I) iodide (7.5 mg, 0.039 mmol), and N,N′-dimethyldiaminoethane (11 mL, 0.10 mmol) were combined and dissolved in 1,4-dioxane (2 mL) under nitrogen. The mixture was degassed by sonicating under vacuum and backfilling with nitrogen twice. The reaction was then stirred at 100° C. for 16h. and sat. ammonium chloride (1 mL) and water 5 mL) was added stirred for 20 minutes. Additional water was added, and the mixture was extracted three times with ethyl acetate. Organic extracts were combined, washed with brine, dried over sodium sulfate, and concentrated. The crude product was purified by silica gel chromatography (0-80% EtOAc/Hex) to provide the title compound (62 mg, 0.15 mmol, 39%) as a solid. LC/MS [M+H]⁺ m/z calc. 409.14, found 409.1. 1H NMR (400 MHz, CDCl3) δ 8.94-8.89 (m, 1H), 8.41 (s, 1H), 7.79 (dd, J=9.4, 1.7 Hz, 1H), 7.54 (d, J=9.4 Hz, 1H), 7.45-7.34 (m, 5H), 5.23 (s, 2H), 4.43 (s, 2H), 4.35-4.30 (m, 2H), 4.00 (s, 3H), 3.92 (t, J=5.5 Hz, 2H).

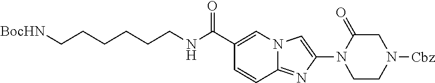 [SMILES: CC(C)(C)OC(=O)NCCCCCCNC(=O)c1ccc2nc(N3CCN(C(=O)OCc4ccccc4)CC3=O)cn2c1]

benzyl 4-(6-((6-((tert-butoxycarbonyl)amino)hexyl)carbamoyl)imidazo[1,2-a]pyridin-2-yl)-3-oxopiperazine-1-carboxylate: methyl 2-(4-((benzyloxy)carbonyl)-2-oxopiperazin-1-yl)imidazo[1,2-a]pyridine-6-carboxylate (60 mg, 0.15 mmol) was dissolved in THF (1.5 mL) and two drops of MeOH. Aqueous LiOH (1.5 mL, 0.75 mmol, 0.5 M) was added and the reaction mixture stirred for 2h. The solution was diluted with water, acidified with HCl (1 mL, 1 M), and extracted three times with DCM. Organic extracts were combined, dried over sodium sulfate, and concentrated to provide the carboxylic acid, which was directly dissolved in DMF (1.5 mL). Tert-butyl (6-aminohexyl)carbamate (39 mg, 0.18 mmol), DIEA (131 mL, 0.75 mmol), and HATU (114 mg, 0.30 mmol) were added and the reaction stirred overnight. Water was added and the mixture extracted with EtOAc three times. Organic extracts were combined, washed with brine, dried over sodium sulfate, and concentrated. The crude product was purified by silica gel chromatography (0-60% EtOAc/Hex) to provide the title compound (28 mg, 0.047 mmol, 31%) as an oil. LC/MS [M+H]⁺ m/z calc. 593.30, found 593.3. 1H NMR (400 MHz, CDCl3) δ 8.82 (s, 1H), 8.35 (s, 1H), 7.59 (d, J=9.3 Hz, 1H), 7.49 (d, J=9.3 Hz, 1H), 7.42-7.29 (m, 5H), 6.80 (s, 1H), 4.59 (s, 1H), 4.38 (s, 2H), 4.28 (s, 2H), 3.87 (t, J=5.5 Hz, 2H), 3.46 (q, J=6.4 Hz, 2H), 3.17 (d, J=6.5 Hz, 2H), 3.00 (s, 2H), 1.51-1.44 (m, 4H), 1.42 (s, 9H), 1.39-1.31 (m, 4H).

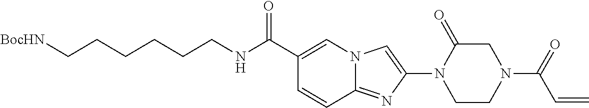 [SMILES: C=CC(=O)N1CCN(c2cn3cc(C(=O)NCCCCCCNC(=O)OC(C)(C)C)ccc3n2)C(=O)C1]

tert-butyl (6-(2-(4-acryloyl-2-oxopiperazin-1-yl)imidazo[1,2-a]pyridine-6-carboxamido)hexyl)carbamate: benzyl 4-(6-((6-((tert-butoxycarbonyl)amino)hexyl)carbamoyl)imidazo[1,2-a]pyridin-2-yl)-3-oxopiperazine-1-carboxylate (25 mg, 0.047 mmol) and Pd/C (6 mg, 10% wt.) were suspended in EtOH (4 mL), the atmosphere exchanged for hydrogen, and the mixture was stirred vigorously overnight. The Pd/C was removed via filtration (PTFE, 0.45 mm) and EtOH was removed under vacuum. The crude amine was then dissolved in DCM (1.5 mL) and the solution cooled to 0° C. DIEA (40 m25L, 0.23 mmol) was added, followed by acryloyl chloride (10 mL, 0.099 mmol) and the reaction was stirred at 0° C. for 20 min. Water was added and the mixture extracted with DCM three times. Organic extracts were combined, dried over sodium sulfate, and concentrated. The crude product was purified by silica gel chromatography (0-8% MeOH/DCM) to provide the title compound (20 mg, 0.039 mmol, 83%) as a solid. LC/MS [M+H]⁺ m/z calc. 513.27, found 513.3. 1H NMR (400 MHz, CDCl3) δ 8.86 (s, 1H), 8.36 (s, 1H), 7.65 (d, J=9.4 Hz, 1H), 7.52 (d, J=9.2 Hz, 1H), 6.98 (s, 1H), 6.69-6.51 (m, 1H), 6.44 (dd, J=16.8, 1.9 Hz, 1H), 5.85 (dd, J=10.3, 1.9 Hz, 1H), 4.67 (s, 1H), 4.52 (d, J=16.6 Hz, 2H), 4.35 (s, 2H), 4.03 (d, J=29.2 Hz, 2H), 3.49 (q, J=6.5 Hz, 2H), 3.25-3.15 (m, 2H), 1.67 (p, J=6.8 Hz, 2H), 1.58-1.35 (m, 15H).

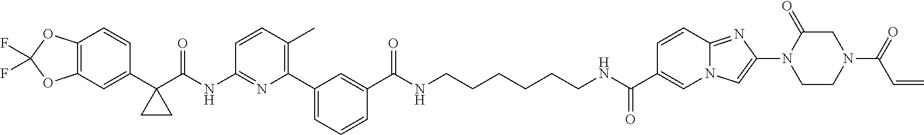 [SMILES: C=CC(=O)N1CCN(c2cn3cc(C(=O)NCCCCCCNC(=O)c4cccc(-c5nc(NC(=O)C6(c7ccc8c(c7)OC(F)(F)O8)CC6)ccc5C)c4)ccc3n2)C(=O)C1]

2-(4-acryloyl-2-oxopiperazin-1-yl)-N-(6-(3-(6-(1-(2,2-difluorobenzo[d][1,3]dioxol-5-yl)cycloprop-2-ene-1-carboxamido)-3-methylpyridin-2-yl)benzamido)hexyl)imidazo[1,2-a]pyridine-6-carboxamide (NJH-2-153): tert-butyl (6-(2-(4-acryloyl-2-oxopiperazin-1-yl)imidazo[1,2-a]pyridine-6-carboxamido)hexyl)carbamate (15 mg, 0.029 mmol) was dissolved in DCM (1 mL) and treated with TFA (0.5 mL) and stirred for 30 min. Volatiles were evaporated and the crude washed with DCM and evaporated twice. The crude amine and lumacaftor (3-(6-(1-(2,2-difluorobenzo[d][1,3]dioxol-5-yl)cyclopropane-1-carboxamido)-3-methylpyridin-2-yl)benzoic acid, 15 mg, 0.032 mmol) was dissolved in DMF (0.5 mL) and DIEA (25 mL, 0.15 mmol) was added followed by HATU (22 mg, 0.058 mmol). The solution was stirred for 20 minutes before water was added and the mixture extracted three times with EtOAc. Organic extracts were combined, washed with brine, dried over sodium sulfate, and concentrated. The crude product was purified by silica gel chromatography (0-7% MeOH/DCM) to provide the title compound (12.7 mg, 0.015 mmol, 52%) as a solid. HRMS (ESI) [M+H]⁺ m/z calc. 847.3301, found 847.3370. H1 NMR (600 MHz, CDCl3) δ 8.84 (s, 1H), 8.31 (s, 1H), 8.07 (d, J=8.4 Hz, 1H), 7.79 (d, J=1.8 Hz, 1H), 7.75 (dt, J=7.8, 1.5 Hz, 1H), 7.66 (s, 1H), 7.60 (d, J=9.2 Hz, 1H), 7.56 (d, J=8.5 Hz, 1H), 7.53 (dt, J=7.7, 1.4 Hz, 1H), 7.49-7.43 (m, 2H), 7.20 (dd, J=8.2, 1.8 Hz, 1H), 7.18 (d, J=1.7 Hz, 1H), 7.05 (d, J=8.2 Hz, 1H), 6.87 (s, 1H), 6.56 (s, 1H), 6.41 (dd, J=16.8, 1.7 Hz, 1H), 6.34 (s, 1H), 5.81 (d, J=10.7 Hz, 1H), 4.47 (d, J=27.0 Hz, 2H), 4.31 (s, 2H), 3.98 (d, J=49.3 Hz, 3H), 3.47 (dq, J=23.0, 6.5 Hz, 4H), 2.21 (s, 3H), 1.73 (q, J=3.9 Hz, 2H), 1.66-1.60 (m, 2H), 1.53-1.38 (m, 5H), 1.15 (q, J=3.9 Hz, 2H).

13C NMR (151 MHz, CDCl3) δ 171.7, 167.6, 164.8, 155.3, 148.9, 144.1, 143.6, 141.3, 141.0, 140.3, 134.9, 134.8, 131.9, 131.7, 128.6, 127.4, 127.0, 126.6, 126.5, 120.8, 115.7, 113.0, 112.4, 110.2, 104.0, 55.8, 43.7, 39.2, 39.1, 31.2, 29.6, 29.1, 25.4, 25.2, 19.1, 18.6, 17.2, 12.5.

### Synthesis of Compound 230

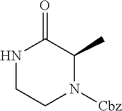 [SMILES: C[C@@H]1C(=O)NCCN1C(=O)OCc1ccccc1]

benzyl (R)-2-methyl-3-oxopiperazine-1-carboxylate: 453 mg (3.28 mmol) of potassium carbonate was dissolved in 3 mL of THE and stirred for 5 minutes. 1 mL of water was added to the reaction mixture, followed by dropwise addition of 310 μL (2.17 mmol) of benzyl chloroformate. 125 mg (1.10 mmol) of (R)-3-methylpiperazin-2-one was added, and the reaction mixture stirred overnight. Water was then added to the reaction, and the reaction mixture was extracted three times with ethyl acetate. The organic layers were combined, washed with brine, dried over sodium sulfate, and concentrated. Crude residues were purified by silica gel chromatography (0% to 80% EtOAc:Hexanes) to yield 160 mg (0.64 mmol, 59% yield) of the title compound as a solid. LC/MS [M+H]⁺ m/z calc. 249.12, found 249.1. 1H NMR (400 MHz, Chloroform-d) δ 7.47-7.34 (m, 5H), 6.16 (s, 1H), 5.21 (s, 2H), 4.83-4.63 (m, 1H), 4.38-4.12 (m, 1H), 3.61-3.42 (m, 1H), 3.31 (d, J=12.6 Hz, 2H), 1.63 (s, 2H).

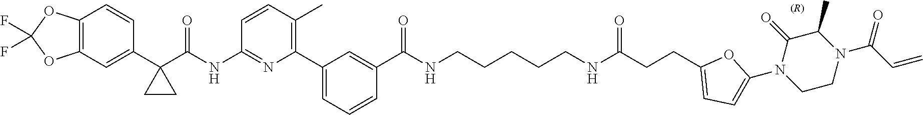

(R)—N-(5-(3-(5-(4-acryloyl-3-methyl-2-oxopiperazin-1-yl)furan-2-yl)propanamido)pentyl)-3-(6-(1-(2,2-difluorobenzo[d][1,3]dioxol-5-yl)cyclopropane-1-carboxamido)-3-methylpyridin-2-yl)benzamide: 160 mg (0.64 mmol) of benzyl (R)-2-methyl-3-oxopiperazine-1-carboxylate, 176 mg (0.64 mmol) of tert-butyl (E)-3-(5-bromofuran-2-yl)acrylate, 268 mg (1.94 mmol) of potassium carbonate, 18 μL (0.16 mmol) of N,N′-dimethylethylenediamine, and 13 mg (0.068 mmol) of copper iodide were dissolved in 3 mL of dioxane, degassed three times, heated to 100° C. and stirred overnight. The following day, water was added to the reaction, and the reaction mixture was extracted three times with ethyl acetate. The organic layers were combined, washed with brine, dried over sodium sulfate, and concentrated to give the crude intermediate benzyl (R)-4-(5-(4,4-dimethyl-3-oxopent-1-en-1-yl)furan-2-yl)-2-methyl-3-oxopiperazine-1-carboxylate. This intermediate, along with 30 mg of Pd/C (10% wt.) were added to 5 mL of EtOH, and the atmosphere was replaced with hydrogen gas. The reaction was stirred vigorously overnight. The following day, the reaction was filtered through celite to remove the Pd/C, concentrated to remove the EtOH, to yield the crude intermediate (R)-1-(5-(4,4-dimethyl-3-oxopentyl)furan-2-yl)-3-methylpiperazin-2-one. This crude intermediate was then immediately dissolved in 500 μL DCM, and 500 μL of TFA was added and the solution stirred for 1h. Volatiles were evaporated under vacuum, and DCM (1 mL) was added and evaporated to give the carboxylic acid intermediate (R)-3-(5-(3-methyl-2-oxopiperazin-1-yl)furan-2-yl)propanoic acid. This intermediate was dissolved in 500 μL DMF, and then 100 μL DIEA and 70 mg (0.13 mmol)N-(4-aminobutyl)-3-(6-(1-(2,2-difluorobenzo[d][1,3]dioxol-5-yl)cyclopropane-1-carboxamido)-3-methylpyridin-2-yl)benzamide were added, followed by 100 mg HATU. The reaction mixture was allowed to stir for 1h at rt. Water was added, and the mixture extracted three times with EtOAc. Organic extracts were combined, washed with brine, dried over sodium sulfate, and concentrated. Crude residues were purified by silica gel chromatography (0% to 4% MeOH in DCM) to yield 11.1 mg (0.013 mmol, 2% yield over three steps) of LEB-03-162 as a solid. HRMS (ESI) [M+H]⁺ m/z calc. 824.3345, found 825.3417. 1H NMR (400 MHz, Chloroform-d) δ 8.12 (d, J=8.4 Hz, 1H), 8.05 (s, 1H), 7.90-7.79 (m, 2H), 7.76 (s, 1H), 7.63 (d, J=8.5 Hz, 1H), 7.56 (dt, J=7.7, 1.5 Hz, 1H), 7.50 (t, J=7.6 Hz, 1H), 7.27 (dd, J=8.2, 1.8 Hz, 1H), 7.23 (d, J=1.8 Hz, 1H), 7.14-7.08 (m, 1H), 6.54 (s, 2H), 6.46 (s, 1H), 6.14 (dd, J=78.1, 3.3 Hz, 2H), 5.91 (s, 1H), 5.83 (d, J=10.1 Hz, 1H), 4.72 (s, 1H), 3.91-3.77 (m, 2H), 3.46 (p, J=6.2 Hz, 2H), 3.25 (q, J=6.6 Hz, 2H), 2.99 (s, 3H), 2.94-2.90 (m, 4H), 2.48 (t, J=7.3 Hz, 2H), 2.28 (s, 3H), 1.78 (q, J=3.9 Hz, 2H), 1.56-1.49 (m, 2H), 1.49-1.44 (m, 2H), 1.37 (q, J=8.0 Hz, 1H), 1.20 (q, J=3.9 Hz, 2H). 13C NMR (151 MHz, DMSO) δ 171.69, 171.00, 166.14, 162.78, 155.91, 150.13, 149.51, 143.31, 142.59, 141.14, 140.02, 136.74, 134.87, 131.74, 128.44, 128.15, 127.98, 127.21, 127.02, 126.79, 113.56, 112.69, 110.59, 106.88, 100.60, 54.08, 42.32, 38.88, 36.25, 33.78, 31.81, 31.24, 31.16, 29.29, 29.23, 24.32, 23.97, 19.18, 18.56, 17.21, 16.16, 12.95.

### Example 4: Synthesis of Exemplary DUB Recruiters

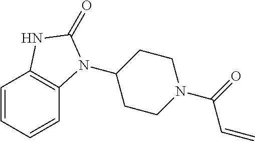 [SMILES: C=CC(=O)N1CCC(n2c(=O)[nH]c3ccccc32)CC1]

1-(1-acryloylpiperidin-4-yl)-1,3-dihydro-2H-benzo[d]imidazol-2-one: 1-(piperidin-4-yl)-1,3-dihydro-2H-benzo[d]imidazol-2-one (50 mg, 0.23 mmol) was acylated via general procedure H and the crude residue was purified by silica gel chromatography (0 to 20% MeOH/DCM) to afford the title compound as a an oil (11.8 mg, 0.043 mmol, 19%). 1H NMR (400 MHz, DMSO) δ 10.87 (s, 1H), 7.29-7.17 (m, 1H), 7.05-6.95 (m, 3H), 6.88 (ddd, J=16.1, 10.5, 3.3 Hz, 1H), 6.16 (d, J=2.4 Hz, 1H), 5.70 (dd, J=10.4, 2.4 Hz, 1H), 4.61 (d, J=13.1 Hz, 1H), 4.44 (tt, J=12.0, 3.9 Hz, 1H), 4.21 (d, J=13.8 Hz, 1H), 3.21 (t, J=13.3 Hz, 1H), 2.76 (t, J=12.9 Hz, 1H), 2.34-2.07 (m, 2H), 1.75 (d, J=12.4 Hz, 2H). 13C NMR (151 MHz, DMSO) δ 164.8, 154.2, 129.7, 129.0, 129.0, 127.7, 121.1, 120.9, 109.3, 109.0, 50.3, 45.1, 41.6, 29.9, 29.0. HRMS (ESI): [M+H]⁺ m/z calc. 272.14, found 272.1394.

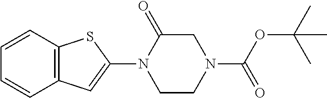 [SMILES: CC(C)(C)OC(=O)N1CCN(c2cc3ccccc3s2)C(=O)C1]

tert-butyl 4-(benzo[b]thiophen-2-yl)-3-oxopiperazine-1-carboxylate: 2-bromobenzo[b]thiophene (100 mg, 0.47 mmol) was coupled to tert-butyl 3-oxopiperazine-1-carboxylate (93.5 mg, 0.47 mmol) via general procedure D and the crude residue was purified by silica gel chromatography (0 to 100% EtOAc/hexane) to yield a solid (22.3 mg, 0.116 mmol, 14%). 1H NMR (400 MHz, CDCl3) δ 7.81 (d, J=7.8 Hz, 1H), 7.72 (d, J=7.7 Hz, 1H), 7.30 (s, 2H), 6.92 (s, 1H), 4.40 (s, 2H), 4.01 (t, J=5.4 Hz, 2H), 3.92 (t, J=5.4 Hz, 2H), 1.54 (s, 9H).

LC/MS: [M+H]⁺ m/z calc. 333.1, found 333.1

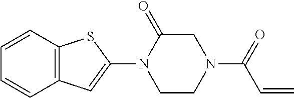 [SMILES: C=CC(=O)N1CCN(c2cc3ccccc3s2)C(=O)C1]

4-acryloyl-1-(benzo[b]thiophen-2-yl)piperazin-2-one: tert-butyl 4-(benzo[b]thiophen-2-yl)-3-oxopiperazine-1-carboxylate (EZ-1-035) (18 mg, 0.05 mmol) was deprotected and acylated via general procedures F and H respectively. The crude residue was purified by silica gel chromatography (0 to 100% EtOAc/Hex) to afford the title compound as a solid (6.6 mg, 0.023 mmol, 46%). 1H NMR (400 MHz, DMSO) δ 7.86 (d, J=7.9 Hz, 1H), 7.74 (t, J=7.2 Hz, 1H), 7.45-7.32 (m, 1H), 7.28 (q, J=6.8 Hz, 1H), 7.11 (s, 1H), 6.98-6.77 (m, 1H), 6.21 (d, J=16.7 Hz, 1H), 5.83-5.74 (m, 1H), 4.50 (d, J=68.5 Hz, 2H), 4.18-3.91 (m, 4H). 13C NMR (151 MHz, DMSO) δ 164.7, 142.0, 136.7, 136.2, 128.9, 128.0, 124.9, 123.9, 122.8, 122.1, 108.0, 49.2, 48.4, 47.6, 46.8. HRMS (ESI): [M+Na]⁺ m/z calc. 309.0674, found 309.0667.

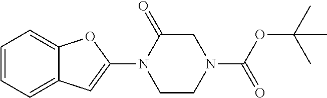 [SMILES: CC(C)(C)OC(=O)N1CCN(c2cc3ccccc3o2)C(=O)C1]

tert-butyl 4-(benzofuran-2-yl)-3-oxopiperazine-1-carboxylate: 2-bromobenzofuran (200 mg, 1.02 mmol) was coupled with tert-butyl 3-oxopiperazine-1-carboxylate (204.24 mg, 1.02 mmol) via general procedure D and purified by silica gel chromatography (0 to 50% EtOAc/hexane) to yield a solid (44.3 mg, 0.14 mmol, 14%). 1H NMR (400 MHz, CDCl3) δ 7.65-7.52 (m, 1H), 7.48-7.39 (m, 1H), 7.26 (dd, J=6.0, 3.3 Hz, 2H), 6.96 (d, J=1.2 Hz, 1H), 4.35 (s, 2H), 4.19-4.05 (m, 2H), 3.86 (d, J=5.6 Hz, 2H), 1.53 (d, J=1.6 Hz, 9H). LC/MS: [M+H]⁺ m/z calc. 316.1, found 316.2.

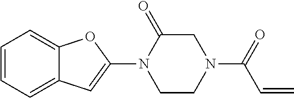 [SMILES: C=CC(=O)N1CCN(c2cc3ccccc3o2)C(=O)C1]

4-acryloyl-1-(benzofuran-2-yl)piperazin-2-one: tert-butyl 4-(benzofuran-2-yl)-3-oxopiperazine-1-carboxylate (EZ-1-044) (44.3 mg, 0.14 mmol) was deprotected and acylated via general procedures F and H and purified by silica gel chromatography (0 to 50% EtOAc/hexane) to afford the title compound as a solid (9.3 mg, 0.034 mmol, 25%). 1H NMR (300 MHz, CDCl3) δ 7.63-7.51 (m, 1H), 7.43 (dt, J=7.1, 3.8 Hz, 1H), 7.29 (td, J=6.3, 2.8 Hz, 2H), 6.98 (d, J=1.0 Hz, 1H), 6.57 (d, J=9.8 Hz, 1H), 6.47 (dd, J=16.7, 2.2 Hz, 1H), 5.88 (dd, J=10.1, 2.2 Hz, 1H), 4.52 (s, 2H), 4.24-3.92 (m, 4H). 13C NMR (151 MHz, DMSO) δ 165.0, 150.1, 149.5, 129.0, 128.8, 128.1, 123.9, 123.9, 121.2, 111.1, 94.6, 49.5, 47.1, 46.6, 42.4. HRMS (ESI): [M+H]⁺ m/z calc. 271.1004, found 271.1078.

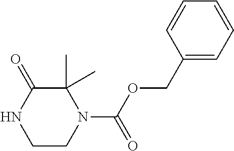 [SMILES: CC1(C)C(=O)NCCN1C(=O)OCc1ccccc1]

benzyl 2,2-dimethyl-3-oxopiperazine-1-carboxylate: 3,3-dimethylpiperazin-2-one (400 mg, 3.12 mmol) was protected with benzyl chloroformate via general procedure E and purified by silica gel chromatography (0 to 10% MeOH/DCM) to yield a powder (492.1 mg, 1.88 mmol, 60%). 1H NMR (300 MHz, CDCl3) δ 7.41 (s, 5H), 6.02 (s, 1H), 5.19 (s, 2H), 3.87-3.74 (m, 2H), 3.49-3.35 (m, 2H), 1.75 (s, 6H). LC/MS: [M+H]⁺ m/z calc. 263.1, found 263.1.

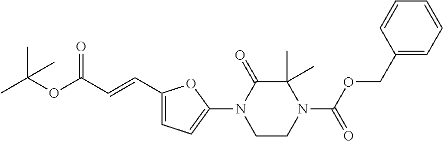 [SMILES: CC(C)(C)OC(=O)/C=C/c1ccc(N2CCN(C(=O)OCc3ccccc3)C(C)(C)C2=O)o1]

benzyl (E)-4-(5-(3-(tert-butoxy)-3-oxoprop-1-en-1-yl)furan-2-yl)-2,2-dimethyl-3-oxopiperazine-1-carboxylate: tert-butyl (E)-3-(5-bromofuran-2-yl)acrylate (Intermediate 2) (104 mg, 0.38 mmol) and benzyl 2,2-dimethyl-3-oxopiperazine-1-carboxylate (EZ-1-050) (100 mg, 0.38 mmol) were coupled via general procedure D and purified by silica gel chromatography (0 to 50% EtOAc/hexane) to yield a an oil that solidified upon standing (133.7 mg, 0.29 mmol, 77%). 1H NMR (400 MHz, CDCl3) δ 7.43 (d, J=5.1 Hz, 6H), 6.66 (q, J=3.6 Hz, 2H), 6.12 (d, J=15.6 Hz, 1H), 5.22 (s, 2H), 4.04-3.98 (m, 2H), 3.91 (d, J=5.2 Hz, 2H), 1.80 (s, 6H), 1.56 (d, J=4.0 Hz, 9H). LC/MS: [M+H]⁺ m/z calc. 455.2, found 455.2

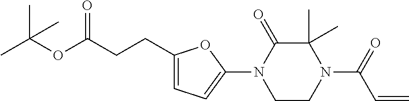 [SMILES: C=CC(=O)N1CCN(c2ccc(CCC(=O)OC(C)(C)C)o2)C(=O)C1(C)C]

tert-butyl 3-(5-(4-acryloyl-3,3-dimethyl-2-oxopiperazin-1-yl)furan-2-yl)propanoate: benzyl (E)-4-(5-(3-(tert-butoxy)-3-oxoprop-1-en-1-yl)furan-2-yl)-2,2-dimethyl-3-oxopiperazine-1-carboxylate (30 mg, 0.066 mmol) was deprotected and acylated via general procedures G and H and purified by silica gel chromatography (0-70% EtOAc/hexane) to afford the title compound as an oil (7.2 mg, 0.019 mmol, 29% over two steps). 1H NMR (400 MHz, CDCl3) δ 6.51 (ddd, J=16.8, 10.6, 2.3 Hz, 1H), 6.29 (t, J=2.9 Hz, 1H), 6.23 (dt, J=16.8, 2.1 Hz, 1H), 6.03 (d, J=3.2 Hz, 1H), 5.70 (dt, J=10.5, 2.1 Hz, 1H), 3.88 (dd, J=6.4, 3.4 Hz, 2H), 3.78 (dd, J=6.1, 3.6 Hz, 2H), 2.87 (t, J=7.6 Hz, 2H), 2.54 (td, J=7.9, 2.3 Hz, 2H), 1.83 (d, J=2.3 Hz, 6H), 1.44 (d, J=2.3 Hz, 9H). 13C NMR (151 MHz, DMSO) δ 171.6, 171.1, 166.3, 149.1, 146.2, 131.5, 127.2, 107.2, 99.7, 80.4, 63.6, 47.5, 42.7, 28.2, 23.8, 23.5. HRMS (ESI): [M+Na]⁺ m/z calc. 399.1896, found 399.1883.

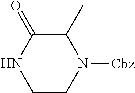 [SMILES: CC1C(=O)NCCN1C(=O)OCc1ccccc1]

Benzyl 2-methyl-3-oxopiperazine-1-carboxylate: 3-methylpiperazin-2-one (400 mg, 3.5 mmol) was protected with benzyl chloroformate via general procedure E and purified by silica gel chromatography (0 to 10% MeOH/DCM) to yield a solid (123.9 mg, 0.5 mmol, 14%). 1H NMR (300 MHz, CDCl3) δ 7.36 (s, 5H), 5.96 (s, 1H), 5.16 (s, 2H), 4.69 (s, 1H), 4.18 (s, 1H), 3.47 (d, J=12.1 Hz, 1H), 3.27 (d, J=12.2 Hz, 2H), 1.46 (d, J=7.1 Hz, 3H). LC/MS: [M+H]⁺ m/z calc. 249.1, found 249.1.

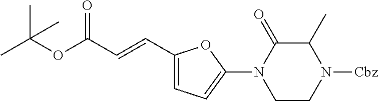 [SMILES: CC1C(=O)N(c2ccc(/C=C/C(=O)OC(C)(C)C)o2)CCN1C(=O)OCc1ccccc1]

benzyl (E)-4-(5-(3-(tert-butoxy)-3-oxoprop-1-en-1-yl)furan-2-yl)-2-methyl-3-oxopiperazine-1-carboxylate: Benzyl 2-methyl-3-oxopiperazine-1-carboxylate (EZ-1-049) (60 mg, 0.24 mmol) and tert-butyl (E)-3-(5-bromofuran-2-yl)acrylate (66 mg, 0.24 mmol) were coupled via general procedure D and purified by silica gel chromatography (0 to 50% EtOAc/hexane) to yield a solid (69.3 mg, 0.16 mmol, 66%). 1H NMR (400 MHz, CDCl3) δ 7.42 (s, 5H), 7.32-7.24 (m, 1H), 6.70-6.62 (m, 2H), 6.12 (d, J=15.4 Hz, 1H), 5.23 (d, J=2.5 Hz, 2H), 4.89 (s, 1H), 4.35 (s, 1H), 4.00 (d, J=13.9 Hz, 2H), 3.50 (s, 1H), 1.72-1.49 (m, 12H). LC/MS: [M+H]⁺ m/z calc. 441.2, found 441.2.

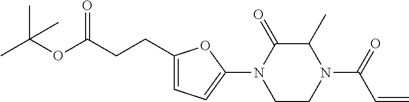 [SMILES: C=CC(=O)N1CCN(c2ccc(CCC(=O)OC(C)(C)C)o2)C(=O)C1C]

tert-butyl 3-(5-(4-acryloyl-3-methyl-2-oxopiperazin-1-yl)furan-2-yl)propanoate: benzyl (E)-4-(5-(3-(tert-butoxy)-3-oxoprop-1-en-1-yl)furan-2-yl)-2-methyl-3-oxopiperazine-1-carboxylate (52.3 mg, 0.12 mmol) was deprotected and acylated via general procedures G and H and purified by silica gel chromatography (0-100% EtOAc/hexane) to yield the title compound as an oil (17.9 mg, 0.05 mmol, 42% over two steps). 1H NMR (400 MHz, CDCl3) δ 6.66-6.51 (m, 1H), 6.46 (d, J=16.7 Hz, 1H), 6.32 (d, J=3.2 Hz, 1H), 6.07 (dd, J=3.2, 1.0 Hz, 1H), 5.84 (d, J=9.8 Hz, 1H), 4.74 (s, 1H), 4.23-3.23 (m, 4H), 2.91 (t, J=7.6 Hz, 2H), 2.57 (dd, J=8.2, 6.9 Hz, 3H), 1.63 (s, 3H), 1.47 (s, 9H). 13C NMR (151 MHz, DMSO) δ 171.6, 167.6, 164.2, 149.5, 145.9, 128.7, 128.2, 107.2, 100.7, 80.4, 60.2, 54.5, 52.0, 48.2, 33.4, 28.2, 23.5, 17.0.

HRMS (ESI): [M+Na]⁺ m/z calc. 385.1739, found 385.1728.

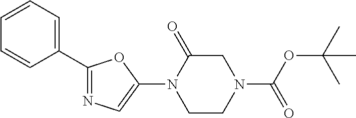 [SMILES: CC(C)(C)OC(=O)N1CCN(c2cnc(-c3ccccc3)o2)C(=O)C1]

tert-butyl 3-oxo-4-(2-phenyloxazol-5-yl)piperazine-1-carboxylate: 5-bromo-2-phenyloxazole (50 mg, 0.22 mmol) was coupled with tert-butyl 3-oxopiperazine-1-carboxylate (44.7 mg, 0.22 mmol) via general procedure D and purified by silica gel chromatography (0 to 60% EtOAc/hexane) to yield a solid (40.4 mg, 0.117 mmol, 54%). 1H NMR (400 MHz, CDCl3) δ 8.05-7.98 (m, 2H), 7.49 (dd, J=5.7, 1.8 Hz, 3H), 7.38 (s, 1H), 4.36 (s, 2H), 4.04 (t, J=5.4 Hz, 2H), 3.89 (t, J=5.3 Hz, 2H), 1.55 (s, 9H). LC/MS: [M+H]⁺ m/z calc. 344.2, found 344.1.

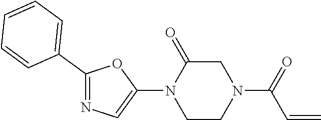 [SMILES: C=CC(=O)N1CCN(c2cnc(-c3ccccc3)o2)C(=O)C1]

4-acryloyl-1-(2-phenyloxazol-5-yl)piperazin-2-one: tert-butyl 3-oxo-4-(2-phenyloxazol-5-yl)piperazine-1-carboxylate (40.4 mg, 0.117 mmol) was deprotected and acylated via general procedures F and H and purified by silica gel chromatography (0 to 80% EtOAc/hexane) to afford the title compound as a solid (34.6 mg, 0.116 mmol, 45% over two steps)₁H NMR (300 MHz, CDCl₃) δ 8.01 (dd, J=6.8, 3.0 Hz, 2H), 7.54-7.46 (m, 3H), 7.39 (s, 1H), 6.59 (s, 1H), 6.54-6.42 (m, 1H), 5.90 (d, J=11.6 Hz, 1H), 4.53 (s, 2H), 4.10 (s, 4H). 13C NMR (151 MHz, DMSO) δ 164.6, 155.1, 146.6, 130.8, 129.6, 128.9, 128.3, 128.1, 127.1, 125.9, 116.2, 49.4, 47.2, 46.9. HRMS (ESI): [M+H]⁺ m/z calc. 298.1113, found 298.1187.

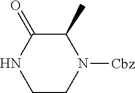 [SMILES: C[C@@H]1C(=O)NCCN1C(=O)OCc1ccccc1]

phenyl (R)-2-methyl-3-oxopiperazine-1-carboxylate: (R)-3-methylpiperazin-2-one (100 mg, 0.88 mmol) was protected with benzyl chloroformate (186 mL, 0.876 mmol) via general procedure E and purified by silica gel chromatography (0 to 100% EtOAc/hexane) to yield a solid (47.2 mg, 0.25 mmol, 22%). 1H NMR (400 MHz, CDCl3) δ 6.15 (s, 1H), 5.21 (s, 2H), 4.73 (s, 1H), 4.24 (s, 1H), 3.51 (d, J=12.5 Hz, 1H), 3.31 (d, J=12.6 Hz, 2H), 1.50 (d, J=7.0 Hz, 3H).LC/MS: [M+H]⁺ m/z calc. 248.1, found 248.1

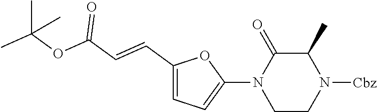 [SMILES: C[C@@H]1C(=O)N(c2ccc(/C=C/C(=O)OC(C)(C)C)o2)CCN1C(=O)OCc1ccccc1]

benzyl (R,E)-4-(5-(3-(tert-butoxy)-3-oxoprop-1-en-1-yl)furan-2-yl)-2-methyl-3-oxopiperazine-1-carboxylate: phenyl (R)-2-methyl-3-oxopiperazine-1-carboxylate (44.6 mg, 0.18 mmol) was coupled to tert-butyl (E)-3-(5-bromofuran-2-yl)acrylate (49.1 mg, 0.18 mmol) via general procedure D and purified by silica gel chromatography (0 to 35% EtOAc/hexane) to yield an oil (56.7 mg, 0.13 mmol, 72%). 1H NMR (400 MHz, CDCl3) δ 7.42 (d, J=5.3 Hz, 5H), 7.30 (s, 1H), 6.74-6.62 (m, 2H), 6.12 (d, J=15.6 Hz, 1H), 5.23 (d, J=2.3 Hz, 2H), 4.89 (s, 1H), 4.34 (s, 1H), 4.02 (s, 2H), 3.49 (s, 1H), 1.61 (s, 3H), 1.56 (d, J=5.5 Hz, 9H). LC/MS: [M+H]⁺ m/z calc. 441.2, found 441.2.

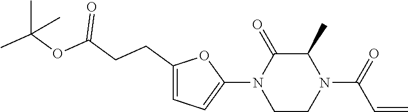 [SMILES: C=CC(=O)N1CCN(c2ccc(CCC(=O)OC(C)(C)C)o2)C(=O)[C@H]1C]

tert-butyl (R)-3-(5-(4-acryloyl-3-methyl-2-oxopiperazin-1-yl)furan-2-yl)propanoate: benzyl (R,E)-4-(5-(3-(tert-butoxy)-3-oxoprop-1-en-1-yl)furan-2-yl)-2-methyl-3-oxopiperazine-1-carboxylate (31.2 mg, 0.07 mmol) was deprotected and acylated via general procedures F and H and purified by silica gel chromatography (0 to 100% EtOAc/hexane) to afford the title compound as a solid (18.9 mg, 0.052 mmol, 68% over two steps). 1H NMR (300 MHz, CDCl3) δ 6.65-6.40 (m, 2H), 6.33 (d, J=3.4 Hz, 1H), 6.08 (d, J=3.3 Hz, 1H), 5.90-5.81 (m, 1H), 4.76 (s, 1H), 3.93-3.34 (m, 4H), 2.92 (t, J=7.5 Hz, 2H), 2.58 (dd, J=8.3, 6.8 Hz, 2H), 2.22 (s, 3H), 1.48 (s, 9H). 13C NMR (151 MHz, DMSO) δ 171.6, 167.6, 164.2, 149.4, 145.9, 128.7, 128.2, 107.2, 100.6, 80.4, 52.0, 48.2, 47.2, 33.4, 28.2, 23.5. HRMS (ESI): [M+Na]⁺ m/z calc. 385.1739, found 385.1730.

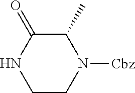 [SMILES: C[C@H]1C(=O)NCCN1C(=O)OCc1ccccc1]

benzyl (S)-2-methyl-3-oxopiperazine-1-carboxylate: (S)-3-methylpiperazin-2-one (100 mg, 0.88 mmol) was protected with benzyl chloroformate (149.4 mg, 0.88 mmol) via general procedure E and purified by silica gel chromatography (0 to 100% EtOAc/hexane) to yield a white solid (89.4 mg, 0.36 mmol, 41%). 1H NMR (400 MHz, CDCl3) δ 7.40 (d, J=4.6 Hz, 5H), 6.13 (s, 1H), 5.21 (s, 2H), 4.72 (s, 1H), 4.24 (s, 1H), 3.53 (s, 1H), 3.31 (d, J=12.5 Hz, 2H).LC/MS: [M+H]⁺ m/z calc. 248.1, found 248.1.

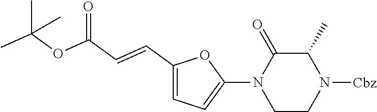 [SMILES: C[C@H]1C(=O)N(c2ccc(/C=C/C(=O)OC(C)(C)C)o2)CCN1C(=O)OCc1ccccc1]

benzyl (S,E)-4-(5-(3-(tert-butoxy)-3-oxoprop-1-en-1-yl)furan-2-yl)-2-methyl-3-oxopiperazine-1-carboxylate: benzyl (S)-2-methyl-3-oxopiperazine-1-carboxylate (EZ-1-063) (41.6 mg, 0.17 mmol) was coupled to tert-butyl (E)-3-(5-bromofuran-2-yl)acrylate (EZ-1-048) (46.8 mg, 0.17 mmol) via general procedure D and purified by silica gel chromatography (0 to 50% EtOAc/hexane) to yield a clear yellow oil (41.3 mg, 0.09 mmol, 56%). 1H NMR (300 MHz, CDCl3) δ 7.45-7.37 (m, 5H), 7.31 (d, J=1.3 Hz, 1H), 6.72-6.61 (m, 2H), 6.12 (d, J=15.6 Hz, 1H), 5.23 (d, J=1.3 Hz, 2H), 4.90 (s, 1H), 4.34 (s, 1H), 4.05-3.92 (m, 2H), 3.49 (s, 1H), 1.62 (s, 3H), 1.56 (d, J=3.1 Hz, 9H).

LC/MS: [M+H]⁺ m/z calc. 441.2, found 441.2.

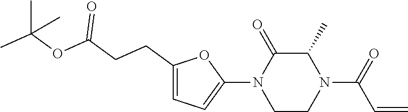 [SMILES: C=CC(=O)N1CCN(c2ccc(CCC(=O)OC(C)(C)C)o2)C(=O)[C@@H]1C]

tert-butyl (S)-3-(5-(4-acryloyl-3-methyl-2-oxopiperazin-1-yl)furan-2-yl)propanoate: benzyl (S,E)-4-(5-(3-(tert-butoxy)-3-oxoprop-1-en-1-yl)furan-2-yl)-2-methyl-3-oxopiperazine-1-carboxylate (35.4 mg, 0.08 mmol) was deprotected and acylated via general procedures F and H and purified by silica gel chromatography (0 to 100% EtOAc/hexane) to afford the title compound as a clear colorless oil (16.9 mg, 0.047 mmol, 58% over two steps). 1H NMR (400 MHz, CDCl3) δ 6.63-6.41 (m, 2H), 6.32 (d, J=3.4 Hz, 1H), 6.07 (d, J=3.5 Hz, 1H), 5.85 (d, J=10.3 Hz, 1H), 4.77 (s, 2H), 3.88 (s, 2H), 3.34 (s, 1H), 2.91 (t, J=7.5 Hz, 2H), 2.58 (dt, J=8.8, 5.2 Hz, 2H), 1.74 (s, 3H), 1.48 (d, J=4.0 Hz, 9H).

13C NMR (151 MHz, DMSO) δ 171.6, 164.2, 149.5, 145.9, 128.7, 128.1, 107.2, 100.7, 80.4, 54.4, 52.0, 48.2, 33.4, 28.2, 23.5.

HRMS (ESI): [M+Na]⁺ m/z calc. 385.1739, found 385.1726.

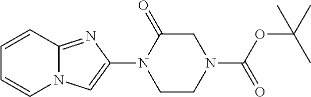 [SMILES: CC(C)(C)OC(=O)N1CCN(c2cn3ccccc3n2)C(=O)C1]

tert-butyl 4-(imidazo[1,2-a]pyridin-2-yl)-3-oxopiperazine-1-carboxylate: 2-bromoimidazo[1,2-a]pyridine (50 mg, 0.25 mmol) was coupled to tert-butyl 3-oxopiperazine-1-carboxylate (50.8 mg, 0.25 mmol) via general procedure D and purified by silica gel chromatography (0 to 80% EtOAc/hexane) to yield a clear colorless oil (35.7 mg, 0.11 mmol, 45%). 1H NMR (400 MHz, CDCl3) δ 8.33 (s, 1H), 8.15 (d, J=6.7 Hz, 1H), 7.54 (d, J=9.1 Hz, 1H), 7.22 (ddd, J=8.7, 6.9, 1.4 Hz, 1H), 6.85 (td, J=6.8, 1.3 Hz, 1H), 4.34 (s, 2H), 4.31 (t, J=5.5 Hz, 2H), 3.83 (t, J=5.4 Hz, 2H), 1.53 (s, 9H).LC/MS: [M+H]⁺ m/z calc. 317.2, found 317.2.

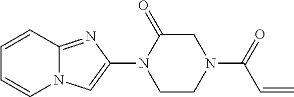 [SMILES: C=CC(=O)N1CCN(c2cn3ccccc3n2)C(=O)C1]

4-acryloyl-1-(imidazo[1,2-a]pyridin-2-yl)piperazin-2-one: tert-butyl 4-(imidazo[1,2-a]pyridin-2-yl)-3-oxopiperazine-1-carboxylate (23.4 mg, 0.074 mmol) was deprotected and acylated via general procedures F and H and purified by silica gel chromatography (0 to 100% EtOAc/hexane) to afford the title compound as an off white solid (3.8 mg, 0.014 mmol, 19% over two steps). 1H NMR (400 MHz, CDCl3) δ 8.32 (s, 1H), 8.15 (d, J=6.9 Hz, 1H), 7.55 (d, J=9.0 Hz, 1H), 7.24 (t, J=7.9 Hz, 1H), 6.87 (t, J=6.8 Hz, 1H), 6.62 (s, 1H), 6.46 (d, J=16.7 Hz, 1H), 5.86 (d, J=10.5 Hz, 1H), 4.53 (d, J=23.7 Hz, 2H), 4.38 (s, 2H), 4.04 (d, J=33.0 Hz, 2H).

HRMS (ESI): [M+H]⁺ m/z calc. 271.1117, found 271.1190.

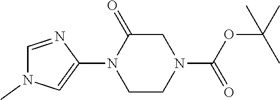 [SMILES: Cn1cnc(N2CCN(C(=O)OC(C)(C)C)CC2=O)c1]

tert-butyl 4-(1-methyl-1H-imidazol-4-yl)-3-oxopiperazine-1-carboxylate: 4-bromo-1-methyl-1H-imidazole (155 mL, 1.55 mmol) was coupled to tert-butyl 3-oxopiperazine-1-carboxylate (311 mg, 1.55 mmol) via general procedure D and the crude residue was purified by silica gel chromatography (0-100% EtOAc/Hex) to yield a solid (412 mg, 1.47 mmol, 95%). 1H NMR (400 MHz, CDCl3) δ 7.58-7.50 (m, 1H), 7.39-7.26 (m, 1H), 4.27 (d, J=9.5 Hz, 2H), 4.18-4.06 (m, 3H), 3.80-3.61 (m, 4H), 1.51 (d, J=4.1 Hz, 9H).

LC/MS: [M+H]⁺ m/z calc. 281.2, found 281.2.

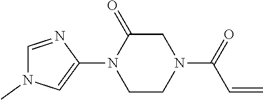 [SMILES: C=CC(=O)N1CCN(c2cn(C)cn2)C(=O)C1]

4-acryloyl-1-(1-methyl-1H-imidazol-4-yl)piperazin-2-one: tert-butyl 4-(1-methyl-1H-imidazol-4-yl)-3-oxopiperazine-1-carboxylate (100 mg, 0.36 mmol) was deprotected and acylated via general procedures F and H and the crude residue was purified by silica gel chromatography (0 to 10% MeOH/DCM) to afford the title compound as a solid (27.7 mg, 0.12 mmol, 33%). 1H NMR (300 MHz, CDCl3) δ 7.54 (s, 1H), 7.27 (s, 1H), 6.58 (s, 1H), 6.44 (dd, J=16.7, 2.0 Hz, 1H), 5.88-5.81 (m, 1H), 4.46 (d, J=16.0 Hz, 2H), 4.18 (s, 2H), 3.99 (d, J=23.0 Hz, 2H), 3.73 (s, 3H). 13C NMR (151 MHz, DMSO) δ 163.4, 162.9, 138.9, 133.9, 128.6, 128.5, 128.2, 46.9, 44.9, 42.6, 33.7. HRMS (ESI): [M+H]⁺ m/z calc. 235.1117, found 235.1190.

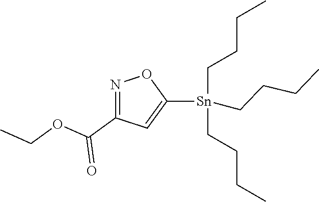 [SMILES: CCC[CH2][Sn]([CH2]CCC)([CH2]CCC)[c]1cc(C(=O)OCC)no1]

ethyl 5-(tributylstannyl)isoxazole-3-carboxylate: To a solution of ethyl-2-chloro-2(hydroxyiminoacetate) (481 mg, 3.17 mmol) dissolved in anhydrous DCM (15 mL), potassium carbonate (482.5 mg, 3.5 mmol) and tributyl(ethynyl)stannane (872 mL, 3.17 mmol) were added and stirred at room temperature overnight. The reaction was then quenched with water, extracted with DCM and dried over anhydrous sodium sulfate. The organic layer was purified via silica gel column chromatography (0 to 10% EtOAc/hexane) to give the product as an oil (753 mg, 1.75 mmol, 55%). 1H NMR (400 MHz, CDCl3) δ 6.84 (s, 1H), 4.48 (q, J=7.1 Hz, 2H), 1.70-1.10 (m, 27H), 0.94 (s, 3H).

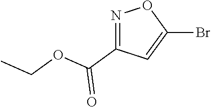

Ethyl 5-bromoisoxazole-3-carboxylate Br₂ (134 mL, 2.62 mmol) was added to a solution of ethyl 5-(tributylstannyl)isoxazole-3-carboxylate (753 mg, 1.74 mmol) and sodium carbonate (203 mg, 1.91 mmol) dissolved in DCM (10 mL), and stirred at room temperature overnight. The reaction mixture was then quenched with saturated sodium thiosulfate (8 mL) before extracting with DCM and washing with brine. The organic layer was dried over anhydrous sodium sulfate and purified via silica gel column chromatography (0 to 15% EtOAc/hexane) to produce a clear colorless oil (241.8 mg, 1.1 mmol, 63%) that crystallized upon standing. 1H NMR (400 MHz, CDCl3) δ 6.76 (s, 1H), 4.49 (q, J=7.1 Hz, 2H), 1.47 (dt, J=9.6, 6.9 Hz, 3H).

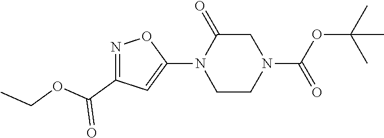 [SMILES: CCOC(=O)c1cc(N2CCN(C(=O)OC(C)(C)C)CC2=O)on1]

Ethyl 5-(4-(tert-butoxycarbonyl)-2-oxopiperazin-1-yl)isoxazole-3-carboxylate Anhydrous dioxane (3 mL) was added to a vial flushed with N₂ containing ethyl 5-bromoisoxazole-3-carboxylate (EZ-1-091) (94.6 mg, 0.43 mmol), tert-butyl 3-oxopiperazine-1-carboxylate (0.43 mmol, 86.1 mg), cesium carbonate (280.2 mg, 0.86 mmol), Xantphos (19 mg, 0.032 mmol), Pd(dba)₃ (10 mg, 0.011 mmol) and the suspension was degassed. The reaction mixture was stirred at 90° C. overnight. The product was extracted with EtOAc, washed with brine, and purified via silica gel column chromatography (0 to 75% EtOAc/hexane) to afford a clear yellow oil (14 mg, 0.04 mmol, 9.6%). 1H NMR (400 MHz, CDCl3) δ 4.48 (q, J=7.1 Hz, 2H), 4.38 (s, 2H), 4.13 (q, J=5.5 Hz, 2H), 3.91-3.84 (m, 2H), 1.54 (d, J=2.8 Hz, 9H), 1.46 (t, J=7.1 Hz, 3H).

LC/MS: [M+H]⁺ m/z calc. 340.1, found 340.

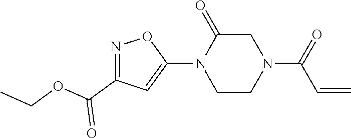 [SMILES: C=CC(=O)N1CCN(c2cc(C(=O)OCC)no2)C(=O)C1]

Ethyl 5-(4-acryloyl-2-oxopiperazin-1-yl)isoxazole-3-carboxylate: Ethyl 5-(4-(tert-butoxycarbonyl)-2-oxopiperazin-1-yl)isoxazole-3-carboxylate (EZ-1-097) (14 mg, 0.04 mmol) was deprotected and acylated via general procedures F and H respectively and the crude residue was purified by silica gel chromatography (0 to 100% EtOAc/hexane) to afford the title compound as a clear colorless oil (5.0 mg, 0.017 mmol, 42%). 1H NMR (400 MHz, CDCl3) δ 7.01 (s, 1H), 6.57 (s, 1H), 6.47 (dd, J=16.8, 2.0 Hz, 1H), 5.90 (dd, J=10.1, 2.0 Hz, 1H), 4.56 (s, 2H), 4.48 (q, J=7.1 Hz, 2H), 4.18 (d, J=5.3 Hz, 2H), 4.07 (s, 2H), 1.46 (t, J=7.1 Hz, 3H). 13C NMR (151 MHz, DMSO) δ 173.4, 144.1, 143.7, 135.1, 121.6, 119.5, 118.2, 117.4, 64.5, 44.7, 27.3, 16.5, 9.9. HRMS (ESI): [M+Na]⁺ m/z calc. 316.0909, found 316.0907.

### Example 5: Bio-NMR Analysis of DUB Recruiter-Deubiquitinase Interactions

All NMR spectra was recorded on a Bruker 600 MHz spectrometer, equipped with a 5 mm QCI-F cryo probe with z-gradient, and the temperature was kept constant at 298K during all experiments. To probe compound and E2 ligase binding to OTUB1, ¹H-1D and ¹³C-SOFAST-HMQC experiments were carried out using 3 mm NMR tubes filled with 160 μL of 50 μM {U}-²H,¹H/¹³C-methyl-Ile/Leu/Val/Ala(ILVA),{U}-¹⁵N labeled OTUB1, 25 mM d-Tris, pH 7.5, 150 mM NaCl, 5% D₂O (to lock), 100 μM DSS (internal standard), 75 μM DUB Recruiter (Compound 100) (dissolved in 100% d₆-DMSO; for compound binding study) and/or 100 μM E2 D2/Ub-E2 D2 (for ligase binding studies). To allow for complete binding of the compound to OTUB1, an incubation period of ˜40 hours was selected. Reference spectra with the adequate volumes of pure d₆-DMSO and/or E2 buffer were recorded to compensate for solvent induced effects, and experiments were repeated after 40 hours to make sure that any spectral changes were not related to protein oxidation.

### Example 6: Native Mass Spectrometry Analysis of Ternary Complex Formation

Native mass spectrometry experiments were performed on a Thermo QE UHMR equipped with a nano-electrospray ionization source (Advion TriVersa NanoMate). Recombinant OTUB1 was first buffer exchanged into 150 mM ammonium acetate, 100 μM MgCl₂, and 100 μM ATP at pH 6.7. 4 μM OTUB1 was then pre-incubated at room temperature for 24 hours with either DMSO, DUB Recruiter Compound 100 (100 μM), or DUBTAc Compound 200 (100 μM). After 24 hours, 4 μM CFTR, in the same buffer, was added to the OTUB1 solution, for final concentrations of 2 μM of each protein with either DMSO or 50 μM compound. The solution was then allowed to incubate for 30 minutes prior to analysis on the mass spectrometer. Mass spectra were recorded in positive ion mode with a mass range of 1000-8000 m/z. Each spectrum was then deconvoluted and relevant peaks were integrated to determine % ternary complex formed. All experiments were performed in triplicate.

### Example 7: Transepithelial Conductance Assays in Human Bronchial Epithelial Cells

Human bronchial epithelial cells (HBECs) from cystic fibrosis (CF) patients bearing the DF508-CFTR mutation were cultured at 37° C. and 5% CO₂ in Bronchial Epithelial Cell Growth Basal Medium (BEGM) with SingleQuots Supplements and Growth Factors (Lonza, #CC-3170). Cells were maintained in cell culture flasks (Corning, #430641U) for one week and media was replaced every two to three days. Cells were washed with Dulbecco's phosphate buffered saline (Thermo Fisher Scientific, #14040141), trypsinized for five to ten minutes with 0.05% Trypsin-EDTA (Thermo Fisher Scientific, #25300120), after which Trypsin Neutralizing Solution (TNS, Thermo Fisher Scientific, #R002100) was added. Cells were pelleted at 300× g for five minutes and resuspended in BEGM with Dulbecco's modified Eagle medium (DMEM, Thermo Fisher Scientific, #11965092) and plated at one million cells per plate in 24-well transwell plates (Corning, #3526). Cells were grown submerged in BEGM with DMEM for one week with media changed every two to three days, at which time they were taken to air liquid interface (ALI) and grown another two weeks before being ready to use.

Cells were treated with either DMSO vehicle, 10 μM lumacaftor or 10 μM DUBTAC 24 hours before the experiment. Cells were then submerged in Ham's F12 buffer (Thermo Fisher Scientific, #21700075) with 20 mM HEPES (Thermo Fisher Scientific, #15630080) at pH 7.4 and mounted into the assay system. Transepithelial resistance was recorded using a 24-channel transepithelial current clamp amplifier (TECC-24, EP Design, Bertem, Belgium). Resistance measurements were taken at intervals of approximately six minutes. Four values were taken to determine baseline resistance, and another four measurements were taken after each of the following additions: 10 μM Amiloride (Millipore Sigma, #A7410) added apically, 20 μM Forskolin (Millipore Sigma, #F6886) added apically, and 0.5 μM ivacaftor added both apically and basolaterally. CFTR Inhibitor 172 (Millipore Sigma, #219672) was then added and a final six measurements taken. Transepithelial conductance (G) was calculated from resistance measurements (G=1/R). Chloride ion transport across the epithelial monolayer is mediated by CFTR, and activation or inhibition of functional CFTR therefore causes changes in transepithelial conductance. In this way, AG can be used to measure functional CFTR expression and the functional rescue of CFTR through compound addition.

1. A bifunctional compound of Formula (I): 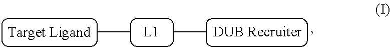
or a pharmaceutically acceptable salt, hydrate, solvate, stereoisomer, or tautomer thereof, wherein:
(i) the Target Ligand comprises a moiety capable of binding to a target protein;
(ii) L1 comprises a linker; and
(iii) the DUB Recruiter comprises a moiety capable of binding to a deubiquitinase.

2. The bifunctional compound or pharmaceutically acceptable salt hydrate, solvate, stereoisomer, or tautomer thereof according to claim 1, wherein the target protein is selected from the group consisting of enzyme, receptor, membrane channel, and a hormone, or a fragment thereof.

3. The bifunctional compound or pharmaceutically acceptable salt hydrate, solvate, stereoisomer, or tautomer thereof according to any one of the preceding claims, wherein the target protein is a soluble protein or a membrane protein.

4. The bifunctional compound or pharmaceutically acceptable salt hydrate, solvate, stereoisomer, or tautomer thereof according to any one of the preceding claims, wherein the target protein is mutated or misfolded.

5. The bifunctional compound or pharmaceutically acceptable salt hydrate, solvate, stereoisomer, or tautomer thereof according to any one of the preceding claims, wherein the target protein is glycosylated.

6. The bifunctional compound or pharmaceutically acceptable salt hydrate, solvate, stereoisomer, or tautomer thereof according to any one of the preceding claims, wherein the target protein is ubiquitinated (e.g., polyubiquitinated).

7. The bifunctional compound or pharmaceutically acceptable salt hydrate, solvate, stereoisomer, or tautomer thereof according to any one of the preceding claims, wherein the target protein is a tumor suppressor.

8. The bifunctional compound or pharmaceutically acceptable salt hydrate, solvate, stereoisomer, or tautomer thereof according to any one of the preceding claims, wherein the target protein is selected from the group consisting of a tumor suppressor, a membrane channel, a kinase, a transcription factor, an ion channel, an apoptotic factor, an oncogenic protein, and an epigenetic regulator.

9. The bifunctional compound or pharmaceutically acceptable salt hydrate, solvate, stereoisomer, or tautomer thereof according to any one of the preceding claims, wherein the target protein is selected from the group consisting of TP53, CDKN1A, CDN1C, BAX, glucokinase, the cystic fibrosis transmembrane conductance regulator (CFTR), WEE1, or a mutant or fragment thereof.

10. The bifunctional compound or pharmaceutically acceptable salt hydrate, solvate, stereoisomer, or tautomer thereof according to any one of the preceding claims, wherein the target protein comprises the cystic fibrosis transmembrane conductance regulator (CFTR) or a mutant or fragment thereof.

11. The bifunctional compound or pharmaceutically acceptable salt hydrate, solvate, stereoisomer, or tautomer thereof according to any one of the preceding claims, wherein the target protein comprises ΔF508-CFTR.

12. The bifunctional compound or pharmaceutically acceptable salt hydrate, solvate, stereoisomer, or tautomer thereof according to any one of claims 1-10 wherein the target protein comprises the tumor suppressor kinase WEE1 or a mutant or fragment thereof.

13. The bifunctional compound or pharmaceutically acceptable salt hydrate, solvate, stereoisomer, or tautomer thereof according to any one of the preceding claims, wherein the deubiquitinase is capable of cleaving a lysine-linked polyubiquitin chain.

14. The bifunctional compound or pharmaceutically acceptable salt hydrate, solvate, stereoisomer, or tautomer thereof according to claim 12, wherein the lysine-linked polyubiquitin chain comprises a K43-linked polyubiquitin chain.

15. The bifunctional compound or pharmaceutically acceptable salt hydrate, solvate, stereoisomer, or tautomer thereof according to any one of the preceding claims, wherein the deubiquitinase is a cysteine protease or a metalloprotease.

16. The bifunctional compound or pharmaceutically acceptable salt hydrate, solvate, stereoisomer, or tautomer thereof according to any one of the preceding claims, wherein the deubiquitinase is a cysteine protease.

17. The bifunctional compound or pharmaceutically acceptable salt hydrate, solvate, stereoisomer, or tautomer thereof according to any one of the preceding claims, wherein the bifunctional compound binds to a site other than a catalytic site within the deubiquitinase.

18. The bifunctional compound or pharmaceutically acceptable salt hydrate, solvate, stereoisomer, or tautomer thereof according to any one of the preceding claims, wherein the bifunctional compound binds to an allosteric site within the deubiquitinase.

19. The bifunctional compound or pharmaceutically acceptable salt hydrate, solvate, stereoisomer, or tautomer thereof according to any one of the preceding claims, wherein the bifunctional compound binds to a cysteine amino acid residue within the deubiquitinase.

20. The bifunctional compound or pharmaceutically acceptable salt hydrate, solvate, stereoisomer, or tautomer thereof according to claim 19, wherein the cysteine amino acid residue is an allosteric cysteine amino acid residue.

21. The bifunctional compound or pharmaceutically acceptable salt hydrate, solvate, stereoisomer, or tautomer thereof according to any one of the preceding claims, wherein the bifunctional compound preferentially binds to an allosteric amino acid residue (e.g., an allosteric amino acid residue) over a catalytic amino acid residue (e.g., a catalytic cysteine amino acid residue).

22. The bifunctional compound or pharmaceutically acceptable salt hydrate, solvate, stereoisomer, or tautomer thereof according to any one of the preceding claims, wherein the bifunctional compound does not substantially bind to a cysteine amino acid residue in the catalytic site of the deubiquitinase (e.g., a catalytic cysteine).

23. The bifunctional compound or pharmaceutically acceptable salt hydrate, solvate, stereoisomer, or tautomer thereof according to according to any one of the preceding claims, wherein the deubiquitinase is selected from Table 1.

24. The bifunctional compound or pharmaceutically acceptable salt hydrate, solvate, stereoisomer, or tautomer thereof according to according to any one of the preceding claims, wherein the deubiquitinase is selected from WDR48, YOD1, OYUD3, OTUB1, OTUD5, USP8, USP5, USP15, USP16, UCHL3, UCHL1, and USP14.

25. The bifunctional compound or pharmaceutically acceptable salt hydrate, solvate, stereoisomer, or tautomer thereof according to according to any one of the preceding claims, wherein the deubiquitinase comprises OTUB1.

26. The bifunctional compound or pharmaceutically acceptable salt hydrate, solvate, stereoisomer, or tautomer thereof according to claim 24, wherein the bifunctional compound binds to cysteine 23 (C₂₃) within the OTUB1 sequence.

27. The bifunctional compound or pharmaceutically acceptable salt hydrate, solvate, stereoisomer, or tautomer thereof according to any one of claims 25-26, wherein the bifunctional compound binds preferentially to cysteine 23 (C23) over cysteine 91 (C91) within the OTUB1 sequence.

28. The bifunctional compound or pharmaceutically acceptable salt hydrate, solvate, stereoisomer, or tautomer thereof according to any one of claims 25-27, wherein the bifunctional compound does not substantially bind to cysteine 91 (C91) within the OTUB1 sequence.

29. The bifunctional compound or pharmaceutically acceptable salt hydrate, solvate, stereoisomer, or tautomer thereof according to any one of claims 1-24, wherein the deubiquitinase comprises OTUD5.

30. The bifunctional compound or pharmaceutically acceptable salt hydrate, solvate, stereoisomer, or tautomer thereof according to claim 29, wherein the bifunctional compound binds to cysteine 434 (C434) within the OTUD5 sequence.

31. The bifunctional compound or pharmaceutically acceptable salt hydrate, solvate, stereoisomer, or tautomer thereof according to any one of claims 28-30, wherein the bifunctional compound binds preferentially to cysteine 434 (C434) over cysteine 244 (C244) within the OTUD5 sequence.

32. The bifunctional compound or pharmaceutically acceptable salt hydrate, solvate, stereoisomer, or tautomer thereof according to any one of claims 28-31, wherein the bifunctional compound does not substantially bind to cysteine 244 (C244) within the OTUD5 sequence.

33. The bifunctional compound or pharmaceutically acceptable salt hydrate, solvate, stereoisomer, or tautomer thereof according to any one of claims 1-24, wherein the deubiquitinase comprises USP15.

34. The bifunctional compound or pharmaceutically acceptable salt hydrate, solvate, stereoisomer, or tautomer thereof according to claim 33, wherein the bifunctional compound binds to cysteine 264 (C264) within the USP15 sequence.

35. The bifunctional compound or pharmaceutically acceptable salt hydrate, solvate, stereoisomer, or tautomer thereof according to any one of claims 33-34, wherein the bifunctional compound binds preferentially to cysteine 264 (C264) over cysteine 298 (C298) within the USP15 sequence.

36. The bifunctional compound or pharmaceutically acceptable salt hydrate, solvate, stereoisomer, or tautomer thereof according to any one of claims 33-35, wherein the bifunctional compound does not substantially bind to cysteine 298 (C298) within the USP15 sequence.

37. The bifunctional compound or pharmaceutically acceptable salt hydrate, solvate, stereoisomer, or tautomer thereof according to any one of the preceding claims, wherein the Target Ligand binds to (e.g., covalently binds to) the target protein.

38. The bifunctional compound or pharmaceutically acceptable salt hydrate, solvate, stereoisomer, or tautomer thereof according to any one of the preceding claims, wherein the Target Ligand is capable of modulating the target protein.

39. The bifunctional compound or pharmaceutically acceptable salt hydrate, solvate, stereoisomer, or tautomer thereof according to claim 38, wherein the modulating comprises one or more of:
(i) modulating the folding of the target protein;
(ii) modulating the half-life of the target protein;
(iii) modulating trafficking of the target protein to the proteasome;
(iv) modulating the level of ubiquitination of the target protein;
(v) modulating degradation (e.g., proteasomal degradation) of the target protein;
(vi) modulating target protein signaling;
(vii) modulating target protein localization;
(viii) modulating trafficking of the target protein to the lysozome; and
(ix) modulating target protein interactions with other proteins.

40. The bifunctional compound or pharmaceutically acceptable salt hydrate, solvate, stereoisomer, or tautomer thereof according to claim 39, comprising (i).

41. The bifunctional compound or pharmaceutically acceptable salt hydrate, solvate, stereoisomer, or tautomer thereof according to claim 39, comprising (ii).

42. The bifunctional compound or pharmaceutically acceptable salt hydrate, solvate, stereoisomer, or tautomer thereof according to claim 39, comprising (iii).

43. The bifunctional compound or pharmaceutically acceptable salt hydrate, solvate, stereoisomer, or tautomer thereof according to claim 39, comprising (iv).

44. The bifunctional compound or pharmaceutically acceptable salt hydrate, solvate, stereoisomer, or tautomer thereof according to claim 39, comprising (v).

45. The bifunctional compound or pharmaceutically acceptable salt hydrate, solvate, stereoisomer, or tautomer thereof according to claim 39, comprising each of (i)-(v).

46. The bifunctional compound or pharmaceutically acceptable salt hydrate, solvate, stereoisomer, or tautomer thereof according to any one of the preceding claims, wherein the Target Ligand is a chemical chaperone.

47. The bifunctional compound or pharmaceutically acceptable salt hydrate, solvate, stereoisomer, or tautomer thereof according to any one of the preceding claims, wherein the Target Ligand has the structure of Formula (I-a): 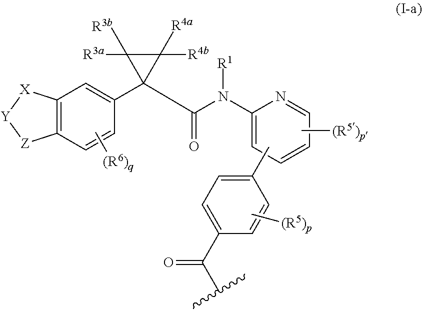
or a pharmaceutically acceptable salt, hydrate, solvate, prodrug, stereoisomer, or tautomer thereof, wherein:
X and Z are each independently O, S, or C(R^{7a})(R^{7b});
Y is C(R^{7a})(R^{7b}) or NR⁷;
R¹ is H or C₁₋₆ alkyl;
R^{3a}, R^{3b}, R^{4a}, R^{4b} are each independently H, C₁₋₆ alkyl, C₁₋₆ haloalkyl, C₁₋₆ heteroalkyl, halo, cyano, or —OR^{A};
each R⁵, R^{5′}, and R⁶ is independently C₁₋₆ alkyl, C₁₋₆ haloalkyl, C₁₋₆ heteroalkyl, halo, cyano, —OR^{A}, —C(O)N(R^{B})(R^{C}), or —N(R^{B})CO(R^{D});
R^{7a} and R^{7b} are each independently H, C₁₋₆ alkyl, C₁₋₆ haloalkyl, C₁₋₆ heteroalkyl, or halo;
R^{7c} is H or C₁₋₆ alkyl;
R^{A}, R^{B}, R^{C}, and R^{D} are each independently H, C₁₋₆ alkyl, C₂₋₆ alkenyl, C₂₋₆ alkynyl, C₁₋₆ haloalkyl, C₁₋₆ heteroalkyl, cycloalkyl, heterocyclyl, aryl, or heteroaryl;
p is 0, 1, 2, 3, or 4;
p′ is 0, 1, 2, 3, or 4;
q is 0, 1, 2, or 3; and [symbol]
denotes the point of attachment to L1 in Formula (I).

48. The bifunctional compound or pharmaceutically acceptable salt hydrate, solvate, stereoisomer, or tautomer thereof according to claim 47, wherein each of X and Z is independently O.

49. The bifunctional compound or pharmaceutically acceptable salt hydrate, solvate, stereoisomer, or tautomer thereof according to any one of claims 47-48, wherein Y is C(R^{7a})(R^{7b}).

50. The bifunctional compound or pharmaceutically acceptable salt hydrate, solvate, stereoisomer, or tautomer thereof according to any one of claims 47-49, wherein each of R^{7a} and R^{7b} is independently halo (e.g., fluoro).

51. The bifunctional compound or pharmaceutically acceptable salt hydrate, solvate, stereoisomer, or tautomer thereof according to any one of claims 47-50, wherein each of R^{3a}, R^{3b}R^{4a}, R^{4b} is independently H.

52. The bifunctional compound or pharmaceutically acceptable salt hydrate, solvate, stereoisomer, or tautomer thereof according to any one of claims 47-51, wherein R¹ is H.

53. The bifunctional compound or pharmaceutically acceptable salt hydrate, solvate, stereoisomer, or tautomer thereof according to any one of claims 47-52, wherein each of p and q is 0.

54. The bifunctional compound or pharmaceutically acceptable salt hydrate, solvate, stereoisomer, or tautomer thereof according to any one of the preceding claims, wherein the Target Ligand has the structure of Formula (I-f): 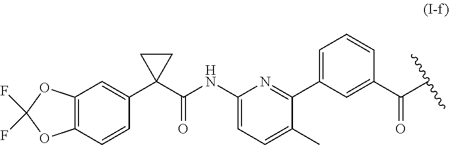 [SMILES: *C(=O)c1cccc(-c2nc(NC(=O)C3(c4ccc5c(c4)OC(F)(F)O5)CC3)ccc2C)c1]
or a pharmaceutically acceptable salt, hydrate, solvate, prodrug, stereoisomer, or tautomer thereof, wherein [symbol] denotes the point of attachment to L1 in Formula (I).

55. The bifunctional compound or pharmaceutically acceptable salt hydrate, solvate, stereoisomer, or tautomer thereof according to any one of the preceding claims, wherein the bifunctional compound of Formula (I) has the structure of Formula (I-h): 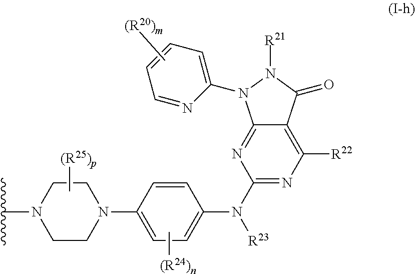
or a pharmaceutically acceptable salt, hydrate, solvate, prodrug, stereoisomer, or tautomer thereof, wherein each R²⁰, R²⁴, and R²⁵ is independently C₁₋₆ alkyl, C₂₋₆ alkenyl, C₂₋₆ alkynyl, C₁₋₆ haloalkyl, C₁₋₆ heteroalkyl, halo, cyano, —OR^{A}, —C(O)N(R^{B})(R^{C}), or —N(R^{B})CO(R^{D}); R²¹ and R²³ are each independently H or C₁₋₆ alkyl; R²² is C₁₋₆ alkyl, C₂₋₆ alkenyl, C₂₋₆ alkynyl, C₁₋₆ haloalkyl, C₁₋₆ heteroalkyl, halo, cyano, —OR^{A}, —C(O)N(R^{B})(R^{C}), or —N(R^{B})CO(R^{D}); R^{A}, R^{B}, R^{C}, and R^{D} are each independently H, C₁₋₆ alkyl, C₂₋₆ alkenyl, C₂₋₆ alkynyl, C₁₋₆ haloalkyl, C₁₋₆ heteroalkyl, cycloalkyl, heterocyclyl, aryl, or heteroaryl; m and n are each independently 0, 1, 2, 3, or 4; p is 0, 1, 2, 3, 4, 5, 6, 7, or 8; and [symbol] denotes the point of attachment to L1 in Formula (I).

56. The bifunctional compound or pharmaceutically acceptable salt hydrate, solvate, stereoisomer, or tautomer thereof according to any one of the preceding claims, wherein the bifunctional compound of Formula (I) has the structure (II-a): 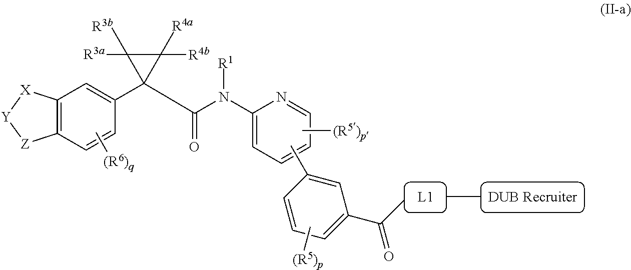
or a pharmaceutically acceptable salt, hydrate, solvate, prodrug, stereoisomer, or tautomer thereof, wherein:
X and Z are each independently O, S, or C(R^{7a})(R^{7b});
Y is C(R^{7a})(R^{7b}) or NR⁷;
R¹ is H or C₁₋₆ alkyl;
R^{3a}, R^{3b}, R^{4a}, R^{4b} are each independently H, C₁₋₆ alkyl, C₁₋₆ haloalkyl, C₁₋₆ heteroalkyl, halo, cyano, or —OR^{A};
each R⁵, R^{5′}, and R⁶ is independently C₁₋₆ alkyl, C₁₋₆ haloalkyl, C₁₋₆ heteroalkyl, halo, cyano, —OR^{A}, —C(O)N(R^{B})(R^{C}), or —N(R^{B})CO(R^{D});
R^{7a} and R^{7b} are each independently H, C₁₋₆ alkyl, C₁₋₆ haloalkyl, C₁₋₆ heteroalkyl, or halo;
R^{7c} is H or C₁₋₆ alkyl;
R^{A}, R^{B}, R^{C}, and R^{D} are each independently H, C₁₋₆ alkyl, C₂₋₆ alkenyl, C₂₋₆ alkynyl, C₁₋₆ haloalkyl, C₁₋₆ heteroalkyl, cycloalkyl, heterocyclyl, aryl, or heteroaryl;
p is 0, 1, 2, 3, or 4;
p′ is 0, 1, 2, 3, or 4;
q is 0, 1, 2, or 3; and
L1 and DUB Recruiter are as defined in claim 1.

57. The bifunctional compound or pharmaceutically acceptable salt hydrate, solvate, stereoisomer, or tautomer thereof according to any one of the preceding claims, wherein the bifunctional compound of Formula (I) has the structure (II-d): 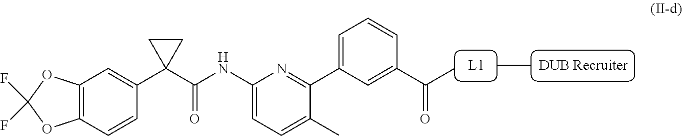
or a pharmaceutically acceptable salt, hydrate, solvate, prodrug, stereoisomer, or tautomer thereof, wherein L1 and DUB Recruiter are as defined in claim 1.

58. The bifunctional compound or pharmaceutically acceptable salt hydrate, solvate, stereoisomer, or tautomer thereof according to any one of the preceding claims, wherein the bifunctional compound of Formula (I) has the structure (II-k): 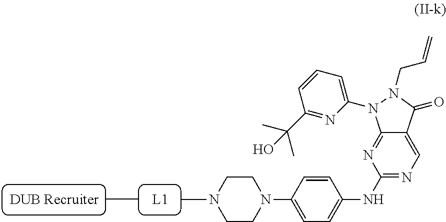
or a pharmaceutically acceptable salt, hydrate, solvate, prodrug, stereoisomer, or tautomer thereof, wherein L1 and DUB Recruiter are as defined for Formula (I).

59. The bifunctional compound or pharmaceutically acceptable salt hydrate, solvate, stereoisomer, or tautomer thereof according to any one of the preceding claims, wherein the DUB Recruiter binds to (e.g., covalently binds to) the deubiquitinase.

60. The bifunctional compound or pharmaceutically acceptable salt hydrate, solvate, stereoisomer, or tautomer thereof according to any one of the preceding claims, wherein the binding of the DUB Recruiter to the deubiquitinase does not substantially inhibit the activity of the deubiquitinase.

61. The bifunctional compound or pharmaceutically acceptable salt hydrate, solvate, stereoisomer, or tautomer thereof according to any one of the preceding claims, wherein the DUB Recruiter binds to a site other than a catalytic site within the deubiquitinase.

62. The bifunctional compound or pharmaceutically acceptable salt hydrate, solvate, stereoisomer, or tautomer thereof according to any one of the preceding claims, wherein the DUB Recruiter binds to an allosteric site within the deubiquitinase.

63. The bifunctional compound or pharmaceutically acceptable salt hydrate, solvate, stereoisomer, or tautomer thereof according to any one of the preceding claims, wherein the DUB Recruiter binds to a cysteine amino acid residue within the deubiquitinase.

64. The bifunctional compound or pharmaceutically acceptable salt hydrate, solvate, stereoisomer, or tautomer thereof according to any one of the preceding claims, wherein the DUB Recruiter preferentially binds to an allosteric amino acid residue (e.g., an allosteric amino acid residue) over a catalytic amino acid residue (e.g., a catalytic cysteine amino acid residue).

65. The bifunctional compound or pharmaceutically acceptable salt hydrate, solvate, stereoisomer, or tautomer thereof according to any one of the preceding claims, wherein the DUB Recruiter does not substantially bind to a cysteine amino acid residue in the catalytic site of the deubiquitinase (e.g., a catalytic cysteine).

66. The bifunctional compound or pharmaceutically acceptable salt hydrate, solvate, stereoisomer, or tautomer thereof according to any one of the preceding claims, wherein the DUB Recruiter comprises an acrylamide moiety.

67. The bifunctional compound or pharmaceutically acceptable salt hydrate, solvate, stereoisomer, or tautomer thereof according to any one of the preceding claims, wherein the DUB Recruiter comprises a furan moiety.

68. The bifunctional compound or pharmaceutically acceptable salt hydrate, solvate, stereoisomer, or tautomer thereof according to any one of the preceding claims, wherein the DUB Recruiter has the structure of Formula (V-b): 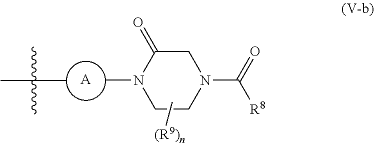
or a pharmaceutically acceptable salt, hydrate, solvate, prodrug, stereoisomer, or tautomer thereof, wherein:
Ring A is cycloalkyl, heterocyclyl, aryl, or heteroaryl, each of which is substituted with 0-12 R¹⁰;
R⁸ is H, C₁₋₆ alkyl, or an electrophilic moiety;
each R⁹ is independently C₁₋₆ alkyl, C₁₋₆ haloalkyl, C₁₋₆ heteroalkyl, halo, or —OR^{A};
each R¹⁰ is independently C₁₋₆ alkyl, C₁₋₆ haloalkyl, C₁₋₆ heteroalkyl, or halo;
R^{A} is H, C₁₋₆ alkyl, C₂₋₆ alkenyl, C₂₋₆ alkynyl, C₁₋₆ haloalkyl, C₁₋₄ heteroalkyl, halo, cycloalkyl, heterocyclyl, aryl, or heteroaryl; and
n is 0, 1, 2, 3, 4, 5, or 6.

69. The bifunctional compound or pharmaceutically acceptable salt hydrate, solvate, stereoisomer, or tautomer thereof according to claim 68, wherein Ring A is heteroaryl (e.g., a monocyclic heteroaryl).

70. The bifunctional compound or pharmaceutically acceptable salt hydrate, solvate, stereoisomer, or tautomer thereof according to any one of claims 68-69, wherein Ring A is a 5-membered heteroaryl (e.g., furanyl).

71. The bifunctional compound or pharmaceutically acceptable salt hydrate, solvate, stereoisomer, or tautomer thereof according any one of claims 68-70, wherein R⁸ is an electrophilic moiety.

72. The bifunctional compound or pharmaceutically acceptable salt hydrate, solvate, stereoisomer, or tautomer thereof according any one of claims 68-71, wherein R⁸ is C₂₋₆ alkenyl (e.g., CH═CH₂).

73. The bifunctional compound or pharmaceutically acceptable salt hydrate, solvate, stereoisomer, or tautomer thereof according to any one of the preceding claims, wherein the DUB Recruiter has the structure of Compound 100: 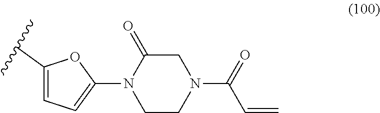 [SMILES: *c1ccc(N2CCN(C(=O)C=C)CC2=O)o1]
or a pharmaceutically acceptable salt, hydrate, solvate, prodrug, stereoisomer, or tautomer thereof, wherein [symbol] denotes the point of attachment to L1 in Formula (I).

74. The bifunctional compound or pharmaceutically acceptable salt hydrate, solvate, stereoisomer, or tautomer thereof according to any one of claims 1-28 and 29-65, wherein the DUB Recruiter binds to cysteine 23 (C₂₃) within the OTUB1 sequence.

75. The bifunctional compound or pharmaceutically acceptable salt hydrate, solvate, stereoisomer, or tautomer thereof according to any one of claims 1-28 and 29-66, wherein the DUB Recruiter binds preferentially to cysteine 23 (C23) over cysteine 91 (C91) within the OTUB1 sequence.

76. The bifunctional compound or pharmaceutically acceptable salt hydrate, solvate, stereoisomer, or tautomer thereof according to any one of claims 1-28 and 29-67, wherein the DUB Recruiter does not substantially bind to cysteine 91 (C91) within the OTUB1 sequence.

77. The bifunctional compound or pharmaceutically acceptable salt hydrate, solvate, stereoisomer, or tautomer thereof according to any one of the preceding claims, wherein the bifunctional compound of Formula (I) has the structure (II-k): 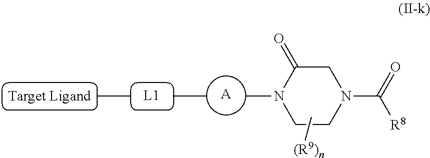
or a pharmaceutically acceptable salt, hydrate, solvate, prodrug, stereoisomer, or tautomer thereof, wherein:
Ring A is cycloalkyl, heterocyclyl, aryl, or heteroaryl, each of which is substituted with 0-12 R¹⁰;
R⁸ is H, C₁₋₆ alkyl, or an electrophilic moiety;
each R⁹ is independently C₁₋₆ alkyl, C₁₋₆ haloalkyl, C₁₋₆ heteroalkyl, halo, or —OR^{A};
each R¹⁰ is independently C₁₋₆ alkyl, C₁₋₆ haloalkyl, C₁₋₆ heteroalkyl, or halo;
R^{A} is H, C₁₋₆ alkyl, C₂₋₆ alkenyl, C₂₋₆ alkynyl, C₁₋₆ haloalkyl, C₁₋₄ heteroalkyl, halo, cycloalkyl, heterocyclyl, aryl, or heteroaryl;
n is 0, 1, 2, 3, 4, 5, or 6;
wherein the Target Ligand and L1 are as defined in claim 1.

78. The bifunctional compound or pharmaceutically acceptable salt hydrate, solvate, stereoisomer, or tautomer thereof according to any one of the preceding claims, wherein the bifunctional compound of Formula (I) has the structure (II-1): 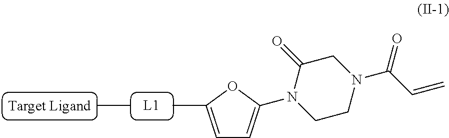
or a pharmaceutically acceptable salt, hydrate, solvate, prodrug, stereoisomer, or tautomer thereof, wherein the Target Ligand and L1 are as defined in claim 1.

79. The bifunctional compound or pharmaceutically acceptable salt hydrate, solvate, stereoisomer, or tautomer thereof according to any one of the preceding claims, wherein L1 is a non-cleavable linker.

80. The bifunctional compound or pharmaceutically acceptable salt hydrate, solvate, stereoisomer, or tautomer thereof according to any one of the preceding claims, wherein L1 comprises an alkylene or heteroalkylene.

81. The bifunctional compound or pharmaceutically acceptable salt hydrate, solvate, stereoisomer, or tautomer thereof according to any one of the preceding claims, wherein L1 has the structure of Formula (III-a): 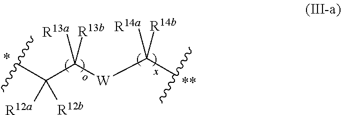
or a pharmaceutically acceptable salt, hydrate, solvate, prodrug, stereoisomer, or tautomer thereof, wherein:
R^{12a}, R^{12b}, R^{13a}, R^{13b}, R^{14a}, and R^{14b} are each independently H, C₁₋₄ alkyl, C₁₋₆ haloalkyl, C₁₋₆ heteroalkyl, halo, cyano, or —OR^{A}; or
each of R^{12a} and R^{12b}, R^{13a} and R^{13b}, and R^{14a} and R^{14b} independently may be taken together with the carbon atom to which they are attached to form an oxo group.
W is C(R^{15a})(R^{15b}), O, N(R¹⁶), or S;
R^{15a} and R^{15b} are each independently H, C₁₋₆ alkyl, C₁₋₆ haloalkyl, C₁₋₆ heteroalkyl, halo, cyano, or —OR^{A}; or
R^{15a} and R^{15b} may be taken together with the carbon atom to which they are attached to form an oxo group;
R¹⁶ is H or C₁₋₆ alkyl;
R^{A} is H, C₁₋₆ alkyl, C₂₋₆ alkenyl, C₂₋₆ alkynyl, C₁₋₄ haloalkyl, C₁₋₄ heteroalkyl, cycloalkyl, heterocyclyl, aryl, or heteroaryl;
and x are each independently an integer between 0 and 10; [symbol]
denotes the point of attachment to the Target Ligand in Formula (I); and [symbol]
denotes the point of attachment to the DUB Recruiter in Formula (I).

82. The bifunctional compound or pharmaceutically acceptable salt hydrate, solvate, stereoisomer, or tautomer thereof according to claim 81, wherein each of R¹², R^{12b}, R^{13a}, and R^{13b} is independently H.

83. The bifunctional compound or pharmaceutically acceptable salt hydrate, solvate, stereoisomer, or tautomer thereof according to any one of claims 81-82, wherein each of R^{14a} and R^{14b} are taken together with the carbon atom to which they are attached form an oxo group.

84. The bifunctional compound or pharmaceutically acceptable salt hydrate, solvate, stereoisomer, or tautomer thereof according to any one of claims 81-83, wherein W is N(R¹⁶) (e.g., NH).

85. The bifunctional compound or pharmaceutically acceptable salt hydrate, solvate, stereoisomer, or tautomer thereof according to any one of claims 81-84, wherein o is selected from 2, 3, 4, 5, and 6.

86. The bifunctional compound or pharmaceutically acceptable salt hydrate, solvate, stereoisomer, or tautomer thereof according to any one of claims 81-85, wherein p is selected from 1, 2, and 3.

87. The bifunctional compound or pharmaceutically acceptable salt hydrate, solvate, stereoisomer, or tautomer thereof according to any one of the preceding claims, wherein L1 has the structure of Formula (III-b): 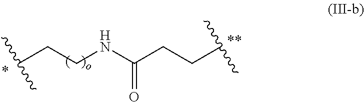
or a pharmaceutically acceptable salt, hydrate, solvate, prodrug, stereoisomer, or tautomer thereof, wherein:
o is an integer between 0 and 10; [symbol]
denotes the point of attachment to the Target Ligand in Formula (I); and [symbol]
denotes the point of attachment to the DUB Recruiter in Formula (I).

88. The bifunctional compound or pharmaceutically acceptable salt hydrate, solvate, stereoisomer, or tautomer thereof according to any one of the preceding claims, wherein L1 has the structure of Formula (III-c): 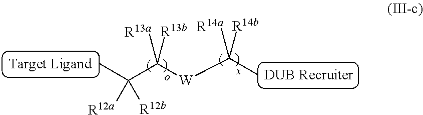
or a pharmaceutically acceptable salt, hydrate, solvate, prodrug, stereoisomer, or tautomer thereof, wherein:
R^{12a}, R^{12b}, R^{13a}, R^{13b}, R^{14a}, and R^{14b} are each independently H, C₁₋₄ alkyl, C₁₋₆ haloalkyl, C₁₋₆ heteroalkyl, halo, cyano, or —OR^{A}; or
each of R^{12a} and R^{12b}, R^{13a} and R^{13b}, and R^{14a} and R^{14b} independently may be taken together with the carbon atom to which they are attached to form an oxo group.
W is C(R^{15a})(R^{15b}), O, N(R¹⁶), or S;
R^{15a} and R^{15b} are each independently H, C₁₋₆ alkyl, C₁₋₆ haloalkyl, C₁₋₆ heteroalkyl, halo, cyano, or —OR^{A}; or
R^{15a} and R^{15b} may be taken together with the carbon atom to which they are attached to form an oxo group;
R¹⁶ is H or C₁₋₆ alkyl;
R^{A} is H, C₁₋₆ alkyl, C₂₋₆ alkenyl, C₂₋₆ alkynyl, C₁₋₆ haloalkyl, C₁₋₄ heteroalkyl, cycloalkyl, heterocyclyl, aryl, or heteroaryl;
o and x are each independently an integer between 0 and 10; and
the Target Ligand and DUB Recruiter are as defined in claim 1.

89. The bifunctional compound or pharmaceutically acceptable salt hydrate, solvate, stereoisomer, or tautomer thereof according to any one of the preceding claims, wherein L1 has the structure of Formula (III-d): 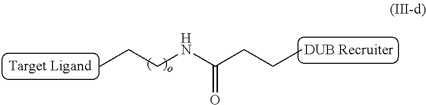
or a pharmaceutically acceptable salt, hydrate, solvate, prodrug, stereoisomer, or tautomer thereof, wherein:
o is an integer between 0 and 10; and
the Target Ligand and DUB Recruiter are as defined in claim 1.

90. The bifunctional compound or pharmaceutically acceptable salt hydrate, solvate, stereoisomer, or tautomer thereof according to any one of the preceding claims, wherein the bifunctional compound of Formula (I) has the structure (II-n): 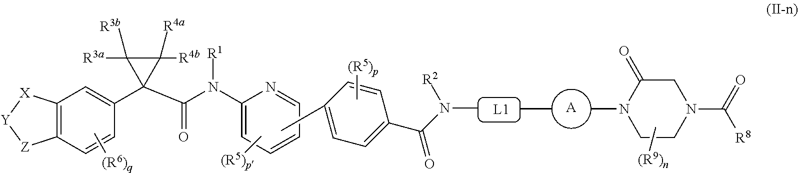
or a pharmaceutically acceptable salt, hydrate, solvate, prodrug, stereoisomer, or tautomer thereof, wherein:
X and Z are each independently O, S, or C(R^{7a})(R^{7b});
Y is C(R^{7a})(R^{7b}) or NR⁷;
Ring A is cycloalkyl, heterocyclyl, aryl, or heteroaryl, each of which is substituted with 0-12 R¹⁰;
R¹ is H or C₁₋₆ alkyl;
R² is H or C₁₋₆ alkyl;
R^{3a}, R^{3b}, R^{4a}, R^{4b} are each independently H, C₁₋₆ alkyl, C₁₋₆ haloalkyl, C₁₋₆ heteroalkyl, halo, cyano, or —OR^{A};
each R⁵, R^{5′}, and R⁶ is independently C₁₋₆ alkyl, C₁₋₆ haloalkyl, C₁₋₆ heteroalkyl, halo, cyano, —OR^{A}, —C(O)N(R^{B})(R^{C}), or —N(R^{B})CO(R^{D});
R^{7a} and R^{7b} are each independently H, C₁₋₆ alkyl, C₁₋₆ haloalkyl, C₁₋₆ heteroalkyl, or halo;
R⁷, is H or C₁₋₆ alkyl;
R⁸ is H, C₁₋₆ alkyl, or an electrophilic moiety;
each R⁹ is independently C₁₋₆ alkyl, C₁₋₆ haloalkyl, C₁₋₆ heteroalkyl, halo, or —OR^{A};
each R¹⁰ is independently C₁₋₆ alkyl, C₁₋₆ haloalkyl, C₁₋₆ heteroalkyl, or halo;
R^{A}, R^{B}, R^{C}, and R^{D} are each independently H, C₁₋₆ alkyl, C₂₋₆ alkenyl, C₂₋₆ alkynyl, C₁₋₆ haloalkyl, C₁₋₆ heteroalkyl, cycloalkyl, heterocyclyl, aryl, or heteroaryl;
n is 0, 1, 2, 3, 4, 5, or 6;
p is 0, 1, 2, 3, or 4;
p′ is 0, 1, 2, 3, or 4;
q is 0, 1, 2, or 3; and
L1 is as defined in claim 1.

91. The bifunctional compound or pharmaceutically acceptable salt hydrate, solvate, stereoisomer, or tautomer thereof according to any one of the preceding claims, wherein the bifunctional compound of Formula (I) has the structure (II-o): 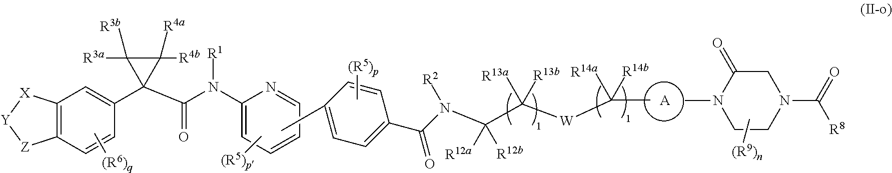
or a pharmaceutically acceptable salt, hydrate, solvate, prodrug, stereoisomer, or tautomer thereof, wherein:
X and Z are each independently O, S, or C(R^{7a})(R^{7b});
Y is C(R^{7a})(R^{7b}) or NR⁷;
Ring A is cycloalkyl, heterocyclyl, aryl, or heteroaryl, each of which is substituted with 0-12 R¹⁰;
R¹ is H or C₁₋₆ alkyl;
R² is H or C₁₋₆ alkyl;
R^{3a}, R^{3b}, R^{4a}, R^{4b} are each independently H, C₁₋₆ alkyl, C₁₋₆ haloalkyl, C₁₋₆ heteroalkyl, halo, cyano, or —OR^{A};
each R⁵, R^{5′}, and R⁶ is independently C₁₋₆ alkyl, C₁₋₆ haloalkyl, C₁₋₆ heteroalkyl, halo, cyano, —OR^{A}, —C(O)N(R^{B})(R^{C}), or —N(R^{B})CO(R^{D});
R^{7a} and R^{7b} are each independently H, C₁₋₆ alkyl, C₁₋₆ haloalkyl, C₁₋₆ heteroalkyl, or halo;
R⁷, is H or C₁₋₆ alkyl;
R⁸ is H, C₁₋₆ alkyl, or an electrophilic moiety;
each R⁹ is independently C₁₋₆ alkyl, C₁₋₆ haloalkyl, C₁₋₆ heteroalkyl, halo, or —OR^{A};
each R¹⁰ is independently C₁₋₆ alkyl, C₁₋₆ haloalkyl, C₁₋₆ heteroalkyl, or halo;
R^{12a}, R^{12b}, R^{13a}, R^{13b}, R^{14a}, and R^{14b} are each independently H, C₁₋₆ alkyl, C₁₋₆ haloalkyl, C₁₋₆ heteroalkyl, halo, cyano, or —OR^{A}; or
each of R^{12a} and R^{12b}, R^{13a} and R^{13b}, and R^{14a} and R^{14b} independently may be taken together with the carbon atom to which they are attached to form an oxo group.
W is C(R^{15a})(R^{15b}), O, N(R¹⁶), or S;
R^{15a} and R^{15b} are each independently H, C₁₋₆ alkyl, C₁₋₆ haloalkyl, C₁₋₆ heteroalkyl, halo, cyano, or —OR^{A}; or
R^{15a} and R^{15b} may be taken together with the carbon atom to which they are attached to form an oxo group;
R¹⁶ is H or C₁₋₆ alkyl;
R^{A}, R^{B}, R^{C}, and R^{D} are each independently H, C₁₋₆ alkyl, C₂₋₆ alkenyl, C₂₋₆ alkynyl, C₁₋₆ haloalkyl, C₁₋₆ heteroalkyl, cycloalkyl, heterocyclyl, aryl, or heteroaryl;
n is 0, 1, 2, 3, 4, 5, or 6;
and x are each independently an integer between 0 and 10;
p is 0, 1, 2, 3, or 4;
p′ is 0, 1, 2, 3, or 4; and
q is 0, 1, 2, or 3.

92. The bifunctional compound or pharmaceutically acceptable salt hydrate, solvate, stereoisomer, or tautomer thereof according to any one of the preceding claims, wherein the bifunctional compound of Formula (I) has the structure (II-q): 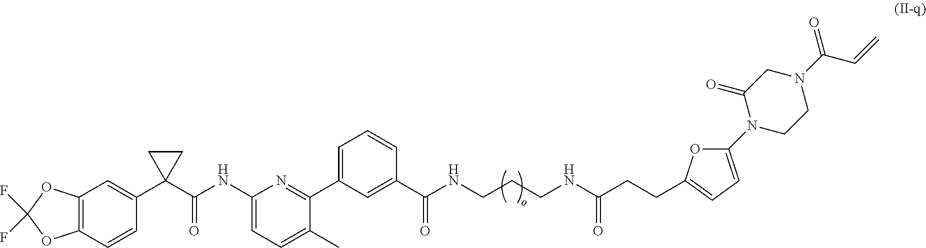
or a pharmaceutically acceptable salt, hydrate, solvate, prodrug, stereoisomer, or tautomer thereof, wherein o is selected from 0, 1, 2, 3, 4, 5, and 6.

93. The bifunctional compound or pharmaceutically acceptable salt hydrate, solvate, stereoisomer, or tautomer thereof according to any one of the preceding claims, wherein the bifunctional compound of Formula (I) is selected from a bifunctional compound provided in Table 2, or a pharmaceutically acceptable salt, hydrate, solvate, prodrug, stereoisomer, or tautomer thereof.

94. A pharmaceutical composition comprising a bifunctional compound, or pharmaceutically acceptable salt, solvate, stereoisomer, or tautomer thereof according to any one of the preceding claims, and one or more pharmaceutically acceptable carriers.

95. A composition for use in providing a compound to a subject, wherein the composition comprises a bifunctional compound of Formula (I): 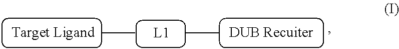
or a pharmaceutically acceptable salt, hydrate, solvate, stereoisomer, or tautomer thereof, wherein:
(i) the Target Ligand comprises a moiety capable of binding to a target protein;
(ii) L1 comprises a linker; and
(iii) the DUB Recruiter comprises a moiety capable of binding to a deubiquitinase.

96. A composition for use in treating a disease, disorder, or condition in a subject, comprising a bifunctional compound of Formula (I): 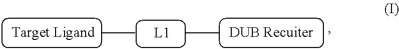
or a pharmaceutically acceptable salt, hydrate, solvate, stereoisomer, or tautomer thereof, wherein:
(i) the Target Ligand comprises a moiety capable of binding to a target protein;
(ii) L1 comprises a linker;
(iii) the DUB Recruiter comprises a moiety capable of binding to a deubiquitinase.

97. The composition for use of claim 96, wherein administering the composition ameliorates a symptom or element of the disease, disorder, or condition.

98. The composition for use of any one of claims 96-97, wherein the disease, disorder, or condition is cystic fibrosis.

99. A composition for use in treating cystic fibrosis in a subject, comprising a bifunctional compound of Formula (I): 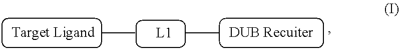
or a pharmaceutically acceptable salt, hydrate, solvate, stereoisomer, or tautomer thereof, wherein:
(i) the Target Ligand comprises a moiety capable of binding to a target protein;
(ii) L1 comprises a linker; and
(iii) the DUB Recruiter comprises a moiety capable of binding to a deubiquitinase.

100. A composition for use in modulating a protein in a cell or subject comprising a bifunctional compound of Formula (I): 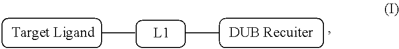
or a pharmaceutically acceptable salt, hydrate, solvate, stereoisomer, or tautomer thereof, wherein:
(i) the Target Ligand comprises a moiety capable of binding to a target protein;
(ii) L1 comprises a linker;
(iii) the DUB Recruiter comprises a moiety capable of binding to a deubiquitinase.

101. A composition for use in recruiting a deubiquitinase to a target protein in a cell or subject, wherein the composition comprises a bifunctional compound of Formula (I): 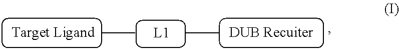
or a pharmaceutically acceptable salt, hydrate, solvate, stereoisomer, or tautomer thereof, wherein:
(i) the Target Ligand comprises a moiety capable of binding to a target protein;
(ii) L1 comprises a linker;
(iii) the DUB Recruiter comprises a moiety capable of binding to a deubiquitinase.

102. A composition for use in deubiquitinating a protein comprising a bifunctional compound of Formula (I): 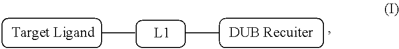
or a pharmaceutically acceptable salt, hydrate, solvate, stereoisomer, or tautomer thereof, wherein:
(i) the Target Ligand comprises a moiety capable of binding to a target protein;
(ii) L1 comprises a linker;
(iii) the DUB Recruiter comprises a moiety capable of binding to a deubiquitinase.

103. A method of providing a compound to a subject, wherein the compound comprises a bifunctional compound of Formula (I): 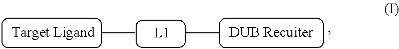
or a pharmaceutically acceptable salt, hydrate, solvate, stereoisomer, or tautomer thereof, wherein:
(i) the Target Ligand comprises a moiety capable of binding to a target protein;
(ii) L1 comprises a linker; and
(iii) the DUB Recruiter comprises a moiety capable of binding to a deubiquitinase.

104. A method of treating a disease, disorder, or condition in a subject, wherein the method comprises administering to the subject a bifunctional compound of Formula(I: 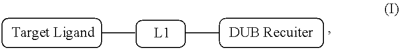
or a pharmaceutically acceptable salt, hydrate, solvate, stereoisomer, or tautomer thereof, wherein:
(i) the Target Ligand comprises a moiety capable of binding to a target protein;
(ii) L1 comprises a linker;
(iii) the DUB Recruiter comprises a moiety capable of binding to a deubiquitinase.

105. The method of claim 104, wherein the method comprises ameliorating a symptom or element of the disease, disorder, or condition.

106. The method of any one of claims 104-105, wherein the disease, disorder, or condition is cystic fibrosis.

107. A method of treating cystic fibrosis in a subject, the method comprising administering to the subject a bifunctional compound of Formula (I): 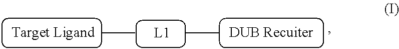
or a pharmaceutically acceptable salt, hydrate, solvate, stereoisomer, or tautomer thereof, wherein:
(i) the Target Ligand comprises a moiety capable of binding to a target protein;
(ii) L1 comprises a linker; and
(iii) the DUB Recruiter comprises a moiety capable of binding to a deubiquitinase, thereby treating cystic fibrosis.

108. A method of modulating a protein in a cell or subject comprising contacting the cell or administering to the subject a bifunctional compound of Formula (I): 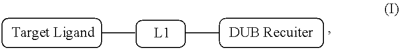
or a pharmaceutically acceptable salt, hydrate, solvate, stereoisomer, or tautomer thereof, wherein:
(i) the Target Ligand comprises a moiety capable of binding to a target protein;
(ii) L1 comprises a linker;
(iii) the DUB Recruiter comprises a moiety capable of binding to a deubiquitinase, thereby modulating a protein in a cell or subject.

109. A method of recruiting a deubiquitinase to a target protein comprising contacting a mixture (e.g., in a cell or sample) with a bifunctional compound of Formula (I): 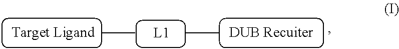
or a pharmaceutically acceptable salt, hydrate, solvate, stereoisomer, or tautomer thereof, wherein:
(i) the Target Ligand comprises a moiety capable of binding to a target protein;
(ii) L1 comprises a linker;
(iii) the DUB Recruiter comprises a moiety capable of binding to a deubiquitinase, thereby recruiting a deubiquitinase to a target protein in a mixture, e.g., a cell or subject.

110. A method of deubiquitinating a protein comprising contacting a cell or sample with a bifunctional compound of Formula (I): 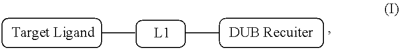
or a pharmaceutically acceptable salt, hydrate, solvate, stereoisomer, or tautomer thereof, wherein:
(i) the Target Ligand comprises a moiety capable of binding to a target protein;
(ii) L1 comprises a linker;
(iii) the DUB Recruiter comprises a moiety capable of binding to a deubiquitinase,
thereby deubiquitinating a protein in a cell or subject.
